(19) United States
(12) Patent Application Publication
(10) Pub. No.: US 2012/0100250 A1
(43) Pub. Date: Apr. 26, 2012

(54) Carbohydrate Oxidases

(75) Inventors: Lars Henrik Oestergaard, Charlottelund (DK); Leonardo De Maria, Frederiksberg (DK)

(73) Assignee: NOVOZYMES A/S, Bagsvaerd (DK)

(21) Appl. No.: 13/380,175

(22) PCT Filed: Jul. 9, 2010
(86) PCT No.: PCT/EP2010/059939

§ 371 Date: Dec. 22, 2011

Related U.S. Application Data
(60) Provisional application No. 61/229,421, filed on Jul. 29, 2009.

(30) Foreign Application Priority Data
Jul. 24, 2009 (EP) 09166407.8

(51) Int. Cl. C12N 9/02; C12N 5/10; A21D 10/00; C12N 1/15; A21D 8/04; C07H 21/00; C12N 1/19

(52) U.S. Cl. 426/18; 435/189; 536/23.2; 435/325; 435/348; 435/419; 435/254.11; 435/254.2; 435/254.22; 435/254.21; 435/254.23; 435/254.8; 435/254.3; 435/254.4; 435/254.5; 435/254.6; 435/254.7; 426/61

(57) ABSTRACT

The present invention relates to carbohydrate oxidases. The present invention also relates to polynucleotides encoding the variant carbohydrate oxidases and to nucleic acid constructs, vectors, and host cells comprising the polynucleotides, and methods of using the variant enzymes, such as, in preparing dough and dough product compositions.

### REFERENCE TO A SEQUENCE LISTING

This application contains a Sequence Listing in computer readable form. The computer readable form is incorporated herein by reference.

### REFERENCE TO STRUCTURAL COORDINATES

This application contains structural coordinates for the solved crystal structure of the inhibited carbohydrate oxidase shown in standard PDB format (Protein Data Bank). The structure is set forth in Appendix 1 and is incorporated herein by reference.

### FIELD OF THE INVENTION

The present invention relates to variants of a carbohydrate oxidase, polynucleotides encoding the variants, methods of producing the variants, and methods of using the variants.

### BACKGROUND OF THE INVENTION

WO 99/31990 discloses the amino acid sequence and nucleic acid sequence of the Microdochium nivale carbohydrate oxidase and its use in baking.

It is an object of the present invention to provide improved carbohydrate oxidases.

### SUMMARY OF THE INVENTION

The present invention provides isolated carbohydrate oxidase variants, comprising a substitution at one or more (several) positions corresponding to positions 4, 15, 19, 21, 22, 27, 29, 30, 31, 43, 48, 52, 54, 57, 58, 59, 60, 62, 69, 70, 71, 72, 77, 80, 81, 85, 91, 93, 98, 105, 112, 114, 118, 122, 129, 130, 131, 132, 133, 134, 135, 138, 140, 146, 147, 148, 152, 153, 157, 169, 170, 174, 184, 188, 201, 213, 221, 222, 223, 224, 227, 228, 231, 235, 248, 249, 250, 251, 252, 253, 256, 258, 260, 268, 278, 287, 288, 300, 301, 302, 303, 304, 305, 307, 314, 315, 316, 317, 318, 319, 320, 321, 322, 323, 327, 330, 332, 342, 347, 349, 352, 353, 354, 355, 356, 357, 358, 363, 366, 368, 374, 382, 383, 385, 386, 387, 388, 389, 390, 391, 392, 393, 400, 403, 411, 415, 419, 420, 424, 425, 427, 428, 429, 433, 437, 440, 445, 456, 460, 472, and/or 473 of SEQ ID NO:2, wherein the variants have carbohydrate oxidase activity.

The present invention also provides isolated carbohydrate oxidase variants or isolated polypeptides having carbohydrate oxidase activity, comprising an amino acid sequence which differs from SEQ ID NO:2 or the mature polypeptide encoded by the nucleic acid sequence of SEQ ID NO:1 by at least one or more (several) positions corresponding to positions 4, 15, 19, 21, 22, 27, 29, 30, 31, 43, 48, 52, 54, 57, 58, 59, 60, 62, 69, 70, 71, 72, 77, 80, 81, 85, 91, 93, 98, 105, 112, 114, 118, 122, 129, 130, 131, 132, 133, 134, 135, 138, 140, 146, 147, 148, 152, 153, 157, 169, 170, 174, 184, 188, 201, 213, 221, 222, 223, 224, 227, 228, 231, 235, 248, 249, 250, 251, 252, 253, 256, 258, 260, 268, 278, 287, 288, 300, 301, 302, 303, 304, 305, 307, 314, 315, 316, 317, 318, 319, 320, 321, 322, 323, 327, 330, 332, 342, 347, 349, 352, 353, 354, 355, 356, 357, 358, 363, 366, 368, 374, 382, 383, 385, 386, 387, 388, 389, 390, 391, 392, 393, 400, 403, 411, 415, 419, 420, 424, 425, 427, 428, 429, 433, 437, 440, 445, 456, 460, 472, and/or 473, using SEQ ID NO:2 for numbering.

The present invention also provides isolated polynucleotides encoding the carbohydrate oxidases of the present invention, and to nucleic acid constructs, vectors, and host cells comprising the polynucleotides, and to methods of producing to producing the carbohydrate oxidases of the present invention.

The present invention further provides the use of a carbohydrate oxidase of the present invention in preparing dough and/or a baked product made from dough comprising adding to the dough a carbohydrate oxidase of the present invention. The present invention also provides dough and/or bread improving compositions comparing a carbohydrate oxidase of the present invention. The dough and/or bread improving compositions may further comprise one or more dough or bread additives, including, e.g., second enzyme, such as, an amylase, cellulose, hemicellulase, lipase or phospholipase.

The present invention further provides methods for converting lactose to lactobionic acid (LBA). The LBA may be used in the production of food and beverage products, e.g., dairy products, such as, cheese, for example, by direct addition of the generated LBA to the food or beverage product and/or ingredient or by in situ generation of LBA in the food or beverage product or ingredient. LBA may also be used in cosmetics.

The present invention also provides enzyme granulates, powders and liquid compositions comprising a carbohydrate oxidase of the present invention.

### DETAILED DESCRIPTION OF THE INVENTION

Carbohydrate oxidase activity: The term “carbohydrate oxidase activity” is defined herein as enzyme activity that catalyzes the oxidation of the primary alcohol in various mono- or oligosaccharides accompanied by reduction of molecular oxygen to hydrogen peroxide. For purposes of the present invention, carbohydrase oxidase activity is determined according to the procedure described by Blake et al., Analytical Biochemistry 177: 156-160 (1989). One unit of carbohydrate oxidase activity equals the amount of enzyme capable of releasing 1 μmole of hydrogen peroxide per minute at pH 6.0, 25 degree Celsius.

Variant: The term “variant” is defined herein as a polypeptide having carbohydrate oxidase activity comprising an alteration, such as a substitution, insertion, and/or deletion, of one or more (several) amino acid residues at one or more (several) specific positions. The altered polynucleotide is obtained through human intervention by modification of a polynucleotide sequence, e.g., the polynucleotide sequence disclosed in SEQ ID NO:1 or a homologous sequence thereof.

Wild-Type Enzyme: The term “wild-type” carbohydrate oxidase denotes a carbohydrate oxidase expressed by a naturally occurring microorganism, such as a bacterial, yeast, or filamentous fungus found in nature, that is, polynucleotide encoding the carbohydrate oxidase is not obtained through human intervention by modification of the polynucleotide sequence.

Parent Enzyme: The term “parent” carbohydrate oxidase as used herein means a carbohydrate oxidase to which a modification, e.g., substitution(s), insertion(s), deletion(s), and/or truncation(s), is made to produce the enzyme variants of the present invention. This term also refers to the polypeptide with which a variant is compared and aligned. The parent may be a naturally occurring (wild-type) polypeptide or a variant. For instance, the parent polypeptide may be a variant of a naturally occurring polypeptide which has been modified or altered in the amino acid sequence. A parent may also be an allelic variant, which is a polypeptide encoded by any of two or more alternative forms of a gene occupying the same chromosomal locus.

Isolated variant or polypeptide: The term “isolated variant” or “isolated polypeptide” as used herein refers to a variant or a polypeptide that is isolated from a source. In one aspect, the variant or polypeptide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by SDS-PAGE.

Substantially pure variant or polypeptide: The term “substantially pure variant” or “substantially pure polypeptide” denotes herein a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which it is natively or recombinantly associated. It is, therefore, preferred that the substantially pure variant or polypeptide is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation. The variants and polypeptides of the present invention are preferably in a substantially pure form. This can be accomplished, for example, by preparing the variant or polypeptide by well-known recombinant methods or by classical purification methods.

Mature polypeptide: The term “mature polypeptide” is defined herein as a polypeptide having carbohydrate oxidase activity that is in its final form following translation and any post-translational modifications, such as N-terminal processing, C-terminal truncation, glycosylation, phosphorylation, etc. In one aspect, the mature polypeptide is the polypeptide of SEQ ID NO:2. The signal program SignalIP3.0 program may be used to predict the mature polypeptide.

Mature polypeptide coding sequence: The term “mature polypeptide coding sequence” is defined herein as a nucleotide sequence that encodes a mature polypeptide having carbohydrate oxidase activity. In one aspect, the mature polypeptide coding sequence is nucleotides encoding SEQ ID NO:2.

Identity: The relatedness between two amino acid sequences or between two nucleotide sequences is described by the parameter “identity”.

For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277; http://emboss.org), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra; http://emboss.org), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Homologous sequence: The term “homologous sequence” is defined herein as a predicted polypeptide that gives an E value (or expectancy score) of less than 0.001 in a tfasty search (Pearson, W. R., 1999, in Bioinformatics Methods and Protocols, S. Misener and S. A. Krawetz, ed., pp. 185-219) with the Micrododhium nivale carbohydrate oxidase CBS 100236.

Polypeptide fragment: The term “polypeptide fragment” is defined herein as a polypeptide having one or more (several) amino acids deleted from the amino and/or carboxyl terminus of the mature polypeptide; or a homologous sequence thereof; wherein the fragment has carbohydrate oxidase activity.

Subsequence: The term “subsequence” is defined herein as a polynucleotide sequence having one or more (several) nucleotides deleted from the 5′ and/or 3′ end of the mature polypeptide coding sequence; or a homologous sequence thereof; wherein the subsequence encodes a polypeptide fragment having carbohydrate oxidase activity.

Allelic variant: The term “allelic variant” denotes herein any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Isolated polynucleotide: The term “isolated polynucleotide” as used herein refers to a polynucleotide that is isolated from a source. In one aspect, the isolated polynucleotide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, and even most preferably at least 95% pure, as determined by agarose electrophoresis.

Substantially pure polynucleotide: The term “substantially pure polynucleotide” as used herein refers to a polynucleotide preparation free of other extraneous or unwanted nucleotides and in a form suitable for use within genetically engineered polypeptide production systems. Thus, a substantially pure polynucleotide contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polynucleotide material with which it is natively or recombinantly associated. A substantially pure polynucleotide may, however, include naturally occurring 5′ and 3′ untranslated regions, such as promoters and terminators. It is preferred that the substantially pure polynucleotide is at least 90% pure, preferably at least 92% pure, more preferably at least 94% pure, more preferably at least 95% pure, more preferably at least 96% pure, more preferably at least 97% pure, even more preferably at least 98% pure, most preferably at least 99%, and even most preferably at least 99.5% pure by weight. The polynucleotides of the present invention are preferably in a substantially pure form, i.e., that the polynucleotide preparation is essentially free of other polynucleotide material with which it is natively or recombinantly associated. The polynucleotides may be of genomic, cDNA, RNA, semisynthetic, synthetic origin, or any combinations thereof.

Coding sequence: When used herein the term “coding sequence” means a polynucleotide, which directly specifies the amino acid sequence of its polypeptide product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant polynucleotide.

cDNA: The term “cDNA” is defined herein as a DNA molecule that can be prepared by reverse transcription from a mature, spliced, mRNA molecule obtained from a eukaryotic cell. cDNA lacks intron sequences that are usually present in the corresponding genomic DNA. The initial, primary RNA transcript is a precursor to mRNA that is processed through a series of steps before appearing as mature spliced mRNA. These steps include the removal of intron sequences by a process called splicing. cDNA derived from mRNA lacks, therefore, any intron sequences

Nucleic acid construct: The term “nucleic acid construct” as used herein refers to a nucleic acid molecule, either single- or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

Control sequences: The term “control sequences” is defined herein to include all components necessary for the expression of a polynucleotide encoding a polypeptide of the present invention. Each control sequence may be native or foreign to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Operably linked: The term “operably linked” denotes herein a configuration in which a control sequence is placed at an appropriate position relative to the coding sequence of the polynucleotide sequence such that the control sequence directs the expression of the coding sequence of a polypeptide.

Expression: The term “expression” includes any step involved in the production of the polypeptide including, but not limited to, transcription, post-transcriptional modification, translation, post-translational modification, and secretion.

Expression vector: The term “expression vector” is defined herein as a linear or circular DNA molecule that comprises a polynucleotide encoding a polypeptide of the present invention and is operably linked to additional nucleotides that provide for its expression.

Host cell: The term “host cell”, as used herein, includes any cell type that is susceptible to transformation, transfection, transduction, and the like with a nucleic acid construct or expression vector comprising a polynucleotide of the present invention. The term “host cell” encompasses any progeny of a parent cell that is not identical to the parent cell due to mutations that occur during replication.

Improved property: The term “improved property” is defined herein as a characteristic associated with a variant that is improved compared to the parent carbohydrate oxidase. Such improved properties include, but are not limited to, altered temperature-dependent activity profile, thermostability, pH activity, pH stability, substrate specificity, product specificity, and chemical stability. In an embodiment, improved properties include stability with respect to one or more of temperature, hydrogen peroxide, pH, deamindation, amide hydrolysis of peptide bonds, and stability against anionic surfactants.

Improved thermal activity: The term “improved thermal activity” is defined herein as a variant enzyme displaying an alteration of the temperature-dependent activity profile of a carbohydrate oxidase variant at a specific temperature relative to the temperature-dependent activity profile of the parent carbohydrate oxidase. The thermal activity value provides a measure of the enzyme's efficiency in performing catalysis of a hydrolysis reaction over a range of temperatures. A carbohydrate oxidase has a specific temperature range wherein the polypeptide is stable and retains its enzymatic activity, but becomes less stable and thus less active with increasing temperature. Furthermore, the initial rate of a reaction catalyzed by a carbohydrate oxidase can be accelerated by an increase in temperature that is measured by determining thermal activity of a variant.

Improved thermostability: The term “improved thermostability” is defined herein as a variant enzyme displaying retention of enzymatic activity after a period of incubation at elevated temperature relative to the parent enzyme. Such a variant may or may not display an altered thermal activity profile relative to the parent. For example, a variant may have an improved ability to refold following incubation at elevated temperature relative to the parent.

A test for thermostability would involve performing Differential Scanning calorimetry (DSC) on a purified sample to determine the melting point of the carbohydrate oxidase, Tm. In DSC the heat consumed to keep a constant temperature increase in the sample-cell is measured relative to a reference cell. A constant heating rate is kept (e.g. 90 degree Celsius/hour). An endo-thermal process (heat consuming process—e.g. the unfolding of an enzyme/protein) is observed as an increase in the heat transferred to the cell in order to keep the constant temperature increase.

DSC can be performed using the MC2-apparatus from MicroCal. Cells are equilibrated 20 minutes at 20 degree Celcius before scanning to 90 degree Celcius at a scan rate of 90 degree/h. Samples of e.g. around 2.5 mg/ml carbohydrate oxidase in 0.1 M sodium acetate, pH 5.5 are loaded.

A variant would be considered improved if its Tm is larger than the parent or wild-type Tm. In one aspect, the thermal activity of the variant carbohydrate oxidase is at least 1.5-fold, preferably at least 2-fold, more preferably at least 5-fold, most preferably at least 7-fold, and even most preferably at least 20-fold more thermally active than the parent enzyme.

Improved product specificity: The term “improved product specificity” is defined herein as a variant enzyme displaying an altered product profile relative to the parent in which the altered product profile improves the performance of the variant in a given application relative to the parent. The term “product profile” is defined herein as the chemical composition of the reaction products produced by enzymatic hydrolysis.

Improved chemical stability: The term “improved chemical stability” is defined herein as a variant enzyme displaying retention of enzymatic activity after a period of incubation in the presence of a chemical or chemicals, either naturally occurring or synthetic, which reduce the enzymatic activity of the parent enzyme. Improved chemical stability may also result in variants better able to catalyze a reaction in the presence of such chemicals. Examples of chemicals include anionic surfactants and H2O2.

Improved resistance to proteolytic degradation: The term “improved resistance to proteolytic degradation” is defined herein as a variant enzyme displaying retention of enzymatic activity after a period of incubation in the presence of a protease. Improved resistance may also result in variants better able to catalyze a reaction in the presence of proteases.

### Conventions for Designation of Variants

For purposes of the present invention, the amino acid sequence of the carbohydrate oxidase disclosed in SEQ ID NO:2 is used to determine the corresponding amino acid residue in another carbohydrate oxidase. The amino acid sequence of another carbohydrate oxidase is aligned with the amino acid sequence of the carbohydrate oxidase disclosed in SEQ ID NO:2, and based on the alignment the amino acid position number corresponding to any amino acid residue in the amino acid sequence of the carbohydrate oxidase disclosed in SEQ ID NO:2 can be determined.

An alignment of polypeptide sequences may be made, for example, using “ClustalW” (Thompson, J. D., Higgins, D. G. and Gibson, T. J., 1994, CLUSTAL W: Improving the sensitivity of progressive multiple sequence alignment through sequence weighting, positions-specific gap penalties and weight matrix choice, Nucleic Acids Research 22: 4673-4680). An alignment of DNA sequences may be done using the polypeptide alignment as a template, replacing the amino acids with the corresponding codon from the DNA sequence.

Pairwise sequence comparison algorithms in common use are adequate to detect similarities between polypeptide sequences that have not diverged beyond the point of approximately 20-30% sequence identity (Doolittle, 1992, Protein Sci. 1: 191-200; Brenner et al., 1998, Proc. Natl. Acad. Sci. USA 95, 6073-6078). However, truly homologous polypeptides with the same fold and similar biological function have often diverged to the point where traditional sequence-based comparison fails to detect their relationship (Lindahl and Elofsson, 2000, J. Mol. Biol. 295: 613-615). Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide of interest has one or more (several) representatives in the protein structure databases. Programs such as GenTHREADER (Jones 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide of interest, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33:88-96) or combinatorial extension (Shindyalov and Bourne, 1998, Protein Eng. 11:739-747), and implementations of these algorithms can additionally be utilized to query structure databases with a structure of interest in order to discover possible structural homologs (e.g. Holm and Park, 2000, Bioinformatics 16:566-567). These structural alignments can be used to predict the structurally and functionally corresponding amino acid residues in proteins within the same structural superfamily. This information, along with information derived from homology modeling and profile searches, can be used to predict which residues to mutate when moving mutations of interest from one protein to a close or remote homolog.

In describing the various carbohydrate oxidase variants of the present invention, the nomenclature described below is adapted for ease of reference. In all cases, the accepted IUPAC single letter or triple letter amino acid abbreviation is employed.

Substitutions. For an amino acid substitution, the following nomenclature is used: Original amino acid, position, substituted amino acid. Accordingly, the substitution of threonine with alanine at position 226 is designated as “Thr226Ala” or “T226A”. Multiple mutations are separated by addition marks (“+”), e.g., “Gly205Arg+Ser411Phe” or “G205R+S411F”, representing mutations at positions 205 and 411 substituting glycine (G) with arginine (R), and serine (S) with phenylalanine (F), respectively.

Deletions. For an amino acid deletion, the following nomenclature is used: Original amino acid, position*. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions. For an amino acid insertion, the following nomenclature is used: Original amino acid, position, original amino acid, new inserted amino acid. Accordingly the insertion of lysine after glycine at position 195 is designated “Gly195GlyLys” or “G195GK”. Multiple insertions of amino acids are designated [Original amino acid, position, original amino acid, new inserted amino acid #1, new inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example the sequences would thus be:

| Parent: | Variant: |
|---|---|
| 195 | 195 195a 195b |
| G | G - K - A |

### Parent Carbohydrate Oxidases

The parent carbohydrate oxidase may be obtained from any suitable sources, such as, a microbial source, such as a fungus, e.g., a filamentous fungus or a yeast, or an artificial sequence prepared from known nucleic acid or amino acid sequence information.

The carbohydrate oxidase may be derived, e.g., from a mitosporic Pyrenomycetes such as Acremonium, in particular, A. strictum, such as ATCC 34717 or T1; A. fusidioides, such as IFO 6813; or A. potronii, such as IFO 31197. In a preferred embodiment, the oligosaccharide oxidase is obtained from the source disclosed by Lin, et al, (1991, Biochim. Biophys. Acta 1118:41-47) and in JP-A 5-84074.

The carbohydrate oxidase may further be obtained from microorganisms of Xylariales; especially mitosporic Xylariales such as the genus Microdochium, particularly the species M. nivale. Such strains are readily accessible to the public in culture collections, such as the American Type Culture Collection (ATCC), Deutsche Sammlung von Mikroorganismen and Zellkulturen GmbH (DSM) and Centraalbureau Voor Schimmelcultures (CBS).

The genus Microdochium is described in Microdochium Syd (Samuels and Hallett, 1983, TBMS 81:473). Some strains of Microdochium have been described under the synonyms Gerlachia, G. nivalis, G. oryzae, Fusarium nivale or Rynchosporium oryzae. They are further described by Monographella (Hyponectr) fide (Muller, 1977, Rev. mycol. 41:129).

The carbohydrate oxidase may be obtained from a strain of Microdochium nivale, NN008551, as described, e.g., in WO 99/31990. In an embodiment, the parent carbohydrate oxidase comprises or consists of carbohydrate oxidase having the amino acid sequence of SEQ ID NO:2 or the mature polypeptide encoded by the nucleic acid of SEQ ID NO:1.

In the present invention, a parent carbohydrate oxidase includes (a) a polypeptide comprising an amino acid sequence having at least 60% identity with SEQ ID NO:2; (b) a polypeptide encoded by a polynucleotide that hybridizes under at least low stringency conditions with (i) the mature polypeptide coding sequence of SEQ ID NO:1, (ii) the cDNA sequence contained in the mature polypeptide coding sequence of SEQ ID NO:1, or (iii) a full-length complementary strand of (i) or (ii); or (c) a polypeptide encoded by a polynucleotide comprising a nucleotide sequence having at least 60% identity with the mature polypeptide coding sequence of SEQ ID NO:1.

In a first aspect, the parent carbohydrate oxidase comprise an amino acid sequence having a degree of amino acid sequence identity to SEQ ID NO:2 of preferably at least 60%, more preferably at least 65%, more preferably at least 70%, more preferably at least 75%, more preferably at least 80%, more preferably at least 85%, even more preferably at least 90%, most preferably at least 95%, and even most preferably at least 96%, at least 97%, at least 98%, or at least 99%, and which have carbohydrate oxidase activity (hereinafter “homologous polypeptides”). In one aspect, the homologous polypeptides have an amino acid sequence that differs from SEQ ID NO:2 or the mature polypeptide encoded by SEQ ID NO:1 by thirty amino acids, twenty-nine amino acids, twenty-eight amino acids, twenty-seven amino acids, twenty-six amino acids, twenty-five amino acids, twenty-four amino acids, twenty-three amino acids, twenty-two amino acids, twenty-one amino acids, twenty amino acids, nineteen amino acids, eighteen amino acids, seventeen amino acids, sixteen amino acids, fifteen amino acids, fourteen amino acids, thirteen amino acids, twelve amino acids, eleven amino acids, ten amino acids, preferably by five amino acids, more preferably by four amino acids, even more preferably by three amino acids, most preferably by two amino acids, and even most preferably by one amino acid.

Substantially homologous parent carbohydrate oxidases may have one or more (several) amino acid substitutions, deletions and/or insertions. These changes are preferably of a minor nature, that is conservative amino acid substitutions as described above and other substitutions that do not significantly affect the three-dimensional folding or activity of the protein or polypeptide; small deletions, typically of one to about 30 amino acids; and small amino- or carboxyl-terminal extensions, such as an amino-terminal methionine residue, a small linker peptide of up to about 20-25 residues, or a small extension that facilitates purification (an affinity tag), such as a poly-histidine tract, or protein A (Nilsson et al., 1985, EMBO J. 4: 1075; Nilsson et al., 1991, Methods Enzymol. 198: 3. See, also, in general, Ford et al., 1991, Protein Expression and Purification 2: 95-107.

Although the changes described above preferably are of a minor nature, such changes may also be of a substantive nature such as fusion of larger polypeptides of up to 300 amino acids or more both as amino- or carboxyl-terminal extensions.

The parent carbohydrate oxidase may comprise or consist of the amino acid sequence of SEQ ID NO:2 or an allelic variant thereof; or a fragment thereof having carbohydrate oxidase activity. In one aspect, the parent carbohydrate oxidase comprises or consists of the amino acid sequence of SEQ ID NO:2. In another aspect, the parent carbohydrate oxidase comprises or consists of the mature polypeptide encoded by SEQ ID NO:1.

A fragment of the polypeptide of SEQ ID NO:2 or the mature polypeptide encoded by SEQ ID NO:1 is a polypeptide having one or more (several) amino acids deleted from the amino and/or carboxyl terminus of this amino acid sequence.

In a second aspect, the parent carbohydrate oxidases are encoded by polynucleotides that hybridize under very low stringency conditions, preferably low stringency conditions, more preferably medium stringency conditions, more preferably medium-high stringency conditions, even more preferably high stringency conditions, and most preferably very high stringency conditions with (i) the mature polypeptide coding sequence of SEQ ID NO:1, (ii) the cDNA sequence contained in the mature polypeptide coding sequence of SEQ ID NO:1, (iii) a subsequence of (i) or (ii), or (iv) a full-length complementary strand of (i), (ii), or (iii) (J. Sambrook, E. F. Fritsch, and T. Maniatis, 1989, Molecular Cloning, A Laboratory Manual, 2d edition, Cold Spring Harbor, N.Y.). The subsequence may encode a polypeptide fragment having carbohydrate oxidase activity. In one aspect, the complementary strand is the full-length complementary strand of the mature polypeptide coding sequence of SEQ ID NO:1.

A subsequence of the mature polypeptide coding sequence of SEQ ID NO:1, or a homolog thereof, is a nucleotide sequence where one or more (several) nucleotides have been deleted from the 5′- and/or 3′-end.

The parent enzymes may also be allelic variants of the polypeptides that have carbohydrate oxidase activity.

The polynucleotide of SEQ ID NO:1; or a subsequence thereof; or a fragment thereof; may be used to design nucleic acid probes to identify and clone DNA encoding parent carbohydrate oxidases from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, preferably at least 25, more preferably at least 35, and most preferably at least 70 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other organisms may be screened for DNA that hybridizes with the probes described above and encodes a parent carbohydrate oxidase. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with SEQ ID NO:1, or a subsequence thereof, the carrier material is used in a Southern blot. For purposes of the present invention, hybridization indicates that the polynucleotide hybridizes to a labeled nucleotide probe corresponding to the polynucleotide shown in SEQ ID NO:1, its complementary strand, or a subsequence thereof, under low to very high stringency conditions. Molecules to which the probe hybridizes can be detected using, for example, X-ray film or any other detection means known in the art.

The parent carbohydrate oxidase may also be encoded by a polynucleotide comprising or consisting of a nucleotide sequence having a degree of identity to the mature polypeptide coding sequence of SEQ ID NO:1 of preferably at least 60%, more preferably at least 65%, more preferably at least 70%, more preferably at least 75%, more preferably at least 80%, more preferably at least 85%, even more preferably at least 90%, most preferably at least 95%, and even most preferably 96%, 97%, 98%, or 99%, which encode an active polypeptide.

### Three-Dimensional Structure

The amino acid sequence of carbohydrate oxidase of SEQ ID NO:2 was used to elucidate a three-dimensional structure. The structure was solved in accordance with the principle for x-ray crystallographic methods, for example, as given in X-Ray Structure Determination, Stout, G. K. and Jensen, L. H., John Wiley & Sons, Inc. NY, 1989.

The structural coordinates for the solved crystal structure of the carbohydrate oxidase of SEQ ID NO:2, as inhibited by ABL=5-amino-5-deoxy-cellobiono-1,5-lactam, are given in standard PDB format (Protein Data Bank, Brookhaven National Laboratory, Brookhaven, Conn.) as set forth in Appendix 1, which forms part of the present application. In the context of Appendix 1, the following abbreviations are used: CA refers to c-alpha (carbon atoms) or to calcium ions, (however to avoid misunderstandings we use the full names “c-alpha atoms” and “calcium” or “ion” in the present specification). Amino acid residues are given in their standard three-letter code. The attached structural coordinates contain the carbohydrate oxidase structure.

### Preparation of Variants

Variants of parent carbohydrate oxidases can be prepared according to any mutagenesis procedure known in the art, such as site-directed mutagenesis, synthetic gene construction, semi-synthetic gene construction, random mutagenesis, shuffling, etc.

Site-directed mutagenesis is a technique in which one or several mutations are created at a defined site in a polynucleotide molecule encoding the parent carbohydrate oxidase. The technique can be performed in vitro or in vivo.

Synthetic gene construction entails in vitro synthesis of a designed polynucleotide molecule to encode a polypeptide molecule of interest. Gene synthesis can be performed utilizing a number of techniques, such as the multiplex microchip-based technology described by Tian, et. al., (Tian, et. al., Nature 432:1050-1054) and similar technologies wherein oligonucleotides are synthesized and assembled upon photo-programmable microfluidic chips.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent carbohydrate oxidase and subsequent ligation of an oligonucleotide containing the mutation in the polynucleotide. Usually the restriction enzyme that digests at the plasmid and the oligonucleotide is the same, permitting sticky ends of the plasmid and insert to ligate to one another. See, for example, Scherer and Davis, 1979, Proc. Natl. Acad. Sci. USA 76: 4949-4955; and Barton et al., 1990, Nucleic Acids Research 18: 7349-4966.

Site-directed mutagenesis can be accomplished in vivo by methods known in the art. See, for example, U.S. Patent Application Publication 2004/0171154; Storici et al., 2001, Nature Biotechnology 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants of a parent carbohydrate oxidase.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30:10832-10837; U.S. Pat. No. 5,223,409; WO 92/06204) and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells. Mutagenized DNA molecules that encode active polypeptides can be recovered from the host cells and rapidly sequenced using standard methods in the art. These methods allow the rapid determination of the importance of individual amino acid residues in a polypeptide of interest.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide fragments may then be shuffled.

### Variants

In the present invention, the isolated carbohydrate oxidase variants comprise a substitution at one or more (several) positions corresponding to positions 4, 15, 19, 21, 22, 27, 29, 30, 31, 43, 48, 52, 54, 57, 58, 59, 60, 62, 69, 70, 71, 72, 77, 80, 81, 85, 91, 93, 98, 105, 112, 114, 118, 122, 129, 130, 131, 132, 133, 134, 135, 138, 140, 146, 147, 148, 152, 153, 157, 169, 170, 174, 184, 188, 201, 213, 221, 222, 223, 224, 227, 228, 231, 235, 248, 249, 250, 251, 252, 253, 256, 258, 260, 268, 278, 287, 288, 300, 301, 302, 303, 304, 305, 307, 314, 315, 316, 317, 318, 319, 320, 321, 322, 323, 327, 330, 332, 342, 347, 349, 352, 353, 354, 355, 356, 357, 358, 363, 366, 368, 374, 382, 383, 385, 386, 387, 388, 389, 390, 391, 392, 393, 400, 403, 411, 415, 419, 420, 424, 425, 427, 428, 429, 433, 437, 440, 445, 456, 460, 472, and/or 473 of SEQ ID NO:2, and have carbohydrate oxidase activity. The carbohydrate oxidase variants may further comprise an amino acid sequence having a degree of amino acid sequence identity of at least 60%, preferably at least 65%, more preferably at least 70%, more preferably at least 75%, more preferably at least 80%, more preferably at least 85%, even more preferably at least 90%, most preferably at least 95%, and even most preferably at least about 96%, 97%, 98% or 99% amino acid sequence identity to the amino acid sequence of SEQ ID NO:2 or the mature polypeptide encoded by SEQ ID NO:1.

The present invention also relates to isolated carbohydrate oxidase variants or isolated polypeptides having carbohydrate oxidase activity which comprises an amino acid sequence which differs from the carbohydrate oxidase of SEQ ID NO:2 by at least one or more (several) positions corresponding to positions 4, 15, 19, 21, 22, 27, 29, 30, 31, 43, 48, 52, 54, 57, 58, 59, 60, 62, 69, 70, 71, 72, 77, 80, 81, 85, 91, 93, 98, 105, 112, 114, 118, 122, 129, 130, 131, 132, 133, 134, 135, 138, 140, 146, 147, 148, 152, 153, 157, 169, 170, 174, 184, 188, 201, 213, 221, 222, 223, 224, 227, 228, 231, 235, 248, 249, 250, 251, 252, 253, 256, 258, 260, 268, 278, 287, 288, 300, 301, 302, 303, 304, 305, 307, 314, 315, 316, 317, 318, 319, 320, 321, 322, 323, 327, 330, 332, 342, 347, 349, 352, 353, 354, 355, 356, 357, 358, 363, 366, 368, 374, 382, 383, 385, 386, 387, 388, 389, 390, 391, 392, 393, 400, 403, 411, 415, 419, 420, 424, 425, 427, 428, 429, 433, 437, 440, 445, 456, 460, 472, and/or 473, using SEQ ID NO:2 for numbering, and which have carbohydrate oxidase activity. The carbohydrate oxidase variants may further comprise an amino acid sequence having a degree of amino acid sequence identity of at least 60%, at least 65%, at least 70%, at least 75%, at least 80%, at least 85%, at least 90%, at least 95%, at least 96%, at least 97%, at least 98% or at least 99% amino acid sequence identity to the amino acid sequence of SEQ ID NO:2 or the mature polypeptide encoded by SEQ ID NO:1.

In one aspect, the number of amino acid alterations in the variants of the present invention comprise or consist of, as compared to the parent (e.g., a parent carbohydrate oxidase having the amino acid sequence shown in SEQ ID NO:2), 20 alterations, 19 alterations, 18 alterations, 17 alterations, 16 alterations, 15 alterations, 14 alterations, 13 alterations, 12 alterations, 11 alterations, 10 alterations, 9 alterations, 8 alterations, 7 alterations, 6 alterations, 5 alterations, 4 alterations, more preferably 3 alterations, even more preferably 2 alterations, and most preferably 1 alteration. In another aspect, the number of amino acid alterations in the variants of the present invention consists of preferably 20 alterations, 19 alterations, 18 alterations, 17 alterations, 16 alterations, 15 alterations, 14 alterations, 13 alterations, 12 alterations, 11 alterations, 10 alterations, 9 alterations, 8 alterations, 7 alterations, 6 alterations, 5 alterations, 4 alterations, 3 alterations, 2 alterations, or 1 alteration.

In one aspect, the number of amino acid alterations in the variants of the present invention comprise or consist of, as compared to the parent (e.g., a parent carbohydrate oxidase having the amino acid sequence shown in SEQ ID NO:2), 20 substitutions, 19 substitutions, 18 substitutions, 17 substitutions, 16 substitutions, 15 substitutions, 14 substitutions, 13 substitutions, 12 substitutions, 11 substitutions, 10 substitutions, 9 substitutions, 8 substitutions, 7 substitutions, 6 substitutions, 5 substitutions, 4 substitutions, more preferably 3 substitutions, even more preferably 2 substitutions, and most preferably 1 substitution. In another aspect, the number of amino acid substitutions in the variants of the present invention consists of 20 substitutions, 19 substitutions, 18 substitutions, 17 substitutions, 16 substitutions, 15 substitutions, 14 substitutions, 13 substitutions, 12 substitutions, 11 substitutions, 10 substitutions, 9 substitutions, 8 substitutions, 7 substitutions, 6 substitutions, 5 substitutions, 4 substitutions, 3 substitutions, 2 substitutions, or 1 substitution.

In one aspect, the variant comprises or consists of a substitution at a position corresponding to position 4 of SEQ ID NO:2. In another aspect, the variant comprises or consists of a substitution at a position corresponding to position 4 of SEQ ID NO:2 with Ala, Arg, Asn, Asp, Cys, Gln, Gly, His, Ile, Leu, Lys, Met, Phe, Pro, Ser, Thr, Trp, Tyr, or Val. In another aspect, the variant comprises or consists of Gln as a substitution at a position corresponding to position 4 of SEQ ID NO:2. In another aspect, the variant comprises or consists of the substitution E4Q.

In another aspect, the variant comprises or consists of a substitution at a position corresponding to position 15 of SEQ ID NO:2. In another aspect, the variant comprises or consists of a substitution at a position corresponding to position 15 of SEQ ID NO:2 with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of Asn as a substitution at a position corresponding to position 15 of SEQ ID NO:2. In another aspect, the variant comprises the substitution D15N.

In another aspect, the variant comprises or consists of a substitution at a position corresponding to position 19 of SEQ ID NO:2. In another aspect, the variant comprises or consists of a substitution at a position corresponding to position 19 of SEQ ID NO:2 with Ala, Arg, Asn, Asp, Cys, Gln, Glu, Gly, His, Ile, Leu, Lys, Met, Phe, Pro, Ser, Trp, Tyr, or Val. In another aspect, the variant comprises or consists of Ser as a substitution at a position corresponding to position 19 of SEQ ID NO:2. In another aspect, the variant comprises or consists of the substitution T195.

In another aspect, the variant comprises or consists of a substitution at a position corresponding to position 21 of SEQ ID NO:2. In another aspect, the variant comprises or consists of a substitution at a position corresponding to position 21 of SEQ ID NO:2 with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of Asn as a substitution at a position corresponding to position 21 of SEQ ID NO:2. In another aspect, the variant comprises or consists of the substitution D21N.

In another aspect, the variant comprises or consists of a substitution at a position corresponding to position 22 of SEQ ID NO:2. In another aspect, the variant comprises or consists of a substitution at a position corresponding to position 22 of SEQ ID NO:2 with Ala, Arg, Asn, Asp, Cys, Gln, Glu, Gly, His, Ile, Leu, Lys, Met, Phe, Pro, Ser, Thr, Trp, or Val. In another aspect, the variant comprises or consists of Trp as a substitution at a position corresponding to position 22 of SEQ ID NO:2. In another aspect, the variant comprises or consists of the substitution Y22W.

In another aspect, the variant comprises or consists of a substitution at a position corresponding to position 27 of SEQ ID NO:2. In another aspect, the variant comprises or consists of a substitution at a position corresponding to position 27 of SEQ ID NO:2 with Ala, Arg, Asn, Asp, Cys, Gln, Gly, His, Ile, Leu, Lys, Met, Phe, Pro, Ser, Thr, Trp, Tyr, or Val. In another aspect, the variant comprises or consists of Arg or Lys as a substitution at a position corresponding to position 21 of SEQ ID NO:2. In another aspect, the variant comprises or consists of the substitution E27R,K.

In another aspect, the variant comprises or consists of a substitution at a position corresponding to position 29 of SEQ ID NO:2. In another aspect, the variant comprises or consists of a substitution at a position corresponding to position 29 of SEQ ID NO:2 with Ala, Arg, Asn, Asp, Cys, Gln, Glu, Gly, His, Ile, Leu, Lys, Met, Pro, Ser, Thr, Trp, Tyr, or Val.

In another aspect, the variant comprises or consists of a substitution at a position corresponding to position 30 of SEQ ID NO:2. In another aspect, the variant comprises or consists of a substitution at a position corresponding to position 30 of SEQ ID NO:2 with Ala, Arg, Asp, Cys, Gln, Glu, Gly, His, Ile, Leu, Lys, Met, Phe, Pro, Ser, Thr, Trp, Tyr, or Val.

In another aspect, the variant comprises or consists of a substitution at a position corresponding to position 31 of SEQ ID NO:2. In another aspect, the variant comprises or consists of a substitution at a position corresponding to position 31 of SEQ ID NO:2 with Ala, Arg, Asn, Asp, Cys, Gln, Glu, Gly, His, Leu, Lys, Met, Phe, Pro, Ser, Thr, Trp, Tyr, or Val. In another aspect, the variant comprises or consists of Arg as a substitution at a position corresponding to position 31 of SEQ ID NO:2. In another aspect, the variant comprises or consists of the substitution 131R.

In another aspect, the variant comprises or consists of a substitution at a position corresponding to position 43 of SEQ ID NO:2. In another aspect, the variant comprises or consists of a substitution at a position corresponding to position 43 of SEQ ID NO:2 with Ala, Arg, Asn, Asp, Cys, Gln, Glu, Gly, His, Ile, Leu, Lys, Met, Phe, Pro, Ser, Trp, Tyr, or Val. In another aspect, the variant comprises or consists of Cys as a substitution at a position corresponding to position 43 of SEQ ID NO:2. In another aspect, the variant comprises the substitution T43C. In another aspect, the variant comprises or consists of the substitution T43C and A52C.

In another aspect, the variant comprises or consists of a substitution at a position corresponding to position 48 of SEQ ID NO:2. In another aspect, the variant comprises or consists of a substitution at a position corresponding to position 48 of SEQ ID NO:2 with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of Arg, Asp, Ser, or Thr as a substitution at a position corresponding to position 48 of SEQ ID NO:2. In another aspect, the variant comprises or consists of the substitution N48R,D,S,T.

In another aspect, the variant comprises or consists of a substitution at a position corresponding to position 52 of SEQ ID NO:2. In another aspect, the variant comprises or consists of a substitution at a position corresponding to position 52 of SEQ ID NO:2 with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of Cys as a substitution at a position corresponding to position 52 of SEQ ID NO:2. In another aspect, the variant comprises or consists of the substitution A52C. In another aspect, the variant comprises the substitution T43C and A52C.

In another aspect, the variant comprises or consists of a substitution at a position corresponding to position 54 of SEQ ID NO:2. In another aspect, the variant comprises or consists of a substitution at a position corresponding to position 54 of SEQ ID NO:2 with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of Glu or Asp as a substitution at a position corresponding to position 54 of SEQ ID NO:2. In another aspect, the variant comprises or consists of the substitution Q54E,D.

In another aspect, the variant comprises or consists of a substitution at a position corresponding to position 57 of SEQ ID NO:2. In another aspect, the variant comprises or consists of a substitution at a position corresponding to position 57 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Asp as a substitution at a position corresponding to position 57 of SEQ ID NO:2. In another aspect, the variant comprises or consists of the substitution K57D.

In another aspect, the variant comprises or consists of a substitution at a position corresponding to position 58 of SEQ ID NO:2. In another aspect, the variant comprises or consists of a substitution at a position corresponding to position 58 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Asp, Glu or Arg as a substitution at a position corresponding to position 58 of SEQ ID NO:2. In another aspect, the variant comprises or consists of the substitution K58D,E,R.

In another aspect, the variant comprises or consists of a substitution at a position corresponding to position 59 of SEQ ID NO:2. In another aspect, the variant comprises or consists of a substitution at a position corresponding to position 59 of SEQ ID NO:2 with Ala, Arg, Asn, Asp, Cys, Gln, Glu, Gly, His, Ile, Lys, Met, Phe, Pro, Ser, Thr, Trp, Tyr, or Val. In another aspect, the variant comprises or consists of Asp as a substitution at a position corresponding to position 59 of SEQ ID NO:2. In another aspect, the variant comprises or consists of the substitution L59D.

In another aspect, the variant comprises or consists of a substitution at a position corresponding to position 60 of SEQ ID NO:2. In another aspect, the variant comprises or consists of a substitution at a position corresponding to position 60 of SEQ ID NO:2 with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of Gly as a substitution at a position corresponding to position 60 of SEQ ID NO:2. In another aspect, the variant comprises or consists of the substitution N60G.

In another aspect, the variant comprises or consists of a substitution at a position corresponding to position 62 of SEQ ID NO:2. In another aspect, the variant comprises or consists of a substitution at a position corresponding to position 62 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Leu or Val as a substitution at a position corresponding to position 62 of SEQ ID NO:2. In another aspect, the variant comprises or consists of the substitution K62L,V.

In another aspect, the variant comprises or consists of a substitution at a position corresponding to position 69 of SEQ ID NO:2. In another aspect, the variant comprises or consists of a substitution at a position corresponding to position 69 of SEQ ID NO:2 with Ala, Arg, Asn, Asp, Cys, Gln, Glu, His, Ile, Leu, Lys, Met, Phe, Pro, Ser, Thr, Trp, Tyr, or Val.

In another aspect, the variant comprises or consists of a substitution at a position corresponding to position 70 of SEQ ID NO:2. In another aspect, the variant comprises or consists of a substitution at a position corresponding to position 70 of SEQ ID NO:2 with Ala, Arg, Asn, Asp, Cys, Gln, Glu, Gly, Ile, Leu, Lys, Met, Phe, Pro, Ser, Thr, Trp, Tyr, or Val.

In another aspect, the variant comprises or consists of a substitution at a position corresponding to position 71 of SEQ ID NO:2. In another aspect, the variant comprises or consists of a substitution at a position corresponding to position 71 of SEQ ID NO:2 with Ala, Arg, Asn, Asp, Cys, Gln, Glu, Gly, His, Ile, Leu, Lys, Met, Phe, Pro, Thr, Trp, Tyr, or Val.

In another aspect, the variant comprises or consists of a substitution at a position corresponding to position 72 of SEQ ID NO:2. In another aspect, the variant comprises or consists of a substitution at a position corresponding to position 72 of SEQ ID NO:2 with Ala, Arg, Asn, Asp, Cys, Gln, Glu, Gly, His, Ile, Leu, Lys, Met, Phe, Pro, Ser, Thr, Trp, or Val.

In another aspect, the variant comprises or consists of a substitution at a position corresponding to position 77 of SEQ ID NO:2. In another aspect, the variant comprises or consists of a substitution at a position corresponding to position 77 of SEQ ID NO:2 with Ala, Arg, Asn, Asp, Cys, Gln, Glu, Gly, His, Ile, Leu, Lys, Met, Pro, Ser, Thr, Trp, Tyr, or Val. In another aspect, the variant comprises or consists of Tyr as a substitution at a position corresponding to position 77 of SEQ ID NO:2. In another aspect, the variant comprises or consists of the substitution F77Y.

In another aspect, the variant comprises or consists of a substitution at a position corresponding to position 80 of SEQ ID NO:2. In another aspect, the variant comprises or consists of a substitution at a position corresponding to position 80 of SEQ ID NO:2 with Ala, Arg, Asn, Asp, Cys, Gln, Gly, His, Ile, Leu, Lys, Met, Phe, Pro, Ser, Thr, Trp, Tyr, or Val. In another aspect, the variant comprises or consists of Ala as a substitution at a position corresponding to position 80 of SEQ ID NO:2. In another aspect, the variant comprises or consists of the substitution E80A.

In another aspect, the variant comprises or consists of a substitution at a position corresponding to position 81 of SEQ ID NO:2. In another aspect, the variant comprises or consists of a substitution at a position corresponding to position 81 of SEQ ID NO:2 with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of Ala, His or Asp as a substitution at a position corresponding to position 81 of SEQ ID NO:2. In another aspect, the variant comprises or consists of the substitution N81A,H,D.

In another aspect, the variant comprises or consists of a substitution at a position corresponding to position 85 of SEQ ID NO:2. In another aspect, the variant comprises or consists of a substitution at a position corresponding to position 85 of SEQ ID NO:2 with Ala, Arg, Asn, Asp, Cys, Gln, Glu, Gly, His, Ile, Leu, Lys, Phe, Pro, Ser, Thr, Trp, Tyr, or Val. In another aspect, the variant comprises or consists of Val or Ala as a substitution at a position corresponding to position 85 of SEQ ID NO:2. In another aspect, the variant comprises or consists of the substitution M85V,A.

In another aspect, the variant comprises or consists of a substitution at a position corresponding to position 91 of SEQ ID NO:2. In another aspect, the variant comprises or consists of a substitution at a position corresponding to position 91 of SEQ ID NO:2 with Ala, Arg, Asn, Asp, Cys, Gln, Glu, Gly, His, Ile, Leu, Lys, Phe, Pro, Ser, Thr, Trp, Tyr, or Val. In another aspect, the variant comprises or consists of Ala as a substitution at a position corresponding to position 91 of SEQ ID NO:2. In another aspect, the variant comprises or consists of the substitution M91A.

In another aspect, the variant comprises or consists of a substitution at a position corresponding to position 48 of SEQ ID NO:2. In another aspect, the variant comprises or consists of a substitution at a position corresponding to position 48 of SEQ ID NO:2 with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of Arg, Asp, Ser, Thr as a substitution at a position corresponding to position 48 of SEQ ID NO:2. In another aspect, the variant comprises or consists of the substitution N48R,D,S,T.

In another aspect, the variant comprises or consists of a substitution at a position corresponding to position 93 of SEQ ID NO:2. In another aspect, the variant comprises or consists of a substitution at a position corresponding to position 93 of SEQ ID NO:2 with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of Ser or Thr as a substitution at a position corresponding to position 93 of SEQ ID NO:2. In another aspect, the variant comprises or consists of the substitution D93S,T.

In another aspect, the variant comprises or consists of a substitution at a position corresponding to position 98 of SEQ ID NO:2. In another aspect, the variant comprises or consists of a substitution at a position corresponding to position 98 of SEQ ID NO:2 with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of Asp as a substitution at a position corresponding to position 98 of SEQ ID NO:2. In another aspect, the variant comprises or consists of the substitution N98D.

In another aspect, the variant comprises or consists of a substitution at a position corresponding to position 105 of SEQ ID NO:2. In another aspect, the variant comprises or consists of a substitution at a position corresponding to position 105 of SEQ ID NO:2 with Ala, Arg, Asn, Asp, Cys, Gln, Glu, Gly, Ile, Leu, Lys, Met, Phe, Pro, Ser, Thr, Trp, Tyr, or Val. In another aspect, the variant comprises or consists of Gln, Asn, Asp, Glu as a substitution at a position corresponding to position 105 of SEQ ID NO:2. In another aspect, the variant comprises or consists of the substitution H105Q,N,D,E.

In another aspect, the variant comprises or consists of a substitution at a position corresponding to position 112 of SEQ ID NO:2. In another aspect, the variant comprises or consists of a substitution at a position corresponding to position 112 of SEQ ID NO:2 with Ala, Arg, Asn, Asp, Cys, Gln, Glu, Gly, His, Ile, Lys, Met, Phe, Pro, Ser, Thr, Trp, Tyr, or Val.

In another aspect, the variant comprises or consists of a substitution at a position corresponding to position 114 of SEQ ID NO:2. In another aspect, the variant comprises or consists of a substitution at a position corresponding to position 114 of SEQ ID NO:2 with Ala, Arg, Asn, Asp, Cys, Gln, Glu, Gly, Ile, Leu, Lys, Met, Phe, Pro, Ser, Thr, Trp, Tyr, or Val. In another aspect, the variant comprises or consists of Arg as a substitution at a position corresponding to position 114 of SEQ ID NO:2. In another aspect, the variant comprises or consists of the substitution H114R.

In another aspect, the variant comprises or consists of a substitution at a position corresponding to position 118 of SEQ ID NO:2. In another aspect, the variant comprises or consists of a substitution at a position corresponding to position 118 of SEQ ID NO:2 with Ala, Arg, Asn, Asp, Cys, Gln, Glu, Gly, His, Ile, Leu, Lys, Met, Phe, Pro, Ser, Thr, Trp, or Tyr. In another aspect, the variant comprises or consists of Arg or Lys as a substitution at a position corresponding to position 118 of SEQ ID NO:2. In another aspect, the variant comprises or consists of the substitution V118R,K.

In another aspect, the variant comprises or consists of a substitution at a position corresponding to position 122 of SEQ ID NO:2. In another aspect, the variant comprises or consists of a substitution at a position corresponding to position 122 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Arg as a substitution at a position corresponding to position 122 of SEQ ID NO:2. In another aspect, the variant comprises or consists of the substitution K122R.

In another aspect, the variant comprises or consists of a substitution at a position corresponding to position 129 of SEQ ID NO:2. In another aspect, the variant comprises or consists of a substitution at a position corresponding to position 129 of SEQ ID NO:2 with Ala, Arg, Asn, Asp, Cys, Gln, Glu, Gly, His, Ile, Leu, Lys, Met, Phe, Pro, Ser, Thr, Trp, Tyr, or Val.

In another aspect, the variant comprises or consists of a substitution at a position corresponding to position 130 of SEQ ID NO:2. In another aspect, the variant comprises or consists of a substitution at a position corresponding to position 130 of SEQ ID NO:2 with Ala, Arg, Asn, Asp, Cys, Gln, Glu, His, Ile, Leu, Lys, Met, Phe, Pro, Ser, Thr, Trp, Tyr, or Val.

In another aspect, the variant comprises or consists of a substitution at a position corresponding to position 131 of SEQ ID NO:2. In another aspect, the variant comprises or consists of a substitution at a position corresponding to position 131 of SEQ ID NO:2 with Ala, Arg, Asn, Asp, Cys, Gln, Glu, Gly, His, Ile, Leu, Lys, Met, Phe, Pro, Ser, Thr, Trp, Tyr, or Val.

In another aspect, the variant comprises or consists of a substitution at a position corresponding to position 132 of SEQ ID NO:2. In another aspect, the variant comprises or consists of a substitution at a position corresponding to position 132 of SEQ ID NO:2 with Ala, Arg, Asn, Asp, Cys, Gln, Glu, Gly, His, Ile, Leu, Lys, Met, Phe, Pro, Ser, Trp, Tyr, or Val.

In another aspect, the variant comprises or consists of a substitution at a position corresponding to position 133 of SEQ ID NO:2. In another aspect, the variant comprises or consists of a substitution at a position corresponding to position 133 of SEQ ID NO:2 with Ala, Arg, Asn, Asp, Cys, Gln, Glu, Gly, His, Ile, Leu, Lys, Met, Phe, Ser, Thr, Trp, Tyr, or Val.

In another aspect, the variant comprises or consists of a substitution at a position corresponding to position 134 of SEQ ID NO:2. In another aspect, the variant comprises or consists of a substitution at a position corresponding to position 134 of SEQ ID NO:2 with Ala, Arg, Asn, Asp, Cys, Gln, Glu, His, Ile, Leu, Lys, Met, Phe, Pro, Ser, Thr, Trp, Tyr, or Val.

In another aspect, the variant comprises or consists of a substitution at a position corresponding to position 135 of SEQ ID NO:2. In another aspect, the variant comprises or consists of a substitution at a position corresponding to position 135 of SEQ ID NO:2 with Ala, Arg, Asn, Asp, Cys, Gln, Glu, Gly, His, Ile, Leu, Lys, Met, Phe, Pro, Ser, Thr, Trp, or Tyr.

In another aspect, the variant comprises or consists of a substitution at a position corresponding to position 138 of SEQ ID NO:2. In another aspect, the variant comprises or consists of a substitution at a position corresponding to position 138 of SEQ ID NO:2 with Ala, Arg, Asn, Asp, Cys, Gln, Glu, Gly, His, Ile, Leu, Lys, Met, Phe, Pro, Thr, Trp, Tyr, or Val. In another aspect, the variant comprises or consists of Gly as a substitution at a position corresponding to position 138 of SEQ ID NO:2. In another aspect, the variant comprises or consists of the substitution S138G.

In another aspect, the variant comprises or consists of a substitution at a position corresponding to position 140 of SEQ ID NO:2. In another aspect, the variant comprises or consists of a substitution at a position corresponding to position 140 of SEQ ID NO:2 with Ala, Arg, Asn, Asp, Cys, Gln, Glu, Gly, Ile, Leu, Lys, Met, Phe, Pro, Ser, Thr, Trp, Tyr, or Val.

In another aspect, the variant comprises or consists of a substitution at a position corresponding to position 146 of SEQ ID NO:2. In another aspect, the variant comprises or consists of a substitution at a position corresponding to position 146 of SEQ ID NO:2 with Ala, Arg, Asn, Asp, Cys, Gln, Glu, Gly, His, Ile, Leu, Lys, Met, Pro, Ser, Thr, Trp, Tyr, or Val. In another aspect, the variant comprises or consists of Tyr as a substitution at a position corresponding to position 146 of SEQ ID NO:2. In another aspect, the variant comprises or consists of the substitution F146Y.

In another aspect, the variant comprises or consists of a substitution at a position corresponding to position 147 of SEQ ID NO:2. In another aspect, the variant comprises or consists of a substitution at a position corresponding to position 147 of SEQ ID NO:2 with Ala, Arg, Asn, Asp, Cys, Gln, Glu, His, Ile, Leu, Lys, Met, Phe, Pro, Ser, Thr, Trp, Tyr, or Val.

In another aspect, the variant comprises or consists of a substitution at a position corresponding to position 148 of SEQ ID NO:2. In another aspect, the variant comprises or consists of a substitution at a position corresponding to position 148 of SEQ ID NO:2 with Ala, Arg, Asn, Asp, Cys, Gln, Glu, Gly, His, Ile, Leu, Lys, Met, Pro, Ser, Thr, Trp, Tyr, or Val. In another aspect, the variant comprises or consists of Asp or Glu as a substitution at a position corresponding to position 148 of SEQ ID NO:2. In another aspect, the variant comprises or consists of the substitution F148D,E.

In another aspect, the variant comprises or consists of a substitution at a position corresponding to position 152 of SEQ ID NO:2. In another aspect, the variant comprises or consists of a substitution at a position corresponding to position 152 of SEQ ID NO:2 with Ala, Arg, Asn, Asp, Cys, Gln, Glu, Gly, His, Ile, Leu, Lys, Phe, Pro, Ser, Thr, Trp, Tyr, or Val. In another aspect, the variant comprises or consists of Thr or Ala as a substitution at a position corresponding to position 152 of SEQ ID NO:2. In another aspect, the variant comprises or consists of the substitution M152AT.

In another aspect, the variant comprises or consists of a substitution at a position corresponding to position 153 of SEQ ID NO:2. In another aspect, the variant comprises or consists of a substitution at a position corresponding to position 153 of SEQ ID NO:2 with Ala, Arg, Asn, Asp, Cys, Gln, Glu, Gly, Ile, Leu, Lys, Met, Phe, Pro, Ser, Thr, Trp, Tyr, or Val. In another aspect, the variant comprises or consists of Asn as a substitution at a position corresponding to position 153 of SEQ ID NO:2. In another aspect, the variant comprises or consists of the substitution H153N.

In another aspect, the variant comprises or consists of a substitution at a position corresponding to position 157 of SEQ ID NO:2. In another aspect, the variant comprises or consists of a substitution at a position corresponding to position 188 of SEQ ID NO:2 with Ala, Arg, Asn, Asp, Cys, Gln, Glu, Gly, His, Ile, Leu, Lys, Met, Phe, Pro, Ser, Thr, Trp, or Tyr. In another aspect, the variant comprises or consists of Cys as a substitution at a position corresponding to position 157 of SEQ ID NO:2. In another aspect, the variant comprises or consists of the substitution V157C. In another aspect, the variant comprises or consists of the substitutions V157C+K188C.

In another aspect, the variant comprises or consists of a substitution at a position corresponding to position 169 of SEQ ID NO:2. In another aspect, the variant comprises or consists of a substitution at a position corresponding to position 169 of SEQ ID NO:2 with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of Ala as a substitution at a position corresponding to position 169 of SEQ ID NO:2. In another aspect, the variant comprises or consists of the substitution D169A.

In another aspect, the variant comprises or consists of a substitution at a position corresponding to position 170 of SEQ ID NO:2. In another aspect, the variant comprises or consists of a substitution at a position corresponding to position 170 of SEQ ID NO:2 with Ala, Arg, Asn, Asp, Cys, Gln, Glu, His, Ile, Leu, Lys, Met, Phe, Pro, Ser, Thr, Trp, Tyr, or Val. In another aspect, the variant comprises or consists of Ser as a substitution at a position corresponding to position 170 of SEQ ID NO:2. In another aspect, the variant comprises or consists of the substitution G170S.

In another aspect, the variant comprises or consists of a substitution at a position corresponding to position 174 of SEQ ID NO:2. In another aspect, the variant comprises or consists of a substitution at a position corresponding to position 174 of SEQ ID NO:2 with Ala, Arg, Asn, Asp, Cys, Gln, Gly, His, Ile, Leu, Lys, Met, Phe, Pro, Ser, Thr, Trp, Tyr, or Val. In another aspect, the variant comprises or consists of Gln as a substitution at a position corresponding to position 174 of SEQ ID NO:2. In another aspect, the variant comprises or consists of the substitution E174Q.

In another aspect, the variant comprises or consists of a substitution at a position corresponding to position 184 of SEQ ID NO:2. In another aspect, the variant comprises or consists of a substitution at a position corresponding to position 184 of SEQ ID NO:2 with Ala, Arg, Asn, Asp, Cys, Gln, Glu, Gly, His, Ile, Leu, Lys, Met, Pro, Ser, Thr, Trp, Tyr, or Val. In another aspect, the variant comprises or consists of Tyr as a substitution at a position corresponding to position 184 of SEQ ID NO:2. In another aspect, the variant comprises or consists of the substitution F184Y.

In another aspect, the variant comprises or consists of a substitution at a position corresponding to position 188 of SEQ ID NO:2. In another aspect, the variant comprises or consists of a substitution at a position corresponding to position 188 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Arg, Cys, Leu, or Val as a substitution at a position corresponding to position 188 of SEQ ID NO:2. In another aspect, the variant comprises or consists of the substitution K188R,C,L,V. In another aspect, the variant comprises or consists of the substitutions V157C+K188C.

In another aspect, the variant comprises or consists of a substitution at a position corresponding to position 201 of SEQ ID NO:2. In another aspect, the variant comprises or consists of a substitution at a position corresponding to position 201 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Arg as a substitution at a position corresponding to position 201 of SEQ ID NO:2. In another aspect, the variant comprises or consists of the substitution K201R.

In another aspect, the variant comprises or consists of a substitution at a position corresponding to position 213 of SEQ ID NO:2. In another aspect, the variant comprises or consists of a substitution at a position corresponding to position 213 of SEQ ID NO:2 with Ala, Arg, Asn, Asp, Cys, Gln, Glu, Gly, His, Ile, Leu, Lys, Met, Phe, Pro, Ser, Thr, Trp, Tyr, or Val.

In another aspect, the variant comprises or consists of a substitution at a position corresponding to position 221 of SEQ ID NO:2. In another aspect, the variant comprises or consists of a substitution at a position corresponding to position 221 of SEQ ID NO:2 with Ala, Asn, Asp, Cys, Gln, Glu, Gly, His, Ile, Leu, Lys, Met, Phe, Pro, Ser, Thr, Trp, Tyr, or Val. In another aspect, the variant comprises or consists of R as a substitution at a position corresponding to position 221 of SEQ ID NO:2. In another aspect, the variant comprises or consists of the substitution K221R.

In another aspect, the variant comprises or consists of a substitution at a position corresponding to position 222 of SEQ ID NO:2. In another aspect, the variant comprises or consists of a substitution at a position corresponding to position 222 of SEQ ID NO:2 with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of Asp as a substitution at a position corresponding to position 222 of SEQ ID NO:2. In another aspect, the variant comprises or consists of the substitution N222D.

In another aspect, the variant comprises or consists of a substitution at a position corresponding to position 223 of SEQ ID NO:2. In another aspect, the variant comprises or consists of a substitution at a position corresponding to position 223 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Arg as a substitution at a position corresponding to position 223 of SEQ ID NO:2. In another aspect, the variant comprises or consists of the substitution K223R.

In another aspect, the variant comprises or consists of a substitution at a position corresponding to position 224 of SEQ ID NO:2. In another aspect, the variant comprises or consists of a substitution at a position corresponding to position 224 of SEQ ID NO:2 with Ala, Arg, Asn, Asp, Cys, Gln, Glu, Gly, His, Ile, Leu, Lys, Met, Phe, Pro, Ser, Trp, Tyr, or Val. In another aspect, the variant comprises or consists of Glu or Asp as a substitution at a position corresponding to position 224 of SEQ ID NO:2. In another aspect, the variant comprises or consists of the substitution T224E,D.

In another aspect, the variant comprises or consists of a substitution at a position corresponding to position 227 of SEQ ID NO:2. In another aspect, the variant comprises or consists of a substitution at a position corresponding to position 227 of SEQ ID NO:2 with Ala, Arg, Asn, Asp, Cys, Gln, Glu, Gly, His, Ile, Lys, Met, Phe, Pro, Ser, Thr, Trp, Tyr, or Val. In another aspect, the variant comprises or consists of Glu or Asp as a substitution at a position corresponding to position 227 of SEQ ID NO:2. In another aspect, the variant comprises or consists of the substitution L227D,E.

In another aspect, the variant comprises or consists of a substitution at a position corresponding to position 228 of SEQ ID NO:2. In another aspect, the variant comprises or consists of a substitution at a position corresponding to position 228 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Arg as a substitution at a position corresponding to position 228 of SEQ ID NO:2. In another aspect, the variant comprises or consists of the substitution K228R.

In another aspect, the variant comprises or consists of a substitution at a position corresponding to position 231 of SEQ ID NO:2. In another aspect, the variant comprises or consists of a substitution at a position corresponding to position 231 of SEQ ID NO:2 with Ala, Arg, Asn, Asp, Cys, Gln, Gly, His, Ile, Leu, Lys, Met, Phe, Pro, Ser, Thr, Trp, Tyr, or Val. In another aspect, the variant comprises or consists of Gln as a substitution at a position corresponding to position 231 of SEQ ID NO:2. In another aspect, the variant comprises or consists of the substitution E231Q.

In another aspect, the variant comprises or consists of a substitution at a position corresponding to position 235 of SEQ ID NO:2. In another aspect, the variant comprises or consists of a substitution at a position corresponding to position 235 of SEQ ID NO:2 with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of Asn as a substitution at a position corresponding to position 235 of SEQ ID NO:2. In another aspect, the variant comprises or consists of the substitution D235N.

In another aspect, the variant comprises or consists of a substitution at a position corresponding to position 248 of SEQ ID NO:2. In another aspect, the variant comprises or consists of a substitution at a position corresponding to position 248 of SEQ ID NO:2 with Ala, Asn, Asp, Cys, Gln, Glu, Gly, His, Ile, Leu, Lys, Met, Phe, Pro, Ser, Thr, Trp, Tyr, or Val.

In another aspect, the variant comprises or consists of a substitution at a position corresponding to position 249 of SEQ ID NO:2. In another aspect, the variant comprises or consists of a substitution at a position corresponding to position 249 of SEQ ID NO:2 with Ala, Arg, Asn, Asp, Cys, Gln, Glu, Gly, His, Leu, Lys, Met, Phe, Pro, Ser, Thr, Trp, Tyr, or Val.

In another aspect, the variant comprises or consists of a substitution at a position corresponding to position 250 of SEQ ID NO:2. In another aspect, the variant comprises or consists of a substitution at a position corresponding to position 250 of SEQ ID NO:2 with Ala, Arg, Asn, Asp, Cys, Gln, Glu, His, Ile, Leu, Lys, Met, Phe, Pro, Ser, Thr, Trp, Tyr, or Val.

In another aspect, the variant comprises or consists of a substitution at a position corresponding to position 251 of SEQ ID NO:2. In another aspect, the variant comprises or consists of a substitution at a position corresponding to position 251 of SEQ ID NO:2 with Ala, Arg, Asn, Cys, Gln, Glu, Gly, His, Ile, Leu, Lys, Met, Phe, Pro, Ser, Thr, Trp, Tyr, or Val.

In another aspect, the variant comprises or consists of a substitution at a position corresponding to position 252 of SEQ ID NO:2. In another aspect, the variant comprises or consists of a substitution at a position corresponding to position 252 of SEQ ID NO:2 with Ala, Arg, Asn, Asp, Cys, Gln, Glu, Gly, His, Ile, Leu, Lys, Met, Phe, Pro, Ser, Thr, Trp, Tyr, or Val.

In another aspect, the variant comprises or consists of a substitution at a position corresponding to position 253 of SEQ ID NO:2. In another aspect, the variant comprises or consists of a substitution at a position corresponding to position 253 of SEQ ID NO:2 with Ala, Arg, Asn, Asp, Cys, Gln, Glu, Gly, His, Ile, Leu, Lys, Met, Phe, Pro, Ser, Thr, Trp, or Val.

In another aspect, the variant comprises or consists of a substitution at a position corresponding to position 256 of SEQ ID NO:2. In another aspect, the variant comprises or consists of a substitution at a position corresponding to position 256 of SEQ ID NO:2 with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of Ala as a substitution at a position corresponding to position 256 of SEQ ID NO:2. In another aspect, the variant comprises or consists of the substitution N256A.

In another aspect, the variant comprises or consists of a substitution at a position corresponding to position 258 of SEQ ID NO:2. In another aspect, the variant comprises or consists of a substitution at a position corresponding to position 258 of SEQ ID NO:2 with Ala, Arg, Asn, Asp, Cys, Gln, Glu, His, Ile, Leu, Lys, Met, Phe, Pro, Ser, Thr, Trp, Tyr, or Val.

In another aspect, the variant comprises or consists of a substitution at a position corresponding to position 260 of SEQ ID NO:2. In another aspect, the variant comprises or consists of a substitution at a position corresponding to position 260 of SEQ ID NO:2 with Ala, Arg, Asn, Asp, Cys, Gln, Gly, His, Ile, Leu, Lys, Met, Phe, Pro, Ser, Thr, Trp, Tyr, or Val.

In another aspect, the variant comprises or consists of a substitution at a position corresponding to position 268 of SEQ ID NO:2. In another aspect, the variant comprises or consists of a substitution at a position corresponding to position 268 of SEQ ID NO:2 with Ala, Arg, Asn, Asp, Cys, Gln, Gly, His, Ile, Leu, Lys, Met, Phe, Pro, Ser, Thr, Trp, Tyr, or Val. In another aspect, the variant comprises or consists of Gln as a substitution at a position corresponding to position 268 of SEQ ID NO:2. In another aspect, the variant comprises or consists of the substitution E268Q.

In another aspect, the variant comprises or consists of a substitution at a position corresponding to position 278 of SEQ ID NO:2. In another aspect, the variant comprises or consists of a substitution at a position corresponding to position 278 of SEQ ID NO:2 with Ala, Arg, Asn, Asp, Cys, Gln, Glu, Gly, His, Ile, Lys, Met, Phe, Pro, Ser, Thr, Trp, Tyr, or Val. In another aspect, the variant comprises or consists of Asn or Asp as a substitution at a position corresponding to position 278 of SEQ ID NO:2. In another aspect, the variant comprises or consists of the substitution L278N,D.

In another aspect, the variant comprises or consists of a substitution at a position corresponding to position 287 of SEQ ID NO:2. In another aspect, the variant comprises or consists of a substitution at a position corresponding to position 287 of SEQ ID NO:2 with Ala, Arg, Asn, Asp, Cys, Gln, Glu, Gly, His, Ile, Leu, Lys, Met, Phe, Pro, Ser, Thr, Trp, or Tyr. In another aspect, the variant comprises or consists of Asp or Glu as a substitution at a position corresponding to position 287 of SEQ ID NO:2. In another aspect, the variant comprises or consists of the substitution V287D,E.

In another aspect, the variant comprises or consists of a substitution at a position corresponding to position 288 of SEQ ID NO:2. In another aspect, the variant comprises or consists of a substitution at a position corresponding to position 288 of SEQ ID NO:2 with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of Asp as a substitution at a position corresponding to position 288 of SEQ ID NO:2. In another aspect, the variant comprises or consists of the substitution N288D.

In another aspect, the variant comprises or consists of a substitution at a position corresponding to position 300 of SEQ ID NO:2. In another aspect, the variant comprises or consists of a substitution at a position corresponding to position 300 of SEQ ID NO:2 with Ala, Arg, Asn, Asp, Cys, Gln, Glu, Gly, His, Ile, Lys, Met, Phe, Pro, Ser, Thr, Trp, Tyr, or Val.

In another aspect, the variant comprises or consists of a substitution at a position corresponding to position 301 of SEQ ID NO:2. In another aspect, the variant comprises or consists of a substitution at a position corresponding to position 301 of SEQ ID NO:2 with Ala, Arg, Asn, Asp, Cys, Gln, Glu, Gly, His, Ile, Leu, Lys, Met, Phe, Pro, Thr, Trp, Tyr, or Val.

In another aspect, the variant comprises or consists of a substitution at a position corresponding to position 302 of SEQ ID NO:2. In another aspect, the variant comprises or consists of a substitution at a position corresponding to position 302 of SEQ ID NO:2 with Ala, Arg, Asn, Asp, Cys, Gln, Glu, Gly, His, Ile, Leu, Lys, Met, Phe, Pro, Ser, Thr, Trp, or Val.

In another aspect, the variant comprises or consists of a substitution at a position corresponding to position 303 of SEQ ID NO:2. In another aspect, the variant comprises or consists of a substitution at a position corresponding to position 303 of SEQ ID NO:2 with Ala, Arg, Asn, Asp, Cys, Gln, Glu, Gly, His, Ile, Leu, Lys, Met, Phe, Pro, Thr, Trp, Tyr, or Val.

In another aspect, the variant comprises or consists of a substitution at a position corresponding to position 304 of SEQ ID NO:2. In another aspect, the variant comprises or consists of a substitution at a position corresponding to position 304 of SEQ ID NO:2 with Ala, Arg, Asp, Cys, Gln, Glu, Gly, His, Ile, Leu, Lys, Met, Phe, Pro, Ser, Thr, Trp, Tyr, or Val.

In another aspect, the variant comprises or consists of a substitution at a position corresponding to position 305 of SEQ ID NO:2. In another aspect, the variant comprises or consists of a substitution at a position corresponding to position 305 of SEQ ID NO:2 with Ala, Arg, Asn, Asp, Cys, Gln, Glu, Gly, His, Ile, Leu, Lys, Met, Pro, Ser, Thr, Trp, Tyr, or Val.

In another aspect, the variant comprises or consists of a substitution at a position corresponding to position 307 of SEQ ID NO:2. In another aspect, the variant comprises or consists of a substitution at a position corresponding to position 307 of SEQ ID NO:2 with Ala, Arg, Asn, Asp, Cys, Gln, Glu, Gly, Ile, Leu, Lys, Met, Phe, Pro, Ser, Thr, Trp, Tyr, or Val. In another aspect, the variant comprises or consists of Arg as a substitution at a position corresponding to position 307 of SEQ ID NO:2. In another aspect, the variant comprises or consists of the substitution H307R.

In another aspect, the variant comprises or consists of a substitution at a position corresponding to position 314 of SEQ ID NO:2. In another aspect, the variant comprises or consists of a substitution at a position corresponding to position 314 of SEQ ID NO:2 with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of Glu as a substitution at a position corresponding to position 314 of SEQ ID NO:2. In another aspect, the variant comprises or consists of the substitution Q314E.

In another aspect, the variant comprises or consists of a substitution at a position corresponding to position 315 of SEQ ID NO:2. In another aspect, the variant comprises or consists of a substitution at a position corresponding to position 315 of SEQ ID NO:2 with Ala, Arg, Asn, Asp, Cys, Gln, Glu, Gly, His, Ile, Leu, Lys, Met, Phe, Ser, Thr, Trp, Tyr, or Val.

In another aspect, the variant comprises or consists of a substitution at a position corresponding to position 316 of SEQ ID NO:2. In another aspect, the variant comprises or consists of a substitution at a position corresponding to position 316 of SEQ ID NO:2 with Ala, Arg, Asn, Asp, Cys, Gln, Glu, Gly, His, Ile, Leu, Lys, Met, Phe, Pro, Ser, Thr, Trp, or Tyr.

In another aspect, the variant comprises or consists of a substitution at a position corresponding to position 317 of SEQ ID NO:2. In another aspect, the variant comprises or consists of a substitution at a position corresponding to position 317 of SEQ ID NO:2 with Ala, Arg, Asn, Asp, Cys, Gln, Gly, His, Ile, Leu, Lys, Met, Phe, Pro, Ser, Thr, Trp, Tyr, or Val.

In another aspect, the variant comprises or consists of a substitution at a position corresponding to position 318 of SEQ ID NO:2. In another aspect, the variant comprises or consists of a substitution at a position corresponding to position 318 of SEQ ID NO:2 with Ala, Arg, Asp, Cys, Gln, Glu, Gly, His, Ile, Leu, Lys, Met, Phe, Pro, Ser, Thr, Trp, Tyr, or Val.

In another aspect, the variant comprises or consists of a substitution at a position corresponding to position 319 of SEQ ID NO:2. In another aspect, the variant comprises or consists of a substitution at a position corresponding to position 319 of SEQ ID NO:2 with Ala, Arg, Asn, Asp, Cys, Gln, Glu, Gly, His, Ile, Leu, Lys, Met, Pro, Ser, Thr, Trp, Tyr, or Val.

In another aspect, the variant comprises or consists of a substitution at a position corresponding to position 320 of SEQ ID NO:2. In another aspect, the variant comprises or consists of a substitution at a position corresponding to position 320 of SEQ ID NO:2 with Ala, Arg, Asn, Asp, Cys, Gln, Glu, Gly, His, Ile, Leu, Lys, Met, Phe, Pro, Ser, Thr, Trp, or Val.

In another aspect, the variant comprises or consists of a substitution at a position corresponding to position 321 of SEQ ID NO:2. In another aspect, the variant comprises or consists of a substitution at a position corresponding to position 321 of SEQ ID NO:2 with Arg, Asn, Asp, Cys, Gln, Glu, Gly, His, Ile, Leu, Lys, Met, Phe, Pro, Ser, Thr, Trp, Tyr, or Val.

In another aspect, the variant comprises or consists of a substitution at a position corresponding to position 322 of SEQ ID NO:2. In another aspect, the variant comprises or consists of a substitution at a position corresponding to position 322 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Arg as a substitution at a position corresponding to position 322 of SEQ ID NO:2. In another aspect, the variant comprises or consists of the substitution K322R.

In another aspect, the variant comprises or consists of a substitution at a position corresponding to position 323 of SEQ ID NO:2. In another aspect, the variant comprises or consists of a substitution at a position corresponding to position 323 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Arg as a substitution at a position corresponding to position 323 of SEQ ID NO:2. In another aspect, the variant comprises or consists of the substitution K323R.

In another aspect, the variant comprises or consists of a substitution at a position corresponding to position 327 of SEQ ID NO:2. In another aspect, the variant comprises or consists of a substitution at a position corresponding to position 327 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Arg as a substitution at a position corresponding to position 327 of SEQ ID NO:2. In another aspect, the variant comprises or consists of the substitution K327R.

In another aspect, the variant comprises or consists of a substitution at a position corresponding to position 330 of SEQ ID NO:2. In another aspect, the variant comprises or consists of a substitution at a position corresponding to position 330 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Arg as a substitution at a position corresponding to position 330 of SEQ ID NO:2. In another aspect, the variant comprises or consists of the substitution K330R.

In another aspect, the variant comprises or consists of a substitution at a position corresponding to position 332 of SEQ ID NO:2. In another aspect, the variant comprises or consists of a substitution at a position corresponding to position 332 of SEQ ID NO:2 with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of Ala or Pro as a substitution at a position corresponding to position 332 of SEQ ID NO:2. In another aspect, the variant comprises or consists of the substitution D332A,P.

In another aspect, the variant comprises or consists of a substitution at a position corresponding to position 342 of SEQ ID NO:2. In another aspect, the variant comprises or consists of a substitution at a position corresponding to position 342 of SEQ ID NO:2 with Ala, Arg, Asn, Asp, Cys, Gln, Glu, Gly, His, Ile, Leu, Lys, Met, Pro, Ser, Thr, Trp, Tyr, or Val. In another aspect, the variant comprises or consists of Tyr as a substitution at a position corresponding to position 342 of SEQ ID NO:2. In another aspect, the variant comprises or consists of the substitution F342Y.

In another aspect, the variant comprises or consists of a substitution at a position corresponding to position 347 of SEQ ID NO:2. In another aspect, the variant comprises or consists of a substitution at a position corresponding to position 347 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Arg as a substitution at a position corresponding to position 347 of SEQ ID NO:2. In another aspect, the variant comprises or consists of the substitution K347R.

In another aspect, the variant comprises or consists of a substitution at a position corresponding to position 349 of SEQ ID NO:2. In another aspect, the variant comprises or consists of a substitution at a position corresponding to position 349 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Arg as a substitution at a position corresponding to position 349 of SEQ ID NO:2. In another aspect, the variant comprises or consists of the substitution K349R.

In another aspect, the variant comprises or consists of a substitution at a position corresponding to position 352 of SEQ ID NO:2. In another aspect, the variant comprises or consists of a substitution at a position corresponding to position 352 of SEQ ID NO:2 with Ala, Arg, Asn, Asp, Cys, Gln, Glu, Gly, His, Ile, Leu, Lys, Met, Pro, Ser, Thr, Trp, Tyr, or Val.

In another aspect, the variant comprises or consists of a substitution at a position corresponding to position 353 of SEQ ID NO:2. In another aspect, the variant comprises or consists of a substitution at a position corresponding to position 353 of SEQ ID NO:2 with Ala, Arg, Asn, Asp, Cys, Gln, Glu, Gly, His, Ile, Leu, Lys, Met, Phe, Pro, Ser, Thr, Tyr, or Val.

In another aspect, the variant comprises or consists of a substitution at a position corresponding to position 354 of SEQ ID NO:2. In another aspect, the variant comprises or consists of a substitution at a position corresponding to position 354 of SEQ ID NO:2 with Ala, Arg, Asn, Asp, Cys, Gln, Glu, Gly, His, Ile, Leu, Lys, Met, Pro, Ser, Thr, Trp, Tyr, or Val. In another aspect, the variant comprises or consists of Tyr as a substitution at a position corresponding to position 354 of SEQ ID NO:2. In another aspect, the variant comprises or consists of the substitution F354Y.

In another aspect, the variant comprises or consists of a substitution at a position corresponding to position 355 of SEQ ID NO:2. In another aspect, the variant comprises or consists of a substitution at a position corresponding to position 355 of SEQ ID NO:2 with Ala, Arg, Asn, Asp, Cys, Gln, Glu, Gly, His, Ile, Leu, Lys, Met, Phe, Pro, Ser, Thr, Trp, Tyr, or Val.

In another aspect, the variant comprises or consists of a substitution at a position corresponding to position 356 of SEQ ID NO:2. In another aspect, the variant comprises or consists of a substitution at a position corresponding to position 356 of SEQ ID NO:2 with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of Glu as a substitution at a position corresponding to position 356 of SEQ ID NO:2. In another aspect, the variant comprises or consists of the substitution Q356E.

In another aspect, the variant comprises or consists of a substitution at a position corresponding to position 357 of SEQ ID NO:2. In another aspect, the variant comprises or consists of a substitution at a position corresponding to position 357 of SEQ ID NO:2 with Ala, Arg, Asn, Asp, Cys, Gln, Glu, Gly, His, Ile, Lys, Met, Phe, Pro, Ser, Thr, Trp, Tyr, or Val.

In another aspect, the variant comprises or consists of a substitution at a position corresponding to position 358 of SEQ ID NO:2. In another aspect, the variant comprises or consists of a substitution at a position corresponding to position 358 of SEQ ID NO:2 with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of Asn as a substitution at a position corresponding to position 358 of SEQ ID NO:2. In another aspect, the variant comprises or consists of the substitution D358N.

In another aspect, the variant comprises or consists of a substitution at a position corresponding to position 363 of SEQ ID NO:2. In another aspect, the variant comprises or consists of a substitution at a position corresponding to position 363 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Arg as a substitution at a position corresponding to position 363 of SEQ ID NO:2. In another aspect, the variant comprises or consists of the substitution K363R.

In another aspect, the variant comprises or consists of a substitution at a position corresponding to position 366 of SEQ ID NO:2. In another aspect, the variant comprises or consists of a substitution at a position corresponding to position 366 of SEQ ID NO:2 with Ala, Arg, Asn, Asp, Cys, Glu, Gly, His, Ile, Leu, Lys, Met, Phe, Pro, Ser, Thr, Trp, Tyr, or Val. In another aspect, the variant comprises or consists of Glu as a substitution at a position corresponding to position 366 of SEQ ID NO:2. In another aspect, the variant comprises or consists of the substitution Q366E.

In another aspect, the variant comprises or consists of a substitution at a position corresponding to position 368 of SEQ ID NO:2. In another aspect, the variant comprises or consists of a substitution at a position corresponding to position 368 of SEQ ID NO:2 with Ala, Arg, Asn, Asp, Cys, Gln, Glu, Gly, His, Ile, Leu, Lys, Met, Phe, Pro, Ser, Trp, Tyr, or Val. In another aspect, the variant comprises or consists of Ser as a substitution at a position corresponding to position 368 of SEQ ID NO:2. In another aspect, the variant comprises or consists of the substitution T368S.

In another aspect, the variant comprises or consists of a substitution at a position corresponding to position 374 of SEQ ID NO:2. In another aspect, the variant comprises or consists of a substitution at a position corresponding to position 374 of SEQ ID NO:2 with Ala, Arg, Asn, Asp, Cys, Gln, Gly, His, Ile, Leu, Lys, Met, Phe, Pro, Ser, Thr, Trp, Tyr, or Val. In another aspect, the variant comprises or consists of Ser or Gln as a substitution at a position corresponding to position 374 of SEQ ID NO:2. In another aspect, the variant comprises or consists of the substitution E374S,Q.

In another aspect, the variant comprises or consists of a substitution at a position corresponding to position 382 of SEQ ID NO:2. In another aspect, the variant comprises or consists of a substitution at a position corresponding to position 382 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Leu or Val as a substitution at a position corresponding to position 382 of SEQ ID NO:2. In another aspect, the variant comprises or consists of the substitution K382L,V.

In another aspect, the variant comprises or consists of a substitution at a position corresponding to position 383 of SEQ ID NO:2. In another aspect, the variant comprises or consists of a substitution at a position corresponding to position 383 of SEQ ID NO:2 with Ala, Arg, Asn, Asp, Cys, Gln, Glu, Gly, His, Ile, Lys, Met, Phe, Pro, Ser, Thr, Trp, Tyr, or Val. In another aspect, the variant comprises or consists of Asp as a substitution at a position corresponding to position 383 of SEQ ID NO:2. In another aspect, the variant comprises or consists of the substitution L383D.

In another aspect, the variant comprises or consists of a substitution at a position corresponding to position 385 of SEQ ID NO:2. In another aspect, the variant comprises or consists of a substitution at a position corresponding to position 385 of SEQ ID NO:2 with Ala, Arg, Asn, Asp, Cys, Gln, Glu, Gly, His, Ile, Lys, Met, Phe, Pro, Ser, Thr, Trp, Tyr, or Val.

In another aspect, the variant comprises or consists of a substitution at a position corresponding to position 386 of SEQ ID NO:2. In another aspect, the variant comprises or consists of a substitution at a position corresponding to position 386 of SEQ ID NO:2 with Ala, Arg, Asn, Asp, Cys, Gln, Glu, Gly, His, Leu, Lys, Met, Phe, Pro, Ser, Thr, Trp, Tyr, or Val.

In another aspect, the variant comprises or consists of a substitution at a position corresponding to position 387 of SEQ ID NO:2. In another aspect, the variant comprises or consists of a substitution at a position corresponding to position 387 of SEQ ID NO:2 with Ala, Arg, Asn, Asp, Cys, Glu, Gly, His, Ile, Leu, Lys, Met, Phe, Pro, Ser, Thr, Trp, Tyr, or Val.

In another aspect, the variant comprises or consists of a substitution at a position corresponding to position 388 of SEQ ID NO:2. In another aspect, the variant comprises or consists of a substitution at a position corresponding to position 388 of SEQ ID NO:2 with Ala, Arg, Asn, Asp, Cys, Gln, Glu, Gly, His, Ile, Leu, Lys, Met, Pro, Ser, Thr, Trp, Tyr, or Val.

In another aspect, the variant comprises or consists of a substitution at a position corresponding to position 389 of SEQ ID NO:2. In another aspect, the variant comprises or consists of a substitution at a position corresponding to position 389 of SEQ ID NO:2 with Ala, Arg, Asn, Asp, Cys, Gln, Glu, Gly, His, Ile, Leu, Lys, Met, Phe, Pro, Ser, Thr, Trp, or Val.

In another aspect, the variant comprises or consists of a substitution at a position corresponding to position 390 of SEQ ID NO:2. In another aspect, the variant comprises or consists of a substitution at a position corresponding to position 390 of SEQ ID NO:2 with Ala, Arg, Asn, Cys, Gln, Glu, Gly, His, Ile, Leu, Lys, Met, Phe, Pro, Ser, Thr, Trp, Tyr, or Val.

In another aspect, the variant comprises or consists of a substitution at a position corresponding to position 391 of SEQ ID NO:2. In another aspect, the variant comprises or consists of a substitution at a position corresponding to position 391 of SEQ ID NO:2 with Ala, Asn, Asp, Cys, Gln, Glu, Gly, His, Ile, Leu, Lys, Met, Phe, Pro, Ser, Thr, Trp, Tyr, or Val.

In another aspect, the variant comprises or consists of a substitution at a position corresponding to position 392 of SEQ ID NO:2. In another aspect, the variant comprises or consists of a substitution at a position corresponding to position 392 of SEQ ID NO:2 with Ala, Arg, Asn, Asp, Cys, Gln, Glu, Gly, His, Ile, Leu, Lys, Met, Phe, Pro, Ser, Thr, Trp, or Val.

In another aspect, the variant comprises or consists of a substitution at a position corresponding to position 393 of SEQ ID NO:2. In another aspect, the variant comprises or consists of a substitution at a position corresponding to position 393 of SEQ ID NO:2 with Ala, Arg, Asn, Cys, Gln, Glu, Gly, His, Ile, Leu, Lys, Met, Phe, Pro, Ser, Thr, Trp, Tyr, or Val. In another aspect, the variant comprises or consists of Ser or Thr as a substitution at a position corresponding to position 393 of SEQ ID NO:2. In another aspect, the variant comprises or consists of the substitution D393S,T

In another aspect, the variant comprises or consists of a substitution at a position corresponding to position 400 of SEQ ID NO:2. In another aspect, the variant comprises or consists of a substitution at a position corresponding to position 400 of SEQ ID NO:2 with Ala, Arg, Asn, Asp, Cys, Gln, Gly, His, Ile, Leu, Lys, Met, Phe, Pro, Ser, Thr, Trp, Tyr, or Val. In another aspect, the variant comprises or consists of Ser or Thr as a substitution at a position corresponding to position 400 of SEQ ID NO:2. In another aspect, the variant comprises or consists of the substitution E400S,T.

In another aspect, the variant comprises or consists of a substitution at a position corresponding to position 403 of SEQ ID NO:2. In another aspect, the variant comprises or consists of a substitution at a position corresponding to position 403 of SEQ ID NO:2 with Ala, Arg, Asn, Asp, Cys, Gln, Glu, Gly, His, Ile, Leu, Lys, Met, Pro, Ser, Thr, Trp, Tyr, or Val. In another aspect, the variant comprises or consists of Glu or Asp as a substitution at a position corresponding to position 403 of SEQ ID NO:2. In another aspect, the variant comprises or consists of the substitution F403E,D.

In another aspect, the variant comprises or consists of a substitution at a position corresponding to position 411 of SEQ ID NO:2. In another aspect, the variant comprises or consists of a substitution at a position corresponding to position 411 of SEQ ID NO:2 with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of Asp or Glu as a substitution at a position corresponding to position 411 of SEQ ID NO:2. In another aspect, the variant comprises or consists of the substitution N411D,E.

In another aspect, the variant comprises or consists of a substitution at a position corresponding to position 415 of SEQ ID NO:2. In another aspect, the variant comprises or consists of a substitution at a position corresponding to position 415 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Arg as a substitution at a position corresponding to position 415 of SEQ ID NO:2. In another aspect, the variant comprises or consists of the substitution K415R.

In another aspect, the variant comprises or consists of a substitution at a position corresponding to position 419 of SEQ ID NO:2. In another aspect, the variant comprises or consists of a substitution at a position corresponding to position 419 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Glu, Asp, or Arg as a substitution at a position corresponding to position 419 of SEQ ID NO:2. In another aspect, the variant comprises or consists of the substitution K419E,D,R. In another aspect, the variant comprises or consists of the substitutions K419E+K440R. In another aspect, the variant comprises or consists of the substitutions K419R+K440E.

In another aspect, the variant comprises or consists of a substitution at a position corresponding to position 420 of SEQ ID NO:2. In another aspect, the variant comprises or consists of a substitution at a position corresponding to position 420 of SEQ ID NO:2 with Ala, Arg, Asn, Asp, Cys, Gln, Glu, Gly, His, Ile, Leu, Lys, Met, Phe, Pro, Thr, Trp, Tyr, or Val. In another aspect, the variant comprises or consists of Glu or Asp as a substitution at a position corresponding to position 420 of SEQ ID NO:2. In another aspect, the variant comprises or consists of the substitution S420E,D.

In another aspect, the variant comprises or consists of a substitution at a position corresponding to position 424 of SEQ ID NO:2. In another aspect, the variant comprises or consists of a substitution at a position corresponding to position 424 of SEQ ID NO:2 with Ala, Arg, Asn, Asp, Cys, Gln, Glu, Gly, His, Ile, Leu, Lys, Phe, Pro, Ser, Thr, Trp, Tyr, or Val. In another aspect, the variant comprises or consists of Val or Ala as a substitution at a position corresponding to position 424 of SEQ ID NO:2. In another aspect, the variant comprises or consists of the substitution M424V,A.

In another aspect, the variant comprises or consists of a substitution at a position corresponding to position 425 of SEQ ID NO:2. In another aspect, the variant comprises or consists of a substitution at a position corresponding to position 425 of SEQ ID NO:2 with Ala, Arg, Asn, Asp, Cys, Gln, Glu, Gly, His, Ile, Leu, Lys, Met, Phe, Pro, Ser, Thr, Trp, or Val.

In another aspect, the variant comprises or consists of a substitution at a position corresponding to position 427 of SEQ ID NO:2. In another aspect, the variant comprises or consists of a substitution at a position corresponding to position 427 of SEQ ID NO:2 with Ala, Arg, Asp, Cys, Gln, Glu, Gly, His, Ile, Leu, Lys, Met, Phe, Pro, Ser, Thr, Trp, Tyr, or Val.

In another aspect, the variant comprises or consists of a substitution at a position corresponding to position 428 of SEQ ID NO:2. In another aspect, the variant comprises or consists of a substitution at a position corresponding to position 428 of SEQ ID NO:2 with Ala, Arg, Asn, Asp, Cys, Gln, Glu, Gly, His, Ile, Leu, Lys, Met, Phe, Pro, Ser, Thr, Trp, or Val.

In another aspect, the variant comprises or consists of a substitution at a position corresponding to position 429 of SEQ ID NO:2. In another aspect, the variant comprises or consists of a substitution at a position corresponding to position 429 of SEQ ID NO:2 with Arg, Asn, Asp, Cys, Gln, Glu, Gly, His, Ile, Leu, Lys, Met, Phe, Pro, Ser, Thr, Trp, Tyr, or Val.

In another aspect, the variant comprises or consists of a substitution at a position corresponding to position 433 of SEQ ID NO:2. In another aspect, the variant comprises or consists of a substitution at a position corresponding to position 433 of SEQ ID NO:2 with Ala, Arg, Asn, Asp, Cys, Gln, Glu, Gly, His, Ile, Leu, Lys, Phe, Pro, Ser, Thr, Trp, Tyr, or Val. In another aspect, the variant comprises or consists of Val or Ala as a substitution at a position corresponding to position 433 of SEQ ID NO:2. In another aspect, the variant comprises or consists of the substitution M433V,A.

In another aspect, the variant comprises or consists of a substitution at a position corresponding to position 437 of SEQ ID NO:2. In another aspect, the variant comprises or consists of a substitution at a position corresponding to position 437 of SEQ ID NO:2 with Ala, Arg, Asn, Asp, Cys, Gln, Glu, Gly, His, Ile, Leu, Lys, Met, Phe, Pro, Ser, Thr, Trp, or Val. In another aspect, the variant comprises or consists of Glu as a substitution at a position corresponding to position 437 of SEQ ID NO:2. In another aspect, the variant comprises or consists of the substitution Y437E.

In another aspect, the variant comprises or consists of a substitution at a position corresponding to position 440 of SEQ ID NO:2. In another aspect, the variant comprises or consists of a substitution at a position corresponding to position 440 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Glu, Asp or Arg as a substitution at a position corresponding to position 440 of SEQ ID NO:2. In another aspect, the variant comprises or consists of the substitution K440E,D,R. In another aspect, the variant comprises or consists of the substitutions K419E+K440R. In another aspect, the variant comprises or consists of the substitutions K419R+K440E.

In another aspect, the variant comprises or consists of a substitution at a position corresponding to position 445 of SEQ ID NO:2. In another aspect, the variant comprises or consists of a substitution at a position corresponding to position 445 of SEQ ID NO:2 with Ala, Arg, Asn, Asp, Cys, Gln, Gly, His, Ile, Leu, Lys, Met, Phe, Pro, Ser, Thr, Trp, Tyr, or Val. In another aspect, the variant comprises or consists of Ala or Pro as a substitution at a position corresponding to position 445 of SEQ ID NO:2. In another aspect, the variant comprises or consists of the substitution E445A,P.

In another aspect, the variant comprises or consists of a substitution at a position corresponding to position 456 of SEQ ID NO:2. In another aspect, the variant comprises or consists of a substitution at a position corresponding to position 456 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Arg as a substitution at a position corresponding to position 456 of SEQ ID NO:2. In another aspect, the variant comprises or consists of the substitution K456R.

In another aspect, the variant comprises or consists of a substitution at a position corresponding to position 460 of SEQ ID NO:2. In another aspect, the variant comprises or consists of a substitution at a position corresponding to position 460 of SEQ ID NO:2 with Ala, Arg, Asn, Asp, Cys, Gln, Glu, Gly, His, Ile, Leu, Lys, Met, Phe, Pro, Ser, Trp, Tyr, or Val. In another aspect, the variant comprises or consists of Glu or Asp as a substitution at a position corresponding to position 460 of SEQ ID NO:2. In another aspect, the variant comprises or consists of the substitution T460E,D.

In another aspect, the variant comprises or consists of a substitution at a position corresponding to position 472 of SEQ ID NO:2. In another aspect, the variant comprises or consists of a substitution at a position corresponding to position 472 of SEQ ID NO:2 with Ala, Arg, Asn, Asp, Cys, Gln, Glu, Gly, His, Ile, Leu, Lys, Met, Phe, Pro, Ser, Thr, Trp, or Tyr. In another aspect, the variant comprises or consists of Thr as a substitution at a position corresponding to position 472 of SEQ ID NO:2. In another aspect, the variant comprises or consists of the substitution V472T.

In another aspect, the variant comprises or consists of a substitution at a position corresponding to position 473 of SEQ ID NO:2. In another aspect, the variant comprises or consists of a substitution at a position corresponding to position 473 of SEQ ID NO:2 with Ala, Arg, Asn, Asp, Cys, Gln, Glu, Gly, His, Ile, Leu, Met, Phe, Pro, Ser, Thr, Trp, Tyr, or Val. In another aspect, the variant comprises or consists of Arg as a substitution at a position corresponding to position 473 of SEQ ID NO:2. In another aspect, the variant comprises or consists of the substitution K473R.

Carbohydrate oxidase variants directed toward having improved the thermal stability as compared to the parent carbohydrate oxidase include variants comprising substitutions at a position corresponding to one or more of the following positions: 19, 22, 60, 81, 98, 118, 138, 146, 152, 170, 188, 354, 368, 374 and 420 using SEQ ID NO:2 for numbering, including, e.g., T19S, Y22W, N60G, N81D, N98D, V118R,K, S138G, F146Y, M152T, G170S, K188R, F354Y, T368S, E374S, and/or S420E,D in SEQ ID NO:2 and homologous carbohydrate oxidases.

Carbohydrate oxidase variants having one or more extra hydrogen bond and directed towards having improved thermal stability as compared to the parent carbohydrate oxidase include variants comprising substitutions at a position corresponding to one or more of the following positions: 77, 184, 342, and 472 using SEQ ID NO:2 for numbering, including, e.g., F77Y, F184Y, F342Y and/or V472T in SEQ ID NO:2 and homologous carbohydrate oxidases.

Carbohydrate oxidase variants having an extra disulfide binding and directed towards having improved thermal stability as compared to the parent carbohydrate oxidase include T43C/A52C and/or V157C/K188C in SEQ ID NO:2 and homologous carbohydrate oxidases.

Additional carbohydrate oxidase variants directed towards having improved thermal stability as compared to the parent carbohydrate oxidase include variants comprising substitutions at a position corresponding to one or more of the following positions: 4, 15, 27, 31, 48, 57, 54, 58, 59, 81, 93, 98, 105, 114, 148, 222, 224, 227, 278, 287, 288, 330, 356, 366, 383, 403, 411, 419, 437, 460 using SEQ ID NO:2 for numbering, including, e.g., E4Q, D15N, Q356E, H48R,D,S,T, K419E,R, K440E,R (including, e.g., K419E+K440R or K419R+K440E), K57D, T460E,D, D93S,T, E27R,K, K58D,E, Q54E,D, K330R, N222D, N81D, N411D,E, N288D, N98D, Q366E, L227D,E, L278N,D, L383D, L59D, V287D,E, T224E,D, F403E,D, Y437E, F148D,E, H105D,E, H114R, and/or 131R in SEQ ID NO:2 and homologous carbohydrate oxidases.

Preferably, the carbohydrate oxidase variants having improved thermal stability as compared to the parent carbohydrate oxidase include variants comprising substitutions at a position corresponding to one or more of the following positions: 22, 27, 54, 98, 222, 460 using SEQ ID NO:2 for numbering, including, e.g., Y22W, E27R, Q54D, N98D, N222D, and/or T460E in SEQ ID NO:2 and homologous carbohydrate oxidases.

Carbohydrate oxidase variants directed toward having reduced oxidation as compared to the parent carbohydrate oxidase, e.g., oxidation caused by H2O2, include variants comprising a substitution of a methinoine residue at a position corresponding to one or more of the following positions 85, 91, 152, 424 and 433 using SEQ ID NO:2 for numbering, including, e.g., M85V,A, M91A, M152A, M424V,A and/or M433V,A in SEQ ID NO:2 and homologous carbohydrate oxidases.

Carbohydrate oxidase variants directed towards having improved stability at low pH (e.g., pH 5 or lower) conditions as compared to the parent carbohydrate oxidase include variants comprising substitution at a position corresponding to one or more of the following positions: 21, 105, 153, 174, 231, 235, 268, 374, 393, and 400 using SEQ ID NO:2 for numbering, including, e.g., E231Q, E400S,T, D393S,T, H105Q,N, H153N, E174Q, E268Q, D235N, D21N, and/or E374Q in SEQ ID NO:2 or homologous carbohydrate oxidases.

Carbohydrate oxidase variants directed towards having improved stability at high pH (e.g., pH 8 or higher) as compared to the parent carbohydrate oxidase include variants comprising a substitution at a position corresponding to one or more of the following positions: 15, 114, 188, 201, 223, and 207; preferably 15, 188, and 201; using SEQ ID NO:2 for numbering, including, e.g., K223R, K201R, K188R, D15N, H114R and/or H307R; preferably D15N, K188R and/or K201R; in SEQ ID NO:2 or homologous carbohydrate oxidases.

Carbohydrate oxidase variants directed towards having reduced demindation (e.g., as a result of high temperature and extreme pH values) as compared to the parent carbohydrate oxidase include variants comprising a substitution at a position corresponding to one or more of the following positions: 81 and 256 using SEQ ID NO:2 for numbering, including, e.g., N81A,H and/or N256A in SEQ ID NO:2 or homologous carbohydrate oxidases.

Carbohydrate oxidase variants directed towards having a reduced susceptibility to amide hydrolysis of peptide bonds, in particular, at low pH include variants comprising a substitution at a position corresponding to one or more of the following positions 80, 169, 332 and 445 using SEQ ID NO:2 for numbering, including, e.g., D169A, D332A,P, E445A,P and/or E80A in SEQ ID NO:2 or homologous carbohydrate oxidases.

Carbohydrate oxidase variants directed towards having an altered specificity to a substrate molecule and/or inhibitor include variants comprising a substitution at a position corresponding to one or more of the following positions: 29, 30, 69, 70, 71, 72, 112, 129, 130, 131, 132, 133, 134, 135, 140, 145, 146, 147, 148, 213, 248, 249, 250, 251, 252, 253, 258, 260, 300, 301, 302, 303, 304, 305, 314, 315, 316, 317, 318, 319, 320, 321, 322, 323, 352, 353, 354, 355, 356, 357, 358, 385, 386, 387, 388, 389, 390, 391, 392, 425, 427, 428, 429, using SEQ ID NO:2 for numbering. The structure of SEQ ID NO:2 with and without an inhibitor has been identified and the atomic coordinates are identified in Appendix 1. The inhibitor present in the structure in Appendix 1 gives a very clear picture of how a disaccharide substrate molecule would look like in the context of the enzyme. The amino acid residues within different distance ranges from the inhibitor are as follows:

Within 3.0 Å: Y72, R391.

Within 4.0 Å: F29, Y72, T131, C132, R248, Y252, N304, E317, F354, Q356, Q387, Y389, R391, Y428.

Within 5.0 Å: F29, Y72, T131, C132, R248, Y252, E260, N304, E317, F352, F354, Q356, Q387, Y389, R391, Y428.

Within 6.0 Å: F29, H70, Y72, T131, C132, F146, R248, Y252, E260, Y302, N304, E317, F319, F352, F354, Q356, Q387, Y389, R391, Y428.

Within 7.0 Å: F29, H70, Y72, G130, T131, C132, V135, F146, F148, R213, R248, Y252, E260, Y302, S303, N304, Q314, E317, F319, F352, F354, Q356, D358, Q387, Y389, R391, Y425, N427, Y428.

Within 8.0 Å: F29, H70, Y72, G130, T131, C132, V135, H140, F146, G147, F148, R213, R248, G250, D251, Y252, G253, E260, S301, Y302, S303, N304, F305, Q314, P315, E317, F319, A321, F352, W353, F354, Y355, Q356, D358, L385, Q387, Y389, R391, Y425, N427, Y428.

Within 9.0 Å: F29, N30, H70, S71, Y72, L112, H129, G130, T131, C132, P133, V135, H140, G145, F146, G147, F148, R213, R248, G250, D251, Y252, G253, E260, S301, Y302, S303, N304, F305, Q314, P315, V316, E317, N318, F319, Y320, A321, F352, W353, F354, Y355, Q356, D358, L385, I386, Q387, F388, Y389, D390, R391, Y425, N427, Y428.

Within 10.0 Å: F29, N30, G69, H70, S71, Y72, L112, H129, G130, T131, C132, P133, G134, V135, H140, G145, F146, G147, F148, R213, R248, I249, G250, D251, Y252, G253, G258, E260, L300, S301, Y302, S303, N304, F305, Q314, P315, V316, E317, N318, F319, Y320, A321, K322, S323, F352, W353, F354, Y355, Q356, L357, D358, L385, I386, Q387, F388, Y389, D390, R391, Y392, Y425, N427, Y428, A429.

Carbohydrate oxidase variants directed towards altering the activity pH include variants comprising a substitution at a position corresponding to one or more of the following positions 148, 324, and/or 385 using SEQ ID NO:2 for numbering, including, e.g., F148D,E, D358N, and/or Q314E in SEQ ID NO:2 or homologous carbohydrate oxidases.

Carbohydrate oxidase variants directed towards having increased stability toward anionic surfactants include variants comprising a substitution at a position corresponding to one or more of the following positions 58, 62, 122, 188, 201, 221, 223, 228, 322, 327, 347, 349, 363, 382, 415, 419, 440, 456, 473, using SEQ ID NO:2 for numbering, including, e.g., K122R, K201R, K188L,V, K62L,V, K58R, K456R, K473R, K363R, K228R, K221R, K349R, K347R, K415R, K322R, K440E,D, K419E,D, K327R, K382L,V, and/or K223R in SEQ ID NO:2 or homologous carbohydrate oxidases.

### Polynucleotides

The present invention relates to isolated polynucleotides that encode the carbohydrate oxidase variants described herein above. The present invention also relates to isolated polynucleotides that encode variants of a parent carbohydrate oxidase, wherein the polynucleotides encode carbohydrate oxidase variants comprising a substitution at one or more (several) positions corresponding to positions 4, 15, 19, 21, 22, 27, 29, 30, 31, 43, 48, 52, 54, 57, 58, 59, 60, 62, 69, 70, 71, 72, 77, 80, 81, 85, 91, 93, 98, 105, 112, 114, 118, 122, 129, 130, 131, 132, 133, 134, 135, 138, 140, 146, 147, 148, 152, 153, 157, 169, 170, 174, 184, 188, 201, 213, 221, 222, 223, 224, 227, 228, 231, 235, 248, 249, 250, 251, 252, 253, 256, 258, 260, 268, 278, 287, 288, 300, 301, 302, 303, 304, 305, 307, 314, 315, 316, 317, 318, 319, 320, 321, 322, 323, 327, 330, 332, 342, 347, 349, 352, 353, 354, 355, 356, 357, 358, 363, 366, 368, 374, 382, 383, 385, 386, 387, 388, 389, 390, 391, 392, 393, 400, 403, 411, 415, 419, 420, 424, 425, 427, 428, 429, 433, 437, 440, 445, 456, 460, 472, and 473 of SEQ ID NO:2, wherein the variant carbohydrate oxidase is (a) a polypeptide comprising an amino acid sequence having at least 60% sequence identity to SEQ ID NO:2, at least 65% identity, at least 70% identity, at least 75% identity, at least 80% identity, at least 85% identity, at least 90% identity, at least 95% identity, at least 96% identity, at least 97% identity, at least 98% identity, at least 99% identity with SEQ ID NO:2 (ii) a polypeptide encoded by a polynucleotide that hybridizes under preferably low, more preferably low-medium, more preferably medium, even more preferably medium-high, most preferably high, or even most preferably very high stringency conditions with (i) the mature polypeptide coding sequence of SEQ ID NO:1, (ii) cDNA sequence encoding the mature polypeptide coding sequence of SEQ ID NO:1, or (iii) a complementary strand of (i) or (ii), wherein the variant has carbohydrate oxidase activity.

### Nucleic Acid Constructs

The present invention also relates to nucleic acid constructs comprising a polynucleotide encoding a carbohydrate oxidase variant of the present invention operably linked to one or more (several) control sequences that direct the expression of the coding sequence in a suitable host cell under conditions compatible with the control sequences.

An isolated polynucleotide encoding a carbohydrate oxidase variant of the present invention may be manipulated in a variety of ways to provide for expression of the variant. Manipulation of the polynucleotide prior to its insertion into a vector may be desirable or necessary depending on the expression vector. The techniques for modifying polynucleotides utilizing recombinant DNA methods are well known in the art.

The control sequence may be an appropriate promoter sequence, which is recognized by a host cell for expression of the polynucleotide. The promoter sequence contains transcriptional control sequences that mediate the expression of the variant carbohydrate oxidase. The promoter may be any nucleic acid sequence that shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25). Further promoters are described in “Useful proteins from recombinant bacteria” in Scientific American, 1980, 242: 74-94; and in Sambrook et al., 1989, supra.

Examples of suitable promoters for directing the transcription of the nucleic acid constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium venenatum amyloglucosidase (WO 00/56900), Fusarium venenatum Daria (WO 00/56900), Fusarium venenatum Quinn (WO 00/56900), Fusarium oxysporum trypsin-like protease (WO 96/00787), Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, which is recognized by a host cell to terminate transcription. The terminator sequence is operably linked to the 3′-terminus of the polynucleotide encoding the variant carbohydrate oxidase. Any terminator that is functional in the host cell of choice may be used in the present invention.

Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, a nontranslated region of an mRNA that is important for translation by the host cell. The leader sequence is operably linked to the 5′-terminus of the polynucleotide encoding the variant carbohydrate oxidase. Any leader sequence that is functional in the host cell of choice may be used in the present invention.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polypeptide-encoding sequence and, when transcribed, is recognized by the host cell as a signal to add polyadenosine residues to transcribed mRNA. Any polyadenylation sequence that is functional in the host cell of choice may be used in the present invention.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Molecular Cellular Biology 15: 5983-5990.

The control sequence may also be a signal peptide coding region that codes for an amino acid sequence linked to the amino terminus of a variant carbohydrate oxidase and directs the encoded polypeptide into the cell's secretory pathway. The 5′-end of the coding sequence of the polynucleotide may inherently contain a signal peptide coding region naturally linked in translation reading frame with the segment of the coding region that encodes the secreted variant carbohydrate oxidase. Alternatively, the 5′-end of the coding sequence may contain a signal peptide coding region that is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the variant carbohydrate oxidase. However, any signal peptide coding region that directs the expressed polypeptide into the secretory pathway of a host cell of choice may be used in the present invention.

Effective signal peptide coding sequences for filamentous fungal host cells are the signal peptide coding sequences obtained from the genes for Aspergillus oryzae TAKA amylase, Aspergillus niger neutral amylase, Aspergillus niger glucoamylase, Rhizomucor miehei aspartic proteinase, Humicola insolens cellulase, Humicola insolens endoglucanase V, and Humicola lanuginosa lipase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding region that codes for an amino acid sequence positioned at the amino terminus of a variant carbohydrate oxidase. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Saccharomyces cerevisiae alpha-factor, Rhizomucor miehei aspartic proteinase, and Myceliophthora thermophila laccase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences that allow the regulation of the expression of the variant carbohydrate oxidase relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant carbohydrate oxidase would be operably linked with the regulatory sequence.

### Expression Vectors

The present invention also relates to recombinant expression vectors comprising a polynucleotide encoding a variant carbohydrate oxidase of the present invention, a promoter, and transcriptional and translational stop signals. The various nucleotide and control sequences described above may be joined together to produce a recombinant expression vector that may include one or more (several) convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about the expression of the polynucleotide. The choice of the vector will typically depend on the compatibility of the vector with the host cell into which the vector is to be introduced. The vectors may be linear or closed circular plasmids.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vectors of the present invention preferably contain one or more (several) selectable markers that permit easy selection of transformed, transfected, transduced, or the like cells. A selectable marker is a gene the product of which provides for biocide or viral resistance, resistance to heavy metals, prototrophy to auxotrophs, and the like.

Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are the amdS and pyrG genes of Aspergillus nidulans or Aspergillus oryzae and the bar gene of Streptomyces hygroscopicus.

The vectors of the present invention preferably contain an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or nonhomologous recombination. Alternatively, the vector may contain additional nucleotide sequences for directing integration by homologous recombination into the genome of the host cell at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which have a high degree of identity to the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” is defined herein as a nucleotide sequence that enables a plasmid or vector to replicate in vivo.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Research 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into the host cell to increase production of a carbohydrate oxidase variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra) to obtain substantially pure carbohydrate oxidase variants.

### Host Cells

The present invention also relates to recombinant host cells, comprising a polynucleotide encoding a variant carbohydrate oxidase, which are advantageously used in the recombinant production of the variant. A vector comprising a polynucleotide of the present invention is introduced into a host cell so that the vector is maintained as a chromosomal integrant or as a self-replicating extra-chromosomal vector as described earlier. The choice of a host cell will to a large extent depend upon the gene encoding the polypeptide and its source.

The host cell may be any cell useful in the recombinant production of a variant carbohydrate oxidase. The host cell may also be a eukaryote, such as a mammalian, insect, plant, or fungal cell.

In one aspect, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In another aspect, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

In another aspect, the yeast host cell is a Candida, Hansenula, Kluyveromyces, Pichia, Saccharomyces, Schizosaccharomyces, or Yarrowia cell.

In another aspect, the yeast host cell is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis cell. In another aspect, the yeast host cell is a Kluyveromyces lactis cell. In another aspect, the yeast host cell is a Yarrowia lipolytica cell.

In another aspect, the fungal host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In another aspect, the filamentous fungal host cell is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

In another aspect, the filamentous fungal host cell is an Aspergillus awamori, Aspergillus fumigatus, Aspergillus foetidus, Aspergillus japonicus, Aspergillus nidulans, Aspergillus niger or Aspergillus oryzae cell. In another aspect, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another aspect, the filamentous fungal host cell is a Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

### Methods of Production

The present invention also relates to methods of producing a carbohydrate oxidase variant, comprising: (a) cultivating a host cell of the present invention under conditions suitable for the expression of the variant; and (b) recovering the variant from the cultivation medium.

In the production methods of the present invention, the host cells are cultivated in a nutrient medium suitable for production of the carbohydrate oxidase variant using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

In an alternative aspect, the carbohydrate oxidase variant is not recovered, but rather a host cell of the present invention expressing a variant is used as a source of the variant.

The carbohydrate oxidase variant may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein in the Examples.

The resulting carbohydrate oxidase variant may be recovered by methods known in the art. For example, the polypeptide may be recovered from the nutrient medium by conventional procedures including, but not limited to, collection, centrifugation, filtration, extraction, spray-drying, evaporation, or precipitation.

A carbohydrate oxidase variant of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989) to obtain substantially pure carbohydrate oxidase variants.

### Compositions

The present invention also relates to compositions comprising a variant carbohydrate oxidase or a polypeptide having carbohydrate oxidase activity of the present invention. Preferably, the compositions are enriched in such a variant or polypeptide. The term “enriched” indicates that the carbohydrate oxidase activity of the composition has been increased, e.g., with an enrichment factor of 1.1.

The composition may comprise a variant or polypeptide of the present invention as the major enzymatic component, e.g., a mono-component composition. Alternatively, the composition may comprise multiple enzymatic activities, such as an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, or xylanase. The additional enzyme(s) may be produced, for example, by a microorganism belonging to the genus Aspergillus, preferably Aspergillus aculeatus, Aspergillus awamori, Aspergillus fumigatus, Aspergillus foetidus, Aspergillus japonicus, Aspergillus nidulans, Aspergillus niger, or Aspergillus oryzae; Fusarium, preferably Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sulphureum, Fusarium toruloseum, Fusarium trichothecioides, or Fusarium venenatum; Humicola, preferably Humicola insolens or Humicola lanuginosa; or Trichoderma, preferably Trichoderma harzianum, Trichoderma koningii, Trichoderma longibrachiatum, Trichoderma reesei, or Trichoderma viride.

The polypeptide compositions may be prepared in accordance with methods known in the art and may be in the form of a liquid or a dry composition. For instance, the polypeptide composition may be in the form of a granulate or a microgranulate. The variant or polypeptide to be included in the composition may be stabilized in accordance with methods known in the art.

Examples are given below of preferred uses of the variant or polypeptide compositions of the invention. The dosage of the composition of the invention and other conditions under which the composition is used may be determined on the basis of methods known in the art.

### Use in Baking

The carbohydrate oxidases of the invention can be used in the preparation of dough, bread and cakes. Thus, the carbohydrate oxidases can be used in a process for making bread, comprising adding the carbohydrate oxidases to the ingredients of a dough, kneading the dough and baking the dough to make the bread. This can be done in analogy with U.S. Pat. No. 4,567,056 or WO 99/53769.

The dough is generally a flour dough comprising wheat meal or wheat flour and/or other types of meal, flour or starch such as corn flour, corn starch, rye meal, rye flour, oat flour, oat meal, soy flour, sorghum meal, sorghum flour, rice starch, rice flour, potato meal, potato flour or potato starch. The dough may be fresh, frozen or par-baked.

The dough can be a leavened dough or a dough to be subjected to leavening. The dough may be leavened in various ways, such as by adding chemical leavening agents, e.g., sodium bicarbonate or by adding a leaven (fermenting dough), but it is preferred to leaven the dough by adding a suitable yeast culture, such as a culture of Saccharomyces cerevisiae (baker's yeast), e.g. a commercially available strain of S. cerevisiae.

The dough may also comprise other conventional dough ingredients, e.g.: proteins, such as milk or milk powder, gluten, and soy; eggs (either whole eggs, egg yolks or egg whites); shortening such as granulated fat or oil; an oxidant such as ascorbic acid, potassium bromate, potassium iodate, azodicarbonamide (ADA) or ammonium persulfate; a reducing agent such as L-cysteine; a sugar; a salt such as sodium chloride, calcium acetate, sodium sulfate or calcium sulfate. The dough may further comprise an emulsifier such as mono- or diglycerides, diacetyl tartaric acid esters of mono- or diglycerides, sugar esters of fatty acids, polyglycerol esters of fatty acids, lactic acid esters of monoglycerides, acetic acid esters of monoglycerides, polyoxyethylene stearates, phospholipids, lecithin and lysolecithin.

The dough may be a pasta dough, preferably prepared from durum flour or a flour of comparable quality. When used in the preparation of pasta and noodles, the carbohydrate oxidase may result in a strengthening of the gluten structure and thereby providing a reduction in stickiness of the dough, an increase in dough strength and a dough product with an improved texture.

The process of the invention may be used for any kind of baked product prepared from dough, either of a soft or a crisp character, either of a white, light or dark type. Examples are bread (in particular white, whole-meal or rye bread), typically in the form of loaves or rolls, French baguette-type bread, pita bread, tortillas, cakes, pancakes, biscuits, cookies, muffins, pie crusts, crisp bread, steamed bread, pizza and the like.

The process may be used to prepare a fried dough or steamed dough product (e.g., steamed bread).

The present invention further relates to a pre-mix, e.g., in the form of a flour composition, of dough and/or baked products made from dough, in which the pre-mix comprises or consists of the carbohydrate oxidase and optionally other enzymes as specified above. The pre-mix may be prepared by mixing enzyme the relevant enzyme(s) with a suitable carrier, such as flour, starch, sugar or a salt. The pre-mix may contain other dough-improving and/or bread-improving additives, e.g. any of the additives, including enzymes, mentioned above.

The carbohydrate oxidase may be provided as a dough and/or bread improving additive in the form of a granulate or agglomerated powder. The dough and/or bread improving additive preferably has a narrow particle size distribution with more than 95% (by weight) of the particles in the range from 25 to 500 um.

Granulates and agglomerated powders may be prepared by conventional methods, e.g. by spraying the amylase onto a carrier in a fluid-bed granulator. The carrier may consist of particulate cores having a suitable particle size. The carrier may be soluble or insoluble, e.g. a salt (such as NaCl or sodium sulfate), a sugar (such as sucrose or lactose), a sugar alcohol (such as sorbitol), starch, rice, corn grits, or soy.

### Other Industrial Applications

The carbohydrate oxidase of the present invention may be used, for example, in personal care products such as toothpaste, in particular, where whitening of the teeth is desirable, mouthwash, denture cleaner, liquid soap, skin care creams and lotions, hair care and body care formulations, and solutions for cleaning contact lenses in an amount effective, such as, to act as an antibacterial agent.

In yet another aspect, the carbohydrate oxidase of the present invention may be used to oxidize an oligosaccharide with a glucose residue at the reducing end into the corresponding acid, e.g. to produce lactobionic acid from lactose, as described, e.g., in U.S. Pat. No. 6,916,496, U.S. Pat. No. 7,186,427, WO 2006/028929 and WO 2005/104859, which are hereby incorporated by reference. The LBA generated may be used in the production of food and beverage products, e.g., dairy products, such as, cheese, milk, yogurt, and ice cream. The LBA may be generated by direct addition of the generated LBA to the food or beverage product or ingredient or by in situ generation of LBA in the food or beverage product or ingredient. In an embodiment of this aspect, the carbohydrate oxidase has improved stability at low pH (at least pH 3.5 and lower). In yet another embodiment of this aspect, the carbohydrate oxidase has improved stability towards alkali agents and conditions, such as, sodium hydroxide and other buffers used to main pH. In another embodiment of this aspect, the carbohydrate oxidase has a lower Km for oxygen. In yet another embodiment of this aspect, the carbohydrate oxidase has improved stability in the present of H2O2. The addition of catalase, however, may be used to reduce the presence of H2O2 in this aspect of the present invention.

The carbohydrate oxidase may also be used as an analytical reagent, for example, to determine the amount of reducing sugars present in a given sample, or the enzyme may be immobilized and inserted into an electrode to provide continuous measurement of starch or cellulose hydrolysis.

### Plants

The present invention also relates to a transgenic plant, plant part, or plant cell that has been transformed with a polynucleotide encoding a variant carbohydrate oxidase or polypeptide of the present invention so as to express and produce the variant or polypeptide in recoverable quantities. The variant or polypeptide may be recovered from the plant or plant part. Alternatively, the plant or plant part containing the recombinant a variant or polypeptide may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilisation of the invention are also considered plant parts, e.g., embryos, endosperms, aleurone and seeds coats.

Also included within the scope of the present invention are the progeny of such plants, plant parts, and plant cells.

The transgenic plant or plant cell expressing a variant or polypeptide of the present invention may be constructed in accordance with methods known in the art. In short, the plant or plant cell is constructed by incorporating one or more (several) expression constructs encoding a a variant or polypeptide of the present invention into the plant host genome and propagating the resulting modified plant or plant cell into a transgenic plant or plant cell.

Conveniently, the expression construct is a nucleic acid construct that comprises a nucleic acid sequence encoding a a variant or polypeptide of the present invention operably linked with appropriate regulatory sequences required for expression of the nucleic acid sequence in the plant or plant part of choice. Furthermore, the expression construct may comprise a selectable marker useful for identifying host cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences, is determined, for example, on the basis of when, where, and how the a variant or polypeptide is desired to be expressed. For example, the expression of the gene encoding a a variant or polypeptide of the present invention may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV, the maize ubiquitin 1, and the rice actin 1 promoter may be used (Franck et al., 1980, Cell 21: 285-294, Christensen et al., 1992, Plant Mo. Biol. 18: 675-689; Zhang et al., 1991, Plant Cell 3: 1155-1165). Organ-specific promoters may be, for example, a promoter from storage sink tissues such as seeds, potato tubers, and fruits (Edwards & Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant and Cell Physiology 39: 885-889), a Vicia faba promoter from the legumin B4 and the unknown seed protein gene from Vicia faba (Conrad et al., 1998, Journal of Plant Physiology 152: 708-711), a promoter from a seed oil body protein (Chen et al., 1998, Plant and Cell Physiology 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiology 102: 991-1000, the chlorella virus adenine methyltransferase gene promoter (Mitra and Higgins, 1994, Plant Molecular Biology 26: 85-93), or the aldP gene promoter from rice (Kagaya et al., 1995, Molecular and General Genetics 248: 668-674), or a wound inducible promoter such as the potato pin2 promoter (Xu et al., 1993, Plant Molecular Biology 22: 573-588). Likewise, the promoter may inducible by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals.

A promoter enhancer element may also be used to achieve higher expression of a polypeptide of the present invention in the plant. For example, the promoter enhancer element may be an intron that is placed between the promoter and the polynucleotide encoding a polypeptide of the present invention. Xu et al., 1993, supra, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Presently, Agrobacterium tumefaciens-mediated gene transfer is the method of choice for generating transgenic dicots (for a review, see Hooykas and Schilperoort, 1992, Plant Molecular Biology 19: 15-38) and can also be used for transforming monocots, although other transformation methods are often used for these plants. Presently, the method of choice for generating transgenic monocots is particle bombardment (microscopic gold or tungsten particles coated with the transforming DNA) of embryonic calli or developing embryos (Christou, 1992, Plant Journal 2: 275-281; Shimamoto, 1994, Current Opinion Biotechnology 5: 158-162; Vasil et al., 1992, Bio/Technology 10: 667-674). An alternative method for transformation of monocots is based on protoplast transformation as described by Omirulleh et al., 1993, Plant Molecular Biology 21: 415-428.

Following transformation, the transformants having incorporated the expression construct are selected and regenerated into whole plants according to methods well-known in the art. Often the transformation procedure is designed for the selective elimination of selection genes either during regeneration or in the following generations by using, for example, co-transformation with two separate T-DNA constructs or site specific excision of the selection gene by a specific recombinase.

The present invention also relates to methods of producing a variant or polypeptide of the present invention comprising (a) cultivating a transgenic plant or a plant cell comprising a nucleic acid sequence encoding a a variant or polypeptide having carbohydrate oxidase activity of the present invention under conditions conducive for production of the variant or polypeptide; and (b) recovering the a variant or polypeptide.

The present invention is further described by the following examples that should not be construed as limiting the scope of the invention.

### Materials and Methods

### 4AA-TOPS Assay (Method for Determination of Carbohydrate Oxidase Activity)

Assays are carried out in 96 well microtiter plates. 170 μl pre-mix (1.5 mM N-ethyl-N-sulfopropyl-m-toluidine (TOPS), 0.6 mM 4-aminoantipyrine (4-AA) and 5 mM lactose (or cellobiose) in 0.05 M acetate/phosphate/borate, pH 6) is mixed with 20 μl peroxidase (0.2 kPOXU/g) and the reaction is started by adding 10 μl of oxidase solution diluted appropriately. Absorption is measured at 550 nm as a function time using the Vmax microliter plate ready from Molecular Devices and the activity is taken as the slope of the linear increase in absorption.

### EXAMPLES

### Example 1

### Substrate Specificity

The substrate specificity for the carbohydrate oxidase can be determined in a microplate at ambient temperature by mixing in the following order:

50 μl 0.4/0.4 M phosphate/citrate buffer (pH 6),
50 μl substrate (360 mM),
50 μl 21.6 mM 3-Dimethylaminobenzoic acid (DMAB),
50 μl 1 mM 3-Methyl-2-benzothiazolinone hydrazone (MBTH),
50 μl 75 μg/ml, rec. Coprinus cinereus peroxidase (rCiP), and
50 μl carbohydrate oxidase.

The absorbance is measured at 595 nm for at least 3 minutes. The increase in absorbance per minute can be used as a measure for relative activity.

### Example 2

### Binding Constant, Km

Steady state kinetics can be conducted by varying the concentration of the carbohydrate substrates and determining the carbohydrate oxidase activity by the 4AA-TOPS assay. Simple Michaelis-Menten kinetics can be assumed, although the reaction is not a simple one substrate-one product mechanism.

Kinetic constants can be obtained from a Lineweaver-Burke plot, assuming simple Michaelis-Menten kinetics (although this is a rather poor assumption) to obtain apparent values “Km” and “Vm” for various substrates.

### Example 3

### pH and Temperature Activity Profiles

The activity of the carbohydrate oxidase over a pH range can be determined in micro plates at ambient temperature using the method described above in Example 1, but with buffers adjusted to the pH being tested; the pH measured in the reaction mixture.

The temperature activity profile for the carbohydrate oxidase can be determined by mixing buffer and substrate in a glass tube and preincubating at various temperatures (30-80 degree Celsius) for at least 5 minutes:

150 μl 0.4/0.4 M phosphate/citrate, pH 6,
150 μl 180 mM maltose, and
150 μl oxidase dilution.

Reactions are started by addition of oxidase and samples can be incubated at the appropriate temperature in a thermostatic bath. After 5 minutes the samples should be placed on ice, and formation of H2O2 can be determined by addition of 450 μl of DMAB:MBTH:rCiP (1:1:1) at the respective concentrations as in Example 1 and the increase in absorbance at 590 nm can be measured after 10 seconds on a HP 8452A diode array spectrophotometer (Hewlett-Packard).

### Example 4

### Thermostability by DSC

Thermostability by DSC can be measured as follows: A sample of carbohydrate oxidase is desalted into 0.1 M MES, pH 6 using the NAP-5 columns from Pharmacia. The sample (containing 6.5 mg/ml of the oxidase) is loaded onto the VP-DSC apparatus (MicroCal) and a linear scan from 20 to 90 degree Celsius is at a scan rate of 90 degrees/h.

### Example 5

### Temperature Stability

The temperature stability of a carbohydrate oxidase can be measured by pre-incubating the carbohydrate oxidase for 1 hour at pH 6 at and varying temperatures before measuring the residual activity by the 4AA-TOPS assay. The ratio of residual activity between sample incubated at the high temperature and sample incubated at room temperature can be used as a measure for the temperature stability for the oxidase.

### Example 6

### pH-Stability

The pH-Stability of a carbohydrate oxidase can be measured by incubating the carbohydrate oxidase for 2 hours at 40 degree Celsius at varying pH before measuring the residual activity by the 4AA-TOPS assay.

### Example 7

### Cloning and Expression of Carbohydrate Oxidase Variants

Cloning of the M. nivale carbohydrate oxidase (wild-type) is described in Xu et al. “A novel carbohydrate:acceptor oxidoreductase from Microdochium nivale”, Eur. J. Biochem. (2001) vol. 268, pp. 1136-1142. The cloned gene was subsequently transferred as a BamHI-Aflll fragment into pENI2516 (see Example 2 of WO 2004/069872) for expression in Aspergillus oryzae. Plasmid DNA was used as template in the PCR reaction. The sequence of the forward primer was 5′ CTGAGGATCCACCatgcgttctgcatttatcttggcc 3′ (SEQ ID NO:3) and the sequence of the reverse primer was 5′ Caggctgtccgccctgtcaaataacttaagacctggt 3′ (SEQ ID NO:4). The Phusion DNA polymerase from FINNZYMES was used for the PCR.

### PCR Mixture:

0.5 μl template DNA (20 ng/μl)
0.5 μl primer fwd
0.5 μl primer rev
0.5 μl Phusion polymerase
4 μl 2.5 mM dNTP
10 μl Phusion HF buffer

### 15 μl Betaine

### 19 μl Water

### PCR Cycle:

Step 1: 30 seconds at 98° C.
Step 2: 10 seconds at 98° C.; 30 seconds at 67° C.; 45 seconds at 72° C.; repeat 25 times
Step 3: 10 minutes at 72° C.

### Step 4: Hold at 4° C.

The PCR reaction resulted in a single band of approximately 1553 basepair size visible on an agarose gel. The band was extracted from the gel by Qiagen's QIAquick Gel Extraction Kit. The recovered DNA was subsequently subjected to restriction at 37° C. for 3 hours:

### 10 μl DNA

### 2 μl NEBuffer 2

### 2 μl 10×BSA

### 1 μl BamHI

### 1 μl Aflll

### 4 μl Water

The gel-cleaned DNA was ligated into pENI2516 as a BamHI-Aflll fragment to create MnCOx-wt. The resulting plasmid was initially transformed into E. coli strain TOP10 and the insert was sequenced to confirm its nucleotide sequence. The plasmid was subsequently transformed into Aspergillus oryzae strain ToC1512 for expression.

A transformed Aspergillus oryzae strain ToC1512 (described in WO 2005/070962, Example 11) was grown for expression of oxidase enzyme. Typically, a 100 mL of YP media was inoculated with spores from a stock in 50% glycerol stored at −80° C. The starter culture was grown in a baffled 250 mL flask for 3-4 days at 37° C. and 180 rpm. Twenty mL of this culture was then used to inoculate 500 mL YP medium added 2% maltose in a 2 L flask with baffles. The flask was placed in an orbital shaker at 180 rpm and grown for 4-5 days at 37° C. before being harvested. The enzymatic activity in the broth was monitored daily using the described assay. The production of active enzyme with the correct molecular mass was demonstrated by assay and SDS-PAGE analysis of the crude broth.

### Example 8

### Site-Directed Mutagenesis of M. nivale Oxidase

All variants were constructed based on the M. nivale oxidase expression vector MnCOx-wt, described in Example 7. The mutation primers used in the constructions are summarized in Table 1. The mutations were introduced by commonly used PCR-based technology for site-directed mutagenesis.

TABLE 1

| | SEQ ID | | |
|---|---|---|---|
| Mutation | NO: | Primer | Sequence (5′ → 3′) |
| D15N | 5 | forw. | CTGCTGCTGGCGTCCCGATCAACATTCCTGGCACTGCCGACTATG |
| | 6 | rev. | GATCGGGACGCCAGCAGCAGACAGGCAGGC |
| Y22W | 7 | forw. | GATATTCCTGGCACTGCCGACtggGAGCGCGATGTCGAGCCCTT |
| | 8 | rev. | GTCGGCAGTGCCAGGAATATCGATCGGGACG |
| E27R | 9 | forw. | GCCGACTATGAGCGCGATGTCcgcCCCTTCAACATCCGCCTGCC |
| | 10 | rev. | GACATCGCGCTCATAGTCGGCAGTGCCAGGAA |
| Q54D | 11 | forw. | GCTCACATCCAGTCGGCAGTCgacTGCGCCAAGAAGCTCAACCTC |
| | 12 | rev. | GACTGCCGACTGGATGTGAGCAGTAGTCTGCG |
| N98D | 13 | forw. | GCATGATTGATGTCATCTCGTACgacGACAAGACTGGCATTGCCCATG |
| | 14 | rev. | GTACGAGATGACATCAATCATGCGGTCGAGCTG |
| K188R | 15 | forw. | GCTGACCTCTTCTGGGGTATCcgcGGCGCTGGCTCCAACTTCGG |
| | 16 | rev. | GATACCCCAGAAGAGGTCAGCATTCTCAGTGGC |
| K201R | 17 | forw. | TTCGGCATCGTTGCTGTCTGGcgcCTCGCCACTTTCCCTGCTCC |
| | 18 | rev. | CCAGACAGCAACGATGCCGAAGTTGGAGCCAG |
| N222D | 19 | forw. | GGCGTCACCCTCAACTGGAAGgacAAGACCTCTGCCCTCAAGGGC |
| | 20 | rev. | CTTCCAGTTGAGGGTGACGCCAAAGCGGGTGA |
| T460E | 21 | forw. | GCTCAAGGCCAAGTTTGATCCCgagGACCGTTTCTACTACCCTCAGG |
| | 22 | rev. | GGGATCAAACTTGGCCTTGAGCTTCTGGAGCCT |

The resulting plasmids were transformed into competent TOP10 Escherichia coli cells, isolated and the inserts were verified by sequencing. The plasmids were subsequently transformed into Aspergillus oryzae strain ToC1512 for expression.

### Temperature Stability of Variants

The temperature stability of variants was evaluated by incubation of fermentation supernatants at room temperature and at 69° C. for 1 hour. The residual activity of the supernatants were then determined as described below and the ratio between the high temperature and room temperature incubated samples was used as a measure for the temperature stability for the variants.

The enzymatic activity of the produced oxidase variants was analyzed (and compared to wild-type) using oxidation of carbohydrate. The generated hydrogen peroxide was then detected in a coupled assay with peroxidase from Coprinus cinereus. All assays were performed in Nunc 96-well plates and the change in absorbance measured in a SpectraMax 384 plus UV-Vis spectrophotometer plate-reader from Molecular Devices.

In the carbohydrate oxidation assay, 10 μl of oxidase enzyme solution and 20 μl peroxidase (0.2 kPOXU/g) were reacted with 170 μl of 0.6 mM 4-aminoantipyrine, 1.5 mM N-ethyl-N-sulfopropyl-m-toluidine and 5 mM lactose (or cellobiose) in 50 mM Borate/phosphate/acetate buffer pH 6.0. The increase in absorbance at 550 nm was monitored for 5 minutes and the slope of the progress curve was used to calculate the initial rate of the reaction.

The results from the activity assays with lactose and cellobiose are shown in Table 3. The activity remaining in a sample after incubation at 69° C. for 1 hour is calculated as percentage of the activity remaining in the same sample but left at room temperature (RT) for 1 hour.

TABLE 3

| | Lactose | | Cellobiose | |
|---|---|---|---|---|
| Substitution | dA/min (RT) | Activity % | dA/min (RT) | Activity % |
| wildtype | 64 | 26 | 70 | 26 |
| Y22W | 48 | 68 | 42 | 61 |
| E27R | 165 | 57 | 94 | 68 |
| Q54D | 26 | 69 | 19 | 63 |
| N98D | 68 | 43 | 56 | 34 |
| N222D | 37 | 68 | 44 | 38 |
| T460E | 234 | 30 | 130 | 39 |

### High pH Stability of Variants

The stability of variants at high pH was evaluated by incubation of fermentation supernatants at pH 6, 7, 8 and 9 for 1 hour. Fermentation supernatants were dilute 10-fold in 50 mM borate/acetate/phosphate buffer and incubated one hour at room temperature. The residual activity was determined as described above in “Temperature stability of variants” using 10 μl sample and using cellobiose as substrate.

The results from the activity assays are shown in table 4, where the remaining activity after incubation at a given pH is expressed as a percentage of the activity measured after incubation at pH 6 for 1 hour at room temperature.

TABLE 4

| Substitution | pH 6 | pH 7 | pH 8 | pH 9 |
|---|---|---|---|---|
| wildtype | 100 | 83 | 65 | 61 |
| D15N | 100 | 82 | 96 | 98 |
| K188R | 100 | 76 | 79 | 73 |
| K201R | 100 | 93 | 97 | 81 |

APPENDIX 1

| | | | | | | | | | |
|---|---|---|---|---|---|---|---|---|---|
| ATOM | 1 | N | GLY | 1 | −36.727 | −29.597 | −11.823 | 1.00 | 20.14 |
| ATOM | 2 | CA | GLY | 1 | −37.942 | −29.546 | −10.975 | 1.00 | 19.15 |
| ATOM | 3 | C | GLY | 1 | −38.080 | −28.168 | −10.354 | 1.00 | 18.42 |
| ATOM | 4 | O | GLY | 1 | −37.300 | −27.256 | −10.641 | 1.00 | 18.61 |
| ATOM | 5 | N | ALA | 2 | −39.066 | −28.025 | −9.480 | 1.00 | 17.34 |
| ATOM | 6 | CA | ALA | 2 | −39.424 | −26.723 | −8.928 | 1.00 | 16.82 |
| ATOM | 7 | C | ALA | 2 | −38.248 | −25.974 | −8.237 | 1.00 | 16.57 |
| ATOM | 8 | O | ALA | 2 | −38.047 | −24.786 | −8.490 | 1.00 | 15.52 |
| ATOM | 9 | CB | ALA | 2 | −40.606 | −26.901 | −7.978 | 1.00 | 17.10 |
| ATOM | 10 | N | ILE | 3 | −37.492 | −26.676 | −7.374 | 1.00 | 15.99 |
| ATOM | 11 | CA | ILE | 3 | −36.387 | −26.056 | −6.616 | 1.00 | 15.78 |
| ATOM | 12 | C | ILE | 3 | −35.251 | −25.626 | −7.532 | 1.00 | 15.89 |
| ATOM | 13 | O | ILE | 3 | −34.676 | −24.550 | −7.347 | 1.00 | 14.49 |
| ATOM | 14 | CB | ILE | 3 | −35.843 | −26.982 | −5.435 | 1.00 | 15.80 |
| ATOM | 15 | CG1 | ILE | 3 | −34.719 | −26.288 | −4.662 | 1.00 | 14.70 |
| ATOM | 16 | CG2 | ILE | 3 | −35.408 | −28.367 | −5.943 | 1.00 | 16.15 |
| ATOM | 17 | CD1 | ILE | 3 | −35.198 | −24.993 | −3.900 | 1.00 | 13.57 |
| ATOM | 18 | N | GLU | 4 | −34.931 | −26.463 | −8.520 | 1.00 | 16.39 |
| ATOM | 19 | CA | GLU | 4 | −33.871 | −26.127 | −9.467 | 1.00 | 17.65 |
| ATOM | 20 | C | GLU | 4 | −34.288 | −24.862 | −10.237 | 1.00 | 17.73 |
| ATOM | 21 | O | GLU | 4 | −33.518 | −23.925 | −10.392 | 1.00 | 17.70 |
| ATOM | 22 | CB | GLU | 4 | −33.611 | −27.283 | −10.435 | 1.00 | 17.53 |
| ATOM | 23 | CG | GLU | 4 | −32.957 | −28.548 | −9.805 | 1.00 | 20.18 |
| ATOM | 24 | CD | GLU | 4 | −33.940 | −29.474 | −9.096 | 1.00 | 20.56 |
| ATOM | 25 | OE1 | GLU | 4 | −35.169 | −29.309 | −9.264 | 1.00 | 17.95 |
| ATOM | 26 | OE2 | GLU | 4 | −33.471 | −30.393 | −8.382 | 1.00 | 24.22 |
| ATOM | 27 | N | ALA | 5 | −35.536 | −24.839 | −10.689 | 1.00 | 18.78 |
| ATOM | 28 | CA | ALA | 5 | −36.063 | −23.698 | −11.457 | 1.00 | 18.56 |
| ATOM | 29 | C | ALA | 5 | −36.005 | −22.445 | −10.599 | 1.00 | 17.53 |
| ATOM | 30 | O | ALA | 5 | −35.574 | −21.391 | −11.070 | 1.00 | 17.94 |
| ATOM | 31 | CB | ALA | 5 | −37.487 | −23.987 | −11.905 | 1.00 | 18.39 |
| ATOM | 32 | N | CYS | 6 | −36.380 | −22.581 | −9.322 | 1.00 | 16.96 |
| ATOM | 33 | CA | CYS | 6 | −36.389 | −21.444 | −8.390 | 1.00 | 16.74 |
| ATOM | 34 | C | CYS | 6 | −34.999 | −20.854 | −8.202 | 1.00 | 16.83 |
| ATOM | 35 | O | CYS | 6 | −34.826 | −19.633 | −8.224 | 1.00 | 17.94 |
| ATOM | 36 | CB | CYS | 6 | −36.947 | −21.847 | −7.019 | 1.00 | 16.77 |
| ATOM | 37 | SG | CYS | 6 | −37.104 | −20.435 | −5.886 | 0.82 | 15.90 |
| ATOM | 38 | N | LEU | 7 | −34.017 | −21.726 | −7.979 | 1.00 | 16.06 |
| ATOM | 39 | CA | LEU | 7 | −32.653 | −21.303 | −7.769 | 1.00 | 16.12 |
| ATOM | 40 | C | LEU | 7 | −32.073 | −20.670 | −9.035 | 1.00 | 16.22 |
| ATOM | 41 | O | LEU | 7 | −31.390 | −19.643 | −8.962 | 1.00 | 15.74 |
| ATOM | 42 | CB | LEU | 7 | −31.788 | −22.500 | −7.335 | 1.00 | 15.86 |
| ATOM | 43 | CG | LEU | 7 | −32.066 | −23.011 | −5.930 | 1.00 | 14.76 |
| ATOM | 44 | CD1 | LEU | 7 | −31.470 | −24.399 | −5.710 | 1.00 | 15.57 |
| ATOM | 45 | CD2 | LEU | 7 | −31.603 | −22.006 | −4.858 | 1.00 | 13.89 |
| ATOM | 46 | N | SER | 8 | −32.321 | −21.296 | −10.186 | 1.00 | 16.75 |
| ATOM | 47 | CA | SER | 8 | −31.808 | −20.752 | −11.448 | 1.00 | 18.52 |
| ATOM | 48 | C | SER | 8 | −32.426 | −19.394 | −11.802 | 1.00 | 18.79 |
| ATOM | 49 | O | SER | 8 | −31.720 | −18.506 | −12.274 | 1.00 | 19.80 |
| ATOM | 50 | CB | SER | 8 | −31.996 | −21.741 | −12.584 | 1.00 | 18.52 |
| ATOM | 51 | OG | SER | 8 | −31.093 | −22.804 | −12.437 | 1.00 | 21.70 |
| ATOM | 52 | N | ALA | 9 | −33.731 | −19.236 | −11.557 | 1.00 | 19.59 |
| ATOM | 53 | CA | ALA | 9 | −34.437 | −17.954 | −11.774 | 1.00 | 19.69 |
| ATOM | 54 | C | ALA | 9 | −33.812 | −16.821 | −10.992 | 1.00 | 19.53 |
| ATOM | 55 | O | ALA | 9 | −33.839 | −15.687 | −11.452 | 1.00 | 19.97 |
| ATOM | 56 | CB | ALA | 9 | −35.925 | −18.071 | −11.411 | 1.00 | 19.68 |
| ATOM | 57 | N | ALA | 10 | −33.242 | −17.137 | −9.820 | 1.00 | 19.00 |
| ATOM | 58 | CA | ALA | 10 | −32.562 | −16.162 | −8.947 | 1.00 | 17.76 |
| ATOM | 59 | C | ALA | 10 | −31.074 | −16.014 | −9.216 | 1.00 | 17.53 |
| ATOM | 60 | O | ALA | 10 | −30.375 | −15.305 | −8.478 | 1.00 | 18.01 |
| ATOM | 61 | CB | ALA | 10 | −32.777 | −16.537 | −7.482 | 1.00 | 17.88 |
| ATOM | 62 | N | GLY | 11 | −30.572 | −16.679 | −10.254 | 1.00 | 17.41 |
| ATOM | 63 | CA | GLY | 11 | −29.143 | −16.660 | −10.566 | 1.00 | 16.95 |
| ATOM | 64 | C | GLY | 11 | −28.237 | −17.487 | −9.638 | 1.00 | 17.23 |
| ATOM | 65 | O | GLY | 11 | −27.013 | −17.321 | −9.655 | 1.00 | 16.96 |
| ATOM | 66 | N | VAL | 12 | −28.811 | −18.367 | −8.816 | 1.00 | 16.87 |
| ATOM | 67 | CA | VAL | 12 | −27.994 | −19.184 | −7.889 | 1.00 | 16.39 |
| ATOM | 68 | C | VAL | 12 | −27.416 | −20.420 | −8.598 | 1.00 | 15.93 |
| ATOM | 69 | O | VAL | 12 | −28.178 | −21.233 | −9.121 | 1.00 | 15.47 |
| ATOM | 70 | CB | VAL | 12 | −28.806 | −19.668 | −6.663 | 1.00 | 16.39 |
| ATOM | 71 | CG1 | VAL | 12 | −27.914 | −20.509 | −5.726 | 1.00 | 16.33 |
| ATOM | 72 | CG2 | VAL | 12 | −29.422 | −18.491 | −5.935 | 1.00 | 15.02 |
| ATOM | 73 | N | PRO | 13 | −26.069 | −20.570 | −8.613 | 1.00 | 15.32 |
| ATOM | 74 | CA | PRO | 13 | −25.513 | −21.785 | −9.245 | 1.00 | 15.26 |
| ATOM | 75 | C | PRO | 13 | −25.955 | −23.032 | −8.497 | 1.00 | 15.15 |
| ATOM | 76 | O | PRO | 13 | −26.098 | −23.021 | −7.264 | 1.00 | 14.49 |
| ATOM | 77 | CB | PRO | 13 | −23.997 | −21.581 | −9.146 | 1.00 | 14.82 |
| ATOM | 78 | CG | PRO | 13 | −23.822 | −20.094 | −8.989 | 1.00 | 14.52 |
| ATOM | 79 | CD | PRO | 13 | −25.003 | −19.667 | −8.146 | 1.00 | 14.43 |
| ATOM | 80 | N | ILE | 14 | −26.250 | −24.081 | −9.250 | 1.00 | 14.66 |
| ATOM | 81 | CA | ILE | 14 | −26.727 | −25.311 | −8.651 | 1.00 | 15.10 |
| ATOM | 82 | C | ILE | 14 | −25.834 | −26.432 | −9.142 | 1.00 | 15.11 |
| ATOM | 83 | O | ILE | 14 | −25.278 | −26.315 | −10.208 | 1.00 | 15.45 |
| ATOM | 84 | CB | ILE | 14 | −28.214 | −25.574 | −8.970 | 1.00 | 15.76 |
| ATOM | 85 | CG1 | ILE | 14 | −28.488 | −25.548 | −10.478 | 1.00 | 16.79 |
| ATOM | 86 | CG2 | ILE | 14 | −29.085 | −24.539 | −8.256 | 1.00 | 15.10 |
| ATOM | 87 | CD1 | ILE | 14 | −29.807 | −26.229 | −10.896 | 1.00 | 17.00 |
| ATOM | 88 | N | ASP | 15 | −25.660 | −27.487 | −8.353 | 1.00 | 14.84 |
| ATOM | 89 | CA | ASP | 15 | −24.909 | −28.642 | −8.825 | 1.00 | 14.89 |
| ATOM | 90 | C | ASP | 15 | −25.823 | −29.534 | −9.675 | 1.00 | 15.29 |
| ATOM | 91 | O | ASP | 15 | −27.022 | −29.617 | −9.440 | 1.00 | 15.15 |
| ATOM | 92 | CB | ASP | 15 | −24.300 | −29.457 | −7.671 | 1.00 | 13.98 |
| ATOM | 93 | CG | ASP | 15 | −23.115 | −28.769 | −7.003 | 1.00 | 13.00 |
| ATOM | 94 | OD1 | ASP | 15 | −22.509 | −27.831 | −7.565 | 1.00 | 10.89 |
| ATOM | 95 | OD2 | ASP | 15 | −22.759 | −29.189 | −5.880 | 1.00 | 15.31 |
| ATOM | 96 | N | ILE | 16 | −25.229 | −30.185 | −10.665 | 1.00 | 15.67 |
| ATOM | 97 | CA | ILE | 16 | −25.958 | −31.095 | −11.544 | 0.50 | 15.97 |
| ATOM | 98 | C | ILE | 16 | −25.549 | −32.526 | −11.207 | 1.00 | 15.68 |
| ATOM | 99 | O | ILE | 16 | −24.378 | −32.886 | −11.346 | 1.00 | 15.80 |
| ATOM | 100 | CB | ILE | 16 | −25.672 | −30.802 | −13.046 | 0.50 | 16.11 |
| ATOM | 101 | CG1 | ILE | 16 | −25.901 | −29.327 | −13.360 | 0.50 | 17.11 |
| ATOM | 102 | CG2 | ILE | 16 | −26.558 | −31.664 | −13.942 | 0.50 | 16.66 |
| ATOM | 103 | CD1 | ILE | 16 | −27.328 | −28.885 | −13.153 | 0.50 | 17.95 |
| ATOM | 104 | N | PRO | 17 | −26.522 | −33.361 | −10.745 | 1.00 | 16.37 |
| ATOM | 105 | CA | PRO | 17 | −26.157 | −34.758 | −10.464 | 1.00 | 17.06 |
| ATOM | 106 | C | PRO | 17 | −25.397 | −35.360 | −11.629 | 1.00 | 18.22 |
| ATOM | 107 | O | PRO | 17 | −25.774 | −35.141 | −12.776 | 1.00 | 18.20 |
| ATOM | 108 | CB | PRO | 17 | −27.512 | −35.451 | −10.287 | 1.00 | 16.71 |
| ATOM | 109 | CG | PRO | 17 | −28.392 | −34.357 | −9.757 | 1.00 | 16.10 |
| ATOM | 110 | CD | PRO | 17 | −27.958 | −33.115 | −10.468 | 1.00 | 15.96 |
| ATOM | 111 | N | GLY | 18 | −24.308 | −36.064 | −11.322 | 1.00 | 19.38 |
| ATOM | 112 | CA | GLY | 18 | −23.527 | −36.755 | −12.337 | 1.00 | 20.66 |
| ATOM | 113 | C | GLY | 18 | −22.241 | −36.033 | −12.725 | 1.00 | 21.22 |
| ATOM | 114 | O | GLY | 18 | −21.406 | −36.608 | −13.426 | 1.00 | 22.52 |
| ATOM | 115 | N | THR | 19 | −22.089 | −34.775 | −12.296 | 1.00 | 20.18 |
| ATOM | 116 | CA | THR | 19 | −20.913 | −33.978 | −12.630 | 1.00 | 18.76 |
| ATOM | 117 | C | THR | 19 | −19.898 | −33.980 | −11.472 | 1.00 | 18.77 |
| ATOM | 118 | O | THR | 19 | −20.262 | −34.247 | −10.305 | 1.00 | 18.55 |
| ATOM | 119 | CB | THR | 19 | −21.274 | −32.516 | −12.951 | 1.00 | 18.53 |
| ATOM | 120 | CG2 | THR | 19 | −22.284 | −32.406 | −14.088 | 1.00 | 18.93 |
| ATOM | 121 | OG1 | THR | 19 | −21.801 | −31.870 | −11.783 | 1.00 | 17.69 |
| ATOM | 122 | N | ALA | 20 | −18.643 | −33.648 | −11.790 | 1.00 | 17.36 |
| ATOM | 123 | CA | ALA | 20 | −17.574 | −33.544 | −10.782 | 1.00 | 17.63 |
| ATOM | 124 | C | ALA | 20 | −17.982 | −32.703 | −9.569 | 1.00 | 16.91 |
| ATOM | 125 | O | ALA | 20 | −17.866 | −33.161 | −8.431 | 1.00 | 18.31 |
| ATOM | 126 | CB | ALA | 20 | −16.285 | −32.968 | −11.408 | 1.00 | 17.89 |
| ATOM | 127 | N | ASP | 21 | −18.451 | −31.483 | −9.814 | 1.00 | 15.76 |
| ATOM | 128 | CA | ASP | 21 | −18.825 | −30.593 | −8.728 | 0.50 | 15.55 |
| ATOM | 129 | C | ASP | 21 | −19.864 | −31.247 | −7.820 | 1.00 | 15.08 |
| ATOM | 130 | O | ASP | 21 | −19.733 | −31.191 | −6.606 | 1.00 | 15.40 |
| ATOM | 131 | CB | ASP | 21 | −19.312 | −29.245 | −9.259 | 0.50 | 15.25 |
| ATOM | 132 | CG | ASP | 21 | −18.161 | −28.296 | −9.574 | 0.50 | 15.91 |
| ATOM | 133 | OD1 | ASP | 21 | −17.584 | −28.390 | −10.678 | 0.50 | 16.39 |
| ATOM | 134 | OD2 | ASP | 21 | −17.822 | −27.453 | −8.716 | 0.50 | 16.43 |
| ATOM | 135 | N | TYR | 22 | −20.865 | −31.895 | −8.415 | 1.00 | 14.07 |
| ATOM | 136 | CA | TYR | 22 | −21.904 | −32.547 | −7.615 | 1.00 | 13.78 |
| ATOM | 137 | C | TYR | 22 | −21.329 | −33.687 | −6.749 | 1.00 | 13.78 |
| ATOM | 138 | O | TYR | 22 | −21.510 | −33.700 | −5.524 | 1.00 | 12.77 |
| ATOM | 139 | CB | TYR | 22 | −23.071 | −33.033 | −8.493 | 1.00 | 13.04 |
| ATOM | 140 | CG | TYR | 22 | −24.179 | −33.710 | −7.704 | 1.00 | 12.75 |
| ATOM | 141 | CD1 | TYR | 22 | −25.266 | −32.987 | −7.253 | 1.00 | 11.98 |
| ATOM | 142 | CD2 | TYR | 22 | −24.137 | −35.071 | −7.431 | 1.00 | 11.09 |
| ATOM | 143 | CE1 | TYR | 22 | −26.278 | −33.587 | −6.517 | 1.00 | 12.72 |
| ATOM | 144 | CE2 | TYR | 22 | −25.153 | −35.695 | −6.700 | 1.00 | 11.03 |
| ATOM | 145 | CZ | TYR | 22 | −26.215 | −34.934 | −6.240 | 1.00 | 13.48 |
| ATOM | 146 | OH | TYR | 22 | −27.250 | −35.516 | −5.526 | 1.00 | 16.71 |
| ATOM | 147 | N | GLU | 23 | −20.618 | −34.621 | −7.386 | 1.00 | 13.63 |
| ATOM | 148 | CA | GLU | 23 | −19.988 | −35.729 | −6.674 | 1.00 | 14.07 |
| ATOM | 149 | C | GLU | 23 | −19.091 | −35.254 | −5.513 | 1.00 | 13.95 |
| ATOM | 150 | O | GLU | 23 | −19.109 | −35.851 | −4.448 | 1.00 | 13.69 |
| ATOM | 151 | CB | GLU | 23 | −19.219 | −36.651 | −7.631 | 1.00 | 14.01 |
| ATOM | 152 | CG | GLU | 23 | −20.112 | −37.307 | −8.718 | 1.00 | 17.67 |
| ATOM | 153 | CD | GLU | 23 | −21.169 | −38.260 | −8.170 | 0.50 | 18.26 |
| ATOM | 154 | OE1 | GLU | 23 | −20.825 | −39.149 | −7.376 | 0.50 | 19.07 |
| ATOM | 155 | OE2 | GLU | 23 | −22.350 | −38.124 | −8.546 | 0.50 | 20.36 |
| ATOM | 156 | N | ARG | 24 | −18.320 | −34.181 | −5.733 | 1.00 | 14.25 |
| ATOM | 157 | CA | ARG | 24 | −17.485 | −33.581 | −4.701 | 1.00 | 13.59 |
| ATOM | 158 | C | ARG | 24 | −18.322 | −32.987 | −3.549 | 1.00 | 13.24 |
| ATOM | 159 | O | ARG | 24 | −18.056 | −33.271 | −2.380 | 1.00 | 12.87 |
| ATOM | 160 | CB | ARG | 24 | −16.609 | −32.468 | −5.295 | 1.00 | 14.42 |
| ATOM | 161 | CG | ARG | 24 | −15.835 | −31.684 | −4.220 | 1.00 | 15.66 |
| ATOM | 162 | CD | ARG | 24 | −14.961 | −30.571 | −4.801 | 1.00 | 18.95 |
| ATOM | 163 | NE | ARG | 24 | −14.815 | −29.497 | −3.828 | 1.00 | 21.20 |
| ATOM | 164 | CZ | ARG | 24 | −13.992 | −28.453 | −3.963 | 1.00 | 24.13 |
| ATOM | 165 | NH1 | ARG | 24 | −13.223 | −28.365 | −5.050 | 1.00 | 23.52 |
| ATOM | 166 | NH2 | ARG | 24 | −13.951 | −27.491 | −3.021 | 1.00 | 18.69 |
| ATOM | 167 | N | ASP | 25 | −19.314 | −32.168 | −3.890 | 1.00 | 12.11 |
| ATOM | 168 | CA | ASP | 25 | −20.084 | −31.435 | −2.898 | 1.00 | 11.92 |
| ATOM | 169 | C | ASP | 25 | −21.027 | −32.331 | −2.059 | 1.00 | 11.75 |
| ATOM | 170 | O | ASP | 25 | −21.324 | −32.015 | −0.902 | 1.00 | 10.30 |
| ATOM | 171 | CB | ASP | 25 | −20.905 | −30.332 | −3.576 | 1.00 | 11.76 |
| ATOM | 172 | CG | ASP | 25 | −20.067 | −29.146 | −4.097 | 1.00 | 10.47 |
| ATOM | 173 | OD1 | ASP | 25 | −18.890 | −28.934 | −3.727 | 1.00 | 11.01 |
| ATOM | 174 | OD2 | ASP | 25 | −20.643 | −28.367 | −4.903 | 1.00 | 10.98 |
| ATOM | 175 | N | VAL | 26 | −21.509 | −33.428 | −2.667 | 1.00 | 11.53 |
| ATOM | 176 | CA | VAL | 26 | −22.486 | −34.307 | −2.043 | 1.00 | 10.95 |
| ATOM | 177 | C | VAL | 26 | −21.812 | −35.380 | −1.150 | 1.00 | 11.24 |
| ATOM | 178 | O | VAL | 26 | −22.490 | −36.097 | −0.408 | 1.00 | 9.51 |
| ATOM | 179 | CB | VAL | 26 | −23.396 | −35.019 | −3.114 | 1.00 | 11.37 |
| ATOM | 180 | CG1 | VAL | 26 | −22.722 | −36.292 | −3.663 | 1.00 | 10.47 |
| ATOM | 181 | CG2 | VAL | 26 | −24.759 | −35.368 | −2.510 | 1.00 | 10.90 |
| ATOM | 182 | N | GLU | 27 | −20.493 | −35.490 | −1.242 | 1.00 | 10.95 |
| ATOM | 183 | CA | GLU | 27 | −19.786 | −36.509 | −0.478 | 1.00 | 12.35 |
| ATOM | 184 | C | GLU | 27 | −19.584 | −36.030 | 0.973 | 1.00 | 11.28 |
| ATOM | 185 | O | GLU | 27 | −18.976 | −34.966 | 1.185 | 1.00 | 11.88 |
| ATOM | 186 | CB | GLU | 27 | −18.421 | −36.804 | −1.126 | 1.00 | 11.93 |
| ATOM | 187 | CG | GLU | 27 | −17.635 | −37.963 | −0.442 | 1.00 | 15.20 |
| ATOM | 188 | CD | GLU | 27 | −16.303 | −38.236 | −1.137 | 1.00 | 19.49 |
| ATOM | 189 | OE1 | GLU | 27 | −15.617 | −37.260 | −1.519 | 1.00 | 18.11 |
| ATOM | 190 | OE2 | GLU | 27 | −15.957 | −39.430 | −1.306 | 1.00 | 22.03 |
| ATOM | 191 | N | PRO | 28 | −20.088 | −36.790 | 1.969 | 1.00 | 11.44 |
| ATOM | 192 | CA | PRO | 28 | −19.877 | −36.415 | 3.415 | 1.00 | 10.98 |
| ATOM | 193 | C | PRO | 28 | −18.439 | −36.661 | 3.845 | 1.00 | 12.16 |
| ATOM | 194 | O | PRO | 28 | −17.780 | −37.533 | 3.254 | 1.00 | 12.30 |
| ATOM | 195 | CB | PRO | 28 | −20.745 | −37.416 | 4.173 | 1.00 | 11.45 |
| ATOM | 196 | CG | PRO | 28 | −20.786 | −38.677 | 3.215 | 1.00 | 10.53 |
| ATOM | 197 | CD | PRO | 28 | −20.803 | −38.080 | 1.817 | 1.00 | 11.53 |
| ATOM | 198 | N | PHE | 29 | −17.952 | −35.955 | 4.881 | 1.00 | 11.60 |
| ATOM | 199 | CA | PHE | 29 | −16.665 | −36.335 | 5.468 | 1.00 | 11.32 |
| ATOM | 200 | C | PHE | 29 | −16.788 | −37.748 | 6.068 | 1.00 | 11.32 |
| ATOM | 201 | O | PHE | 29 | −15.893 | −38.588 | 5.908 | 1.00 | 11.13 |
| ATOM | 202 | CB | PHE | 29 | −16.178 | −35.323 | 6.541 | 1.00 | 10.10 |
| ATOM | 203 | CG | PHE | 29 | −15.009 | −35.837 | 7.376 | 1.00 | 10.94 |
| ATOM | 204 | CD1 | PHE | 29 | −13.738 | −36.010 | 6.799 | 1.00 | 11.04 |
| ATOM | 205 | CD2 | PHE | 29 | −15.182 | −36.170 | 8.717 | 1.00 | 9.83 |
| ATOM | 206 | CE1 | PHE | 29 | −12.667 | −36.497 | 7.555 | 1.00 | 12.06 |
| ATOM | 207 | CE2 | PHE | 29 | −14.111 | −36.665 | 9.492 | 1.00 | 9.95 |
| ATOM | 208 | CZ | PHE | 29 | −12.864 | −36.831 | 8.911 | 1.00 | 10.98 |
| ATOM | 209 | N | ASN | 30 | −17.889 | −37.986 | 6.795 | 1.00 | 11.42 |
| ATOM | 210 | CA | ASN | 30 | −18.203 | −39.293 | 7.372 | 1.00 | 11.34 |
| ATOM | 211 | C | ASN | 30 | −19.061 | −40.106 | 6.388 | 1.00 | 11.74 |
| ATOM | 212 | O | ASN | 30 | −20.289 | −39.923 | 6.304 | 1.00 | 10.27 |
| ATOM | 213 | CB | ASN | 30 | −18.945 | −39.162 | 8.722 | 1.00 | 11.07 |
| ATOM | 214 | CG | ASN | 30 | −18.948 | −40.468 | 9.493 | 1.00 | 13.40 |
| ATOM | 215 | ND2 | ASN | 30 | −19.373 | −40.438 | 10.759 | 1.00 | 11.28 |
| ATOM | 216 | OD1 | ASN | 30 | −18.568 | −41.510 | 8.937 | 1.00 | 14.26 |
| ATOM | 217 | N | ILE | 31 | −18.413 | −41.000 | 5.645 | 1.00 | 13.17 |
| ATOM | 218 | CA | ILE | 31 | −19.131 | −41.786 | 4.634 | 1.00 | 14.45 |
| ATOM | 219 | C | ILE | 31 | −20.065 | −42.840 | 5.227 | 1.00 | 14.41 |
| ATOM | 220 | O | ILE | 31 | −20.865 | −43.449 | 4.506 | 1.00 | 14.61 |
| ATOM | 221 | CB | ILE | 31 | −18.198 | −42.394 | 3.581 | 1.00 | 15.29 |
| ATOM | 222 | CG1 | ILE | 31 | −17.198 | −43.360 | 4.241 | 1.00 | 16.69 |
| ATOM | 223 | CG2 | ILE | 31 | −17.552 | −41.275 | 2.792 | 1.00 | 16.08 |
| ATOM | 224 | CD1 | ILE | 31 | −16.710 | −44.454 | 3.314 | 1.00 | 23.25 |
| ATOM | 225 | N | ARG | 32 | −19.998 | −43.031 | 6.546 | 1.00 | 13.64 |
| ATOM | 226 | CA | ARG | 32 | −21.011 | −43.816 | 7.218 | 1.00 | 13.16 |
| ATOM | 227 | C | ARG | 32 | −22.366 | −43.149 | 7.023 | 1.00 | 13.58 |
| ATOM | 228 | O | ARG | 32 | −23.407 | −43.804 | 7.113 | 1.00 | 12.88 |
| ATOM | 229 | CB | ARG | 32 | −20.717 | −43.899 | 8.723 | 1.00 | 12.74 |
| ATOM | 230 | CG | ARG | 32 | −21.535 | −44.964 | 9.431 | 1.00 | 13.79 |
| ATOM | 231 | CD | ARG | 32 | −21.125 | −45.169 | 10.870 | 1.00 | 14.79 |
| ATOM | 232 | NE | ARG | 32 | −21.395 | −44.004 | 11.706 | 1.00 | 14.38 |
| ATOM | 233 | CZ | ARG | 32 | −22.586 | −43.679 | 12.208 | 1.00 | 15.14 |
| ATOM | 234 | NH1 | ARG | 32 | −22.701 | −42.605 | 12.976 | 1.00 | 15.15 |
| ATOM | 235 | NH2 | ARG | 32 | −23.662 | −44.411 | 11.957 | 1.00 | 15.34 |
| ATOM | 236 | N | LEU | 33 | −22.358 | −41.826 | 6.811 | 1.00 | 12.86 |
| ATOM | 237 | CA | LEU | 33 | −23.600 | −41.064 | 6.872 | 1.00 | 12.62 |
| ATOM | 238 | C | LEU | 33 | −23.883 | −40.189 | 5.642 | 1.00 | 11.99 |
| ATOM | 239 | O | LEU | 33 | −23.960 | −38.963 | 5.781 | 1.00 | 12.67 |
| ATOM | 240 | CB | LEU | 33 | −23.660 | −40.242 | 8.186 | 1.00 | 12.09 |
| ATOM | 241 | CG | LEU | 33 | −23.814 | −41.044 | 9.491 | 1.00 | 13.10 |
| ATOM | 242 | CD1 | LEU | 33 | −23.618 | −40.150 | 10.726 | 1.00 | 11.94 |
| ATOM | 243 | CD2 | LEU | 33 | −25.165 | −41.783 | 9.584 | 1.00 | 13.98 |
| ATOM | 244 | N | PRO | 34 | −24.063 | −40.804 | 4.443 | 1.00 | 11.70 |
| ATOM | 245 | CA | PRO | 34 | −24.487 | −39.977 | 3.309 | 1.00 | 11.24 |
| ATOM | 246 | C | PRO | 34 | −25.964 | −39.596 | 3.336 | 1.00 | 11.34 |
| ATOM | 247 | O | PRO | 34 | −26.823 | −40.374 | 3.775 | 1.00 | 11.30 |
| ATOM | 248 | CB | PRO | 34 | −24.195 | −40.863 | 2.087 | 1.00 | 11.93 |
| ATOM | 249 | CG | PRO | 34 | −24.336 | −42.284 | 2.599 | 1.00 | 11.94 |
| ATOM | 250 | CD | PRO | 34 | −23.865 | −42.225 | 4.052 | 1.00 | 12.20 |
| ATOM | 251 | N | TYR | 35 | −26.252 | −38.384 | 2.877 | 1.00 | 10.96 |
| ATOM | 252 | CA | TYR | 35 | −27.630 | −37.953 | 2.656 | 1.00 | 11.18 |
| ATOM | 253 | C | TYR | 35 | −27.794 | −37.366 | 1.268 | 1.00 | 11.01 |
| ATOM | 254 | O | TYR | 35 | −26.814 | −36.907 | 0.664 | 1.00 | 10.02 |
| ATOM | 255 | CB | TYR | 35 | −28.084 | −36.952 | 3.711 | 1.00 | 10.92 |
| ATOM | 256 | CG | TYR | 35 | −28.130 | −37.571 | 5.077 | 1.00 | 11.59 |
| ATOM | 257 | CD1 | TYR | 35 | −27.200 | −37.202 | 6.063 | 1.00 | 11.62 |
| ATOM | 258 | CD2 | TYR | 35 | −29.078 | −38.551 | 5.382 | 1.00 | 11.05 |
| ATOM | 259 | CE1 | TYR | 35 | −27.238 | −37.768 | 7.329 | 1.00 | 9.77 |
| ATOM | 260 | CE2 | TYR | 35 | −29.113 | −39.151 | 6.637 | 1.00 | 13.06 |
| ATOM | 261 | CZ | TYR | 35 | −28.200 | −38.747 | 7.612 | 1.00 | 13.43 |
| ATOM | 262 | OH | TYR | 35 | −28.234 | −39.328 | 8.864 | 1.00 | 12.34 |
| ATOM | 263 | N | ILE | 36 | −29.030 | −37.411 | 0.767 | 1.00 | 11.19 |
| ATOM | 264 | CA | ILE | 36 | −29.342 | −36.948 | −0.576 | 1.00 | 11.87 |
| ATOM | 265 | C | ILE | 36 | −30.173 | −35.669 | −0.465 | 1.00 | 12.39 |
| ATOM | 266 | O | ILE | 36 | −31.373 | −35.733 | −0.151 | 1.00 | 11.37 |
| ATOM | 267 | CB | ILE | 36 | −30.094 | −38.023 | −1.393 | 1.00 | 12.30 |
| ATOM | 268 | CG1 | ILE | 36 | −29.460 | −39.417 | −1.203 | 1.00 | 12.10 |
| ATOM | 269 | CG2 | ILE | 36 | −30.130 | −37.645 | −2.883 | 1.00 | 12.65 |
| ATOM | 270 | CD1 | ILE | 36 | −28.076 | −39.584 | −1.813 | 1.00 | 11.35 |
| ATOM | 271 | N | PRO | 37 | −29.539 | −34.499 | −0.717 | 1.00 | 12.11 |
| ATOM | 272 | CA | PRO | 37 | −30.289 | −33.238 | −0.683 | 1.00 | 12.26 |
| ATOM | 273 | C | PRO | 37 | −31.245 | −33.186 | −1.869 | 1.00 | 12.24 |
| ATOM | 274 | O | PRO | 37 | −30.995 | −33.829 | −2.911 | 1.00 | 12.08 |
| ATOM | 275 | CB | PRO | 37 | −29.208 | −32.157 | −0.839 | 1.00 | 11.00 |
| ATOM | 276 | CG | PRO | 37 | −27.928 | −32.837 | −0.610 | 1.00 | 13.41 |
| ATOM | 277 | CD | PRO | 37 | −28.104 | −34.286 | −0.957 | 1.00 | 12.57 |
| ATOM | 278 | N | THR | 38 | −32.340 | −32.451 | −1.741 | 1.00 | 11.95 |
| ATOM | 279 | CA | THR | 38 | −33.161 | −32.291 | −2.932 | 1.00 | 12.25 |
| ATOM | 280 | C | THR | 38 | −32.408 | −31.508 | −4.046 | 1.00 | 12.14 |
| ATOM | 281 | O | THR | 38 | −32.607 | −31.755 | −5.245 | 1.00 | 12.26 |
| ATOM | 282 | CB | THR | 38 | −34.549 | −31.696 | −2.642 | 1.00 | 12.01 |
| ATOM | 283 | CG2 | THR | 38 | −34.448 | −30.269 | −2.126 | 1.00 | 11.61 |
| ATOM | 284 | OG1 | THR | 38 | −35.301 | −31.713 | −3.855 | 1.00 | 14.22 |
| ATOM | 285 | N | ALA | 39 | −31.553 | −30.572 | −3.630 | 1.00 | 11.15 |
| ATOM | 286 | CA | ALA | 39 | −30.734 | −29.784 | −4.539 | 1.00 | 10.98 |
| ATOM | 287 | C | ALA | 39 | −29.563 | −29.184 | −3.784 | 1.00 | 11.07 |
| ATOM | 288 | O | ALA | 39 | −29.658 | −28.967 | −2.571 | 1.00 | 11.04 |
| ATOM | 289 | CB | ALA | 39 | −31.578 | −28.645 | −5.194 | 1.00 | 10.54 |
| ATOM | 290 | N | ILE | 40 | −28.481 | −28.883 | −4.511 | 1.00 | 11.11 |
| ATOM | 291 | CA | ILE | 40 | −27.308 | −28.244 | −3.933 | 1.00 | 11.57 |
| ATOM | 292 | C | ILE | 40 | −27.074 | −26.879 | −4.601 | 1.00 | 11.90 |
| ATOM | 293 | O | ILE | 40 | −26.774 | −26.827 | −5.797 | 1.00 | 12.46 |
| ATOM | 294 | CB | ILE | 40 | −26.044 | −29.106 | −4.088 | 1.00 | 11.91 |
| ATOM | 295 | CG1 | ILE | 40 | −26.266 | −30.533 | −3.558 | 1.00 | 12.18 |
| ATOM | 296 | CG2 | ILE | 40 | −24.834 | −28.450 | −3.355 | 1.00 | 11.25 |
| ATOM | 297 | CD1 | ILE | 40 | −24.974 | −31.418 | −3.648 | 1.00 | 13.15 |
| ATOM | 298 | N | ALA | 41 | −27.231 | −25.796 | −3.818 | 1.00 | 11.56 |
| ATOM | 299 | CA | ALA | 41 | −26.955 | −24.417 | −4.231 | 1.00 | 11.44 |
| ATOM | 300 | C | ALA | 41 | −25.485 | −24.134 | −3.882 | 1.00 | 12.11 |
| ATOM | 301 | O | ALA | 41 | −25.131 | −24.085 | −2.701 | 1.00 | 12.42 |
| ATOM | 302 | CB | ALA | 41 | −27.865 | −23.437 | −3.485 | 1.00 | 11.07 |
| ATOM | 303 | N | GLN | 42 | −24.636 | −24.004 | −4.902 | 1.00 | 11.71 |
| ATOM | 304 | CA | GLN | 42 | −23.211 | −23.774 | −4.708 | 1.00 | 12.47 |
| ATOM | 305 | C | GLN | 42 | −23.017 | −22.250 | −4.769 | 1.00 | 12.86 |
| ATOM | 306 | O | GLN | 42 | −22.799 | −21.694 | −5.855 | 1.00 | 13.77 |
| ATOM | 307 | CB | GLN | 42 | −22.402 | −24.478 | −5.805 | 1.00 | 12.20 |
| ATOM | 308 | CG | GLN | 42 | −20.904 | −24.613 | −5.493 | 1.00 | 12.82 |
| ATOM | 309 | CD | GLN | 42 | −20.088 | −25.029 | −6.707 | 1.00 | 13.67 |
| ATOM | 310 | NE2 | GLN | 42 | −19.777 | −24.070 | −7.565 | 1.00 | 10.12 |
| ATOM | 311 | OE1 | GLN | 42 | −19.717 | −26.206 | −6.851 | 1.00 | 17.14 |
| ATOM | 312 | N | THR | 43 | −23.159 | −21.583 | −3.615 | 1.00 | 12.05 |
| ATOM | 313 | CA | THR | 43 | −23.268 | −20.128 | −3.552 | 1.00 | 11.98 |
| ATOM | 314 | C | THR | 43 | −21.940 | −19.381 | −3.727 | 1.00 | 12.74 |
| ATOM | 315 | O | THR | 43 | −20.903 | −19.848 | −3.276 | 1.00 | 13.00 |
| ATOM | 316 | CB | THR | 43 | −23.911 | −19.668 | −2.225 | 1.00 | 11.97 |
| ATOM | 317 | CG2 | THR | 43 | −25.369 | −20.174 | −2.126 | 1.00 | 10.47 |
| ATOM | 318 | OG1 | THR | 43 | −23.147 | −20.143 | −1.100 | 1.00 | 9.27 |
| ATOM | 319 | N | GLN | 44 | −21.990 | −18.194 | −4.326 | 1.00 | 13.33 |
| ATOM | 320 | CA | GLN | 44 | −20.792 | −17.359 | −4.486 | 1.00 | 13.30 |
| ATOM | 321 | C | GLN | 44 | −20.854 | −16.054 | −3.678 | 1.00 | 13.59 |
| ATOM | 322 | O | GLN | 44 | −19.822 | −15.462 | −3.352 | 1.00 | 14.16 |
| ATOM | 323 | CB | GLN | 44 | −20.558 | −17.067 | −5.978 | 1.00 | 13.31 |
| ATOM | 324 | CG | GLN | 44 | −20.146 | −18.314 | −6.776 | 1.00 | 14.00 |
| ATOM | 325 | CD | GLN | 44 | −18.687 | −18.727 | −6.504 | 1.00 | 16.14 |
| ATOM | 326 | NE2 | GLN | 44 | −18.364 | −19.985 | −6.756 | 1.00 | 15.17 |
| ATOM | 327 | OE1 | GLN | 44 | −17.877 | −17.913 | −6.054 | 1.00 | 15.99 |
| ATOM | 328 | N | THR | 45 | −22.061 | −15.601 | −3.354 | 1.00 | 13.19 |
| ATOM | 329 | CA | THR | 45 | −22.255 | −14.291 | −2.725 | 1.00 | 12.19 |
| ATOM | 330 | C | THR | 45 | −23.294 | −14.404 | −1.596 | 1.00 | 12.64 |
| ATOM | 331 | O | THR | 45 | −24.016 | −15.404 | −1.513 | 1.00 | 12.08 |
| ATOM | 332 | CB | THR | 45 | −22.822 | −13.267 | −3.736 | 1.00 | 12.96 |
| ATOM | 333 | CG2 | THR | 45 | −21.957 | −13.139 | −5.021 | 1.00 | 11.80 |
| ATOM | 334 | OG1 | THR | 45 | −24.152 | −13.664 | −4.101 | 1.00 | 10.54 |
| ATOM | 335 | N | THR | 46 | −23.388 | −13.372 | −0.752 | 1.00 | 12.03 |
| ATOM | 336 | CA | THR | 46 | −24.443 | −13.292 | 0.269 | 1.00 | 12.30 |
| ATOM | 337 | C | THR | 46 | −25.843 | −13.351 | −0.340 | 1.00 | 12.51 |
| ATOM | 338 | O | THR | 46 | −26.731 | −14.061 | 0.178 | 1.00 | 13.15 |
| ATOM | 339 | CB | THR | 46 | −24.282 | −12.058 | 1.161 | 1.00 | 12.38 |
| ATOM | 340 | CG2 | THR | 46 | −25.365 | −12.023 | 2.281 | 1.00 | 11.25 |
| ATOM | 341 | OG1 | THR | 46 | −22.986 | −12.110 | 1.781 | 1.00 | 13.33 |
| ATOM | 342 | N | ALA | 47 | −26.020 | −12.645 | −1.461 | 1.00 | 12.26 |
| ATOM | 343 | CA | ALA | 47 | −27.271 | −12.605 | −2.194 | 1.00 | 12.09 |
| ATOM | 344 | C | ALA | 47 | −27.733 | −14.000 | −2.629 | 1.00 | 12.53 |
| ATOM | 345 | O | ALA | 47 | −28.929 | −14.309 | −2.561 | 1.00 | 13.92 |
| ATOM | 346 | CB | ALA | 47 | −27.129 | −11.666 | −3.417 | 1.00 | 12.28 |
| ATOM | 347 | N | HIS | 48 | −26.806 | −14.842 | −3.092 | 1.00 | 11.94 |
| ATOM | 348 | CA | HIS | 48 | −27.151 | −16.240 | −3.405 | 1.00 | 11.96 |
| ATOM | 349 | C | HIS | 48 | −27.710 | −16.992 | −2.191 | 1.00 | 12.36 |
| ATOM | 350 | O | HIS | 48 | −28.722 | −17.714 | −2.297 | 1.00 | 11.83 |
| ATOM | 351 | CB | HIS | 48 | −25.934 | −16.993 | −3.913 | 1.00 | 11.65 |
| ATOM | 352 | CG | HIS | 48 | −25.485 | −16.582 | −5.284 | 1.00 | 12.86 |
| ATOM | 353 | CD2 | HIS | 48 | −26.133 | −15.938 | −6.289 | 1.00 | 12.42 |
| ATOM | 354 | ND1 | HIS | 48 | −24.215 | −16.845 | −5.749 | 1.00 | 10.80 |
| ATOM | 355 | CE1 | HIS | 48 | −24.093 | −16.368 | −6.979 | 1.00 | 14.04 |
| ATOM | 356 | NE2 | HIS | 48 | −25.238 | −15.804 | −7.327 | 1.00 | 11.11 |
| ATOM | 357 | N | ILE | 49 | −27.029 | −16.848 | −1.051 | 1.00 | 11.85 |
| ATOM | 358 | CA | ILE | 49 | −27.460 | −17.492 | 0.185 | 1.00 | 12.01 |
| ATOM | 359 | C | ILE | 49 | −28.885 | −17.055 | 0.514 | 1.00 | 12.51 |
| ATOM | 360 | O | ILE | 49 | −29.743 | −17.889 | 0.792 | 1.00 | 13.33 |
| ATOM | 361 | CB | ILE | 49 | −26.494 | −17.191 | 1.387 | 1.00 | 11.53 |
| ATOM | 362 | CG1 | ILE | 49 | −25.081 | −17.734 | 1.091 | 1.00 | 10.80 |
| ATOM | 363 | CG2 | ILE | 49 | −27.040 | −17.804 | 2.697 | 1.00 | 11.52 |
| ATOM | 364 | CD1 | ILE | 49 | −24.021 | −17.260 | 2.069 | 1.00 | 8.65 |
| ATOM | 365 | N | GLN | 50 | −29.114 | −15.740 | 0.473 | 1.00 | 12.16 |
| ATOM | 366 | CA | GLN | 50 | −30.431 | −15.161 | 0.667 | 1.00 | 12.54 |
| ATOM | 367 | C | GLN | 50 | −31.479 | −15.714 | −0.316 | 1.00 | 12.16 |
| ATOM | 368 | O | GLN | 50 | −32.573 | −16.085 | 0.092 | 1.00 | 12.63 |
| ATOM | 369 | CB | GLN | 50 | −30.324 | −13.630 | 0.525 | 1.00 | 13.02 |
| ATOM | 370 | CG | GLN | 50 | −31.649 | −12.869 | 0.678 | 1.00 | 13.78 |
| ATOM | 371 | CD | GLN | 50 | −31.433 | −11.381 | 0.514 | 1.00 | 13.77 |
| ATOM | 372 | NE2 | GLN | 50 | −31.980 | −10.602 | 1.431 | 1.00 | 11.96 |
| ATOM | 373 | OE1 | GLN | 50 | −30.745 | −10.940 | −0.411 | 1.00 | 12.17 |
| ATOM | 374 | N | SER | 51 | −31.153 | −15.770 | −1.601 | 1.00 | 11.48 |
| ATOM | 375 | CA | SER | 51 | −32.106 | −16.275 | −2.595 | 1.00 | 11.65 |
| ATOM | 376 | C | SER | 51 | −32.434 | −17.757 | −2.359 | 1.00 | 11.96 |
| ATOM | 377 | O | SER | 51 | −33.575 | −18.192 | −2.577 | 1.00 | 11.28 |
| ATOM | 378 | CB | SER | 51 | −31.540 | −16.117 | −4.015 | 1.00 | 11.66 |
| ATOM | 379 | OG | SER | 51 | −31.360 | −14.739 | −4.326 | 1.00 | 13.41 |
| ATOM | 380 | N | ALA | 52 | −31.426 | −18.525 | −1.937 | 1.00 | 10.50 |
| ATOM | 381 | CA | ALA | 52 | −31.629 | −19.941 | −1.646 | 1.00 | 11.49 |
| ATOM | 382 | C | ALA | 52 | −32.612 | −20.074 | −0.480 | 1.00 | 11.14 |
| ATOM | 383 | O | ALA | 52 | −33.512 | −20.891 | −0.534 | 1.00 | 11.58 |
| ATOM | 384 | CB | ALA | 52 | −30.287 | −20.653 | −1.325 | 1.00 | 9.97 |
| ATOM | 385 | N | VAL | 53 | −32.451 | −19.259 | 0.550 | 1.00 | 11.43 |
| ATOM | 386 | CA | VAL | 53 | −33.365 | −19.298 | 1.701 | 1.00 | 12.10 |
| ATOM | 387 | C | VAL | 53 | −34.787 | −18.893 | 1.254 | 1.00 | 13.26 |
| ATOM | 388 | O | VAL | 53 | −35.764 | −19.493 | 1.695 | 1.00 | 13.77 |
| ATOM | 389 | CB | VAL | 53 | −32.816 | −18.465 | 2.901 | 1.00 | 12.20 |
| ATOM | 390 | CG1 | VAL | 53 | −33.871 | −18.196 | 3.993 | 1.00 | 11.33 |
| ATOM | 391 | CG2 | VAL | 53 | −31.585 | −19.164 | 3.526 | 1.00 | 11.25 |
| ATOM | 392 | N | GLN | 54 | −34.907 | −17.914 | 0.353 | 1.00 | 13.83 |
| ATOM | 393 | CA | GLN | 54 | −36.210 | −17.580 | −0.249 | 1.00 | 14.49 |
| ATOM | 394 | C | GLN | 54 | −36.857 | −18.776 | −0.960 | 1.00 | 14.87 |
| ATOM | 395 | O | GLN | 54 | −38.048 | −19.028 | −0.784 | 1.00 | 15.52 |
| ATOM | 396 | CB | GLN | 54 | −36.059 | −16.454 | −1.285 | 1.00 | 15.21 |
| ATOM | 397 | CG | GLN | 54 | −35.974 | −15.046 | −0.748 | 1.00 | 15.89 |
| ATOM | 398 | CD | GLN | 54 | −36.140 | −14.051 | −1.879 | 1.00 | 19.28 |
| ATOM | 399 | NE2 | GLN | 54 | −37.307 | −13.398 | −1.915 | 1.00 | 17.53 |
| ATOM | 400 | OE1 | GLN | 54 | −35.244 | −13.892 | −2.744 | 1.00 | 18.43 |
| ATOM | 401 | N | CYS | 55 | −36.095 | −19.501 | −1.787 | 1.00 | 14.59 |
| ATOM | 402 | CA | CYS | 55 | −36.638 | −20.699 | −2.477 | 1.00 | 14.69 |
| ATOM | 403 | C | CYS | 55 | −37.166 | −21.778 | −1.509 | 1.00 | 15.03 |
| ATOM | 404 | O | CYS | 55 | −38.218 | −22.409 | −1.755 | 1.00 | 14.95 |
| ATOM | 405 | CB | CYS | 55 | −35.588 | −21.297 | −3.434 | 1.00 | 14.50 |
| ATOM | 406 | SG | CYS | 55 | −35.321 | −20.290 | −4.894 | 0.90 | 13.77 |
| ATOM | 407 | N | ALA | 56 | −36.427 | −21.994 | −0.425 | 1.00 | 15.41 |
| ATOM | 408 | CA | ALA | 56 | −36.813 | −22.958 | 0.596 | 1.00 | 16.23 |
| ATOM | 409 | C | ALA | 56 | −38.172 | −22.563 | 1.180 | 1.00 | 16.42 |
| ATOM | 410 | O | ALA | 56 | −39.062 | −23.416 | 1.327 | 1.00 | 16.96 |
| ATOM | 411 | CB | ALA | 56 | −35.750 | −23.017 | 1.698 | 1.00 | 15.72 |
| ATOM | 412 | N | LYS | 57 | −38.332 | −21.275 | 1.501 | 1.00 | 17.04 |
| ATOM | 413 | CA | LYS | 57 | −39.624 | −20.752 | 1.987 | 1.00 | 17.95 |
| ATOM | 414 | C | LYS | 57 | −40.754 | −20.979 | 0.974 | 1.00 | 18.33 |
| ATOM | 415 | O | LYS | 57 | −41.849 | −21.450 | 1.330 | 1.00 | 18.52 |
| ATOM | 416 | CB | LYS | 57 | −39.537 | −19.258 | 2.356 | 1.00 | 18.39 |
| ATOM | 417 | CG | LYS | 57 | −40.894 | −18.705 | 2.813 | 1.00 | 18.98 |
| ATOM | 418 | CD | LYS | 57 | −40.779 | −17.329 | 3.429 | 1.00 | 24.79 |
| ATOM | 419 | CE | LYS | 57 | −42.115 | −16.618 | 3.412 | 1.00 | 25.27 |
| ATOM | 420 | NZ | LYS | 57 | −41.954 | −15.245 | 3.949 | 1.00 | 29.04 |
| ATOM | 421 | N | LYS | 58 | −40.479 | −20.660 | −0.288 | 1.00 | 19.03 |
| ATOM | 422 | CA | LYS | 58 | −41.473 | −20.801 | −1.356 | 1.00 | 20.02 |
| ATOM | 423 | C | LYS | 58 | −41.975 | −22.252 | −1.525 | 1.00 | 19.61 |
| ATOM | 424 | O | LYS | 58 | −43.167 | −22.476 | −1.748 | 1.00 | 19.22 |
| ATOM | 425 | CB | LYS | 58 | −40.886 | −20.287 | −2.678 | 1.00 | 20.91 |
| ATOM | 426 | CG | LYS | 58 | −41.860 | −20.313 | −3.844 | 1.00 | 25.19 |
| ATOM | 427 | CD | LYS | 58 | −41.132 | −20.308 | −5.205 | 1.00 | 31.01 |
| ATOM | 428 | CE | LYS | 58 | −42.075 | −19.899 | −6.347 | 1.00 | 33.04 |
| ATOM | 429 | NZ | LYS | 58 | −42.410 | −18.421 | −6.258 | 1.00 | 37.73 |
| ATOM | 430 | N | LEU | 59 | −41.068 | −23.227 | −1.403 | 1.00 | 18.59 |
| ATOM | 431 | CA | LEU | 59 | −41.374 | −24.616 | −1.704 | 1.00 | 17.98 |
| ATOM | 432 | C | LEU | 59 | −41.548 | −25.465 | −0.443 | 1.00 | 17.84 |
| ATOM | 433 | O | LEU | 59 | −41.625 | −26.687 | −0.521 | 1.00 | 17.93 |
| ATOM | 434 | CB | LEU | 59 | −40.318 | −25.201 | −2.644 | 1.00 | 18.14 |
| ATOM | 435 | CG | LEU | 59 | −40.237 | −24.439 | −3.989 | 1.00 | 19.84 |
| ATOM | 436 | CD1 | LEU | 59 | −38.947 | −24.776 | −4.729 | 1.00 | 18.82 |
| ATOM | 437 | CD2 | LEU | 59 | −41.472 | −24.675 | −4.874 | 1.00 | 19.50 |
| ATOM | 438 | N | ASN | 60 | −41.644 | −24.804 | 0.704 | 1.00 | 17.44 |
| ATOM | 439 | CA | ASN | 60 | −41.715 | −25.484 | 2.008 | 1.00 | 18.01 |
| ATOM | 440 | C | ASN | 60 | −40.622 | −26.559 | 2.223 | 1.00 | 16.68 |
| ATOM | 441 | O | ASN | 60 | −40.911 | −27.707 | 2.554 | 1.00 | 15.98 |
| ATOM | 442 | CB | ASN | 60 | −43.118 | −26.056 | 2.277 | 1.00 | 18.63 |
| ATOM | 443 | CG | ASN | 60 | −43.379 | −26.285 | 3.777 | 1.00 | 23.91 |
| ATOM | 444 | ND2 | ASN | 60 | −44.398 | −27.079 | 4.071 | 1.00 | 28.98 |
| ATOM | 445 | OD1 | ASN | 60 | −42.672 | −25.748 | 4.659 | 1.00 | 28.48 |
| ATOM | 446 | N | LEU | 61 | −39.364 | −26.153 | 2.057 | 1.00 | 15.69 |
| ATOM | 447 | CA | LEU | 61 | −38.223 | −27.050 | 2.246 | 1.00 | 14.45 |
| ATOM | 448 | C | LEU | 61 | −37.443 | −26.654 | 3.497 | 1.00 | 13.82 |
| ATOM | 449 | O | LEU | 61 | −37.410 | −25.476 | 3.854 | 1.00 | 14.28 |
| ATOM | 450 | CB | LEU | 61 | −37.310 | −26.974 | 1.022 | 1.00 | 14.16 |
| ATOM | 451 | CG | LEU | 61 | −37.886 | −27.363 | −0.346 | 1.00 | 14.63 |
| ATOM | 452 | CD1 | LEU | 61 | −36.895 | −27.058 | −1.507 | 1.00 | 12.25 |
| ATOM | 453 | CD2 | LEU | 61 | −38.264 | −28.846 | −0.378 | 1.00 | 15.77 |
| ATOM | 454 | N | LYS | 62 | −36.819 | −27.623 | 4.157 | 1.00 | 13.43 |
| ATOM | 455 | CA | LYS | 62 | −35.777 | −27.337 | 5.159 | 1.00 | 12.94 |
| ATOM | 456 | C | LYS | 62 | −34.491 | −27.000 | 4.419 | 1.00 | 13.42 |
| ATOM | 457 | O | LYS | 62 | −34.184 | −27.566 | 3.336 | 1.00 | 11.78 |
| ATOM | 458 | CB | LYS | 62 | −35.543 | −28.526 | 6.099 | 1.00 | 13.26 |
| ATOM | 459 | CG | LYS | 62 | −36.803 | −29.020 | 6.838 | 1.00 | 13.67 |
| ATOM | 460 | CD | LYS | 62 | −37.592 | −27.860 | 7.431 | 1.00 | 14.65 |
| ATOM | 461 | CE | LYS | 62 | −38.716 | −28.359 | 8.352 | 1.00 | 18.37 |
| ATOM | 462 | NZ | LYS | 62 | −39.364 | −27.243 | 9.101 | 1.00 | 16.83 |
| ATOM | 463 | N | VAL | 63 | −33.741 | −26.071 | 4.995 | 1.00 | 12.83 |
| ATOM | 464 | CA | VAL | 63 | −32.477 | −25.683 | 4.385 | 1.00 | 13.11 |
| ATOM | 465 | C | VAL | 63 | −31.305 | −25.866 | 5.368 | 1.00 | 12.76 |
| ATOM | 466 | O | VAL | 63 | −31.414 | −25.546 | 6.551 | 1.00 | 11.71 |
| ATOM | 467 | CB | VAL | 63 | −32.571 | −24.296 | 3.655 | 1.00 | 13.23 |
| ATOM | 468 | CG1 | VAL | 63 | −31.187 | −23.739 | 3.298 | 1.00 | 14.06 |
| ATOM | 469 | CG2 | VAL | 63 | −33.338 | −23.317 | 4.453 | 1.00 | 13.32 |
| ATOM | 470 | N | SER | 64 | −30.215 | −26.469 | 4.882 | 1.00 | 12.12 |
| ATOM | 471 | CA | SER | 64 | −29.046 | −26.670 | 5.739 | 1.00 | 11.18 |
| ATOM | 472 | C | SER | 64 | −27.771 | −26.173 | 5.037 | 1.00 | 10.99 |
| ATOM | 473 | O | SER | 64 | −27.560 | −26.415 | 3.845 | 1.00 | 10.71 |
| ATOM | 474 | CB | SER | 64 | −28.961 | −28.124 | 6.195 | 1.00 | 10.29 |
| ATOM | 475 | OG | SER | 64 | −30.132 | −28.482 | 7.121 | 1.00 | 8.79 |
| ATOM | 476 | N | ALA | 65 | −26.927 | −25.456 | 5.773 | 1.00 | 11.12 |
| ATOM | 477 | CA | ALA | 65 | −25.656 | −24.968 | 5.206 | 1.00 | 10.48 |
| ATOM | 478 | C | ALA | 65 | −24.539 | −25.991 | 5.435 | 1.00 | 10.17 |
| ATOM | 479 | O | ALA | 65 | −24.465 | −26.593 | 6.504 | 1.00 | 10.74 |
| ATOM | 480 | CB | ALA | 65 | −25.296 | −23.665 | 5.821 | 1.00 | 9.74 |
| ATOM | 481 | N | LYS | 66 | −23.691 | −26.197 | 4.432 | 1.00 | 9.68 |
| ATOM | 482 | CA | LYS | 66 | −22.498 | −27.036 | 4.597 | 1.00 | 9.61 |
| ATOM | 483 | C | LYS | 66 | −21.317 | −26.152 | 4.287 | 1.00 | 9.69 |
| ATOM | 484 | O | LYS | 66 | −21.274 | −25.567 | 3.216 | 1.00 | 9.21 |
| ATOM | 485 | CB | LYS | 66 | −22.495 | −28.240 | 3.644 | 1.00 | 9.09 |
| ATOM | 486 | CG | LYS | 66 | −21.406 | −29.295 | 3.960 | 1.00 | 8.97 |
| ATOM | 487 | CD | LYS | 66 | −21.845 | −30.726 | 3.571 | 1.00 | 7.77 |
| ATOM | 488 | CE | LYS | 66 | −21.726 | −30.963 | 2.055 | 1.00 | 7.78 |
| ATOM | 489 | NZ | LYS | 66 | −20.333 | −30.822 | 1.530 | 1.00 | 8.50 |
| ATOM | 490 | N | SER | 67 | −20.368 | −26.063 | 5.228 | 1.00 | 9.42 |
| ATOM | 491 | CA | SER | 67 | −19.214 | −25.190 | 5.108 | 1.00 | 9.98 |
| ATOM | 492 | C | SER | 67 | −18.011 | −26.097 | 4.762 | 1.00 | 10.54 |
| ATOM | 493 | O | SER | 67 | −17.707 | −26.278 | 3.593 | 1.00 | 10.39 |
| ATOM | 494 | CB | SER | 67 | −19.025 | −24.445 | 6.434 | 1.00 | 10.44 |
| ATOM | 495 | OG | SER | 67 | −17.845 | −23.681 | 6.468 | 1.00 | 11.68 |
| ATOM | 496 | N | GLY | 68 | −17.371 | −26.709 | 5.763 | 1.00 | 10.27 |
| ATOM | 497 | CA | GLY | 68 | −16.275 | −27.645 | 5.490 | 1.00 | 10.98 |
| ATOM | 498 | C | GLY | 68 | −16.742 | −29.088 | 5.408 | 1.00 | 10.75 |
| ATOM | 499 | O | GLY | 68 | −15.982 | −29.978 | 4.996 | 1.00 | 12.07 |
| ATOM | 500 | N | GLY | 69 | −17.967 | −29.332 | 5.870 | 1.00 | 11.37 |
| ATOM | 501 | CA | GLY | 69 | −18.594 | −30.648 | 5.780 | 1.00 | 10.66 |
| ATOM | 502 | C | GLY | 69 | −18.091 | −31.623 | 6.820 | 1.00 | 11.22 |
| ATOM | 503 | O | GLY | 69 | −18.353 | −32.842 | 6.724 | 1.00 | 11.00 |
| ATOM | 504 | N | HIS | 70 | −17.400 | −31.104 | 7.829 | 1.00 | 10.21 |
| ATOM | 505 | CA | HIS | 70 | −16.829 | −31.955 | 8.880 | 1.00 | 10.53 |
| ATOM | 506 | C | HIS | 70 | −17.759 | −32.470 | 9.984 | 1.00 | 10.32 |
| ATOM | 507 | O | HIS | 70 | −17.326 | −33.254 | 10.834 | 1.00 | 9.69 |
| ATOM | 508 | CB | HIS | 70 | −15.558 | −31.332 | 9.456 | 1.00 | 10.05 |
| ATOM | 509 | CG | HIS | 70 | −14.334 | −31.769 | 8.723 | 1.00 | 12.30 |
| ATOM | 510 | CD2 | HIS | 70 | −13.871 | −31.426 | 7.494 | 1.00 | 10.84 |
| ATOM | 511 | ND1 | HIS | 70 | −13.489 | −32.751 | 9.209 | 1.00 | 9.53 |
| ATOM | 512 | CE1 | HIS | 70 | −12.531 | −32.954 | 8.313 | 1.00 | 14.79 |
| ATOM | 513 | NE2 | HIS | 70 | −12.739 | −32.164 | 7.271 | 1.00 | 13.69 |
| ATOM | 514 | N | SER | 71 | −19.032 | −32.071 | 9.956 | 1.00 | 9.78 |
| ATOM | 515 | CA | SER | 71 | −20.001 | −32.645 | 10.906 | 1.00 | 9.57 |
| ATOM | 516 | C | SER | 71 | −19.871 | −34.174 | 10.985 | 1.00 | 9.92 |
| ATOM | 517 | O | SER | 71 | −19.986 | −34.883 | 9.956 | 1.00 | 8.45 |
| ATOM | 518 | CB | SER | 71 | −21.436 | −32.342 | 10.511 | 1.00 | 9.08 |
| ATOM | 519 | OG | SER | 71 | −22.297 | −32.943 | 11.458 | 1.00 | 8.51 |
| ATOM | 520 | N | TYR | 72 | −19.656 | −34.683 | 12.206 | 1.00 | 10.00 |
| ATOM | 521 | CA | TYR | 72 | −19.519 | −36.131 | 12.413 | 1.00 | 9.20 |
| ATOM | 522 | C | TYR | 72 | −20.853 | −36.797 | 12.133 | 1.00 | 9.79 |
| ATOM | 523 | O | TYR | 72 | −20.913 | −37.998 | 11.862 | 1.00 | 9.97 |
| ATOM | 524 | CB | TYR | 72 | −19.068 | −36.435 | 13.844 | 1.00 | 9.47 |
| ATOM | 525 | CG | TYR | 72 | −17.641 | −36.019 | 14.174 | 1.00 | 10.37 |
| ATOM | 526 | CD1 | TYR | 72 | −16.774 | −35.494 | 13.194 | 1.00 | 9.54 |
| ATOM | 527 | CD2 | TYR | 72 | −17.137 | −36.180 | 15.470 | 1.00 | 9.09 |
| ATOM | 528 | CE1 | TYR | 72 | −15.433 | −35.141 | 13.515 | 1.00 | 10.62 |
| ATOM | 529 | CE2 | TYR | 72 | −15.834 | −35.834 | 15.788 | 1.00 | 9.58 |
| ATOM | 530 | CZ | TYR | 72 | −14.978 | −35.319 | 14.816 | 1.00 | 11.55 |
| ATOM | 531 | OH | TYR | 72 | −13.681 | −34.981 | 15.176 | 1.00 | 11.99 |
| ATOM | 532 | N | ALA | 73 | −21.932 | −36.008 | 12.206 | 1.00 | 9.50 |
| ATOM | 533 | CA | ALA | 73 | −23.301 | −36.486 | 11.941 | 1.00 | 9.88 |
| ATOM | 534 | C | ALA | 73 | −23.858 | −36.167 | 10.536 | 1.00 | 10.33 |
| ATOM | 535 | O | ALA | 73 | −25.021 | −36.508 | 10.220 | 1.00 | 11.21 |
| ATOM | 536 | CB | ALA | 73 | −24.275 | −35.969 | 13.052 | 1.00 | 9.83 |
| ATOM | 537 | N | SER | 74 | −23.033 | −35.564 | 9.677 | 1.00 | 10.17 |
| ATOM | 538 | CA | SER | 74 | −23.470 | −35.041 | 8.362 | 1.00 | 9.89 |
| ATOM | 539 | C | SER | 74 | −24.612 | −34.035 | 8.489 | 1.00 | 10.12 |
| ATOM | 540 | O | SER | 74 | −25.504 | −34.007 | 7.634 | 1.00 | 10.11 |
| ATOM | 541 | CB | SER | 74 | −23.882 | −36.160 | 7.386 | 1.00 | 10.12 |
| ATOM | 542 | OG | SER | 74 | −22.828 | −37.066 | 7.184 | 1.00 | 10.47 |
| ATOM | 543 | N | PHE | 75 | −24.612 | −33.235 | 9.569 | 1.00 | 9.74 |
| ATOM | 544 | CA | PHE | 75 | −25.649 | −32.218 | 9.735 | 1.00 | 10.02 |
| ATOM | 545 | C | PHE | 75 | −25.591 | −31.067 | 8.700 | 1.00 | 10.34 |
| ATOM | 546 | O | PHE | 75 | −26.541 | −30.287 | 8.580 | 1.00 | 11.03 |
| ATOM | 547 | CB | PHE | 75 | −25.659 | −31.676 | 11.167 | 1.00 | 9.55 |
| ATOM | 548 | CG | PHE | 75 | −26.192 | −32.644 | 12.199 | 1.00 | 9.05 |
| ATOM | 549 | CD1 | PHE | 75 | −27.081 | −33.670 | 11.846 | 1.00 | 8.04 |
| ATOM | 550 | CD2 | PHE | 75 | −25.824 | −32.502 | 13.545 | 1.00 | 8.19 |
| ATOM | 551 | CE1 | PHE | 75 | −27.604 | −34.543 | 12.832 | 1.00 | 8.47 |
| ATOM | 552 | CE2 | PHE | 75 | −26.331 | −33.368 | 14.539 | 1.00 | 9.33 |
| ATOM | 553 | CZ | PHE | 75 | −27.224 | −34.402 | 14.168 | 1.00 | 8.62 |
| ATOM | 554 | N | GLY | 76 | −24.487 | −30.962 | 7.950 | 1.00 | 10.30 |
| ATOM | 555 | CA | GLY | 76 | −24.401 | −29.961 | 6.877 | 1.00 | 9.88 |
| ATOM | 556 | C | GLY | 76 | −25.430 | −30.271 | 5.796 | 1.00 | 10.78 |
| ATOM | 557 | O | GLY | 76 | −25.872 | −29.365 | 5.082 | 1.00 | 11.26 |
| ATOM | 558 | N | PHE | 77 | −25.828 | −31.546 | 5.695 | 1.00 | 10.41 |
| ATOM | 559 | CA | PHE | 77 | −26.877 | −32.004 | 4.771 | 1.00 | 10.41 |
| ATOM | 560 | C | PHE | 77 | −28.252 | −31.834 | 5.374 | 1.00 | 10.17 |
| ATOM | 561 | O | PHE | 77 | −29.260 | −31.932 | 4.677 | 1.00 | 10.68 |
| ATOM | 562 | CB | PHE | 77 | −26.699 | −33.502 | 4.466 | 1.00 | 10.26 |
| ATOM | 563 | CG | PHE | 77 | −25.452 | −33.824 | 3.678 | 1.00 | 10.59 |
| ATOM | 564 | CD1 | PHE | 77 | −25.516 | −33.994 | 2.299 | 1.00 | 8.54 |
| ATOM | 565 | CD2 | PHE | 77 | −24.225 | −33.951 | 4.316 | 1.00 | 9.59 |
| ATOM | 566 | CE1 | PHE | 77 | −24.401 | −34.327 | 1.565 | 1.00 | 8.38 |
| ATOM | 567 | CE2 | PHE | 77 | −23.097 | −34.274 | 3.581 | 1.00 | 10.00 |
| ATOM | 568 | CZ | PHE | 77 | −23.189 | −34.465 | 2.204 | 1.00 | 9.98 |
| ATOM | 569 | N | GLY | 78 | −28.298 | −31.608 | 6.674 | 1.00 | 10.28 |
| ATOM | 570 | CA | GLY | 78 | −29.547 | −31.640 | 7.383 | 1.00 | 9.74 |
| ATOM | 571 | C | GLY | 78 | −29.782 | −32.891 | 8.210 | 1.00 | 10.82 |
| ATOM | 572 | O | GLY | 78 | −30.811 | −32.992 | 8.878 | 1.00 | 10.84 |
| ATOM | 573 | N | GLY | 79 | −28.834 | −33.829 | 8.184 | 1.00 | 10.66 |
| ATOM | 574 | CA | GLY | 79 | −28.957 | −35.091 | 8.925 | 1.00 | 11.65 |
| ATOM | 575 | C | GLY | 79 | −30.109 | −35.989 | 8.483 | 1.00 | 11.71 |
| ATOM | 576 | O | GLY | 79 | −30.545 | −36.859 | 9.231 | 1.00 | 12.19 |
| ATOM | 577 | N | GLU | 80 | −30.598 | −35.773 | 7.266 | 1.00 | 12.30 |
| ATOM | 578 | CA | GLU | 80 | −31.678 | −36.579 | 6.671 | 1.00 | 12.28 |
| ATOM | 579 | C | GLU | 80 | −31.683 | −36.192 | 5.213 | 1.00 | 12.39 |
| ATOM | 580 | O | GLU | 80 | −30.985 | −35.233 | 4.827 | 1.00 | 11.98 |
| ATOM | 581 | CB | GLU | 80 | −33.039 | −36.245 | 7.292 | 1.00 | 13.30 |
| ATOM | 582 | CG | GLU | 80 | −33.464 | −34.783 | 7.089 | 1.00 | 12.93 |
| ATOM | 583 | CD | GLU | 80 | −34.759 | −34.434 | 7.816 | 1.00 | 17.90 |
| ATOM | 584 | OE1 | GLU | 80 | −35.364 | −35.336 | 8.427 | 1.00 | 17.90 |
| ATOM | 585 | OE2 | GLU | 80 | −35.167 | −33.256 | 7.787 | 1.00 | 18.58 |
| ATOM | 586 | N | ASN | 81 | −32.443 | −36.929 | 4.402 | 1.00 | 11.37 |
| ATOM | 587 | CA | ASN | 81 | −32.570 | −36.610 | 2.965 | 1.00 | 11.74 |
| ATOM | 588 | C | ASN | 81 | −33.517 | −35.472 | 2.694 | 1.00 | 11.45 |
| ATOM | 589 | O | ASN | 81 | −34.415 | −35.203 | 3.513 | 1.00 | 11.17 |
| ATOM | 590 | CB | ASN | 81 | −33.041 | −37.850 | 2.201 | 1.00 | 11.56 |
| ATOM | 591 | CG | ASN | 81 | −32.058 | −39.005 | 2.337 | 1.00 | 12.44 |
| ATOM | 592 | ND2 | ASN | 81 | −32.567 | −40.210 | 2.640 | 1.00 | 11.36 |
| ATOM | 593 | OD1 | ASN | 81 | −30.843 | −38.795 | 2.221 | 1.00 | 10.11 |
| ATOM | 594 | N | GLY | 82 | −33.336 | −34.818 | 1.547 | 1.00 | 10.20 |
| ATOM | 595 | CA | GLY | 82 | −34.365 | −33.898 | 1.026 | 1.00 | 10.61 |
| ATOM | 596 | C | GLY | 82 | −34.329 | −32.416 | 1.386 | 1.00 | 11.24 |
| ATOM | 597 | O | GLY | 82 | −35.301 | −31.656 | 1.117 | 1.00 | 11.33 |
| ATOM | 598 | N | HIS | 83 | −33.235 | −31.973 | 2.004 | 1.00 | 10.86 |
| ATOM | 599 | CA | HIS | 83 | −33.107 | −30.550 | 2.315 | 1.00 | 11.17 |
| ATOM | 600 | C | HIS | 83 | −32.528 | −29.843 | 1.115 | 1.00 | 11.60 |
| ATOM | 601 | O | HIS | 83 | −31.889 | −30.456 | 0.243 | 1.00 | 11.20 |
| ATOM | 602 | CB | HIS | 83 | −32.178 | −30.311 | 3.521 | 1.00 | 10.75 |
| ATOM | 603 | CG | HIS | 83 | −32.755 | −30.737 | 4.838 | 1.00 | 10.50 |
| ATOM | 604 | CD2 | HIS | 83 | −33.631 | −31.722 | 5.155 | 1.00 | 9.65 |
| ATOM | 605 | ND1 | HIS | 83 | −32.388 | −30.153 | 6.034 | 1.00 | 8.96 |
| ATOM | 606 | CE1 | HIS | 83 | −33.024 | −30.746 | 7.028 | 1.00 | 10.80 |
| ATOM | 607 | NE2 | HIS | 83 | −33.792 | −31.694 | 6.523 | 1.00 | 11.18 |
| ATOM | 608 | N | LEU | 84 | −32.730 | −28.536 | 1.074 | 1.00 | 11.92 |
| ATOM | 609 | CA | LEU | 84 | −31.946 | −27.716 | 0.187 | 1.00 | 12.32 |
| ATOM | 610 | C | LEU | 84 | −30.580 | −27.558 | 0.886 | 1.00 | 12.06 |
| ATOM | 611 | O | LEU | 84 | −30.521 | −27.147 | 2.054 | 1.00 | 12.39 |
| ATOM | 612 | CB | LEU | 84 | −32.623 | −26.342 | −0.013 | 1.00 | 12.88 |
| ATOM | 613 | CG | LEU | 84 | −31.868 | −25.298 | −0.857 | 1.00 | 12.71 |
| ATOM | 614 | CD1 | LEU | 84 | −31.508 | −25.841 | −2.234 | 1.00 | 12.90 |
| ATOM | 615 | CD2 | LEU | 84 | −32.747 | −24.055 | −1.023 | 1.00 | 13.66 |
| ATOM | 616 | N | MET | 85 | −29.490 | −27.926 | 0.216 | 1.00 | 11.13 |
| ATOM | 617 | CA | MET | 85 | −28.175 | −27.756 | 0.849 | 1.00 | 10.54 |
| ATOM | 618 | C | MET | 85 | −27.507 | −26.523 | 0.218 | 1.00 | 10.62 |
| ATOM | 619 | O | MET | 85 | −27.385 | −26.425 | −1.006 | 1.00 | 10.30 |
| ATOM | 620 | CB | MET | 85 | −27.305 | −29.030 | 0.720 | 1.00 | 9.96 |
| ATOM | 621 | CG | MET | 85 | −26.040 | −29.017 | 1.619 | 1.00 | 8.24 |
| ATOM | 622 | SD | MET | 85 | −25.110 | −30.565 | 1.596 | 0.93 | 8.95 |
| ATOM | 623 | CE | MET | 85 | −24.375 | −30.551 | −0.058 | 1.00 | 5.31 |
| ATOM | 624 | N | VAL | 86 | −27.140 | −25.575 | 1.074 | 1.00 | 11.51 |
| ATOM | 625 | CA | VAL | 86 | −26.416 | −24.371 | 0.696 | 1.00 | 11.19 |
| ATOM | 626 | C | VAL | 86 | −24.948 | −24.689 | 0.887 | 1.00 | 11.87 |
| ATOM | 627 | O | VAL | 86 | −24.439 | −24.701 | 2.031 | 1.00 | 12.24 |
| ATOM | 628 | CB | VAL | 86 | −26.849 | −23.156 | 1.543 | 1.00 | 11.70 |
| ATOM | 629 | CG1 | VAL | 86 | −26.092 | −21.894 | 1.160 | 1.00 | 11.01 |
| ATOM | 630 | CG2 | VAL | 86 | −28.379 | −22.928 | 1.428 | 1.00 | 11.58 |
| ATOM | 631 | N | GLN | 87 | −24.296 | −25.009 | −0.241 | 1.00 | 11.03 |
| ATOM | 632 | CA | GLN | 87 | −22.907 | −25.457 | −0.267 | 1.00 | 11.57 |
| ATOM | 633 | C | GLN | 87 | −22.001 | −24.220 | −0.321 | 1.00 | 11.34 |
| ATOM | 634 | O | GLN | 87 | −22.033 | −23.484 | −1.313 | 1.00 | 10.39 |
| ATOM | 635 | CB | GLN | 87 | −22.656 | −26.341 | −1.492 | 1.00 | 10.99 |
| ATOM | 636 | CG | GLN | 87 | −21.192 | −26.735 | −1.693 | 1.00 | 11.10 |
| ATOM | 637 | CD | GLN | 87 | −20.655 | −27.582 | −0.534 | 1.00 | 13.42 |
| ATOM | 638 | NE2 | GLN | 87 | −19.356 | −27.526 | −0.308 | 1.00 | 13.56 |
| ATOM | 639 | OE1 | GLN | 87 | −21.411 | −28.282 | 0.135 | 1.00 | 12.98 |
| ATOM | 640 | N | LEU | 88 | −21.206 | −24.010 | 0.744 | 1.00 | 11.52 |
| ATOM | 641 | CA | LEU | 88 | −20.516 | −22.721 | 0.963 | 1.00 | 11.44 |
| ATOM | 642 | C | LEU | 88 | −19.019 | −22.725 | 0.610 | 1.00 | 11.72 |
| ATOM | 643 | O | LEU | 88 | −18.392 | −21.663 | 0.554 | 1.00 | 11.80 |
| ATOM | 644 | CB | LEU | 88 | −20.693 | −22.249 | 2.411 | 1.00 | 11.23 |
| ATOM | 645 | CG | LEU | 88 | −22.115 | −21.948 | 2.894 | 1.00 | 9.62 |
| ATOM | 646 | CD1 | LEU | 88 | −22.128 | −21.696 | 4.404 | 1.00 | 9.38 |
| ATOM | 647 | CD2 | LEU | 88 | −22.692 | −20.717 | 2.179 | 1.00 | 5.60 |
| ATOM | 648 | N | ASP | 89 | −18.465 | −23.904 | 0.357 | 1.00 | 11.10 |
| ATOM | 649 | CA | ASP | 89 | −17.014 | −24.071 | 0.322 | 1.00 | 11.84 |
| ATOM | 650 | C | ASP | 89 | −16.262 | −23.431 | −0.854 | 1.00 | 12.47 |
| ATOM | 651 | O | ASP | 89 | −15.023 | −23.543 | −0.932 | 1.00 | 13.84 |
| ATOM | 652 | CB | ASP | 89 | −16.618 | −25.536 | 0.559 | 1.00 | 11.70 |
| ATOM | 653 | CG | ASP | 89 | −16.463 | −26.336 | −0.732 | 1.00 | 11.18 |
| ATOM | 654 | OD1 | ASP | 89 | −17.201 | −26.076 | −1.695 | 1.00 | 10.66 |
| ATOM | 655 | OD2 | ASP | 89 | −15.613 | −27.265 | −0.755 | 1.00 | 11.27 |
| ATOM | 656 | N | ARG | 90 | −16.973 | −22.740 | −1.750 | 1.00 | 12.85 |
| ATOM | 657 | CA | ARG | 90 | −16.311 | −21.959 | −2.806 | 0.50 | 13.19 |
| ATOM | 658 | C | ARG | 90 | −16.162 | −20.499 | −2.369 | 1.00 | 13.00 |
| ATOM | 659 | O | ARG | 90 | −15.499 | −19.720 | −3.036 | 1.00 | 13.66 |
| ATOM | 660 | CB | ARG | 90 | −17.071 | −22.028 | −4.139 | 0.50 | 13.34 |
| ATOM | 661 | CG | ARG | 90 | −17.284 | −23.418 | −4.708 | 0.50 | 14.06 |
| ATOM | 662 | CD | ARG | 90 | −16.025 | −24.280 | −4.604 | 0.50 | 17.95 |
| ATOM | 663 | NE | ARG | 90 | −15.998 | −25.270 | −5.672 | 0.50 | 20.49 |
| ATOM | 664 | CZ | ARG | 90 | −16.556 | −26.473 | −5.583 | 0.50 | 22.29 |
| ATOM | 665 | NH1 | ARG | 90 | −17.179 | −26.838 | −4.467 | 0.50 | 22.94 |
| ATOM | 666 | NH2 | ARG | 90 | −16.494 | −27.308 | −6.610 | 0.50 | 23.46 |
| ATOM | 667 | N | MET | 91 | −16.805 | −20.131 | −1.250 | 1.00 | 12.40 |
| ATOM | 668 | CA | MET | 91 | −16.681 | −18.782 | −0.677 | 1.00 | 12.46 |
| ATOM | 669 | C | MET | 91 | −15.599 | −18.813 | 0.414 | 1.00 | 12.94 |
| ATOM | 670 | O | MET | 91 | −15.875 | −19.067 | 1.592 | 1.00 | 12.83 |
| ATOM | 671 | CB | MET | 91 | −18.015 | −18.298 | −0.103 | 1.00 | 11.57 |
| ATOM | 672 | CG | MET | 91 | −19.169 | −18.314 | −1.122 | 1.00 | 12.14 |
| ATOM | 673 | SD | MET | 91 | −20.764 | −18.121 | −0.287 | 0.93 | 14.65 |
| ATOM | 674 | CE | MET | 91 | −20.722 | −16.345 | −0.015 | 1.00 | 13.86 |
| ATOM | 675 | N | ILE | 92 | −14.366 | −18.554 | 0.008 | 1.00 | 12.68 |
| ATOM | 676 | CA | ILE | 92 | −13.217 | −18.871 | 0.832 | 1.00 | 13.19 |
| ATOM | 677 | C | ILE | 92 | −12.376 | −17.669 | 1.226 | 1.00 | 13.28 |
| ATOM | 678 | O | ILE | 92 | −11.314 | −17.818 | 1.832 | 1.00 | 12.66 |
| ATOM | 679 | CB | ILE | 92 | −12.285 | −19.881 | 0.079 | 1.00 | 13.59 |
| ATOM | 680 | CG1 | ILE | 92 | −11.801 | −19.291 | −1.260 | 1.00 | 13.91 |
| ATOM | 681 | CG2 | ILE | 92 | −12.991 | −21.213 | −0.105 | 1.00 | 11.74 |
| ATOM | 682 | CD1 | ILE | 92 | −10.689 | −20.153 | −1.939 | 1.00 | 17.20 |
| ATOM | 683 | N | ASP | 93 | −12.813 | −16.476 | 0.860 | 1.00 | 13.49 |
| ATOM | 684 | CA | ASP | 93 | −11.902 | −15.331 | 0.983 | 1.00 | 14.42 |
| ATOM | 685 | C | ASP | 93 | −11.876 | −14.666 | 2.341 | 1.00 | 13.84 |
| ATOM | 686 | O | ASP | 93 | −12.901 | −14.567 | 3.010 | 1.00 | 13.05 |
| ATOM | 687 | CB | ASP | 93 | −12.257 | −14.284 | −0.048 | 1.00 | 15.91 |
| ATOM | 688 | CG | ASP | 93 | −11.896 | −14.717 | −1.453 | 1.00 | 17.71 |
| ATOM | 689 | OD1 | ASP | 93 | −11.125 | −15.685 | −1.637 | 1.00 | 21.56 |
| ATOM | 690 | OD2 | ASP | 93 | −12.380 | −14.062 | −2.371 | 1.00 | 23.18 |
| ATOM | 691 | N | VAL | 94 | −10.688 | −14.213 | 2.728 | 1.00 | 13.80 |
| ATOM | 692 | CA | VAL | 94 | −10.564 | −13.107 | 3.681 | 1.00 | 14.74 |
| ATOM | 693 | C | VAL | 94 | −10.883 | −11.829 | 2.891 | 1.00 | 15.46 |
| ATOM | 694 | O | VAL | 94 | −10.099 | −11.393 | 2.031 | 1.00 | 15.98 |
| ATOM | 695 | CB | VAL | 94 | −9.175 | −13.049 | 4.347 | 1.00 | 14.34 |
| ATOM | 696 | CG1 | VAL | 94 | −9.061 | −11.816 | 5.265 | 1.00 | 13.36 |
| ATOM | 697 | CG2 | VAL | 94 | −8.909 | −14.365 | 5.100 | 1.00 | 14.26 |
| ATOM | 698 | N | ILE | 95 | −12.081 | −11.313 | 3.142 | 1.00 | 16.32 |
| ATOM | 699 | CA | ILE | 95 | −12.699 | −10.204 | 2.403 | 1.00 | 17.74 |
| ATOM | 700 | C | ILE | 95 | −11.951 | −8.894 | 2.678 | 1.00 | 18.33 |
| ATOM | 701 | O | ILE | 95 | −11.722 | −8.099 | 1.774 | 1.00 | 18.30 |
| ATOM | 702 | CB | ILE | 95 | −14.177 | −9.998 | 2.843 | 1.00 | 17.96 |
| ATOM | 703 | CG1 | ILE | 95 | −15.020 | −11.253 | 2.548 | 1.00 | 18.83 |
| ATOM | 704 | CG2 | ILE | 95 | −14.784 | −8.760 | 2.165 | 1.00 | 18.72 |
| ATOM | 705 | CD1 | ILE | 95 | −16.345 | −11.326 | 3.392 | 1.00 | 20.36 |
| ATOM | 706 | N | SER | 96 | −11.597 | −8.667 | 3.939 | 1.00 | 17.91 |
| ATOM | 707 | CA | SER | 96 | −10.880 | −7.461 | 4.318 | 1.00 | 17.92 |
| ATOM | 708 | C | SER | 96 | −10.141 | −7.728 | 5.618 | 1.00 | 17.99 |
| ATOM | 709 | O | SER | 96 | −10.449 | −8.682 | 6.348 | 1.00 | 17.21 |
| ATOM | 710 | CB | SER | 96 | −11.844 | −6.286 | 4.483 | 1.00 | 18.29 |
| ATOM | 711 | OG | SER | 96 | −12.766 | −6.551 | 5.543 | 1.00 | 20.02 |
| ATOM | 712 | N | TYR | 97 | −9.138 | −6.900 | 5.873 | 1.00 | 17.34 |
| ATOM | 713 | CA | TYR | 97 | −8.398 | −6.934 | 7.104 | 1.00 | 17.65 |
| ATOM | 714 | C | TYR | 97 | −8.120 | −5.485 | 7.446 | 1.00 | 18.38 |
| ATOM | 715 | O | TYR | 97 | −7.670 | −4.722 | 6.594 | 1.00 | 18.42 |
| ATOM | 716 | CB | TYR | 97 | −7.082 | −7.713 | 6.959 | 1.00 | 17.00 |
| ATOM | 717 | CG | TYR | 97 | −6.223 | −7.592 | 8.185 | 1.00 | 16.65 |
| ATOM | 718 | CD1 | TYR | 97 | −6.540 | −8.285 | 9.338 | 1.00 | 16.55 |
| ATOM | 719 | CD2 | TYR | 97 | −5.118 | −6.721 | 8.212 | 1.00 | 18.63 |
| ATOM | 720 | CE1 | TYR | 97 | −5.768 | −8.152 | 10.504 | 1.00 | 17.19 |
| ATOM | 721 | CE2 | TYR | 97 | −4.348 | −6.572 | 9.356 | 1.00 | 17.95 |
| ATOM | 722 | CZ | TYR | 97 | −4.678 | −7.291 | 10.504 | 1.00 | 18.99 |
| ATOM | 723 | OH | TYR | 97 | −3.929 | −7.145 | 11.645 | 1.00 | 18.21 |
| ATOM | 724 | N | ASN | 98 | −8.421 | −5.108 | 8.680 | 1.00 | 18.65 |
| ATOM | 725 | CA | ASN | 98 | −8.156 | −3.770 | 9.155 | 1.00 | 19.73 |
| ATOM | 726 | C | ASN | 98 | −6.904 | −3.745 | 10.026 | 1.00 | 19.92 |
| ATOM | 727 | O | ASN | 98 | −6.885 | −4.319 | 11.107 | 1.00 | 19.41 |
| ATOM | 728 | CB | ASN | 98 | −9.359 | −3.247 | 9.941 | 1.00 | 20.09 |
| ATOM | 729 | CG | ASN | 98 | −9.218 | −1.764 | 10.313 | 1.00 | 22.66 |
| ATOM | 730 | ND2 | ASN | 98 | −10.323 | −1.045 | 10.263 | 1.00 | 24.19 |
| ATOM | 731 | OD1 | ASN | 98 | −8.137 | −1.288 | 10.657 | 1.00 | 24.30 |
| ATOM | 732 | N | ASP | 99 | −5.862 | −3.062 | 9.568 | 1.00 | 20.64 |
| ATOM | 733 | CA | ASP | 99 | −4.585 | −3.118 | 10.291 | 1.00 | 22.15 |
| ATOM | 734 | C | ASP | 99 | −4.513 | −2.246 | 11.549 | 1.00 | 21.68 |
| ATOM | 735 | O | ASP | 99 | −3.546 | −2.341 | 12.294 | 1.00 | 22.05 |
| ATOM | 736 | CB | ASP | 99 | −3.370 | −2.888 | 9.357 | 1.00 | 22.87 |
| ATOM | 737 | CG | ASP | 99 | −3.387 | −1.528 | 8.691 | 0.80 | 26.16 |
| ATOM | 738 | OD1 | ASP | 99 | −4.155 | −0.621 | 9.127 | 0.80 | 28.43 |
| ATOM | 739 | OD2 | ASP | 99 | −2.623 | −1.369 | 7.707 | 0.80 | 30.39 |
| ATOM | 740 | N | LYS | 100 | −5.536 | −1.427 | 11.786 | 1.00 | 21.32 |
| ATOM | 741 | CA | LYS | 100 | −5.635 | −0.661 | 13.029 | 1.00 | 21.92 |
| ATOM | 742 | C | LYS | 100 | −6.261 | −1.501 | 14.146 | 1.00 | 20.71 |
| ATOM | 743 | O | LYS | 100 | −5.909 | −1.358 | 15.315 | 1.00 | 21.19 |
| ATOM | 744 | CB | LYS | 100 | −6.489 | 0.588 | 12.829 | 1.00 | 22.03 |
| ATOM | 745 | CG | LYS | 100 | −5.894 | 1.658 | 11.948 | 1.00 | 25.59 |
| ATOM | 746 | CD | LYS | 100 | −6.960 | 2.719 | 11.693 | 1.00 | 29.78 |
| ATOM | 747 | CE | LYS | 100 | −6.666 | 3.556 | 10.454 | 1.00 | 33.34 |
| ATOM | 748 | NZ | LYS | 100 | −7.960 | 4.008 | 9.812 | 1.00 | 36.02 |
| ATOM | 749 | N | THR | 101 | −7.207 | −2.361 | 13.789 | 1.00 | 19.45 |
| ATOM | 750 | CA | THR | 101 | −7.940 | −3.133 | 14.810 | 1.00 | 18.17 |
| ATOM | 751 | C | THR | 101 | −7.544 | −4.606 | 14.835 | 1.00 | 17.72 |
| ATOM | 752 | O | THR | 101 | −7.818 | −5.311 | 15.810 | 1.00 | 17.67 |
| ATOM | 753 | CB | THR | 101 | −9.414 | −3.051 | 14.553 | 1.00 | 17.95 |
| ATOM | 754 | CG2 | THR | 101 | −9.862 | −1.591 | 14.531 | 1.00 | 19.00 |
| ATOM | 755 | OG1 | THR | 101 | −9.690 | −3.629 | 13.274 | 1.00 | 15.77 |
| ATOM | 756 | N | GLY | 102 | −6.914 | −5.076 | 13.761 | 1.00 | 16.85 |
| ATOM | 757 | CA | GLY | 102 | −6.591 | −6.503 | 13.636 | 1.00 | 17.00 |
| ATOM | 758 | C | GLY | 102 | −7.801 | −7.363 | 13.272 | 1.00 | 16.73 |
| ATOM | 759 | O | GLY | 102 | −7.731 | −8.601 | 13.321 | 1.00 | 17.39 |
| ATOM | 760 | N | ILE | 103 | −8.914 | −6.729 | 12.918 | 1.00 | 15.64 |
| ATOM | 761 | CA | ILE | 103 | −10.127 | −7.481 | 12.598 | 1.00 | 15.36 |
| ATOM | 762 | C | ILE | 103 | −10.200 | −7.897 | 11.124 | 1.00 | 15.68 |
| ATOM | 763 | O | ILE | 103 | −9.989 | −7.071 | 10.230 | 1.00 | 15.09 |
| ATOM | 764 | CB | ILE | 103 | −11.392 | −6.707 | 13.024 | 1.00 | 16.09 |
| ATOM | 765 | CG1 | ILE | 103 | −11.403 | −6.550 | 14.564 | 1.00 | 14.73 |
| ATOM | 766 | CG2 | ILE | 103 | −12.647 | −7.402 | 12.528 | 1.00 | 14.09 |
| ATOM | 767 | CD1 | ILE | 103 | −12.496 | −5.633 | 15.097 | 1.00 | 15.75 |
| ATOM | 768 | N | ALA | 104 | −10.508 | −9.178 | 10.890 | 1.00 | 15.12 |
| ATOM | 769 | CA | ALA | 104 | −10.671 | −9.746 | 9.542 | 1.00 | 15.43 |
| ATOM | 770 | C | ALA | 104 | −12.131 | −10.052 | 9.271 | 1.00 | 15.49 |
| ATOM | 771 | O | ALA | 104 | −12.837 | −10.543 | 10.158 | 1.00 | 15.74 |
| ATOM | 772 | CB | ALA | 104 | −9.877 | −11.054 | 9.420 | 1.00 | 15.12 |
| ATOM | 773 | N | HIS | 105 | −12.575 | −9.787 | 8.046 | 1.00 | 14.26 |
| ATOM | 774 | CA | HIS | 105 | −13.877 | −10.233 | 7.598 | 1.00 | 13.90 |
| ATOM | 775 | C | HIS | 105 | −13.644 | −11.426 | 6.696 | 1.00 | 13.95 |
| ATOM | 776 | O | HIS | 105 | −12.798 | −11.358 | 5.783 | 1.00 | 13.13 |
| ATOM | 777 | CB | HIS | 105 | −14.621 | −9.121 | 6.864 | 1.00 | 14.83 |
| ATOM | 778 | CG | HIS | 105 | −15.037 | −7.987 | 7.753 | 1.00 | 18.16 |
| ATOM | 779 | CD2 | HIS | 105 | −14.508 | −7.521 | 8.911 | 1.00 | 20.88 |
| ATOM | 780 | ND1 | HIS | 105 | −16.137 | −7.200 | 7.495 | 1.00 | 19.81 |
| ATOM | 781 | CE1 | HIS | 105 | −16.257 | −6.289 | 8.441 | 1.00 | 19.57 |
| ATOM | 782 | NE2 | HIS | 105 | −15.290 | −6.470 | 9.319 | 1.00 | 21.03 |
| ATOM | 783 | N | VAL | 106 | −14.354 | −12.526 | 6.987 | 1.00 | 12.21 |
| ATOM | 784 | CA | VAL | 106 | −14.130 | −13.821 | 6.327 | 1.00 | 11.37 |
| ATOM | 785 | C | VAL | 106 | −15.410 | −14.488 | 5.807 | 1.00 | 10.81 |
| ATOM | 786 | O | VAL | 106 | −16.439 | −14.527 | 6.489 | 1.00 | 11.01 |
| ATOM | 787 | CB | VAL | 106 | −13.341 | −14.817 | 7.246 | 1.00 | 11.09 |
| ATOM | 788 | CG1 | VAL | 106 | −12.858 | −16.047 | 6.443 | 1.00 | 10.54 |
| ATOM | 789 | CG2 | VAL | 106 | −12.150 | −14.089 | 7.897 | 1.00 | 9.58 |
| ATOM | 790 | N | GLU | 107 | −15.330 | −14.998 | 4.583 | 1.00 | 10.56 |
| ATOM | 791 | CA | GLU | 107 | −16.416 | −15.760 | 3.966 | 1.00 | 10.70 |
| ATOM | 792 | C | GLU | 107 | −16.606 | −17.112 | 4.669 | 1.00 | 10.60 |
| ATOM | 793 | O | GLU | 107 | −15.647 | −17.643 | 5.255 | 1.00 | 10.62 |
| ATOM | 794 | CB | GLU | 107 | −16.135 | −15.932 | 2.459 | 1.00 | 11.23 |
| ATOM | 795 | CG | GLU | 107 | −16.509 | −14.675 | 1.662 | 1.00 | 11.29 |
| ATOM | 796 | CD | GLU | 107 | −16.279 | −14.811 | 0.166 | 1.00 | 13.82 |
| ATOM | 797 | OE1 | GLU | 107 | −15.351 | −15.532 | −0.264 | 1.00 | 13.35 |
| ATOM | 798 | OE2 | GLU | 107 | −17.026 | −14.173 | −0.591 | 1.00 | 14.46 |
| ATOM | 799 | N | PRO | 108 | −17.834 | −17.671 | 4.609 | 1.00 | 10.10 |
| ATOM | 800 | CA | PRO | 108 | −18.228 | −18.762 | 5.492 | 1.00 | 9.56 |
| ATOM | 801 | C | PRO | 108 | −17.812 | −20.180 | 5.025 | 1.00 | 10.27 |
| ATOM | 802 | O | PRO | 108 | −18.116 | −21.175 | 5.713 | 1.00 | 10.05 |
| ATOM | 803 | CB | PRO | 108 | −19.761 | −18.643 | 5.515 | 1.00 | 9.81 |
| ATOM | 804 | CG | PRO | 108 | −20.132 | −18.068 | 4.176 | 1.00 | 9.29 |
| ATOM | 805 | CD | PRO | 108 | −18.955 | −17.209 | 3.749 | 1.00 | 9.52 |
| ATOM | 806 | N | GLY | 109 | −17.117 | −20.257 | 3.890 | 1.00 | 10.00 |
| ATOM | 807 | CA | GLY | 109 | −16.659 | −21.512 | 3.316 | 1.00 | 9.78 |
| ATOM | 808 | C | GLY | 109 | −15.151 | −21.716 | 3.431 | 1.00 | 9.65 |
| ATOM | 809 | O | GLY | 109 | −14.637 | −22.752 | 3.005 | 1.00 | 8.60 |
| ATOM | 810 | N | ALA | 110 | −14.448 | −20.730 | 3.993 | 1.00 | 8.59 |
| ATOM | 811 | CA | ALA | 110 | −13.003 | −20.828 | 4.243 | 1.00 | 9.11 |
| ATOM | 812 | C | ALA | 110 | −12.715 | −21.931 | 5.241 | 1.00 | 9.77 |
| ATOM | 813 | O | ALA | 110 | −13.338 | −21.972 | 6.326 | 1.00 | 9.46 |
| ATOM | 814 | CB | ALA | 110 | −12.478 | −19.488 | 4.797 | 1.00 | 7.94 |
| ATOM | 815 | N | ARG | 111 | −11.795 | −22.826 | 4.890 | 1.00 | 9.62 |
| ATOM | 816 | CA | ARG | 111 | −11.342 | −23.874 | 5.807 | 1.00 | 10.64 |
| ATOM | 817 | C | ARG | 111 | −10.179 | −23.411 | 6.698 | 1.00 | 11.34 |
| ATOM | 818 | O | ARG | 111 | −9.443 | −22.479 | 6.343 | 1.00 | 12.15 |
| ATOM | 819 | CB | ARG | 111 | −10.997 | −25.164 | 5.033 | 1.00 | 10.53 |
| ATOM | 820 | CG | ARG | 111 | −12.233 | −25.766 | 4.328 | 1.00 | 11.03 |
| ATOM | 821 | CD | ARG | 111 | −11.831 | −26.726 | 3.165 | 1.00 | 11.05 |
| ATOM | 822 | NE | ARG | 111 | −12.981 | −27.138 | 2.356 | 1.00 | 10.64 |
| ATOM | 823 | CZ | ARG | 111 | −13.749 | −28.203 | 2.616 | 1.00 | 12.21 |
| ATOM | 824 | NH1 | ARG | 111 | −13.506 | −28.979 | 3.667 | 1.00 | 9.17 |
| ATOM | 825 | NH2 | ARG | 111 | −14.753 | −28.504 | 1.805 | 1.00 | 10.31 |
| ATOM | 826 | N | LEU | 112 | −10.028 | −24.047 | 7.863 | 1.00 | 11.36 |
| ATOM | 827 | CA | LEU | 112 | −9.083 | −23.579 | 8.876 | 1.00 | 11.52 |
| ATOM | 828 | C | LEU | 112 | −7.667 | −23.392 | 8.327 | 1.00 | 11.63 |
| ATOM | 829 | O | LEU | 112 | −7.023 | −22.387 | 8.608 | 1.00 | 11.05 |
| ATOM | 830 | CB | LEU | 112 | −9.061 | −24.501 | 10.106 | 1.00 | 10.89 |
| ATOM | 831 | CG | LEU | 112 | −10.370 | −24.695 | 10.883 | 1.00 | 11.49 |
| ATOM | 832 | CD1 | LEU | 112 | −10.058 | −25.414 | 12.169 | 1.00 | 11.56 |
| ATOM | 833 | CD2 | LEU | 112 | −11.096 | −23.363 | 11.190 | 1.00 | 11.75 |
| ATOM | 834 | N | GLY | 113 | −7.183 | −24.356 | 7.551 | 1.00 | 11.87 |
| ATOM | 835 | CA | GLY | 113 | −5.820 | −24.259 | 6.993 | 1.00 | 11.96 |
| ATOM | 836 | C | GLY | 113 | −5.679 | −23.084 | 6.049 | 1.00 | 12.74 |
| ATOM | 837 | O | GLY | 113 | −4.644 | −22.409 | 6.051 | 1.00 | 13.16 |
| ATOM | 838 | N | HIS | 114 | −6.715 | −22.840 | 5.235 | 1.00 | 12.55 |
| ATOM | 839 | CA | HIS | 114 | −6.715 | −21.718 | 4.302 | 1.00 | 12.74 |
| ATOM | 840 | C | HIS | 114 | −6.781 | −20.376 | 5.054 | 1.00 | 12.26 |
| ATOM | 841 | O | HIS | 114 | −6.007 | −19.466 | 4.771 | 1.00 | 12.18 |
| ATOM | 842 | CB | HIS | 114 | −7.862 | −21.873 | 3.275 | 1.00 | 12.44 |
| ATOM | 843 | CG | HIS | 114 | −8.084 | −20.670 | 2.398 | 1.00 | 13.83 |
| ATOM | 844 | CD2 | HIS | 114 | −9.077 | −19.743 | 2.391 | 1.00 | 14.73 |
| ATOM | 845 | ND1 | HIS | 114 | −7.234 | −20.318 | 1.374 | 1.00 | 12.69 |
| ATOM | 846 | CE1 | HIS | 114 | −7.688 | −19.233 | 0.766 | 1.00 | 13.28 |
| ATOM | 847 | NE2 | HIS | 114 | −8.802 | −18.857 | 1.371 | 1.00 | 17.68 |
| ATOM | 848 | N | LEU | 115 | −7.720 | −20.264 | 5.998 | 1.00 | 12.27 |
| ATOM | 849 | CA | LEU | 115 | −7.806 | −19.108 | 6.893 | 1.00 | 11.69 |
| ATOM | 850 | C | LEU | 115 | −6.447 | −18.801 | 7.518 | 1.00 | 11.82 |
| ATOM | 851 | O | LEU | 115 | −5.977 | −17.660 | 7.458 | 1.00 | 11.60 |
| ATOM | 852 | CB | LEU | 115 | −8.808 | −19.390 | 8.021 | 1.00 | 11.64 |
| ATOM | 853 | CG | LEU | 115 | −8.890 | −18.348 | 9.136 | 1.00 | 11.29 |
| ATOM | 854 | CD1 | LEU | 115 | −9.826 | −18.859 | 10.254 | 1.00 | 9.52 |
| ATOM | 855 | CD2 | LEU | 115 | −9.379 | −16.993 | 8.557 | 1.00 | 12.91 |
| ATOM | 856 | N | ALA | 116 | −5.830 | −19.817 | 8.122 | 1.00 | 11.42 |
| ATOM | 857 | CA | ALA | 116 | −4.549 | −19.632 | 8.810 | 1.00 | 11.80 |
| ATOM | 858 | C | ALA | 116 | −3.458 | −19.230 | 7.828 | 1.00 | 12.34 |
| ATOM | 859 | O | ALA | 116 | −2.599 | −18.394 | 8.153 | 1.00 | 13.17 |
| ATOM | 860 | CB | ALA | 116 | −4.149 | −20.881 | 9.554 | 1.00 | 11.04 |
| ATOM | 861 | N | THR | 117 | −3.473 | −19.821 | 6.638 | 1.00 | 12.71 |
| ATOM | 862 | CA | THR | 117 | −2.463 | −19.489 | 5.622 | 1.00 | 13.48 |
| ATOM | 863 | C | THR | 117 | −2.564 | −18.038 | 5.168 | 1.00 | 13.36 |
| ATOM | 864 | O | THR | 117 | −1.569 | −17.322 | 5.190 | 1.00 | 13.87 |
| ATOM | 865 | CB | THR | 117 | −2.540 | −20.418 | 4.409 | 1.00 | 13.41 |
| ATOM | 866 | CG2 | THR | 117 | −1.518 | −19.984 | 3.342 | 1.00 | 13.46 |
| ATOM | 867 | OG1 | THR | 117 | −2.228 | −21.742 | 4.831 | 1.00 | 13.75 |
| ATOM | 868 | N | VAL | 118 | −3.769 | −17.603 | 4.792 | 1.00 | 13.77 |
| ATOM | 869 | CA | VAL | 118 | −4.008 | −16.225 | 4.359 | 1.00 | 13.81 |
| ATOM | 870 | C | VAL | 118 | −3.695 | −15.219 | 5.465 | 1.00 | 14.47 |
| ATOM | 871 | O | VAL | 118 | −2.928 | −14.268 | 5.233 | 1.00 | 14.89 |
| ATOM | 872 | CB | VAL | 118 | −5.446 | −16.007 | 3.793 | 1.00 | 14.68 |
| ATOM | 873 | CG1 | VAL | 118 | −5.704 | −16.933 | 2.597 | 1.00 | 14.07 |
| ATOM | 874 | CG2 | VAL | 118 | −5.656 | −14.533 | 3.377 | 1.00 | 15.20 |
| ATOM | 875 | N | LEU | 119 | −4.234 | −15.417 | 6.663 | 1.00 | 13.46 |
| ATOM | 876 | CA | LEU | 119 | −3.936 | −14.480 | 7.753 | 1.00 | 14.32 |
| ATOM | 877 | C | LEU | 119 | −2.429 | −14.419 | 8.067 | 1.00 | 14.55 |
| ATOM | 878 | O | LEU | 119 | −1.884 | −13.346 | 8.344 | 1.00 | 13.85 |
| ATOM | 879 | CB | LEU | 119 | −4.717 | −14.827 | 9.040 | 1.00 | 14.12 |
| ATOM | 880 | CG | LEU | 119 | −6.241 | −14.658 | 9.105 | 1.00 | 15.09 |
| ATOM | 881 | CD1 | LEU | 119 | −6.751 | −15.100 | 10.496 | 1.00 | 13.34 |
| ATOM | 882 | CD2 | LEU | 119 | −6.719 | −13.209 | 8.782 | 1.00 | 14.58 |
| ATOM | 883 | N | ASN | 120 | −1.748 | −15.560 | 8.012 | 1.00 | 14.91 |
| ATOM | 884 | CA | ASN | 120 | −0.292 | −15.550 | 8.263 | 1.00 | 16.41 |
| ATOM | 885 | C | ASN | 120 | 0.543 | −14.835 | 7.172 | 1.00 | 16.80 |
| ATOM | 886 | O | ASN | 120 | 1.252 | −13.846 | 7.434 | 1.00 | 17.62 |
| ATOM | 887 | CB | ASN | 120 | 0.241 | −16.964 | 8.477 | 1.00 | 15.88 |
| ATOM | 888 | CG | ASN | 120 | 1.721 | −16.968 | 8.740 | 1.00 | 17.20 |
| ATOM | 889 | ND2 | ASN | 120 | 2.117 | −16.603 | 9.963 | 1.00 | 12.93 |
| ATOM | 890 | OD1 | ASN | 120 | 2.512 | −17.249 | 7.828 | 1.00 | 17.95 |
| ATOM | 891 | N | ASP | 121 | 0.417 | −15.341 | 5.953 | 1.00 | 17.66 |
| ATOM | 892 | CA | ASP | 121 | 1.263 | −14.980 | 4.823 | 1.00 | 18.38 |
| ATOM | 893 | C | ASP | 121 | 0.969 | −13.557 | 4.337 | 1.00 | 19.34 |
| ATOM | 894 | O | ASP | 121 | 1.877 | −12.813 | 3.953 | 1.00 | 18.91 |
| ATOM | 895 | CB | ASP | 121 | 1.034 | −15.985 | 3.675 | 1.00 | 18.55 |
| ATOM | 896 | CG | ASP | 121 | 1.550 | −17.396 | 3.999 | 1.00 | 20.30 |
| ATOM | 897 | OD1 | ASP | 121 | 2.008 | −17.654 | 5.129 | 1.00 | 23.34 |
| ATOM | 898 | OD2 | ASP | 121 | 1.493 | −18.275 | 3.115 | 1.00 | 22.87 |
| ATOM | 899 | N | LYS | 122 | −0.306 | −13.182 | 4.352 | 1.00 | 19.27 |
| ATOM | 900 | CA | LYS | 122 | −0.710 | −11.882 | 3.836 | 1.00 | 20.09 |
| ATOM | 901 | C | LYS | 122 | −0.680 | −10.802 | 4.926 | 1.00 | 19.60 |
| ATOM | 902 | O | LYS | 122 | −0.368 | −9.633 | 4.654 | 1.00 | 19.59 |
| ATOM | 903 | CB | LYS | 122 | −2.095 | −11.996 | 3.172 | 1.00 | 20.30 |
| ATOM | 904 | CG | LYS | 122 | −2.399 | −10.905 | 2.169 | 1.00 | 24.23 |
| ATOM | 905 | CD | LYS | 122 | −3.709 | −11.185 | 1.423 | 1.00 | 27.60 |
| ATOM | 906 | CE | LYS | 122 | −4.249 | −9.889 | 0.816 | 1.00 | 32.22 |
| ATOM | 907 | NZ | LYS | 122 | −3.460 | −9.475 | −0.392 | 1.00 | 35.56 |
| ATOM | 908 | N | TYR | 123 | −0.967 | −11.176 | 6.168 | 1.00 | 18.59 |
| ATOM | 909 | CA | TYR | 123 | −1.120 | −10.141 | 7.202 | 1.00 | 17.62 |
| ATOM | 910 | C | TYR | 123 | −0.273 | −10.300 | 8.459 | 1.00 | 17.05 |
| ATOM | 911 | O | TYR | 123 | −0.255 | −9.410 | 9.306 | 1.00 | 17.05 |
| ATOM | 912 | CB | TYR | 123 | −2.584 | −10.002 | 7.591 | 1.00 | 17.83 |
| ATOM | 913 | CG | TYR | 123 | −3.523 | −9.715 | 6.436 | 1.00 | 17.54 |
| ATOM | 914 | CD1 | TYR | 123 | −3.457 | −8.502 | 5.738 | 1.00 | 18.66 |
| ATOM | 915 | CD2 | TYR | 123 | −4.504 | −10.648 | 6.059 | 1.00 | 18.46 |
| ATOM | 916 | CE1 | TYR | 123 | −4.337 | −8.219 | 4.685 | 1.00 | 17.51 |
| ATOM | 917 | CE2 | TYR | 123 | −5.393 | −10.377 | 4.995 | 1.00 | 17.65 |
| ATOM | 918 | CZ | TYR | 123 | −5.298 | −9.160 | 4.324 | 1.00 | 16.73 |
| ATOM | 919 | OH | TYR | 123 | −6.156 | −8.873 | 3.289 | 1.00 | 18.47 |
| ATOM | 920 | N | GLY | 124 | 0.429 | −11.416 | 8.579 | 1.00 | 16.16 |
| ATOM | 921 | CA | GLY | 124 | 1.189 | −11.708 | 9.782 | 1.00 | 16.03 |
| ATOM | 922 | C | GLY | 124 | 0.327 | −11.946 | 11.018 | 1.00 | 16.21 |
| ATOM | 923 | O | GLY | 124 | 0.744 | −11.610 | 12.127 | 1.00 | 16.82 |
| ATOM | 924 | N | ARG | 125 | −0.865 | −12.528 | 10.844 | 1.00 | 15.33 |
| ATOM | 925 | CA | ARG | 125 | −1.821 | −12.628 | 11.953 | 1.00 | 14.14 |
| ATOM | 926 | C | ARG | 125 | −2.293 | −14.060 | 12.172 | 1.00 | 14.18 |
| ATOM | 927 | O | ARG | 125 | −2.114 | −14.921 | 11.298 | 1.00 | 12.77 |
| ATOM | 928 | CB | ARG | 125 | −3.025 | −11.704 | 11.712 | 1.00 | 14.80 |
| ATOM | 929 | CG | ARG | 125 | −2.700 | −10.219 | 11.413 | 1.00 | 13.21 |
| ATOM | 930 | CD | ARG | 125 | −2.104 | −9.507 | 12.628 | 1.00 | 14.81 |
| ATOM | 931 | NE | ARG | 125 | −3.084 | −9.277 | 13.692 | 1.00 | 14.92 |
| ATOM | 932 | CZ | ARG | 125 | −2.893 | −8.478 | 14.743 | 1.00 | 17.12 |
| ATOM | 933 | NH1 | ARG | 125 | −1.766 | −7.774 | 14.884 | 1.00 | 16.35 |
| ATOM | 934 | NH2 | ARG | 125 | −3.844 | −8.362 | 15.654 | 1.00 | 17.33 |
| ATOM | 935 | N | ALA | 126 | −2.901 | −14.301 | 13.342 | 1.00 | 13.16 |
| ATOM | 936 | CA | ALA | 126 | −3.296 | −15.630 | 13.762 | 1.00 | 12.99 |
| ATOM | 937 | C | ALA | 126 | −4.610 | −15.590 | 14.540 | 1.00 | 13.08 |
| ATOM | 938 | O | ALA | 126 | −4.990 | −14.548 | 15.076 | 1.00 | 13.30 |
| ATOM | 939 | CB | ALA | 126 | −2.201 | −16.255 | 14.636 | 1.00 | 12.72 |
| ATOM | 940 | N | ILE | 127 | −5.297 | −16.732 | 14.567 | 1.00 | 12.73 |
| ATOM | 941 | CA | ILE | 127 | −6.468 | −16.983 | 15.413 | 1.00 | 11.88 |
| ATOM | 942 | C | ILE | 127 | −6.317 | −18.357 | 16.049 | 1.00 | 11.81 |
| ATOM | 943 | O | ILE | 127 | −5.757 | −19.293 | 15.442 | 1.00 | 10.79 |
| ATOM | 944 | CB | ILE | 127 | −7.809 | −16.943 | 14.599 | 1.00 | 12.14 |
| ATOM | 945 | CG1 | ILE | 127 | −8.052 | −15.533 | 14.047 | 1.00 | 11.93 |
| ATOM | 946 | CG2 | ILE | 127 | −9.026 | −17.387 | 15.455 | 1.00 | 9.89 |
| ATOM | 947 | CD1 | ILE | 127 | −9.187 | −15.463 | 13.065 | 1.00 | 14.43 |
| ATOM | 948 | N | SER | 128 | −6.837 | −18.481 | 17.270 | 1.00 | 11.61 |
| ATOM | 949 | CA | SER | 128 | −6.837 | −19.748 | 17.974 | 1.00 | 11.83 |
| ATOM | 950 | C | SER | 128 | −8.009 | −20.642 | 17.502 | 1.00 | 11.96 |
| ATOM | 951 | O | SER | 128 | −9.177 | −20.353 | 17.807 | 1.00 | 11.06 |
| ATOM | 952 | CB | SER | 128 | −6.932 | −19.486 | 19.491 | 1.00 | 12.12 |
| ATOM | 953 | OG | SER | 128 | −7.040 | −20.712 | 20.197 | 1.00 | 9.24 |
| ATOM | 954 | N | HIS | 129 | −7.705 | −21.725 | 16.778 | 1.00 | 11.28 |
| ATOM | 955 | CA | HIS | 129 | −8.757 | −22.620 | 16.267 | 1.00 | 11.73 |
| ATOM | 956 | C | HIS | 129 | −8.227 | −24.068 | 16.234 | 1.00 | 12.39 |
| ATOM | 957 | O | HIS | 129 | −7.098 | −24.336 | 16.671 | 1.00 | 12.29 |
| ATOM | 958 | CB | HIS | 129 | −9.277 | −22.154 | 14.871 | 1.00 | 11.13 |
| ATOM | 959 | CG | HIS | 129 | −8.186 | −21.900 | 13.862 | 1.00 | 11.62 |
| ATOM | 960 | CD2 | HIS | 129 | −7.763 | −20.754 | 13.270 | 1.00 | 10.88 |
| ATOM | 961 | ND1 | HIS | 129 | −7.395 | −22.909 | 13.345 | 1.00 | 11.77 |
| ATOM | 962 | CE1 | HIS | 129 | −6.526 | −22.393 | 12.492 | 1.00 | 12.45 |
| ATOM | 963 | NE2 | HIS | 129 | −6.732 | −21.086 | 12.424 | 1.00 | 12.07 |
| ATOM | 964 | N | GLY | 130 | −9.038 | −24.978 | 15.703 | 1.00 | 12.61 |
| ATOM | 965 | CA | GLY | 130 | −8.705 | −26.390 | 15.639 | 1.00 | 13.13 |
| ATOM | 966 | C | GLY | 130 | −7.532 | −26.703 | 14.726 | 1.00 | 14.28 |
| ATOM | 967 | O | GLY | 130 | −7.054 | −25.846 | 13.964 | 1.00 | 13.90 |
| ATOM | 968 | N | THR | 131 | −7.077 | −27.947 | 14.793 | 1.00 | 15.38 |
| ATOM | 969 | CA | THR | 131 | −5.852 | −28.361 | 14.124 | 1.00 | 16.25 |
| ATOM | 970 | C | THR | 131 | −6.081 | −28.903 | 12.708 | 1.00 | 17.03 |
| ATOM | 971 | O | THR | 131 | −5.141 | −28.944 | 11.921 | 1.00 | 17.37 |
| ATOM | 972 | CB | THR | 131 | −5.130 | −29.485 | 14.917 | 1.00 | 16.64 |
| ATOM | 973 | CG2 | THR | 131 | −4.784 | −29.028 | 16.339 | 1.00 | 16.68 |
| ATOM | 974 | OG1 | THR | 131 | −5.983 | −30.641 | 14.973 | 1.00 | 16.13 |
| ATOM | 975 | N | CYS | 132 | −7.298 | −29.364 | 12.403 | 1.00 | 16.39 |
| ATOM | 976 | CA | CYS | 132 | −7.546 | −30.022 | 11.123 | 1.00 | 15.93 |
| ATOM | 977 | C | CYS | 132 | −7.699 | −28.992 | 9.973 | 1.00 | 15.16 |
| ATOM | 978 | O | CYS | 132 | −8.574 | −28.128 | 10.043 | 1.00 | 14.76 |
| ATOM | 979 | CB | CYS | 132 | −8.806 | −30.902 | 11.247 | 1.00 | 16.57 |
| ATOM | 980 | SG | CYS | 132 | −8.725 | −32.138 | 12.571 | 0.93 | 16.02 |
| ATOM | 981 | N | PRO | 133 | −6.855 | −29.082 | 8.918 | 1.00 | 14.85 |
| ATOM | 982 | CA | PRO | 133 | −6.838 | −28.015 | 7.895 | 1.00 | 14.33 |
| ATOM | 983 | C | PRO | 133 | −8.104 | −27.911 | 7.022 | 1.00 | 13.90 |
| ATOM | 984 | O | PRO | 133 | −8.404 | −26.834 | 6.502 | 1.00 | 13.20 |
| ATOM | 985 | CB | PRO | 133 | −5.602 | −28.360 | 7.033 | 1.00 | 14.62 |
| ATOM | 986 | CG | PRO | 133 | −5.329 | −29.831 | 7.323 | 1.00 | 14.83 |
| ATOM | 987 | CD | PRO | 133 | −5.751 | −30.050 | 8.733 | 1.00 | 14.50 |
| ATOM | 988 | N | GLY | 134 | −8.836 | −29.019 | 6.884 | 1.00 | 13.39 |
| ATOM | 989 | CA | GLY | 134 | −10.038 | −29.059 | 6.051 | 1.00 | 12.44 |
| ATOM | 990 | C | GLY | 134 | −11.342 | −28.668 | 6.738 | 1.00 | 12.07 |
| ATOM | 991 | O | GLY | 134 | −12.357 | −28.490 | 6.063 | 1.00 | 11.91 |
| ATOM | 992 | N | VAL | 135 | −11.308 | −28.506 | 8.066 | 1.00 | 11.66 |
| ATOM | 993 | CA | VAL | 135 | −12.474 | −28.058 | 8.837 | 1.00 | 10.33 |
| ATOM | 994 | C | VAL | 135 | −12.969 | −26.694 | 8.340 | 1.00 | 10.61 |
| ATOM | 995 | O | VAL | 135 | −12.180 | −25.787 | 8.097 | 1.00 | 10.97 |
| ATOM | 996 | CB | VAL | 135 | −12.189 | −28.065 | 10.358 | 1.00 | 10.20 |
| ATOM | 997 | CG1 | VAL | 135 | −13.344 | −27.423 | 11.171 | 1.00 | 9.16 |
| ATOM | 998 | CG2 | VAL | 135 | −12.043 | −29.505 | 10.828 | 1.00 | 9.30 |
| ATOM | 999 | N | GLY | 136 | −14.278 | −26.550 | 8.173 | 1.00 | 10.34 |
| ATOM | 1000 | CA | GLY | 136 | −14.830 | −25.274 | 7.743 | 1.00 | 9.83 |
| ATOM | 1001 | C | GLY | 136 | −14.891 | −24.289 | 8.903 | 1.00 | 10.00 |
| ATOM | 1002 | O | GLY | 136 | −15.165 | −24.667 | 10.047 | 1.00 | 8.61 |
| ATOM | 1003 | N | ILE | 137 | −14.610 | −23.021 | 8.602 | 1.00 | 9.63 |
| ATOM | 1004 | CA | ILE | 137 | −14.774 | −21.940 | 9.561 | 1.00 | 9.21 |
| ATOM | 1005 | C | ILE | 137 | −16.184 | −21.902 | 10.206 | 1.00 | 10.10 |
| ATOM | 1006 | O | ILE | 137 | −16.293 | −21.693 | 11.427 | 1.00 | 10.09 |
| ATOM | 1007 | CB | ILE | 137 | −14.421 | −20.555 | 8.916 | 1.00 | 8.97 |
| ATOM | 1008 | CG1 | ILE | 137 | −14.279 | −19.467 | 9.980 | 1.00 | 10.46 |
| ATOM | 1009 | CG2 | ILE | 137 | −15.412 | −20.136 | 7.848 | 1.00 | 6.72 |
| ATOM | 1010 | CD1 | ILE | 137 | −14.011 | −18.081 | 9.371 | 1.00 | 12.73 |
| ATOM | 1011 | N | SER | 138 | −17.251 | −22.115 | 9.421 | 1.00 | 9.00 |
| ATOM | 1012 | CA | SER | 138 | −18.598 | −21.849 | 9.974 | 1.00 | 9.29 |
| ATOM | 1013 | C | SER | 138 | −19.087 | −22.848 | 11.016 | 1.00 | 8.91 |
| ATOM | 1014 | O | SER | 138 | −19.523 | −22.442 | 12.086 | 1.00 | 9.02 |
| ATOM | 1015 | CB | SER | 138 | −19.649 | −21.624 | 8.885 | 1.00 | 8.07 |
| ATOM | 1016 | OG | SER | 138 | −19.264 | −20.565 | 8.040 | 1.00 | 9.32 |
| ATOM | 1017 | N | GLY | 139 | −19.053 | −24.146 | 10.712 | 1.00 | 9.27 |
| ATOM | 1018 | CA | GLY | 139 | −19.422 | −25.142 | 11.732 | 1.00 | 8.16 |
| ATOM | 1019 | C | GLY | 139 | −18.523 | −25.093 | 12.957 | 1.00 | 8.60 |
| ATOM | 1020 | O | GLY | 139 | −18.986 | −25.236 | 14.098 | 1.00 | 9.31 |
| ATOM | 1021 | N | HIS | 140 | −17.232 | −24.891 | 12.723 | 1.00 | 8.49 |
| ATOM | 1022 | CA | HIS | 140 | −16.227 | −24.838 | 13.788 | 1.00 | 8.47 |
| ATOM | 1023 | C | HIS | 140 | −16.457 | −23.656 | 14.747 | 1.00 | 9.13 |
| ATOM | 1024 | O | HIS | 140 | −16.650 | −23.863 | 15.958 | 1.00 | 9.31 |
| ATOM | 1025 | CB | HIS | 140 | −14.835 | −24.768 | 13.174 | 1.00 | 8.67 |
| ATOM | 1026 | CG | HIS | 140 | −13.711 | −24.947 | 14.155 | 1.00 | 7.72 |
| ATOM | 1027 | CD2 | HIS | 140 | −12.770 | −24.078 | 14.606 | 1.00 | 3.82 |
| ATOM | 1028 | ND1 | HIS | 140 | −13.426 | −26.161 | 14.737 | 1.00 | 5.56 |
| ATOM | 1029 | CE1 | HIS | 140 | −12.361 | −26.035 | 15.515 | 1.00 | 7.93 |
| ATOM | 1030 | NE2 | HIS | 140 | −11.956 | −24.774 | 15.466 | 1.00 | 7.82 |
| ATOM | 1031 | N | PHE | 141 | −16.472 | −22.435 | 14.208 | 1.00 | 8.89 |
| ATOM | 1032 | CA | PHE | 141 | −16.620 | −21.241 | 15.047 | 1.00 | 9.83 |
| ATOM | 1033 | C | PHE | 141 | −18.020 | −21.115 | 15.665 | 1.00 | 10.20 |
| ATOM | 1034 | O | PHE | 141 | −18.164 | −20.561 | 16.744 | 1.00 | 11.60 |
| ATOM | 1035 | CB | PHE | 141 | −16.293 | −19.958 | 14.250 | 1.00 | 9.98 |
| ATOM | 1036 | CG | PHE | 141 | −14.812 | −19.719 | 13.995 | 1.00 | 9.40 |
| ATOM | 1037 | CD1 | PHE | 141 | −14.225 | −18.519 | 14.397 | 1.00 | 10.75 |
| ATOM | 1038 | CD2 | PHE | 141 | −14.017 | −20.664 | 13.320 | 1.00 | 9.93 |
| ATOM | 1039 | CE1 | PHE | 141 | −12.867 | −18.253 | 14.151 | 1.00 | 10.27 |
| ATOM | 1040 | CE2 | PHE | 141 | −12.662 | −20.420 | 13.074 | 1.00 | 10.15 |
| ATOM | 1041 | CZ | PHE | 141 | −12.081 | −19.210 | 13.481 | 1.00 | 10.43 |
| ATOM | 1042 | N | ALA | 142 | −19.058 | −21.611 | 14.985 | 1.00 | 10.07 |
| ATOM | 1043 | CA | ALA | 142 | −20.421 | −21.408 | 15.475 | 1.00 | 9.65 |
| ATOM | 1044 | C | ALA | 142 | −20.695 | −22.179 | 16.767 | 1.00 | 9.55 |
| ATOM | 1045 | O | ALA | 142 | −21.688 | −21.909 | 17.444 | 1.00 | 9.24 |
| ATOM | 1046 | CB | ALA | 142 | −21.452 | −21.800 | 14.396 | 1.00 | 8.75 |
| ATOM | 1047 | N | HIS | 143 | −19.846 | −23.168 | 17.069 | 1.00 | 9.60 |
| ATOM | 1048 | CA | HIS | 143 | −20.097 | −24.080 | 18.191 | 1.00 | 10.25 |
| ATOM | 1049 | C | HIS | 143 | −18.928 | −24.201 | 19.160 | 1.00 | 10.89 |
| ATOM | 1050 | O | HIS | 143 | −18.944 | −25.078 | 20.021 | 1.00 | 12.25 |
| ATOM | 1051 | CB | HIS | 143 | −20.510 | −25.480 | 17.684 | 1.00 | 9.67 |
| ATOM | 1052 | CG | HIS | 143 | −21.641 | −25.459 | 16.693 | 1.00 | 9.04 |
| ATOM | 1053 | CD2 | HIS | 143 | −22.983 | −25.348 | 16.873 | 1.00 | 8.88 |
| ATOM | 1054 | ND1 | HIS | 143 | −21.438 | −25.497 | 15.323 | 1.00 | 10.07 |
| ATOM | 1055 | CE1 | HIS | 143 | −22.609 | −25.445 | 14.706 | 1.00 | 8.40 |
| ATOM | 1056 | NE2 | HIS | 143 | −23.563 | −25.346 | 15.625 | 1.00 | 9.55 |
| ATOM | 1057 | N | GLY | 144 | −17.927 | −23.329 | 19.037 | 1.00 | 11.03 |
| ATOM | 1058 | CA | GLY | 144 | −16.769 | −23.343 | 19.941 | 1.00 | 11.28 |
| ATOM | 1059 | C | GLY | 144 | −15.436 | −23.296 | 19.188 | 1.00 | 11.22 |
| ATOM | 1060 | O | GLY | 144 | −14.986 | −22.221 | 18.771 | 1.00 | 10.71 |
| ATOM | 1061 | N | GLY | 145 | −14.810 | −24.467 | 19.036 | 1.00 | 10.50 |
| ATOM | 1062 | CA | GLY | 145 | −13.563 | −24.600 | 18.310 | 1.00 | 10.02 |
| ATOM | 1063 | C | GLY | 145 | −12.357 | −24.579 | 19.214 | 1.00 | 10.85 |
| ATOM | 1064 | O | GLY | 145 | −11.891 | −23.500 | 19.637 | 1.00 | 11.11 |
| ATOM | 1065 | N | PHE | 146 | −11.829 | −25.765 | 19.492 | 1.00 | 9.85 |
| ATOM | 1066 | CA | PHE | 146 | −10.752 | −25.935 | 20.443 | 1.00 | 10.78 |
| ATOM | 1067 | C | PHE | 146 | −9.485 | −26.455 | 19.741 | 1.00 | 11.22 |
| ATOM | 1068 | O | PHE | 146 | −9.567 | −27.345 | 18.889 | 1.00 | 10.78 |
| ATOM | 1069 | CB | PHE | 146 | −11.186 | −26.948 | 21.538 | 1.00 | 10.72 |
| ATOM | 1070 | CG | PHE | 146 | −10.168 | −27.101 | 22.640 | 1.00 | 12.04 |
| ATOM | 1071 | CD1 | PHE | 146 | −10.165 | −26.213 | 23.733 | 1.00 | 10.55 |
| ATOM | 1072 | CD2 | PHE | 146 | −9.175 | −28.079 | 22.562 | 1.00 | 11.12 |
| ATOM | 1073 | CE1 | PHE | 146 | −9.224 | −26.344 | 24.744 | 1.00 | 9.78 |
| ATOM | 1074 | CE2 | PHE | 146 | −8.209 | −28.197 | 23.542 | 1.00 | 10.92 |
| ATOM | 1075 | CZ | PHE | 146 | −8.247 | −27.329 | 24.660 | 1.00 | 11.71 |
| ATOM | 1076 | N | GLY | 147 | −8.320 | −25.938 | 20.118 | 1.00 | 10.83 |
| ATOM | 1077 | CA | GLY | 147 | −7.074 | −26.440 | 19.572 | 1.00 | 10.63 |
| ATOM | 1078 | C | GLY | 147 | −5.890 | −26.166 | 20.494 | 1.00 | 12.04 |
| ATOM | 1079 | O | GLY | 147 | −6.074 | −25.852 | 21.699 | 1.00 | 10.95 |
| ATOM | 1080 | N | PHE | 148 | −4.682 | −26.222 | 19.909 | 1.00 | 10.99 |
| ATOM | 1081 | CA | PHE | 148 | −3.450 | −26.085 | 20.678 | 1.00 | 11.63 |
| ATOM | 1082 | C | PHE | 148 | −3.027 | −24.649 | 21.045 | 1.00 | 11.79 |
| ATOM | 1083 | O | PHE | 148 | −1.932 | −24.449 | 21.564 | 1.00 | 11.70 |
| ATOM | 1084 | CB | PHE | 148 | −2.297 | −26.801 | 19.968 | 1.00 | 11.77 |
| ATOM | 1085 | CG | PHE | 148 | −2.320 | −28.289 | 20.129 | 1.00 | 12.14 |
| ATOM | 1086 | CD1 | PHE | 148 | −2.018 | −28.880 | 21.362 | 1.00 | 12.71 |
| ATOM | 1087 | CD2 | PHE | 148 | −2.630 | −29.112 | 19.050 | 1.00 | 11.80 |
| ATOM | 1088 | CE1 | PHE | 148 | −2.026 | −30.295 | 21.514 | 1.00 | 12.38 |
| ATOM | 1089 | CE2 | PHE | 148 | −2.637 | −30.522 | 19.196 | 1.00 | 12.94 |
| ATOM | 1090 | CZ | PHE | 148 | −2.329 | −31.104 | 20.432 | 1.00 | 11.01 |
| ATOM | 1091 | N | SER | 149 | −3.880 | −23.658 | 20.782 | 1.00 | 12.00 |
| ATOM | 1092 | CA | SER | 149 | −3.689 | −22.311 | 21.341 | 1.00 | 12.19 |
| ATOM | 1093 | C | SER | 149 | −4.822 | −21.912 | 22.297 | 1.00 | 12.61 |
| ATOM | 1094 | O | SER | 149 | −4.812 | −20.797 | 22.851 | 1.00 | 13.64 |
| ATOM | 1095 | CB | SER | 149 | −3.564 | −21.266 | 20.230 | 1.00 | 12.41 |
| ATOM | 1096 | OG | SER | 149 | −2.439 | −21.523 | 19.415 | 1.00 | 13.00 |
| ATOM | 1097 | N | SER | 150 | −5.785 | −22.808 | 22.520 | 1.00 | 11.67 |
| ATOM | 1098 | CA | SER | 150 | −6.966 | −22.459 | 23.325 | 1.00 | 12.32 |
| ATOM | 1099 | C | SER | 150 | −6.651 | −22.146 | 24.782 | 1.00 | 12.00 |
| ATOM | 1100 | O | SER | 150 | −7.303 | −21.286 | 25.370 | 1.00 | 12.45 |
| ATOM | 1101 | CB | SER | 150 | −8.048 | −23.532 | 23.251 | 1.00 | 11.71 |
| ATOM | 1102 | OG | SER | 150 | −8.550 | −23.644 | 21.936 | 1.00 | 13.72 |
| ATOM | 1103 | N | HIS | 151 | −5.653 | −22.808 | 25.370 | 1.00 | 11.82 |
| ATOM | 1104 | CA | HIS | 151 | −5.328 | −22.494 | 26.779 | 1.00 | 12.10 |
| ATOM | 1105 | C | HIS | 151 | −4.773 | −21.064 | 26.855 | 1.00 | 12.57 |
| ATOM | 1106 | O | HIS | 151 | −5.079 | −20.301 | 27.776 | 1.00 | 13.00 |
| ATOM | 1107 | CB | HIS | 151 | −4.336 | −23.503 | 27.319 | 1.00 | 11.17 |
| ATOM | 1108 | CG | HIS | 151 | −4.294 | −23.598 | 28.817 | 1.00 | 12.17 |
| ATOM | 1109 | CD2 | HIS | 151 | −4.932 | −24.430 | 29.679 | 1.00 | 11.34 |
| ATOM | 1110 | ND1 | HIS | 151 | −3.434 | −22.836 | 29.585 | 1.00 | 10.72 |
| ATOM | 1111 | CE1 | HIS | 151 | −3.583 | −23.157 | 30.859 | 1.00 | 14.48 |
| ATOM | 1112 | NE2 | HIS | 151 | −4.477 | −24.127 | 30.946 | 1.00 | 13.55 |
| ATOM | 1113 | N | MET | 152 | −3.937 | −20.724 | 25.879 | 1.00 | 12.53 |
| ATOM | 1114 | CA | MET | 152 | −3.351 | −19.397 | 25.772 | 1.00 | 13.34 |
| ATOM | 1115 | C | MET | 152 | −4.326 | −18.303 | 25.278 | 1.00 | 13.29 |
| ATOM | 1116 | O | MET | 152 | −4.308 | −17.177 | 25.775 | 1.00 | 13.46 |
| ATOM | 1117 | CB | MET | 152 | −2.149 | −19.477 | 24.816 | 1.00 | 12.58 |
| ATOM | 1118 | CG | MET | 152 | −1.457 | −18.151 | 24.592 | 1.00 | 12.90 |
| ATOM | 1119 | SD | MET | 152 | −0.476 | −17.693 | 26.031 | 1.00 | 14.17 |
| ATOM | 1120 | CE | MET | 152 | 0.140 | −16.083 | 25.468 | 1.00 | 14.03 |
| ATOM | 1121 | N | HIS | 153 | −5.162 | −18.624 | 24.289 | 1.00 | 13.60 |
| ATOM | 1122 | CA | HIS | 153 | −5.899 | −17.576 | 23.560 | 1.00 | 13.29 |
| ATOM | 1123 | C | HIS | 153 | −7.401 | −17.805 | 23.427 | 1.00 | 12.60 |
| ATOM | 1124 | O | HIS | 153 | −8.081 | −17.022 | 22.783 | 1.00 | 13.04 |
| ATOM | 1125 | CB | HIS | 153 | −5.304 | −17.373 | 22.145 | 1.00 | 13.46 |
| ATOM | 1126 | CG | HIS | 153 | −4.038 | −16.569 | 22.116 | 1.00 | 14.85 |
| ATOM | 1127 | CD2 | HIS | 153 | −2.819 | −16.851 | 21.593 | 1.00 | 15.87 |
| ATOM | 1128 | ND1 | HIS | 153 | −3.942 | −15.296 | 22.643 | 1.00 | 15.97 |
| ATOM | 1129 | CE1 | HIS | 153 | −2.710 | −14.848 | 22.486 | 1.00 | 14.70 |
| ATOM | 1130 | NE2 | HIS | 153 | −2.014 | −15.768 | 21.840 | 1.00 | 17.05 |
| ATOM | 1131 | N | GLY | 154 | −7.926 | −18.870 | 23.995 | 1.00 | 11.96 |
| ATOM | 1132 | CA | GLY | 154 | −9.375 | −19.088 | 23.936 | 1.00 | 12.59 |
| ATOM | 1133 | C | GLY | 154 | −9.864 | −19.898 | 22.737 | 1.00 | 12.25 |
| ATOM | 1134 | O | GLY | 154 | −9.069 | −20.441 | 21.957 | 1.00 | 12.32 |
| ATOM | 1135 | N | LEU | 155 | −11.184 | −20.034 | 22.643 | 1.00 | 12.50 |
| ATOM | 1136 | CA | LEU | 155 | −11.837 | −20.784 | 21.560 | 1.00 | 11.34 |
| ATOM | 1137 | C | LEU | 155 | −11.871 | −19.955 | 20.274 | 1.00 | 11.77 |
| ATOM | 1138 | O | LEU | 155 | −11.789 | −18.711 | 20.311 | 1.00 | 11.14 |
| ATOM | 1139 | CB | LEU | 155 | −13.262 | −21.180 | 21.975 | 1.00 | 11.13 |
| ATOM | 1140 | CG | LEU | 155 | −13.445 | −21.973 | 23.300 | 1.00 | 10.14 |
| ATOM | 1141 | CD1 | LEU | 155 | −14.924 | −22.175 | 23.550 | 1.00 | 9.65 |
| ATOM | 1142 | CD2 | LEU | 155 | −12.757 | −23.350 | 23.286 | 1.00 | 6.95 |
| ATOM | 1143 | N | ALA | 156 | −11.992 | −20.635 | 19.135 | 1.00 | 11.37 |
| ATOM | 1144 | CA | ALA | 156 | −12.242 | −19.946 | 17.869 | 1.00 | 11.61 |
| ATOM | 1145 | C | ALA | 156 | −13.401 | −18.975 | 18.035 | 1.00 | 11.66 |
| ATOM | 1146 | O | ALA | 156 | −13.311 | −17.835 | 17.615 | 1.00 | 12.31 |
| ATOM | 1147 | CB | ALA | 156 | −12.526 | −20.946 | 16.740 | 1.00 | 11.56 |
| ATOM | 1148 | N | VAL | 157 | −14.476 | −19.433 | 18.669 | 1.00 | 11.73 |
| ATOM | 1149 | CA | VAL | 157 | −15.657 | −18.599 | 18.910 | 1.00 | 11.28 |
| ATOM | 1150 | C | VAL | 157 | −15.333 | −17.299 | 19.679 | 1.00 | 11.40 |
| ATOM | 1151 | O | VAL | 157 | −16.011 | −16.280 | 19.485 | 1.00 | 11.45 |
| ATOM | 1152 | CB | VAL | 157 | −16.808 | −19.403 | 19.586 | 1.00 | 10.54 |
| ATOM | 1153 | CG1 | VAL | 157 | −16.513 | −19.694 | 21.072 | 1.00 | 9.90 |
| ATOM | 1154 | CG2 | VAL | 157 | −18.139 | −18.684 | 19.449 | 1.00 | 10.66 |
| ATOM | 1155 | N | ASP | 158 | −14.314 | −17.335 | 20.533 | 1.00 | 11.20 |
| ATOM | 1156 | CA | ASP | 158 | −13.918 | −16.144 | 21.293 | 1.00 | 11.68 |
| ATOM | 1157 | C | ASP | 158 | −13.228 | −15.034 | 20.442 | 1.00 | 12.49 |
| ATOM | 1158 | O | ASP | 158 | −13.083 | −13.890 | 20.902 | 1.00 | 12.17 |
| ATOM | 1159 | CB | ASP | 158 | −13.068 | −16.561 | 22.490 | 1.00 | 12.33 |
| ATOM | 1160 | CG | ASP | 158 | −13.847 | −17.426 | 23.490 | 1.00 | 12.35 |
| ATOM | 1161 | OD1 | ASP | 158 | −15.067 | −17.170 | 23.660 | 1.00 | 13.36 |
| ATOM | 1162 | OD2 | ASP | 158 | −13.249 | −18.369 | 24.086 | 1.00 | 12.11 |
| ATOM | 1163 | N | SER | 159 | −12.815 | −15.354 | 19.211 | 1.00 | 11.70 |
| ATOM | 1164 | CA | SER | 159 | −12.335 | −14.325 | 18.272 | 1.00 | 11.53 |
| ATOM | 1165 | C | SER | 159 | −13.496 | −13.568 | 17.578 | 1.00 | 11.07 |
| ATOM | 1166 | O | SER | 159 | −13.277 | −12.560 | 16.937 | 1.00 | 10.54 |
| ATOM | 1167 | CB | SER | 159 | −11.446 | −14.962 | 17.181 | 1.00 | 12.06 |
| ATOM | 1168 | OG | SER | 159 | −12.244 | −15.700 | 16.273 | 1.00 | 11.58 |
| ATOM | 1169 | N | VAL | 160 | −14.718 | −14.085 | 17.683 | 1.00 | 11.07 |
| ATOM | 1170 | CA | VAL | 160 | −15.846 | −13.540 | 16.927 | 1.00 | 10.93 |
| ATOM | 1171 | C | VAL | 160 | −16.323 | −12.214 | 17.544 | 1.00 | 11.34 |
| ATOM | 1172 | O | VAL | 160 | −16.800 | −12.186 | 18.683 | 1.00 | 11.89 |
| ATOM | 1173 | CB | VAL | 160 | −17.032 | −14.567 | 16.815 | 1.00 | 10.76 |
| ATOM | 1174 | CG1 | VAL | 160 | −18.171 | −13.979 | 16.008 | 1.00 | 7.97 |
| ATOM | 1175 | CG2 | VAL | 160 | −16.571 | −15.914 | 16.192 | 1.00 | 9.84 |
| ATOM | 1176 | N | VAL | 161 | −16.180 | −11.128 | 16.794 | 1.00 | 12.21 |
| ATOM | 1177 | CA | VAL | 161 | −16.648 | −9.791 | 17.225 | 1.00 | 13.29 |
| ATOM | 1178 | C | VAL | 161 | −17.845 | −9.305 | 16.414 | 1.00 | 13.97 |
| ATOM | 1179 | O | VAL | 161 | −18.462 | −8.299 | 16.763 | 1.00 | 15.36 |
| ATOM | 1180 | CB | VAL | 161 | −15.539 | −8.714 | 17.214 | 1.00 | 13.35 |
| ATOM | 1181 | CG1 | VAL | 161 | −14.415 | −9.129 | 18.187 | 1.00 | 14.32 |
| ATOM | 1182 | CG2 | VAL | 161 | −14.999 | −8.477 | 15.775 | 1.00 | 11.47 |
| ATOM | 1183 | N | GLY | 162 | −18.180 | −10.009 | 15.341 | 1.00 | 14.00 |
| ATOM | 1184 | CA | GLY | 162 | −19.411 | −9.696 | 14.602 | 1.00 | 14.15 |
| ATOM | 1185 | C | GLY | 162 | −19.715 | −10.730 | 13.529 | 1.00 | 13.71 |
| ATOM | 1186 | O | GLY | 162 | −18.830 | −11.493 | 13.131 | 1.00 | 13.05 |
| ATOM | 1187 | N | VAL | 163 | −20.966 | −10.757 | 13.065 | 1.00 | 12.83 |
| ATOM | 1188 | CA | VAL | 163 | −21.345 | −11.595 | 11.926 | 1.00 | 12.42 |
| ATOM | 1189 | C | VAL | 163 | −22.376 | −10.879 | 11.066 | 1.00 | 13.19 |
| ATOM | 1190 | O | VAL | 163 | −23.108 | −10.021 | 11.557 | 1.00 | 12.49 |
| ATOM | 1191 | CB | VAL | 163 | −21.939 | −12.997 | 12.343 | 1.00 | 12.51 |
| ATOM | 1192 | CG1 | VAL | 163 | −23.019 | −12.857 | 13.415 | 1.00 | 12.36 |
| ATOM | 1193 | CG2 | VAL | 163 | −20.834 | −13.948 | 12.816 | 1.00 | 10.54 |
| ATOM | 1194 | N | THR | 164 | −22.400 | −11.216 | 9.775 | 1.00 | 12.35 |
| ATOM | 1195 | CA | THR | 164 | −23.521 | −10.905 | 8.910 | 1.00 | 12.67 |
| ATOM | 1196 | C | THR | 164 | −24.284 | −12.220 | 8.802 | 1.00 | 12.23 |
| ATOM | 1197 | O | THR | 164 | −23.686 | −13.284 | 8.593 | 1.00 | 11.69 |
| ATOM | 1198 | CB | THR | 164 | −23.029 | −10.433 | 7.522 | 1.00 | 12.73 |
| ATOM | 1199 | CG2 | THR | 164 | −24.204 | −10.067 | 6.598 | 1.00 | 12.70 |
| ATOM | 1200 | OG1 | THR | 164 | −22.187 | −9.278 | 7.703 | 1.00 | 14.84 |
| ATOM | 1201 | N | VAL | 165 | −25.594 | −12.167 | 8.953 | 1.00 | 12.06 |
| ATOM | 1202 | CA | VAL | 165 | −26.390 | −13.409 | 9.044 | 1.00 | 11.56 |
| ATOM | 1203 | C | VAL | 165 | −27.604 | −13.327 | 8.117 | 1.00 | 11.43 |
| ATOM | 1204 | O | VAL | 165 | −28.339 | −12.321 | 8.124 | 1.00 | 12.37 |
| ATOM | 1205 | CB | VAL | 165 | −26.870 | −13.668 | 10.494 | 1.00 | 11.45 |
| ATOM | 1206 | CG1 | VAL | 165 | −27.552 | −15.036 | 10.622 | 1.00 | 10.90 |
| ATOM | 1207 | CG2 | VAL | 165 | −25.727 | −13.547 | 11.496 | 1.00 | 11.00 |
| ATOM | 1208 | N | VAL | 166 | −27.826 | −14.378 | 7.335 | 1.00 | 11.55 |
| ATOM | 1209 | CA | VAL | 166 | −29.091 | −14.546 | 6.582 | 1.00 | 10.53 |
| ATOM | 1210 | C | VAL | 166 | −30.088 | −15.277 | 7.467 | 1.00 | 11.25 |
| ATOM | 1211 | O | VAL | 166 | −29.833 | −16.426 | 7.881 | 1.00 | 10.59 |
| ATOM | 1212 | CB | VAL | 166 | −28.898 | −15.339 | 5.262 | 1.00 | 11.23 |
| ATOM | 1213 | CG1 | VAL | 166 | −30.255 | −15.521 | 4.510 | 1.00 | 9.24 |
| ATOM | 1214 | CG2 | VAL | 166 | −27.875 | −14.679 | 4.394 | 1.00 | 9.36 |
| ATOM | 1215 | N | LEU | 167 | −31.219 | −14.627 | 7.766 | 1.00 | 11.16 |
| ATOM | 1216 | CA | LEU | 167 | −32.240 | −15.219 | 8.638 | 1.00 | 12.57 |
| ATOM | 1217 | C | LEU | 167 | −33.223 | −16.102 | 7.882 | 1.00 | 13.04 |
| ATOM | 1218 | O | LEU | 167 | −33.254 | −16.066 | 6.637 | 1.00 | 13.00 |
| ATOM | 1219 | CB | LEU | 167 | −33.015 | −14.136 | 9.406 | 1.00 | 12.23 |
| ATOM | 1220 | CG | LEU | 167 | −32.219 | −13.207 | 10.304 | 1.00 | 12.97 |
| ATOM | 1221 | CD1 | LEU | 167 | −33.174 | −12.220 | 11.044 | 1.00 | 12.74 |
| ATOM | 1222 | CD2 | LEU | 167 | −31.284 | −13.977 | 11.285 | 1.00 | 12.59 |
| ATOM | 1223 | N | ALA | 168 | −34.049 | −16.846 | 8.629 | 1.00 | 13.54 |
| ATOM | 1224 | CA | ALA | 168 | −35.030 | −17.779 | 8.024 | 1.00 | 14.21 |
| ATOM | 1225 | C | ALA | 168 | −36.057 | −17.077 | 7.132 | 1.00 | 14.95 |
| ATOM | 1226 | O | ALA | 168 | −36.577 | −17.669 | 6.160 | 1.00 | 15.35 |
| ATOM | 1227 | CB | ALA | 168 | −35.736 | −18.603 | 9.098 | 1.00 | 14.32 |
| ATOM | 1228 | N | ASP | 169 | −36.342 | −15.815 | 7.450 | 1.00 | 15.45 |
| ATOM | 1229 | CA | ASP | 169 | −37.305 | −15.044 | 6.672 | 1.00 | 16.19 |
| ATOM | 1230 | C | ASP | 169 | −36.693 | −14.382 | 5.441 | 1.00 | 16.55 |
| ATOM | 1231 | O | ASP | 169 | −37.393 | −13.658 | 4.725 | 1.00 | 17.42 |
| ATOM | 1232 | CB | ASP | 169 | −38.007 | −14.007 | 7.551 | 1.00 | 16.39 |
| ATOM | 1233 | CG | ASP | 169 | −37.074 | −12.903 | 8.058 | 1.00 | 17.30 |
| ATOM | 1234 | OD1 | ASP | 169 | −35.850 | −12.904 | 7.769 | 1.00 | 15.79 |
| ATOM | 1235 | OD2 | ASP | 169 | −37.595 | −12.006 | 8.767 | 1.00 | 17.60 |
| ATOM | 1236 | N | GLY | 170 | −35.391 | −14.589 | 5.226 | 1.00 | 16.03 |
| ATOM | 1237 | CA | GLY | 170 | −34.673 | −14.014 | 4.068 | 1.00 | 15.49 |
| ATOM | 1238 | C | GLY | 170 | −33.949 | −12.686 | 4.307 | 1.00 | 15.05 |
| ATOM | 1239 | O | GLY | 170 | −33.215 | −12.200 | 3.443 | 1.00 | 15.26 |
| ATOM | 1240 | N | ARG | 171 | −34.148 | −12.080 | 5.471 | 1.00 | 14.19 |
| ATOM | 1241 | CA | ARG | 171 | −33.473 | −10.824 | 5.760 | 1.00 | 14.13 |
| ATOM | 1242 | C | ARG | 171 | −31.984 | −11.010 | 6.054 | 1.00 | 14.64 |
| ATOM | 1243 | O | ARG | 171 | −31.553 | −12.036 | 6.596 | 1.00 | 14.84 |
| ATOM | 1244 | CB | ARG | 171 | −34.117 | −10.115 | 6.940 | 1.00 | 13.43 |
| ATOM | 1245 | CG | ARG | 171 | −35.488 | −9.554 | 6.655 | 1.00 | 13.80 |
| ATOM | 1246 | CD | ARG | 171 | −35.984 | −8.756 | 7.818 | 1.00 | 15.46 |
| ATOM | 1247 | NE | ARG | 171 | −36.221 | −9.571 | 9.015 | 1.00 | 16.02 |
| ATOM | 1248 | CZ | ARG | 171 | −35.743 | −9.300 | 10.232 | 1.00 | 17.97 |
| ATOM | 1249 | NH1 | ARG | 171 | −34.956 | −8.240 | 10.441 | 1.00 | 16.41 |
| ATOM | 1250 | NH2 | ARG | 171 | −36.063 | −10.099 | 11.252 | 1.00 | 19.95 |
| ATOM | 1251 | N | ILE | 172 | −31.198 | −10.011 | 5.705 | 1.00 | 14.68 |
| ATOM | 1252 | CA | ILE | 172 | −29.777 | −10.022 | 6.056 | 1.00 | 14.77 |
| ATOM | 1253 | C | ILE | 172 | −29.597 | −9.044 | 7.211 | 1.00 | 15.38 |
| ATOM | 1254 | O | ILE | 172 | −30.059 | −7.903 | 7.115 | 1.00 | 15.53 |
| ATOM | 1255 | CB | ILE | 172 | −28.919 | −9.613 | 4.850 | 1.00 | 14.92 |
| ATOM | 1256 | CG1 | ILE | 172 | −29.096 | −10.634 | 3.707 | 1.00 | 14.25 |
| ATOM | 1257 | CG2 | ILE | 172 | −27.439 | −9.412 | 5.281 | 1.00 | 14.47 |
| ATOM | 1258 | CD1 | ILE | 172 | −28.627 | −10.146 | 2.314 | 1.00 | 14.82 |
| ATOM | 1259 | N | VAL | 173 | −28.983 | −9.494 | 8.313 | 1.00 | 15.20 |
| ATOM | 1260 | CA | VAL | 173 | −28.795 | −8.641 | 9.498 | 1.00 | 15.41 |
| ATOM | 1261 | C | VAL | 173 | −27.370 | −8.750 | 10.045 | 1.00 | 15.94 |
| ATOM | 1262 | O | VAL | 173 | −26.682 | −9.755 | 9.827 | 1.00 | 15.11 |
| ATOM | 1263 | CB | VAL | 173 | −29.798 | −8.972 | 10.668 | 1.00 | 15.50 |
| ATOM | 1264 | CG1 | VAL | 173 | −31.243 | −8.850 | 10.219 | 1.00 | 15.81 |
| ATOM | 1265 | CG2 | VAL | 173 | −29.514 | −10.348 | 11.279 | 1.00 | 13.03 |
| ATOM | 1266 | N | GLU | 174 | −26.937 | −7.713 | 10.749 | 1.00 | 16.56 |
| ATOM | 1267 | CA | GLU | 174 | −25.670 | −7.728 | 11.468 | 1.00 | 17.62 |
| ATOM | 1268 | C | GLU | 174 | −25.933 | −8.106 | 12.925 | 1.00 | 17.64 |
| ATOM | 1269 | O | GLU | 174 | −26.982 | −7.767 | 13.487 | 1.00 | 17.97 |
| ATOM | 1270 | CB | GLU | 174 | −24.988 | −6.350 | 11.407 | 1.00 | 18.20 |
| ATOM | 1271 | CG | GLU | 174 | −24.836 | −5.726 | 10.005 | 1.00 | 22.81 |
| ATOM | 1272 | CD | GLU | 174 | −23.983 | −6.564 | 9.062 | 1.00 | 28.76 |
| ATOM | 1273 | OE1 | GLU | 174 | −22.983 | −7.144 | 9.524 | 1.00 | 29.38 |
| ATOM | 1274 | OE2 | GLU | 174 | −24.329 | −6.643 | 7.862 | 1.00 | 33.22 |
| ATOM | 1275 | N | ALA | 175 | −24.990 | −8.827 | 13.525 | 1.00 | 17.40 |
| ATOM | 1276 | CA | ALA | 175 | −25.037 | −9.128 | 14.953 | 1.00 | 16.68 |
| ATOM | 1277 | C | ALA | 175 | −23.681 | −8.872 | 15.596 | 1.00 | 17.12 |
| ATOM | 1278 | O | ALA | 175 | −22.630 | −9.147 | 15.002 | 1.00 | 17.20 |
| ATOM | 1279 | CB | ALA | 175 | −25.516 | −10.565 | 15.217 | 1.00 | 16.09 |
| ATOM | 1280 | N | SER | 176 | −23.698 | −8.345 | 16.817 | 1.00 | 17.12 |
| ATOM | 1281 | CA | SER | 176 | −22.472 | −7.980 | 17.512 | 1.00 | 17.74 |
| ATOM | 1282 | C | SER | 176 | −22.877 | −7.624 | 18.923 | 1.00 | 18.08 |
| ATOM | 1283 | O | SER | 176 | −24.035 | −7.781 | 19.279 | 1.00 | 18.47 |
| ATOM | 1284 | CB | SER | 176 | −21.755 | −6.793 | 16.824 | 1.00 | 17.79 |
| ATOM | 1285 | OG | SER | 176 | −22.387 | −5.565 | 17.175 | 1.00 | 18.32 |
| ATOM | 1286 | N | ALA | 177 | −21.930 | −7.150 | 19.728 | 1.00 | 18.91 |
| ATOM | 1287 | CA | ALA | 177 | −22.261 | −6.661 | 21.075 | 1.00 | 19.71 |
| ATOM | 1288 | C | ALA | 177 | −23.233 | −5.469 | 21.048 | 1.00 | 20.62 |
| ATOM | 1289 | O | ALA | 177 | −24.036 | −5.297 | 21.957 | 1.00 | 21.26 |
| ATOM | 1290 | CB | ALA | 177 | −20.993 | −6.314 | 21.855 | 1.00 | 18.67 |
| ATOM | 1291 | N | THR | 178 | −23.162 | −4.669 | 19.994 | 1.00 | 22.44 |
| ATOM | 1292 | CA | THR | 178 | −23.907 | −3.410 | 19.888 | 1.00 | 24.09 |
| ATOM | 1293 | C | THR | 178 | −25.177 | −3.528 | 19.018 | 1.00 | 24.45 |
| ATOM | 1294 | O | THR | 178 | −25.966 | −2.590 | 18.931 | 1.00 | 25.93 |
| ATOM | 1295 | CB | THR | 178 | −23.000 | −2.254 | 19.325 | 1.00 | 24.21 |
| ATOM | 1296 | CG2 | THR | 178 | −21.813 | −2.003 | 20.228 | 1.00 | 23.67 |
| ATOM | 1297 | OG1 | THR | 178 | −22.523 | −2.583 | 18.008 | 1.00 | 26.71 |
| ATOM | 1298 | N | GLU | 179 | −25.381 | −4.668 | 18.369 | 1.00 | 23.13 |
| ATOM | 1299 | CA | GLU | 179 | −26.503 | −4.791 | 17.450 | 1.00 | 21.69 |
| ATOM | 1300 | C | GLU | 179 | −26.983 | −6.240 | 17.433 | 1.00 | 20.88 |
| ATOM | 1301 | O | GLU | 179 | −26.183 | −7.160 | 17.208 | 1.00 | 20.52 |
| ATOM | 1302 | CB | GLU | 179 | −26.061 | −4.375 | 16.045 | 1.00 | 21.33 |
| ATOM | 1303 | CG | GLU | 179 | −27.214 | −4.109 | 15.111 | 1.00 | 20.63 |
| ATOM | 1304 | CD | GLU | 179 | −26.783 | −3.790 | 13.683 | 0.50 | 18.61 |
| ATOM | 1305 | OE1 | GLU | 179 | −25.574 | −3.522 | 13.436 | 0.50 | 16.73 |
| ATOM | 1306 | OE2 | GLU | 179 | −27.671 | −3.812 | 12.812 | 0.50 | 16.81 |
| ATOM | 1307 | N | ASN | 180 | −28.280 | −6.448 | 17.643 | 1.00 | 19.55 |
| ATOM | 1308 | CA | ASN | 180 | −28.818 | −7.804 | 17.744 | 1.00 | 18.29 |
| ATOM | 1309 | C | ASN | 180 | −27.916 | −8.679 | 18.638 | 1.00 | 17.26 |
| ATOM | 1310 | O | ASN | 180 | −27.495 | −9.775 | 18.231 | 1.00 | 17.80 |
| ATOM | 1311 | CB | ASN | 180 | −28.968 | −8.431 | 16.343 | 1.00 | 17.96 |
| ATOM | 1312 | CG | ASN | 180 | −30.058 | −7.764 | 15.507 | 1.00 | 19.10 |
| ATOM | 1313 | ND2 | ASN | 180 | −29.708 | −7.345 | 14.278 | 1.00 | 15.84 |
| ATOM | 1314 | OD1 | ASN | 180 | −31.200 | −7.638 | 15.953 | 1.00 | 18.68 |
| ATOM | 1315 | N | ALA | 181 | −27.616 | −8.189 | 19.840 | 1.00 | 15.46 |
| ATOM | 1316 | CA | ALA | 181 | −26.749 | −8.898 | 20.793 | 1.00 | 15.53 |
| ATOM | 1317 | C | ALA | 181 | −27.310 | −10.253 | 21.266 | 1.00 | 15.30 |
| ATOM | 1318 | O | ALA | 181 | −26.536 | −11.165 | 21.616 | 1.00 | 15.29 |
| ATOM | 1319 | CB | ALA | 181 | −26.367 | −7.993 | 21.989 | 1.00 | 14.62 |
| ATOM | 1320 | N | ASP | 182 | −28.639 | −10.383 | 21.226 | 1.00 | 14.39 |
| ATOM | 1321 | CA | ASP | 182 | −29.292 | −11.634 | 21.523 | 1.00 | 14.28 |
| ATOM | 1322 | C | ASP | 182 | −28.978 | −12.691 | 20.443 | 1.00 | 14.26 |
| ATOM | 1323 | O | ASP | 182 | −28.630 | −13.834 | 20.771 | 1.00 | 14.08 |
| ATOM | 1324 | CB | ASP | 182 | −30.803 | −11.450 | 21.761 | 1.00 | 14.39 |
| ATOM | 1325 | CG | ASP | 182 | −31.567 | −10.842 | 20.550 | 1.00 | 16.12 |
| ATOM | 1326 | OD1 | ASP | 182 | −31.006 | −10.099 | 19.699 | 1.00 | 17.74 |
| ATOM | 1327 | OD2 | ASP | 182 | −32.776 | −11.097 | 20.483 | 1.00 | 16.62 |
| ATOM | 1328 | N | LEU | 183 | −29.056 | −12.294 | 19.172 | 1.00 | 13.86 |
| ATOM | 1329 | CA | LEU | 183 | −28.685 | −13.177 | 18.067 | 1.00 | 13.43 |
| ATOM | 1330 | C | LEU | 183 | −27.198 | −13.534 | 18.154 | 1.00 | 13.58 |
| ATOM | 1331 | O | LEU | 183 | −26.825 | −14.712 | 18.039 | 1.00 | 13.89 |
| ATOM | 1332 | CB | LEU | 183 | −28.999 | −12.519 | 16.726 | 1.00 | 12.51 |
| ATOM | 1333 | CG | LEU | 183 | −28.638 | −13.271 | 15.453 | 1.00 | 13.38 |
| ATOM | 1334 | CD1 | LEU | 183 | −29.192 | −14.701 | 15.458 | 1.00 | 10.65 |
| ATOM | 1335 | CD2 | LEU | 183 | −29.122 | −12.495 | 14.208 | 1.00 | 11.98 |
| ATOM | 1336 | N | PHE | 184 | −26.379 | −12.507 | 18.376 | 1.00 | 13.33 |
| ATOM | 1337 | CA | PHE | 184 | −24.926 | −12.624 | 18.567 | 1.00 | 13.40 |
| ATOM | 1338 | C | PHE | 184 | −24.554 | −13.699 | 19.605 | 1.00 | 13.53 |
| ATOM | 1339 | O | PHE | 184 | −23.666 | −14.535 | 19.362 | 1.00 | 13.18 |
| ATOM | 1340 | CB | PHE | 184 | −24.342 | −11.255 | 18.960 | 1.00 | 12.88 |
| ATOM | 1341 | CG | PHE | 184 | −22.840 | −11.171 | 18.873 | 1.00 | 13.14 |
| ATOM | 1342 | CD1 | PHE | 184 | −22.173 | −11.486 | 17.675 | 1.00 | 14.04 |
| ATOM | 1343 | CD2 | PHE | 184 | −22.098 | −10.763 | 19.968 | 1.00 | 13.10 |
| ATOM | 1344 | CE1 | PHE | 184 | −20.801 | −11.406 | 17.584 | 1.00 | 13.58 |
| ATOM | 1345 | CE2 | PHE | 184 | −20.711 | −10.678 | 19.897 | 1.00 | 12.99 |
| ATOM | 1346 | CZ | PHE | 184 | −20.061 | −10.980 | 18.698 | 1.00 | 12.86 |
| ATOM | 1347 | N | TRP | 185 | −25.243 | −13.675 | 20.743 | 1.00 | 12.85 |
| ATOM | 1348 | CA | TRP | 185 | −25.026 | −14.630 | 21.829 | 1.00 | 13.18 |
| ATOM | 1349 | C | TRP | 185 | −25.279 | −16.066 | 21.338 | 1.00 | 13.31 |
| ATOM | 1350 | O | TRP | 185 | −24.475 | −16.979 | 21.595 | 1.00 | 12.99 |
| ATOM | 1351 | CB | TRP | 185 | −25.974 | −14.287 | 22.993 | 1.00 | 13.46 |
| ATOM | 1352 | CG | TRP | 185 | −25.844 | −15.131 | 24.250 | 1.00 | 12.24 |
| ATOM | 1353 | CD1 | TRP | 185 | −25.022 | −14.884 | 25.303 | 1.00 | 11.88 |
| ATOM | 1354 | CD2 | TRP | 185 | −26.576 | −16.329 | 24.585 | 1.00 | 12.93 |
| ATOM | 1355 | CE2 | TRP | 185 | −26.147 | −16.736 | 25.875 | 1.00 | 10.98 |
| ATOM | 1356 | CE3 | TRP | 185 | −27.564 | −17.086 | 23.933 | 1.00 | 12.97 |
| ATOM | 1357 | NE1 | TRP | 185 | −25.205 | −15.838 | 26.285 | 1.00 | 11.77 |
| ATOM | 1358 | CZ2 | TRP | 185 | −26.666 | −17.880 | 26.535 | 1.00 | 12.06 |
| ATOM | 1359 | CZ3 | TRP | 185 | −28.101 | −18.231 | 24.606 | 1.00 | 11.87 |
| ATOM | 1360 | CH2 | TRP | 185 | −27.633 | −18.616 | 25.872 | 1.00 | 12.63 |
| ATOM | 1361 | N | GLY | 186 | −26.397 | −16.245 | 20.623 | 1.00 | 13.73 |
| ATOM | 1362 | CA | GLY | 186 | −26.779 | −17.540 | 20.074 | 1.00 | 13.18 |
| ATOM | 1363 | C | GLY | 186 | −25.809 | −18.037 | 19.017 | 1.00 | 13.92 |
| ATOM | 1364 | O | GLY | 186 | −25.499 | −19.230 | 18.966 | 1.00 | 14.21 |
| ATOM | 1365 | N | ILE | 187 | −25.331 | −17.122 | 18.167 | 1.00 | 13.33 |
| ATOM | 1366 | CA | ILE | 187 | −24.409 | −17.497 | 17.100 | 1.00 | 12.71 |
| ATOM | 1367 | C | ILE | 187 | −23.098 | −18.026 | 17.690 | 1.00 | 12.28 |
| ATOM | 1368 | O | ILE | 187 | −22.470 | −18.970 | 17.154 | 1.00 | 11.58 |
| ATOM | 1369 | CB | ILE | 187 | −24.131 | −16.297 | 16.156 | 1.00 | 12.79 |
| ATOM | 1370 | CG1 | ILE | 187 | −25.393 | −15.905 | 15.367 | 1.00 | 12.72 |
| ATOM | 1371 | CG2 | ILE | 187 | −22.942 | −16.562 | 15.217 | 1.00 | 11.47 |
| ATOM | 1372 | CD1 | ILE | 187 | −25.911 | −16.920 | 14.363 | 1.00 | 15.75 |
| ATOM | 1373 | N | LYS | 188 | −22.706 | −17.424 | 18.808 | 1.00 | 10.99 |
| ATOM | 1374 | CA | LYS | 188 | −21.478 | −17.793 | 19.488 | 1.00 | 11.23 |
| ATOM | 1375 | C | LYS | 188 | −21.603 | −19.012 | 20.395 | 1.00 | 10.94 |
| ATOM | 1376 | O | LYS | 188 | −21.478 | −18.913 | 21.613 | 1.00 | 11.11 |
| ATOM | 1377 | CB | LYS | 188 | −20.881 | −16.592 | 20.211 | 1.00 | 10.31 |
| ATOM | 1378 | CG | LYS | 188 | −20.319 | −15.567 | 19.240 | 1.00 | 10.97 |
| ATOM | 1379 | CD | LYS | 188 | −19.800 | −14.322 | 19.950 | 1.00 | 11.41 |
| ATOM | 1380 | CE | LYS | 188 | −18.564 | −14.690 | 20.765 | 1.00 | 12.37 |
| ATOM | 1381 | NZ | LYS | 188 | −17.842 | −13.471 | 21.180 | 1.00 | 13.18 |
| ATOM | 1382 | N | GLY | 189 | −21.837 | −20.176 | 19.778 | 1.00 | 11.12 |
| ATOM | 1383 | CA | GLY | 189 | −21.860 | −21.448 | 20.490 | 1.00 | 10.69 |
| ATOM | 1384 | C | GLY | 189 | −22.955 | −22.396 | 20.030 | 1.00 | 11.00 |
| ATOM | 1385 | O | GLY | 189 | −22.893 | −23.598 | 20.287 | 1.00 | 10.82 |
| ATOM | 1386 | N | ALA | 190 | −23.967 | −21.857 | 19.356 | 1.00 | 11.24 |
| ATOM | 1387 | CA | ALA | 190 | −25.086 | −22.658 | 18.890 | 1.00 | 11.16 |
| ATOM | 1388 | C | ALA | 190 | −25.513 | −22.152 | 17.530 | 1.00 | 11.62 |
| ATOM | 1389 | O | ALA | 190 | −26.674 | −22.274 | 17.159 | 1.00 | 11.40 |
| ATOM | 1390 | CB | ALA | 190 | −26.264 | −22.596 | 19.913 | 1.00 | 11.43 |
| ATOM | 1391 | N | GLY | 191 | −24.533 | −21.648 | 16.765 | 1.00 | 11.94 |
| ATOM | 1392 | CA | GLY | 191 | −24.768 | −20.815 | 15.591 | 1.00 | 12.05 |
| ATOM | 1393 | C | GLY | 191 | −25.566 | −21.450 | 14.462 | 1.00 | 13.63 |
| ATOM | 1394 | O | GLY | 191 | −26.253 | −20.735 | 13.712 | 1.00 | 14.71 |
| ATOM | 1395 | N | SER | 192 | −25.502 | −22.778 | 14.343 | 1.00 | 12.18 |
| ATOM | 1396 | CA | SER | 192 | −26.267 | −23.487 | 13.333 | 1.00 | 11.99 |
| ATOM | 1397 | C | SER | 192 | −27.775 | −23.315 | 13.474 | 1.00 | 12.64 |
| ATOM | 1398 | O | SER | 192 | −28.512 | −23.585 | 12.533 | 1.00 | 13.26 |
| ATOM | 1399 | CB | SER | 192 | −25.913 | −24.985 | 13.335 | 1.00 | 11.65 |
| ATOM | 1400 | OG | SER | 192 | −26.031 | −25.545 | 14.639 | 1.00 | 10.40 |
| ATOM | 1401 | N | ASN | 193 | −28.220 | −22.927 | 14.678 | 1.00 | 13.56 |
| ATOM | 1402 | CA | ASN | 193 | −29.641 | −22.787 | 15.010 | 1.00 | 12.69 |
| ATOM | 1403 | C | ASN | 193 | −30.362 | −21.558 | 14.468 | 1.00 | 12.03 |
| ATOM | 1404 | O | ASN | 193 | −31.591 | −21.597 | 14.326 | 1.00 | 11.56 |
| ATOM | 1405 | CB | ASN | 193 | −29.852 | −22.898 | 16.535 | 1.00 | 12.55 |
| ATOM | 1406 | CG | ASN | 193 | −29.704 | −24.303 | 17.017 | 1.00 | 12.56 |
| ATOM | 1407 | ND2 | ASN | 193 | −28.563 | −24.610 | 17.642 | 1.00 | 12.03 |
| ATOM | 1408 | OD1 | ASN | 193 | −30.573 | −25.132 | 16.780 | 1.00 | 14.33 |
| ATOM | 1409 | N | PHE | 194 | −29.622 | −20.492 | 14.145 | 1.00 | 11.14 |
| ATOM | 1410 | CA | PHE | 194 | −30.249 | −19.165 | 14.012 | 1.00 | 10.79 |
| ATOM | 1411 | C | PHE | 194 | −30.223 | −18.487 | 12.653 | 1.00 | 11.14 |
| ATOM | 1412 | O | PHE | 194 | −30.856 | −17.452 | 12.473 | 1.00 | 10.99 |
| ATOM | 1413 | CB | PHE | 194 | −29.714 | −18.228 | 15.104 | 1.00 | 9.78 |
| ATOM | 1414 | CG | PHE | 194 | −29.830 | −18.835 | 16.485 | 1.00 | 9.95 |
| ATOM | 1415 | CD1 | PHE | 194 | −31.090 | −19.023 | 17.057 | 1.00 | 10.40 |
| ATOM | 1416 | CD2 | PHE | 194 | −28.685 | −19.300 | 17.168 | 1.00 | 11.15 |
| ATOM | 1417 | CE1 | PHE | 194 | −31.236 | −19.625 | 18.319 | 1.00 | 10.37 |
| ATOM | 1418 | CE2 | PHE | 194 | −28.818 | −19.920 | 18.445 | 1.00 | 10.26 |
| ATOM | 1419 | CZ | PHE | 194 | −30.079 | −20.076 | 19.009 | 1.00 | 12.16 |
| ATOM | 1420 | N | GLY | 195 | −29.513 | −19.083 | 11.698 | 1.00 | 11.66 |
| ATOM | 1421 | CA | GLY | 195 | −29.482 | −18.552 | 10.330 | 1.00 | 10.50 |
| ATOM | 1422 | C | GLY | 195 | −28.284 | −19.108 | 9.638 | 1.00 | 9.96 |
| ATOM | 1423 | O | GLY | 195 | −27.679 | −20.067 | 10.134 | 1.00 | 10.74 |
| ATOM | 1424 | N | ILE | 196 | −27.939 | −18.544 | 8.480 | 1.00 | 9.29 |
| ATOM | 1425 | CA | ILE | 196 | −26.702 | −18.943 | 7.782 | 1.00 | 8.18 |
| ATOM | 1426 | C | ILE | 196 | −25.754 | −17.732 | 7.877 | 1.00 | 8.30 |
| ATOM | 1427 | O | ILE | 196 | −26.067 | −16.635 | 7.362 | 1.00 | 9.38 |
| ATOM | 1428 | CB | ILE | 196 | −26.970 | −19.382 | 6.325 | 1.00 | 7.41 |
| ATOM | 1429 | CG1 | ILE | 196 | −28.004 | −20.525 | 6.330 | 1.00 | 8.52 |
| ATOM | 1430 | CG2 | ILE | 196 | −25.612 | −19.773 | 5.584 | 1.00 | 6.37 |
| ATOM | 1431 | CD1 | ILE | 196 | −28.491 | −21.022 | 4.979 | 1.00 | 10.15 |
| ATOM | 1432 | N | VAL | 197 | −24.662 | −17.899 | 8.616 | 1.00 | 7.13 |
| ATOM | 1433 | CA | VAL | 197 | −23.677 | −16.839 | 8.740 | 1.00 | 7.98 |
| ATOM | 1434 | C | VAL | 197 | −23.021 | −16.635 | 7.373 | 1.00 | 8.89 |
| ATOM | 1435 | O | VAL | 197 | −22.538 | −17.612 | 6.743 | 1.00 | 8.64 |
| ATOM | 1436 | CB | VAL | 197 | −22.602 | −17.128 | 9.812 | 1.00 | 7.38 |
| ATOM | 1437 | CG1 | VAL | 197 | −21.563 | −15.990 | 9.800 | 1.00 | 6.41 |
| ATOM | 1438 | CG2 | VAL | 197 | −23.266 | −17.279 | 11.197 | 1.00 | 6.84 |
| ATOM | 1439 | N | ALA | 198 | −23.046 | −15.380 | 6.903 | 1.00 | 9.47 |
| ATOM | 1440 | CA | ALA | 198 | −22.548 | −15.020 | 5.564 | 1.00 | 10.35 |
| ATOM | 1441 | C | ALA | 198 | −21.170 | −14.357 | 5.596 | 1.00 | 10.91 |
| ATOM | 1442 | O | ALA | 198 | −20.432 | −14.400 | 4.600 | 1.00 | 11.32 |
| ATOM | 1443 | CB | ALA | 198 | −23.577 | −14.097 | 4.835 | 1.00 | 9.62 |
| ATOM | 1444 | N | VAL | 199 | −20.847 | −13.709 | 6.720 | 1.00 | 10.63 |
| ATOM | 1445 | CA | VAL | 199 | −19.512 | −13.132 | 6.944 | 1.00 | 10.35 |
| ATOM | 1446 | C | VAL | 199 | −19.190 | −13.313 | 8.423 | 1.00 | 10.34 |
| ATOM | 1447 | O | VAL | 199 | −20.025 | −12.994 | 9.291 | 1.00 | 10.14 |
| ATOM | 1448 | CB | VAL | 199 | −19.453 | −11.612 | 6.590 | 1.00 | 10.52 |
| ATOM | 1449 | CG1 | VAL | 199 | −18.052 | −11.019 | 6.810 | 1.00 | 10.78 |
| ATOM | 1450 | CG2 | VAL | 199 | −19.933 | −11.348 | 5.144 | 1.00 | 12.14 |
| ATOM | 1451 | N | TRP | 200 | −17.997 | −13.831 | 8.718 | 1.00 | 10.36 |
| ATOM | 1452 | CA | TRP | 200 | −17.468 | −13.821 | 10.097 | 1.00 | 10.40 |
| ATOM | 1453 | C | TRP | 200 | −16.556 | −12.608 | 10.275 | 1.00 | 10.73 |
| ATOM | 1454 | O | TRP | 200 | −15.671 | −12.372 | 9.449 | 1.00 | 11.28 |
| ATOM | 1455 | CB | TRP | 200 | −16.684 | −15.109 | 10.402 | 1.00 | 10.07 |
| ATOM | 1456 | CG | TRP | 200 | −17.525 | −16.350 | 10.343 | 1.00 | 10.01 |
| ATOM | 1457 | CD1 | TRP | 200 | −17.766 | −17.150 | 9.245 | 1.00 | 10.33 |
| ATOM | 1458 | CD2 | TRP | 200 | −18.284 | −16.908 | 11.427 | 1.00 | 11.16 |
| ATOM | 1459 | CE2 | TRP | 200 | −18.951 | −18.057 | 10.933 | 1.00 | 9.48 |
| ATOM | 1460 | CE3 | TRP | 200 | −18.444 | −16.554 | 12.779 | 1.00 | 9.47 |
| ATOM | 1461 | NE1 | TRP | 200 | −18.636 | −18.182 | 9.597 | 1.00 | 9.60 |
| ATOM | 1462 | CZ2 | TRP | 200 | −19.791 | −18.845 | 11.748 | 1.00 | 9.80 |
| ATOM | 1463 | CZ3 | TRP | 200 | −19.248 | −17.338 | 13.587 | 1.00 | 7.62 |
| ATOM | 1464 | CH2 | TRP | 200 | −19.925 | −18.475 | 13.069 | 1.00 | 7.32 |
| ATOM | 1465 | N | LYS | 201 | −16.755 | −11.850 | 11.349 | 1.00 | 10.91 |
| ATOM | 1466 | CA | LYS | 201 | −15.816 | −10.750 | 11.701 | 1.00 | 11.35 |
| ATOM | 1467 | C | LYS | 201 | −14.998 | −11.147 | 12.920 | 1.00 | 11.00 |
| ATOM | 1468 | O | LYS | 201 | −15.534 | −11.366 | 14.013 | 1.00 | 10.07 |
| ATOM | 1469 | CB | LYS | 201 | −16.574 | −9.425 | 11.895 | 1.00 | 11.13 |
| ATOM | 1470 | CG | LYS | 201 | −17.527 | −9.161 | 10.731 | 1.00 | 14.33 |
| ATOM | 1471 | CD | LYS | 201 | −18.520 | −8.072 | 11.023 | 1.00 | 21.53 |
| ATOM | 1472 | CE | LYS | 201 | −19.457 | −7.829 | 9.835 | 1.00 | 24.94 |
| ATOM | 1473 | NZ | LYS | 201 | −20.599 | −6.924 | 10.301 | 1.00 | 27.91 |
| ATOM | 1474 | N | LEU | 202 | −13.693 | −11.253 | 12.733 | 1.00 | 11.57 |
| ATOM | 1475 | CA | LEU | 202 | −12.854 | −11.984 | 13.694 | 1.00 | 12.90 |
| ATOM | 1476 | C | LEU | 202 | −11.685 | −11.154 | 14.147 | 1.00 | 13.58 |
| ATOM | 1477 | O | LEU | 202 | −10.941 | −10.637 | 13.291 | 1.00 | 14.87 |
| ATOM | 1478 | CB | LEU | 202 | −12.293 | −13.259 | 13.031 | 1.00 | 12.46 |
| ATOM | 1479 | CG | LEU | 202 | −13.294 | −14.241 | 12.404 | 1.00 | 12.09 |
| ATOM | 1480 | CD1 | LEU | 202 | −12.562 | −15.444 | 11.717 | 1.00 | 7.41 |
| ATOM | 1481 | CD2 | LEU | 202 | −14.256 | −14.736 | 13.476 | 1.00 | 9.95 |
| ATOM | 1482 | N | ALA | 203 | −11.508 | −11.040 | 15.462 | 1.00 | 12.85 |
| ATOM | 1483 | CA | ALA | 203 | −10.328 | −10.395 | 16.046 | 1.00 | 13.97 |
| ATOM | 1484 | C | ALA | 203 | −9.157 | −11.345 | 15.829 | 1.00 | 14.44 |
| ATOM | 1485 | O | ALA | 203 | −9.331 | −12.558 | 15.970 | 1.00 | 15.88 |
| ATOM | 1486 | CB | ALA | 203 | −10.530 | −10.173 | 17.585 | 1.00 | 12.67 |
| ATOM | 1487 | N | THR | 204 | −7.981 | −10.817 | 15.500 | 1.00 | 14.84 |
| ATOM | 1488 | CA | THR | 204 | −6.785 | −11.652 | 15.326 | 1.00 | 14.94 |
| ATOM | 1489 | C | THR | 204 | −5.686 | −11.181 | 16.295 | 1.00 | 15.51 |
| ATOM | 1490 | O | THR | 204 | −5.796 | −10.088 | 16.869 | 1.00 | 15.61 |
| ATOM | 1491 | CB | THR | 204 | −6.238 | −11.599 | 13.887 | 1.00 | 14.91 |
| ATOM | 1492 | CG2 | THR | 204 | −7.326 | −11.973 | 12.868 | 1.00 | 13.12 |
| ATOM | 1493 | OG1 | THR | 204 | −5.748 | −10.273 | 13.615 | 1.00 | 14.88 |
| ATOM | 1494 | N | PHE | 205 | −4.657 | −12.013 | 16.483 | 1.00 | 15.00 |
| ATOM | 1495 | CA | PHE | 205 | −3.439 | −11.629 | 17.213 | 1.00 | 16.10 |
| ATOM | 1496 | C | PHE | 205 | −2.198 | −11.761 | 16.302 | 1.00 | 17.13 |
| ATOM | 1497 | O | PHE | 205 | −2.244 | −12.485 | 15.283 | 1.00 | 17.10 |
| ATOM | 1498 | CB | PHE | 205 | −3.277 | −12.400 | 18.543 | 1.00 | 15.71 |
| ATOM | 1499 | CG | PHE | 205 | −3.325 | −13.908 | 18.417 | 1.00 | 15.76 |
| ATOM | 1500 | CD1 | PHE | 205 | −2.153 | −14.655 | 18.201 | 1.00 | 16.50 |
| ATOM | 1501 | CD2 | PHE | 205 | −4.534 | −14.588 | 18.587 | 1.00 | 14.35 |
| ATOM | 1502 | CE1 | PHE | 205 | −2.186 | −16.044 | 18.102 | 1.00 | 14.44 |
| ATOM | 1503 | CE2 | PHE | 205 | −4.596 | −15.972 | 18.505 | 1.00 | 14.10 |
| ATOM | 1504 | CZ | PHE | 205 | −3.423 | −16.716 | 18.248 | 1.00 | 16.41 |
| ATOM | 1505 | N | PRO | 206 | −1.094 | −11.055 | 16.645 | 1.00 | 17.33 |
| ATOM | 1506 | CA | PRO | 206 | 0.097 | −11.190 | 15.812 | 1.00 | 17.18 |
| ATOM | 1507 | C | PRO | 206 | 0.554 | −12.645 | 15.837 | 1.00 | 17.49 |
| ATOM | 1508 | O | PRO | 206 | 0.542 | −13.274 | 16.904 | 1.00 | 17.95 |
| ATOM | 1509 | CB | PRO | 206 | 1.135 | −10.269 | 16.497 | 1.00 | 17.08 |
| ATOM | 1510 | CG | PRO | 206 | 0.356 | −9.377 | 17.402 | 1.00 | 15.97 |
| ATOM | 1511 | CD | PRO | 206 | −0.869 | −10.159 | 17.800 | 1.00 | 17.45 |
| ATOM | 1512 | N | ALA | 207 | 0.947 | −13.166 | 14.679 | 1.00 | 17.69 |
| ATOM | 1513 | CA | ALA | 207 | 1.347 | −14.557 | 14.568 | 1.00 | 18.43 |
| ATOM | 1514 | C | ALA | 207 | 2.591 | −14.778 | 15.431 | 1.00 | 18.73 |
| ATOM | 1515 | O | ALA | 207 | 3.583 | −14.096 | 15.237 | 1.00 | 18.48 |
| ATOM | 1516 | CB | ALA | 207 | 1.602 | −14.934 | 13.098 | 1.00 | 17.69 |
| ATOM | 1517 | N | PRO | 208 | 2.525 | −15.715 | 16.406 | 1.00 | 19.28 |
| ATOM | 1518 | CA | PRO | 208 | 3.646 | −15.955 | 17.351 | 1.00 | 19.51 |
| ATOM | 1519 | C | PRO | 208 | 4.923 | −16.369 | 16.632 | 1.00 | 19.99 |
| ATOM | 1520 | O | PRO | 208 | 4.861 | −17.132 | 15.653 | 1.00 | 19.42 |
| ATOM | 1521 | CB | PRO | 208 | 3.165 | −17.134 | 18.192 | 1.00 | 19.27 |
| ATOM | 1522 | CG | PRO | 208 | 1.700 | −17.169 | 18.040 | 1.00 | 19.20 |
| ATOM | 1523 | CD | PRO | 208 | 1.306 | −16.461 | 16.779 | 1.00 | 19.08 |
| ATOM | 1524 | N | LYS | 209 | 6.064 | −15.875 | 17.112 | 1.00 | 20.39 |
| ATOM | 1525 | CA | LYS | 209 | 7.356 | −16.189 | 16.498 | 1.00 | 21.16 |
| ATOM | 1526 | C | LYS | 209 | 8.046 | −17.359 | 17.194 | 1.00 | 20.50 |
| ATOM | 1527 | O | LYS | 209 | 8.824 | −18.081 | 16.582 | 1.00 | 20.76 |
| ATOM | 1528 | CB | LYS | 209 | 8.272 | −14.964 | 16.552 | 1.00 | 22.55 |
| ATOM | 1529 | CG | LYS | 209 | 7.861 | −13.814 | 15.634 | 1.00 | 25.32 |
| ATOM | 1530 | CD | LYS | 209 | 8.818 | −12.636 | 15.812 | 1.00 | 30.50 |
| ATOM | 1531 | CE | LYS | 209 | 8.369 | −11.443 | 14.977 | 1.00 | 35.33 |
| ATOM | 1532 | NZ | LYS | 209 | 8.009 | −11.892 | 13.589 | 1.00 | 37.77 |
| ATOM | 1533 | N | VAL | 210 | 7.759 | −17.535 | 18.477 | 1.00 | 19.65 |
| ATOM | 1534 | CA | VAL | 210 | 8.462 | −18.512 | 19.314 | 1.00 | 19.18 |
| ATOM | 1535 | C | VAL | 210 | 7.464 | −19.480 | 19.973 | 1.00 | 18.64 |
| ATOM | 1536 | O | VAL | 210 | 6.684 | −19.100 | 20.863 | 1.00 | 18.21 |
| ATOM | 1537 | CB | VAL | 210 | 9.338 | −17.793 | 20.402 | 1.00 | 19.44 |
| ATOM | 1538 | CG1 | VAL | 210 | 10.288 | −16.791 | 19.764 | 1.00 | 19.82 |
| ATOM | 1539 | CG2 | VAL | 210 | 10.099 | −18.793 | 21.274 | 1.00 | 19.91 |
| ATOM | 1540 | N | LEU | 211 | 7.497 | −20.735 | 19.530 | 1.00 | 17.67 |
| ATOM | 1541 | CA | LEU | 211 | 6.587 | −21.760 | 20.037 | 1.00 | 16.70 |
| ATOM | 1542 | C | LEU | 211 | 7.375 | −23.036 | 20.172 | 1.00 | 17.05 |
| ATOM | 1543 | O | LEU | 211 | 8.328 | −23.294 | 19.399 | 1.00 | 16.46 |
| ATOM | 1544 | CB | LEU | 211 | 5.416 | −21.990 | 19.078 | 1.00 | 16.29 |
| ATOM | 1545 | CG | LEU | 211 | 4.455 | −20.838 | 18.791 | 1.00 | 16.17 |
| ATOM | 1546 | CD1 | LEU | 211 | 3.625 | −21.120 | 17.560 | 1.00 | 14.36 |
| ATOM | 1547 | CD2 | LEU | 211 | 3.575 | −20.477 | 20.037 | 1.00 | 14.91 |
| ATOM | 1548 | N | THR | 212 | 6.986 | −23.845 | 21.146 | 1.00 | 16.25 |
| ATOM | 1549 | CA | THR | 212 | 7.635 | −25.128 | 21.318 | 1.00 | 16.56 |
| ATOM | 1550 | C | THR | 212 | 6.577 | −26.219 | 21.357 | 1.00 | 17.07 |
| ATOM | 1551 | O | THR | 212 | 5.639 | −26.180 | 22.187 | 1.00 | 16.63 |
| ATOM | 1552 | CB | THR | 212 | 8.464 | −25.176 | 22.620 | 1.00 | 16.31 |
| ATOM | 1553 | CG2 | THR | 212 | 9.122 | −26.526 | 22.786 | 1.00 | 16.20 |
| ATOM | 1554 | OG1 | THR | 212 | 9.486 | −24.185 | 22.570 | 1.00 | 16.18 |
| ATOM | 1555 | N | ARG | 213 | 6.735 | −27.176 | 20.456 | 1.00 | 16.19 |
| ATOM | 1556 | CA | ARG | 213 | 5.966 | −28.392 | 20.489 | 1.00 | 16.23 |
| ATOM | 1557 | C | ARG | 213 | 6.643 | −29.359 | 21.447 | 1.00 | 16.25 |
| ATOM | 1558 | O | ARG | 213 | 7.857 | −29.552 | 21.399 | 1.00 | 15.54 |
| ATOM | 1559 | CB | ARG | 213 | 5.886 | −29.019 | 19.104 | 1.00 | 16.29 |
| ATOM | 1560 | CG | ARG | 213 | 5.370 | −30.467 | 19.123 | 1.00 | 16.67 |
| ATOM | 1561 | CD | ARG | 213 | 4.820 | −30.873 | 17.772 | 1.00 | 19.32 |
| ATOM | 1562 | NE | ARG | 213 | 4.192 | −32.184 | 17.907 | 1.00 | 22.87 |
| ATOM | 1563 | CZ | ARG | 213 | 3.462 | −32.775 | 16.976 | 1.00 | 23.11 |
| ATOM | 1564 | NH1 | ARG | 213 | 3.226 | −32.180 | 15.810 | 1.00 | 21.61 |
| ATOM | 1565 | NH2 | ARG | 213 | 2.951 | −33.974 | 17.228 | 1.00 | 26.25 |
| ATOM | 1566 | N | PHE | 214 | 5.840 | −29.977 | 22.303 | 1.00 | 15.74 |
| ATOM | 1567 | CA | PHE | 214 | 6.358 | −30.920 | 23.264 | 1.00 | 14.83 |
| ATOM | 1568 | C | PHE | 214 | 5.518 | −32.173 | 23.316 | 1.00 | 15.25 |
| ATOM | 1569 | O | PHE | 214 | 4.394 | −32.206 | 22.791 | 1.00 | 14.46 |
| ATOM | 1570 | CB | PHE | 214 | 6.485 | −30.267 | 24.648 | 1.00 | 14.37 |
| ATOM | 1571 | CG | PHE | 214 | 5.188 | −29.843 | 25.255 | 1.00 | 12.58 |
| ATOM | 1572 | CD1 | PHE | 214 | 4.482 | −30.704 | 26.098 | 1.00 | 14.26 |
| ATOM | 1573 | CD2 | PHE | 214 | 4.703 | −28.562 | 25.044 | 1.00 | 9.93 |
| ATOM | 1574 | CE1 | PHE | 214 | 3.299 | −30.286 | 26.711 | 1.00 | 12.12 |
| ATOM | 1575 | CE2 | PHE | 214 | 3.545 | −28.144 | 25.624 | 1.00 | 10.87 |
| ATOM | 1576 | CZ | PHE | 214 | 2.819 | −29.011 | 26.456 | 1.00 | 14.61 |
| ATOM | 1577 | N | GLY | 215 | 6.078 | −33.210 | 23.937 | 1.00 | 15.64 |
| ATOM | 1578 | CA | GLY | 215 | 5.445 | −34.511 | 23.977 | 1.00 | 16.87 |
| ATOM | 1579 | C | GLY | 215 | 6.063 | −35.361 | 25.073 | 1.00 | 17.72 |
| ATOM | 1580 | O | GLY | 215 | 7.266 | −35.236 | 25.337 | 1.00 | 17.40 |
| ATOM | 1581 | N | VAL | 216 | 5.230 | −36.189 | 25.719 | 1.00 | 17.22 |
| ATOM | 1582 | CA | VAL | 216 | 5.680 | −37.135 | 26.736 | 1.00 | 17.75 |
| ATOM | 1583 | C | VAL | 216 | 5.067 | −38.518 | 26.482 | 1.00 | 17.96 |
| ATOM | 1584 | O | VAL | 216 | 3.849 | −38.676 | 26.513 | 1.00 | 18.36 |
| ATOM | 1585 | CB | VAL | 216 | 5.303 | −36.649 | 28.198 | 1.00 | 17.69 |
| ATOM | 1586 | CG1 | VAL | 216 | 5.833 | −37.620 | 29.233 | 1.00 | 16.93 |
| ATOM | 1587 | CG2 | VAL | 216 | 5.846 | −35.219 | 28.462 | 1.00 | 15.32 |
| ATOM | 1588 | N | THR | 217 | 5.906 | −39.521 | 26.241 | 1.00 | 18.16 |
| ATOM | 1589 | CA | THR | 217 | 5.429 | −40.906 | 26.190 | 1.00 | 18.31 |
| ATOM | 1590 | C | THR | 217 | 5.211 | −41.356 | 27.629 | 1.00 | 17.90 |
| ATOM | 1591 | O | THR | 217 | 6.162 | −41.417 | 28.408 | 1.00 | 18.34 |
| ATOM | 1592 | CB | THR | 217 | 6.463 | −41.820 | 25.513 | 1.00 | 18.82 |
| ATOM | 1593 | CG2 | THR | 217 | 5.887 | −43.219 | 25.240 | 1.00 | 18.79 |
| ATOM | 1594 | OG1 | THR | 217 | 6.894 | −41.218 | 24.289 | 1.00 | 20.23 |
| ATOM | 1595 | N | LEU | 218 | 3.968 | −41.649 | 27.992 | 1.00 | 17.05 |
| ATOM | 1596 | CA | LEU | 218 | 3.623 | −41.776 | 29.400 | 1.00 | 17.16 |
| ATOM | 1597 | C | LEU | 218 | 4.034 | −43.106 | 29.997 | 1.00 | 18.14 |
| ATOM | 1598 | O | LEU | 218 | 4.351 | −43.176 | 31.178 | 1.00 | 18.41 |
| ATOM | 1599 | CB | LEU | 218 | 2.129 | −41.532 | 29.626 | 1.00 | 15.88 |
| ATOM | 1600 | CG | LEU | 218 | 1.735 | −40.079 | 29.385 | 1.00 | 14.56 |
| ATOM | 1601 | CD1 | LEU | 218 | 0.240 | −39.997 | 29.134 | 1.00 | 11.03 |
| ATOM | 1602 | CD2 | LEU | 218 | 2.166 | −39.218 | 30.589 | 1.00 | 12.28 |
| ATOM | 1603 | N | ASN | 219 | 4.020 | −44.144 | 29.164 | 1.00 | 18.83 |
| ATOM | 1604 | CA | ASN | 219 | 4.240 | −45.527 | 29.584 | 1.00 | 20.17 |
| ATOM | 1605 | C | ASN | 219 | 3.386 | −46.027 | 30.755 | 1.00 | 21.47 |
| ATOM | 1606 | O | ASN | 219 | 3.910 | −46.642 | 31.686 | 1.00 | 22.02 |
| ATOM | 1607 | CB | ASN | 219 | 5.738 | −45.789 | 29.800 | 1.00 | 19.64 |
| ATOM | 1608 | CG | ASN | 219 | 6.526 | −45.627 | 28.522 | 1.00 | 18.03 |
| ATOM | 1609 | ND2 | ASN | 219 | 7.593 | −44.848 | 28.565 | 1.00 | 18.17 |
| ATOM | 1610 | OD1 | ASN | 219 | 6.166 | −46.190 | 27.499 | 1.00 | 18.08 |
| ATOM | 1611 | N | TRP | 220 | 2.073 | −45.768 | 30.697 | 1.00 | 22.29 |
| ATOM | 1612 | CA | TRP | 220 | 1.138 | −46.331 | 31.661 | 1.00 | 23.04 |
| ATOM | 1613 | C | TRP | 220 | 0.753 | −47.747 | 31.202 | 1.00 | 24.99 |
| ATOM | 1614 | O | TRP | 220 | −0.104 | −47.915 | 30.315 | 1.00 | 25.91 |
| ATOM | 1615 | CB | TRP | 220 | −0.121 | −45.459 | 31.815 | 1.00 | 22.34 |
| ATOM | 1616 | CG | TRP | 220 | 0.122 | −44.043 | 32.238 | 1.00 | 20.07 |
| ATOM | 1617 | CD1 | TRP | 220 | 1.221 | −43.550 | 32.884 | 1.00 | 19.74 |
| ATOM | 1618 | CD2 | TRP | 220 | −0.757 | −42.930 | 32.039 | 1.00 | 19.94 |
| ATOM | 1619 | CE2 | TRP | 220 | −0.124 | −41.789 | 32.602 | 1.00 | 20.89 |
| ATOM | 1620 | CE3 | TRP | 220 | −2.024 | −42.783 | 31.450 | 1.00 | 18.93 |
| ATOM | 1621 | NE1 | TRP | 220 | 1.088 | −42.194 | 33.104 | 1.00 | 19.78 |
| ATOM | 1622 | CZ2 | TRP | 220 | −0.716 | −40.523 | 32.586 | 1.00 | 20.21 |
| ATOM | 1623 | CZ3 | TRP | 220 | −2.612 | −41.531 | 31.439 | 1.00 | 19.78 |
| ATOM | 1624 | CH2 | TRP | 220 | −1.954 | −40.408 | 31.998 | 1.00 | 21.94 |
| ATOM | 1625 | N | LYS | 221 | 1.398 | −48.749 | 31.793 | 1.00 | 26.02 |
| ATOM | 1626 | CA | LYS | 221 | 1.227 | −50.140 | 31.385 | 1.00 | 27.22 |
| ATOM | 1627 | C | LYS | 221 | 0.162 | −50.857 | 32.193 | 1.00 | 27.17 |
| ATOM | 1628 | O | LYS | 221 | −0.122 | −52.009 | 31.914 | 1.00 | 27.88 |
| ATOM | 1629 | CB | LYS | 221 | 2.555 | −50.925 | 31.511 | 1.00 | 28.27 |
| ATOM | 1630 | CG | LYS | 221 | 3.790 | −50.292 | 30.819 | 1.00 | 30.07 |
| ATOM | 1631 | CD | LYS | 221 | 3.522 | −50.006 | 29.339 | 1.00 | 33.42 |
| ATOM | 1632 | CE | LYS | 221 | 4.818 | −49.749 | 28.578 | 1.00 | 36.27 |
| ATOM | 1633 | NZ | LYS | 221 | 4.522 | −49.054 | 27.281 | 1.00 | 36.68 |
| ATOM | 1634 | N | ASN | 222 | −0.422 | −50.206 | 33.200 | 1.00 | 26.61 |
| ATOM | 1635 | CA | ASN | 222 | −1.509 | −50.836 | 33.947 | 1.00 | 26.30 |
| ATOM | 1636 | C | ASN | 222 | −2.491 | −49.827 | 34.522 | 1.00 | 25.29 |
| ATOM | 1637 | O | ASN | 222 | −2.228 | −48.617 | 34.500 | 1.00 | 25.00 |
| ATOM | 1638 | CB | ASN | 222 | −0.977 | −51.765 | 35.054 | 1.00 | 27.39 |
| ATOM | 1639 | CG | ASN | 222 | −0.009 | −51.061 | 35.990 | 1.00 | 29.74 |
| ATOM | 1640 | ND2 | ASN | 222 | 1.088 | −51.737 | 36.321 | 1.00 | 31.51 |
| ATOM | 1641 | OD1 | ASN | 222 | −0.246 | −49.924 | 36.419 | 1.00 | 31.80 |
| ATOM | 1642 | N | LYS | 223 | −3.619 | −50.336 | 35.012 | 1.00 | 23.58 |
| ATOM | 1643 | CA | LYS | 223 | −4.680 | −49.514 | 35.564 | 1.00 | 22.65 |
| ATOM | 1644 | C | LYS | 223 | −4.175 | −48.553 | 36.622 | 1.00 | 22.28 |
| ATOM | 1645 | O | LYS | 223 | −4.550 | −47.381 | 36.610 | 1.00 | 21.73 |
| ATOM | 1646 | CB | LYS | 223 | −5.797 | −50.381 | 36.151 | 1.00 | 22.42 |
| ATOM | 1647 | CG | LYS | 223 | −6.673 | −51.050 | 35.104 | 1.00 | 20.37 |
| ATOM | 1648 | CD | LYS | 223 | −7.525 | −52.073 | 35.801 | 1.00 | 20.73 |
| ATOM | 1649 | CE | LYS | 223 | −8.296 | −52.935 | 34.843 | 1.00 | 18.46 |
| ATOM | 1650 | NZ | LYS | 223 | −9.548 | −53.303 | 35.508 | 1.00 | 19.46 |
| ATOM | 1651 | N | THR | 224 | −3.326 | −49.050 | 37.527 | 1.00 | 21.88 |
| ATOM | 1652 | CA | THR | 224 | −2.817 | −48.233 | 38.632 | 1.00 | 22.00 |
| ATOM | 1653 | C | THR | 224 | −2.010 | −47.031 | 38.178 | 1.00 | 21.24 |
| ATOM | 1654 | O | THR | 224 | −2.292 | −45.929 | 38.641 | 1.00 | 20.74 |
| ATOM | 1655 | CB | THR | 224 | −2.119 | −49.086 | 39.729 | 1.00 | 22.75 |
| ATOM | 1656 | CG2 | THR | 224 | −1.492 | −48.212 | 40.810 | 1.00 | 22.88 |
| ATOM | 1657 | OG1 | THR | 224 | −3.124 | −49.889 | 40.359 | 1.00 | 24.24 |
| ATOM | 1658 | N | SER | 225 | −1.068 | −47.231 | 37.248 | 1.00 | 20.89 |
| ATOM | 1659 | CA | SER | 225 | −0.309 | −46.122 | 36.675 | 0.50 | 20.75 |
| ATOM | 1660 | C | SER | 225 | −1.212 | −45.101 | 35.954 | 1.00 | 21.14 |
| ATOM | 1661 | O | SER | 225 | −1.053 | −43.876 | 36.135 | 1.00 | 20.83 |
| ATOM | 1662 | CB | SER | 225 | 0.792 | −46.641 | 35.744 | 0.50 | 20.97 |
| ATOM | 1663 | OG | SER | 225 | 1.648 | −47.555 | 36.421 | 0.50 | 20.34 |
| ATOM | 1664 | N | ALA | 226 | −2.162 | −45.591 | 35.153 | 1.00 | 20.99 |
| ATOM | 1665 | CA | ALA | 226 | −3.091 | −44.710 | 34.437 | 1.00 | 21.68 |
| ATOM | 1666 | C | ALA | 226 | −3.942 | −43.854 | 35.387 | 1.00 | 22.09 |
| ATOM | 1667 | O | ALA | 226 | −4.157 | −42.660 | 35.154 | 1.00 | 22.17 |
| ATOM | 1668 | CB | ALA | 226 | −3.995 | −45.530 | 33.516 | 1.00 | 21.90 |
| ATOM | 1669 | N | LEU | 227 | −4.417 | −44.482 | 36.456 | 1.00 | 21.92 |
| ATOM | 1670 | CA | LEU | 227 | −5.253 | −43.842 | 37.460 | 1.00 | 22.75 |
| ATOM | 1671 | C | LEU | 227 | −4.488 | −42.705 | 38.154 | 1.00 | 22.50 |
| ATOM | 1672 | O | LEU | 227 | −4.983 | −41.573 | 38.255 | 1.00 | 22.23 |
| ATOM | 1673 | CB | LEU | 227 | −5.629 | −44.918 | 38.468 | 1.00 | 23.73 |
| ATOM | 1674 | CG | LEU | 227 | −7.019 | −45.185 | 39.022 | 1.00 | 27.39 |
| ATOM | 1675 | CD1 | LEU | 227 | −8.101 | −44.903 | 38.018 | 1.00 | 29.27 |
| ATOM | 1676 | CD2 | LEU | 227 | −7.072 | −46.662 | 39.525 | 1.00 | 29.85 |
| ATOM | 1677 | N | LYS | 228 | −3.263 | −43.004 | 38.598 | 1.00 | 21.88 |
| ATOM | 1678 | CA | LYS | 228 | −2.396 | −42.008 | 39.229 | 1.00 | 21.67 |
| ATOM | 1679 | C | LYS | 228 | −1.986 | −40.915 | 38.241 | 1.00 | 20.34 |
| ATOM | 1680 | O | LYS | 228 | −1.791 | −39.773 | 38.626 | 1.00 | 20.34 |
| ATOM | 1681 | CB | LYS | 228 | −1.126 | −42.652 | 39.802 | 1.00 | 22.11 |
| ATOM | 1682 | CG | LYS | 228 | −1.300 | −43.657 | 40.973 | 1.00 | 26.00 |
| ATOM | 1683 | CD | LYS | 228 | 0.108 | −44.119 | 41.478 | 1.00 | 30.77 |
| ATOM | 1684 | CE | LYS | 228 | 0.146 | −45.517 | 42.183 | 1.00 | 34.19 |
| ATOM | 1685 | NZ | LYS | 228 | −0.826 | −45.685 | 43.317 | 1.00 | 33.98 |
| ATOM | 1686 | N | GLY | 229 | −1.808 | −41.285 | 36.978 | 1.00 | 19.34 |
| ATOM | 1687 | CA | GLY | 229 | −1.350 | −40.346 | 35.971 | 1.00 | 18.11 |
| ATOM | 1688 | C | GLY | 229 | −2.424 | −39.342 | 35.605 | 1.00 | 17.41 |
| ATOM | 1689 | O | GLY | 229 | −2.158 | −38.153 | 35.536 | 1.00 | 17.16 |
| ATOM | 1690 | N | ILE | 230 | −3.643 | −39.819 | 35.387 | 1.00 | 16.93 |
| ATOM | 1691 | CA | ILE | 230 | −4.755 | −38.930 | 35.070 | 1.00 | 16.56 |
| ATOM | 1692 | C | ILE | 230 | −5.087 | −38.009 | 36.246 | 1.00 | 17.08 |
| ATOM | 1693 | O | ILE | 230 | −5.442 | −36.845 | 36.035 | 1.00 | 17.22 |
| ATOM | 1694 | CB | ILE | 230 | −6.014 | −39.728 | 34.622 | 1.00 | 16.53 |
| ATOM | 1695 | CG1 | ILE | 230 | −5.721 | −40.453 | 33.307 | 1.00 | 15.01 |
| ATOM | 1696 | CG2 | ILE | 230 | −7.274 | −38.795 | 34.520 | 1.00 | 14.36 |
| ATOM | 1697 | CD1 | ILE | 230 | −6.687 | −41.610 | 33.008 | 1.00 | 16.26 |
| ATOM | 1698 | N | GLU | 231 | −4.956 | −38.516 | 37.478 | 1.00 | 16.98 |
| ATOM | 1699 | CA | GLU | 231 | −5.147 | −37.678 | 38.682 | 1.00 | 16.41 |
| ATOM | 1700 | C | GLU | 231 | −4.109 | −36.558 | 38.669 | 1.00 | 15.77 |
| ATOM | 1701 | O | GLU | 231 | −4.439 | −35.388 | 38.848 | 1.00 | 16.65 |
| ATOM | 1702 | CB | GLU | 231 | −5.021 | −38.518 | 39.977 | 1.00 | 17.10 |
| ATOM | 1703 | CG | GLU | 231 | −4.655 | −37.679 | 41.251 | 1.00 | 16.98 |
| ATOM | 1704 | CD | GLU | 231 | −5.898 | −37.029 | 41.877 | 1.00 | 19.65 |
| ATOM | 1705 | OE1 | GLU | 231 | −7.031 | −37.453 | 41.540 | 1.00 | 21.23 |
| ATOM | 1706 | OE2 | GLU | 231 | −5.757 | −36.104 | 42.710 | 1.00 | 16.59 |
| ATOM | 1707 | N | ALA | 232 | −2.847 | −36.922 | 38.449 | 1.00 | 15.43 |
| ATOM | 1708 | CA | ALA | 232 | −1.750 | −35.944 | 38.439 | 1.00 | 14.49 |
| ATOM | 1709 | C | ALA | 232 | −1.930 | −34.873 | 37.348 | 1.00 | 14.23 |
| ATOM | 1710 | O | ALA | 232 | −1.645 | −33.691 | 37.550 | 1.00 | 13.68 |
| ATOM | 1711 | CB | ALA | 232 | −0.393 | −36.663 | 38.295 | 1.00 | 14.29 |
| ATOM | 1712 | N | VAL | 233 | −2.424 | −35.281 | 36.189 | 1.00 | 13.67 |
| ATOM | 1713 | CA | VAL | 233 | −2.642 | −34.310 | 35.098 | 1.00 | 12.28 |
| ATOM | 1714 | C | VAL | 233 | −3.815 | −33.383 | 35.460 | 1.00 | 12.71 |
| ATOM | 1715 | O | VAL | 233 | −3.729 | −32.176 | 35.230 | 1.00 | 11.74 |
| ATOM | 1716 | CB | VAL | 233 | −2.876 | −35.008 | 33.760 | 1.00 | 12.27 |
| ATOM | 1717 | CG1 | VAL | 233 | −3.190 | −33.971 | 32.641 | 1.00 | 11.62 |
| ATOM | 1718 | CG2 | VAL | 233 | −1.649 | −35.899 | 33.398 | 1.00 | 10.83 |
| ATOM | 1719 | N | GLU | 234 | −4.880 | −33.911 | 36.080 | 1.00 | 12.28 |
| ATOM | 1720 | CA | GLU | 234 | −5.984 | −33.023 | 36.463 | 1.00 | 12.63 |
| ATOM | 1721 | C | GLU | 234 | −5.531 | −31.984 | 37.472 | 1.00 | 12.90 |
| ATOM | 1722 | O | GLU | 234 | −5.959 | −30.809 | 37.384 | 1.00 | 12.90 |
| ATOM | 1723 | CB | GLU | 234 | −7.232 | −33.747 | 36.981 | 1.00 | 12.90 |
| ATOM | 1724 | CG | GLU | 234 | −8.350 | −32.727 | 37.328 | 1.00 | 12.05 |
| ATOM | 1725 | CD | GLU | 234 | −9.717 | −33.333 | 37.583 | 1.00 | 15.98 |
| ATOM | 1726 | OE1 | GLU | 234 | −10.130 | −34.246 | 36.811 | 1.00 | 18.09 |
| ATOM | 1727 | OE2 | GLU | 234 | −10.409 | −32.877 | 38.545 | 1.00 | 14.46 |
| ATOM | 1728 | N | ASP | 235 | −4.676 | −32.391 | 38.425 | 1.00 | 12.56 |
| ATOM | 1729 | CA | ASP | 235 | −4.178 | −31.446 | 39.441 | 1.00 | 13.32 |
| ATOM | 1730 | C | ASP | 235 | −3.427 | −30.302 | 38.792 | 1.00 | 13.62 |
| ATOM | 1731 | O | ASP | 235 | −3.634 | −29.123 | 39.121 | 1.00 | 14.30 |
| ATOM | 1732 | CB | ASP | 235 | −3.217 | −32.115 | 40.439 | 1.00 | 13.20 |
| ATOM | 1733 | CG | ASP | 235 | −3.896 | −33.149 | 41.319 | 1.00 | 12.33 |
| ATOM | 1734 | OD1 | ASP | 235 | −5.118 | −33.071 | 41.525 | 1.00 | 14.11 |
| ATOM | 1735 | OD2 | ASP | 235 | −3.186 | −34.050 | 41.818 | 1.00 | 12.42 |
| ATOM | 1736 | N | TYR | 236 | −2.504 | −30.671 | 37.910 | 1.00 | 13.54 |
| ATOM | 1737 | CA | TYR | 236 | −1.673 | −29.702 | 37.234 | 1.00 | 14.30 |
| ATOM | 1738 | C | TYR | 236 | −2.566 | −28.768 | 36.403 | 1.00 | 15.06 |
| ATOM | 1739 | O | TYR | 236 | −2.423 | −27.540 | 36.454 | 1.00 | 16.06 |
| ATOM | 1740 | CB | TYR | 236 | −0.630 | −30.428 | 36.376 | 1.00 | 13.32 |
| ATOM | 1741 | CG | TYR | 236 | 0.240 | −29.494 | 35.564 | 1.00 | 14.63 |
| ATOM | 1742 | CD1 | TYR | 236 | 1.315 | −28.800 | 36.157 | 1.00 | 14.92 |
| ATOM | 1743 | CD2 | TYR | 236 | 0.017 | −29.323 | 34.200 | 1.00 | 12.55 |
| ATOM | 1744 | CE1 | TYR | 236 | 2.133 | −27.941 | 35.397 | 1.00 | 13.48 |
| ATOM | 1745 | CE2 | TYR | 236 | 0.839 | −28.483 | 33.427 | 1.00 | 13.01 |
| ATOM | 1746 | CZ | TYR | 236 | 1.888 | −27.800 | 34.027 | 1.00 | 14.79 |
| ATOM | 1747 | OH | TYR | 236 | 2.669 | −26.961 | 33.257 | 1.00 | 13.00 |
| ATOM | 1748 | N | ALA | 237 | −3.518 | −29.351 | 35.677 | 1.00 | 15.31 |
| ATOM | 1749 | CA | ALA | 237 | −4.469 | −28.555 | 34.885 | 1.00 | 15.12 |
| ATOM | 1750 | C | ALA | 237 | −5.242 | −27.555 | 35.758 | 1.00 | 15.31 |
| ATOM | 1751 | O | ALA | 237 | −5.307 | −26.341 | 35.456 | 1.00 | 14.97 |
| ATOM | 1752 | CB | ALA | 237 | −5.435 | −29.485 | 34.161 | 1.00 | 14.76 |
| ATOM | 1753 | N | ARG | 238 | −5.807 | −28.054 | 36.853 | 1.00 | 15.10 |
| ATOM | 1754 | CA | ARG | 238 | −6.730 | −27.251 | 37.629 | 1.00 | 14.92 |
| ATOM | 1755 | C | ARG | 238 | −5.991 | −26.101 | 38.297 | 1.00 | 14.84 |
| ATOM | 1756 | O | ARG | 238 | −6.416 | −24.951 | 38.217 | 1.00 | 14.79 |
| ATOM | 1757 | CB | ARG | 238 | −7.477 | −28.112 | 38.656 | 1.00 | 15.32 |
| ATOM | 1758 | CG | ARG | 238 | −8.496 | −27.331 | 39.451 | 1.00 | 15.66 |
| ATOM | 1759 | CD | ARG | 238 | −9.332 | −28.214 | 40.352 | 1.00 | 19.68 |
| ATOM | 1760 | NE | ARG | 238 | −9.997 | −29.344 | 39.688 | 1.00 | 20.83 |
| ATOM | 1761 | CZ | ARG | 238 | −11.050 | −29.228 | 38.868 | 1.00 | 19.94 |
| ATOM | 1762 | NH1 | ARG | 238 | −11.536 | −28.039 | 38.561 | 1.00 | 20.24 |
| ATOM | 1763 | NH2 | ARG | 238 | −11.618 | −30.299 | 38.340 | 1.00 | 16.96 |
| ATOM | 1764 | N | TRP | 239 | −4.845 | −26.399 | 38.892 | 1.00 | 15.02 |
| ATOM | 1765 | CA | TRP | 239 | −4.211 | −25.452 | 39.803 | 1.00 | 15.71 |
| ATOM | 1766 | C | TRP | 239 | −2.893 | −24.809 | 39.402 | 1.00 | 16.18 |
| ATOM | 1767 | O | TRP | 239 | −2.524 | −23.796 | 39.996 | 1.00 | 16.52 |
| ATOM | 1768 | CB | TRP | 239 | −4.034 | −26.117 | 41.164 | 1.00 | 15.46 |
| ATOM | 1769 | CG | TRP | 239 | −5.341 | −26.388 | 41.881 | 1.00 | 16.50 |
| ATOM | 1770 | CD1 | TRP | 239 | −6.279 | −25.459 | 42.280 | 1.00 | 15.99 |
| ATOM | 1771 | CD2 | TRP | 239 | −5.826 | −27.669 | 42.317 | 1.00 | 16.31 |
| ATOM | 1772 | CE2 | TRP | 239 | −7.057 | −27.451 | 42.982 | 1.00 | 15.85 |
| ATOM | 1773 | CE3 | TRP | 239 | −5.330 | −28.986 | 42.216 | 1.00 | 16.49 |
| ATOM | 1774 | NE1 | TRP | 239 | −7.318 | −26.100 | 42.947 | 1.00 | 16.14 |
| ATOM | 1775 | CZ2 | TRP | 239 | −7.813 | −28.509 | 43.531 | 1.00 | 16.23 |
| ATOM | 1776 | CZ3 | TRP | 239 | −6.074 | −30.036 | 42.770 | 1.00 | 15.86 |
| ATOM | 1777 | CH2 | TRP | 239 | −7.308 | −29.790 | 43.414 | 1.00 | 17.78 |
| ATOM | 1778 | N | VAL | 240 | −2.189 | −25.373 | 38.412 | 1.00 | 16.41 |
| ATOM | 1779 | CA | VAL | 240 | −0.790 | −24.996 | 38.136 | 1.00 | 16.17 |
| ATOM | 1780 | C | VAL | 240 | −0.501 | −24.535 | 36.703 | 1.00 | 16.46 |
| ATOM | 1781 | O | VAL | 240 | 0.220 | −23.549 | 36.498 | 1.00 | 15.24 |
| ATOM | 1782 | CB | VAL | 240 | 0.178 | −26.176 | 38.480 | 1.00 | 16.62 |
| ATOM | 1783 | CG1 | VAL | 240 | −0.130 | −26.740 | 39.886 | 1.00 | 15.73 |
| ATOM | 1784 | CG2 | VAL | 240 | 1.682 | −25.762 | 38.363 | 1.00 | 16.47 |
| ATOM | 1785 | N | ALA | 241 | −1.016 | −25.277 | 35.719 | 1.00 | 16.46 |
| ATOM | 1786 | CA | ALA | 241 | −0.670 | −25.048 | 34.309 | 1.00 | 16.74 |
| ATOM | 1787 | C | ALA | 241 | −0.735 | −23.560 | 33.923 | 1.00 | 17.03 |
| ATOM | 1788 | O | ALA | 241 | −1.813 | −22.924 | 33.995 | 1.00 | 17.32 |
| ATOM | 1789 | CB | ALA | 241 | −1.572 | −25.897 | 33.381 | 1.00 | 16.42 |
| ATOM | 1790 | N | PRO | 242 | 0.428 | −22.974 | 33.559 | 1.00 | 16.79 |
| ATOM | 1791 | CA | PRO | 242 | 0.406 | −21.576 | 33.140 | 1.00 | 16.92 |
| ATOM | 1792 | C | PRO | 242 | −0.414 | −21.425 | 31.870 | 1.00 | 16.41 |
| ATOM | 1793 | O | PRO | 242 | −0.665 | −22.402 | 31.176 | 1.00 | 15.64 |
| ATOM | 1794 | CB | PRO | 242 | 1.884 | −21.251 | 32.883 | 1.00 | 17.37 |
| ATOM | 1795 | CG | PRO | 242 | 2.633 | −22.230 | 33.740 | 1.00 | 18.10 |
| ATOM | 1796 | CD | PRO | 242 | 1.799 | −23.487 | 33.717 | 1.00 | 17.03 |
| ATOM | 1797 | N | ARG | 243 | −0.840 | −20.201 | 31.610 | 1.00 | 16.30 |
| ATOM | 1798 | CA | ARG | 243 | −1.557 | −19.835 | 30.402 | 1.00 | 16.60 |
| ATOM | 1799 | C | ARG | 243 | −0.868 | −20.341 | 29.137 | 1.00 | 15.95 |
| ATOM | 1800 | O | ARG | 243 | −1.531 | −20.861 | 28.244 | 1.00 | 15.43 |
| ATOM | 1801 | CB | ARG | 243 | −1.645 | −18.308 | 30.332 | 1.00 | 17.47 |
| ATOM | 1802 | CG | ARG | 243 | −2.635 | −17.808 | 29.326 | 1.00 | 19.41 |
| ATOM | 1803 | CD | ARG | 243 | −2.539 | −16.291 | 29.193 | 1.00 | 22.82 |
| ATOM | 1804 | NE | ARG | 243 | −3.341 | −15.829 | 28.070 | 1.00 | 24.39 |
| ATOM | 1805 | CZ | ARG | 243 | −3.187 | −14.660 | 27.457 | 1.00 | 29.99 |
| ATOM | 1806 | NH1 | ARG | 243 | −2.233 | −13.816 | 27.843 | 1.00 | 31.00 |
| ATOM | 1807 | NH2 | ARG | 243 | −3.985 | −14.338 | 26.442 | 1.00 | 30.80 |
| ATOM | 1808 | N | GLU | 244 | 0.455 | −20.193 | 29.081 | 1.00 | 14.74 |
| ATOM | 1809 | CA | GLU | 244 | 1.233 | −20.516 | 27.875 | 1.00 | 14.97 |
| ATOM | 1810 | C | GLU | 244 | 1.237 | −22.012 | 27.484 | 1.00 | 14.42 |
| ATOM | 1811 | O | GLU | 244 | 1.695 | −22.363 | 26.379 | 1.00 | 14.14 |
| ATOM | 1812 | CB | GLU | 244 | 2.694 | −20.068 | 28.048 | 1.00 | 14.69 |
| ATOM | 1813 | CG | GLU | 244 | 2.939 | −18.551 | 28.075 | 1.00 | 16.80 |
| ATOM | 1814 | CD | GLU | 244 | 2.465 | −17.897 | 29.397 | 1.00 | 19.20 |
| ATOM | 1815 | OE1 | GLU | 244 | 2.396 | −18.593 | 30.439 | 1.00 | 17.19 |
| ATOM | 1816 | OE2 | GLU | 244 | 2.166 | −16.682 | 29.371 | 1.00 | 20.57 |
| ATOM | 1817 | N | VAL | 245 | 0.798 | −22.889 | 28.386 | 1.00 | 13.52 |
| ATOM | 1818 | CA | VAL | 245 | 0.848 | −24.326 | 28.096 | 0.50 | 13.83 |
| ATOM | 1819 | C | VAL | 245 | −0.488 | −24.825 | 27.564 | 1.00 | 13.62 |
| ATOM | 1820 | O | VAL | 245 | −1.537 | −24.598 | 28.160 | 1.00 | 12.63 |
| ATOM | 1821 | CB | VAL | 245 | 1.374 | −25.186 | 29.272 | 0.50 | 13.41 |
| ATOM | 1822 | CG1 | VAL | 245 | 0.252 | −25.595 | 30.238 | 0.50 | 14.96 |
| ATOM | 1823 | CG2 | VAL | 245 | 2.078 | −26.419 | 28.737 | 0.50 | 14.03 |
| ATOM | 1824 | N | ASN | 246 | −0.434 | −25.488 | 26.406 | 1.00 | 13.41 |
| ATOM | 1825 | CA | ASN | 246 | −1.647 | −26.053 | 25.791 | 1.00 | 13.49 |
| ATOM | 1826 | C | ASN | 246 | −1.408 | −27.530 | 25.591 | 1.00 | 13.64 |
| ATOM | 1827 | O | ASN | 246 | −0.415 | −27.907 | 24.950 | 1.00 | 14.03 |
| ATOM | 1828 | CB | ASN | 246 | −1.944 | −25.421 | 24.433 | 1.00 | 13.24 |
| ATOM | 1829 | CG | ASN | 246 | −1.971 | −23.920 | 24.477 | 1.00 | 14.11 |
| ATOM | 1830 | ND2 | ASN | 246 | −0.792 | −23.306 | 24.513 | 1.00 | 11.36 |
| ATOM | 1831 | OD1 | ASN | 246 | −3.044 | −23.312 | 24.455 | 1.00 | 14.81 |
| ATOM | 1832 | N | PHE | 247 | −2.269 | −28.385 | 26.134 | 1.00 | 13.04 |
| ATOM | 1833 | CA | PHE | 247 | −2.003 | −29.812 | 25.952 | 1.00 | 13.29 |
| ATOM | 1834 | C | PHE | 247 | −3.200 | −30.729 | 26.139 | 1.00 | 12.81 |
| ATOM | 1835 | O | PHE | 247 | −4.292 | −30.314 | 26.614 | 1.00 | 12.70 |
| ATOM | 1836 | CB | PHE | 247 | −0.786 | −30.284 | 26.813 | 1.00 | 13.47 |
| ATOM | 1837 | CG | PHE | 247 | −1.088 | −30.453 | 28.292 | 1.00 | 13.91 |
| ATOM | 1838 | CD1 | PHE | 247 | −1.186 | −31.734 | 28.854 | 1.00 | 16.11 |
| ATOM | 1839 | CD2 | PHE | 247 | −1.255 | −29.339 | 29.123 | 1.00 | 14.29 |
| ATOM | 1840 | CE1 | PHE | 247 | −1.457 | −31.906 | 30.238 | 1.00 | 15.82 |
| ATOM | 1841 | CE2 | PHE | 247 | −1.513 | −29.492 | 30.508 | 1.00 | 16.63 |
| ATOM | 1842 | CZ | PHE | 247 | −1.618 | −30.777 | 31.058 | 1.00 | 15.20 |
| ATOM | 1843 | N | ARG | 248 | −2.945 | −31.986 | 25.780 | 1.00 | 12.57 |
| ATOM | 1844 | CA | ARG | 248 | −3.895 | −33.075 | 25.994 | 0.50 | 13.20 |
| ATOM | 1845 | C | ARG | 248 | −3.187 | −34.405 | 26.227 | 1.00 | 12.76 |
| ATOM | 1846 | O | ARG | 248 | −2.015 | −34.551 | 25.923 | 1.00 | 13.54 |
| ATOM | 1847 | CB | ARG | 248 | −4.872 | −33.200 | 24.807 | 0.50 | 12.98 |
| ATOM | 1848 | CG | ARG | 248 | −4.239 | −33.312 | 23.408 | 0.50 | 15.22 |
| ATOM | 1849 | CD | ARG | 248 | −5.289 | −33.816 | 22.396 | 0.50 | 17.29 |
| ATOM | 1850 | NE | ARG | 248 | −4.895 | −33.609 | 21.004 | 0.50 | 18.71 |
| ATOM | 1851 | CZ | ARG | 248 | −5.521 | −32.789 | 20.156 | 0.50 | 19.80 |
| ATOM | 1852 | NH1 | ARG | 248 | −5.083 | −32.684 | 18.909 | 0.50 | 20.31 |
| ATOM | 1853 | NH2 | ARG | 248 | −6.583 | −32.088 | 20.541 | 0.50 | 18.67 |
| ATOM | 1854 | N | ILE | 249 | −3.913 | −35.362 | 26.798 | 1.00 | 13.13 |
| ATOM | 1855 | CA | ILE | 249 | −3.498 | −36.764 | 26.804 | 1.00 | 12.36 |
| ATOM | 1856 | C | ILE | 249 | −4.242 | −37.394 | 25.632 | 1.00 | 13.57 |
| ATOM | 1857 | O | ILE | 249 | −5.456 | −37.181 | 25.470 | 1.00 | 13.42 |
| ATOM | 1858 | CB | ILE | 249 | −3.960 | −37.529 | 28.071 | 1.00 | 12.28 |
| ATOM | 1859 | CG1 | ILE | 249 | −3.391 | −36.898 | 29.338 | 1.00 | 12.69 |
| ATOM | 1860 | CG2 | ILE | 249 | −3.558 | −39.035 | 28.004 | 1.00 | 9.93 |
| ATOM | 1861 | CD1 | ILE | 249 | −4.184 | −37.263 | 30.610 | 1.00 | 10.47 |
| ATOM | 1862 | N | GLY | 250 | −3.534 | −38.188 | 24.829 | 1.00 | 13.96 |
| ATOM | 1863 | CA | GLY | 250 | −4.135 | −38.882 | 23.710 | 1.00 | 14.67 |
| ATOM | 1864 | C | GLY | 250 | −3.555 | −40.282 | 23.641 | 1.00 | 15.16 |
| ATOM | 1865 | O | GLY | 250 | −2.413 | −40.491 | 24.036 | 1.00 | 15.41 |
| ATOM | 1866 | N | ASP | 251 | −4.343 | −41.236 | 23.159 | 1.00 | 14.69 |
| ATOM | 1867 | CA | ASP | 251 | −3.847 | −42.580 | 22.884 | 1.00 | 14.72 |
| ATOM | 1868 | C | ASP | 251 | −4.370 | −43.030 | 21.532 | 1.00 | 15.02 |
| ATOM | 1869 | O | ASP | 251 | −5.579 | −43.186 | 21.365 | 1.00 | 15.57 |
| ATOM | 1870 | CB | ASP | 251 | −4.280 | −43.556 | 23.966 | 1.00 | 14.02 |
| ATOM | 1871 | CG | ASP | 251 | −3.599 | −44.935 | 23.836 | 1.00 | 14.41 |
| ATOM | 1872 | OD1 | ASP | 251 | −2.781 | −45.160 | 22.897 | 1.00 | 13.34 |
| ATOM | 1873 | OD2 | ASP | 251 | −3.900 | −45.794 | 24.684 | 1.00 | 13.83 |
| ATOM | 1874 | N | TYR | 252 | −3.442 | −43.202 | 20.588 | 1.00 | 15.14 |
| ATOM | 1875 | CA | TYR | 252 | −3.687 | −43.663 | 19.214 | 1.00 | 15.98 |
| ATOM | 1876 | C | TYR | 252 | −3.113 | −45.081 | 18.993 | 1.00 | 16.11 |
| ATOM | 1877 | O | TYR | 252 | −3.137 | −45.590 | 17.876 | 1.00 | 16.15 |
| ATOM | 1878 | CB | TYR | 252 | −3.040 | −42.681 | 18.217 | 1.00 | 15.06 |
| ATOM | 1879 | CG | TYR | 252 | −3.313 | −41.258 | 18.629 | 1.00 | 17.48 |
| ATOM | 1880 | CD1 | TYR | 252 | −4.583 | −40.696 | 18.429 | 1.00 | 15.92 |
| ATOM | 1881 | CD2 | TYR | 252 | −2.342 | −40.493 | 19.278 | 1.00 | 15.89 |
| ATOM | 1882 | CE1 | TYR | 252 | −4.878 | −39.419 | 18.854 | 1.00 | 15.71 |
| ATOM | 1883 | CE2 | TYR | 252 | −2.639 | −39.196 | 19.710 | 1.00 | 16.33 |
| ATOM | 1884 | CZ | TYR | 252 | −3.910 | −38.675 | 19.477 | 1.00 | 15.85 |
| ATOM | 1885 | OH | TYR | 252 | −4.253 | −37.414 | 19.873 | 1.00 | 17.55 |
| ATOM | 1886 | N | GLY | 253 | −2.608 | −45.696 | 20.058 | 1.00 | 16.57 |
| ATOM | 1887 | CA | GLY | 253 | −1.933 | −46.998 | 19.997 | 1.00 | 17.95 |
| ATOM | 1888 | C | GLY | 253 | −2.519 | −48.059 | 20.919 | 1.00 | 18.52 |
| ATOM | 1889 | O | GLY | 253 | −1.799 | −48.946 | 21.364 | 1.00 | 19.85 |
| ATOM | 1890 | N | ALA | 254 | −3.815 | −47.973 | 21.222 | 1.00 | 18.35 |
| ATOM | 1891 | CA | ALA | 254 | −4.491 | −48.986 | 22.045 | 1.00 | 18.90 |
| ATOM | 1892 | C | ALA | 254 | −3.632 | −49.406 | 23.245 | 1.00 | 19.38 |
| ATOM | 1893 | O | ALA | 254 | −3.429 | −50.608 | 23.483 | 1.00 | 19.25 |
| ATOM | 1894 | CB | ALA | 254 | −4.870 | −50.230 | 21.182 | 1.00 | 19.01 |
| ATOM | 1895 | N | GLY | 255 | −3.108 | −48.414 | 23.970 | 1.00 | 18.91 |
| ATOM | 1896 | CA | GLY | 255 | −2.328 | −48.660 | 25.185 | 1.00 | 18.73 |
| ATOM | 1897 | C | GLY | 255 | −0.997 | −47.930 | 25.203 | 1.00 | 18.71 |
| ATOM | 1898 | O | GLY | 255 | −0.114 | −48.257 | 25.999 | 1.00 | 18.97 |
| ATOM | 1899 | N | ASN | 256 | −0.832 | −46.949 | 24.323 | 1.00 | 18.09 |
| ATOM | 1900 | CA | ASN | 256 | 0.382 | −46.132 | 24.320 | 1.00 | 18.01 |
| ATOM | 1901 | C | ASN | 256 | 0.068 | −44.636 | 24.533 | 1.00 | 17.07 |
| ATOM | 1902 | O | ASN | 256 | 0.347 | −43.805 | 23.661 | 1.00 | 17.40 |
| ATOM | 1903 | CB | ASN | 256 | 1.186 | −46.385 | 23.029 | 1.00 | 18.99 |
| ATOM | 1904 | CG | ASN | 256 | 2.620 | −45.789 | 23.065 | 1.00 | 22.36 |
| ATOM | 1905 | ND2 | ASN | 256 | 3.056 | −45.278 | 21.916 | 1.00 | 25.22 |
| ATOM | 1906 | OD1 | ASN | 256 | 3.327 | −45.802 | 24.099 | 1.00 | 26.91 |
| ATOM | 1907 | N | PRO | 257 | −0.502 | −44.279 | 25.703 | 1.00 | 15.72 |
| ATOM | 1908 | CA | PRO | 257 | −0.865 | −42.870 | 25.883 | 1.00 | 15.11 |
| ATOM | 1909 | C | PRO | 257 | 0.337 | −41.900 | 25.876 | 1.00 | 15.29 |
| ATOM | 1910 | O | PRO | 257 | 1.477 | −42.260 | 26.260 | 1.00 | 13.94 |
| ATOM | 1911 | CB | PRO | 257 | −1.557 | −42.850 | 27.257 | 1.00 | 15.12 |
| ATOM | 1912 | CG | PRO | 257 | −1.002 | −44.021 | 27.992 | 1.00 | 14.79 |
| ATOM | 1913 | CD | PRO | 257 | −0.776 | −45.077 | 26.919 | 1.00 | 15.48 |
| ATOM | 1914 | N | GLY | 258 | 0.071 | −40.675 | 25.444 | 1.00 | 13.98 |
| ATOM | 1915 | CA | GLY | 258 | 1.086 | −39.657 | 25.462 | 1.00 | 14.14 |
| ATOM | 1916 | C | GLY | 258 | 0.475 | −38.312 | 25.770 | 1.00 | 14.38 |
| ATOM | 1917 | O | GLY | 258 | −0.719 | −38.083 | 25.535 | 1.00 | 14.66 |
| ATOM | 1918 | N | ILE | 259 | 1.291 | −37.425 | 26.324 | 1.00 | 14.71 |
| ATOM | 1919 | CA | ILE | 259 | 0.939 | −36.018 | 26.347 | 1.00 | 15.28 |
| ATOM | 1920 | C | ILE | 259 | 1.436 | −35.396 | 25.043 | 1.00 | 15.46 |
| ATOM | 1921 | O | ILE | 259 | 2.579 | −35.652 | 24.638 | 1.00 | 16.65 |
| ATOM | 1922 | CB | ILE | 259 | 1.545 | −35.305 | 27.587 | 1.00 | 15.23 |
| ATOM | 1923 | CG1 | ILE | 259 | 0.782 | −35.742 | 28.857 | 1.00 | 15.35 |
| ATOM | 1924 | CG2 | ILE | 259 | 1.546 | −33.805 | 27.403 | 1.00 | 14.61 |
| ATOM | 1925 | CD1 | ILE | 259 | 1.332 | −35.150 | 30.185 | 1.00 | 13.23 |
| ATOM | 1926 | N | GLU | 260 | 0.575 | −34.635 | 24.363 | 1.00 | 15.08 |
| ATOM | 1927 | CA | GLU | 260 | 1.041 | −33.769 | 23.272 | 1.00 | 16.15 |
| ATOM | 1928 | C | GLU | 260 | 0.624 | −32.348 | 23.570 | 1.00 | 14.81 |
| ATOM | 1929 | O | GLU | 260 | −0.469 | −32.109 | 24.081 | 1.00 | 14.66 |
| ATOM | 1930 | CB | GLU | 260 | 0.441 | −34.138 | 21.909 | 1.00 | 16.65 |
| ATOM | 1931 | CG | GLU | 260 | −0.016 | −35.548 | 21.743 | 1.00 | 21.07 |
| ATOM | 1932 | CD | GLU | 260 | −1.039 | −35.660 | 20.608 | 1.00 | 25.33 |
| ATOM | 1933 | OE1 | GLU | 260 | −0.680 | −35.211 | 19.488 | 1.00 | 27.38 |
| ATOM | 1934 | OE2 | GLU | 260 | −2.179 | −36.152 | 20.863 | 1.00 | 22.26 |
| ATOM | 1935 | N | GLY | 261 | 1.476 | −31.404 | 23.213 | 1.00 | 14.91 |
| ATOM | 1936 | CA | GLY | 261 | 1.242 | −30.027 | 23.579 | 1.00 | 14.02 |
| ATOM | 1937 | C | GLY | 261 | 1.948 | −29.004 | 22.734 | 1.00 | 14.58 |
| ATOM | 1938 | O | GLY | 261 | 2.831 | −29.342 | 21.908 | 1.00 | 14.65 |
| ATOM | 1939 | N | LEU | 262 | 1.525 | −27.759 | 22.948 | 1.00 | 14.19 |
| ATOM | 1940 | CA | LEU | 262 | 2.096 | −26.558 | 22.355 | 1.00 | 15.14 |
| ATOM | 1941 | C | LEU | 262 | 2.311 | −25.538 | 23.470 | 1.00 | 15.20 |
| ATOM | 1942 | O | LEU | 262 | 1.389 | −25.245 | 24.252 | 1.00 | 15.36 |
| ATOM | 1943 | CB | LEU | 262 | 1.191 | −25.968 | 21.253 | 1.00 | 14.56 |
| ATOM | 1944 | CG | LEU | 262 | 1.863 | −24.893 | 20.370 | 1.00 | 15.00 |
| ATOM | 1945 | CD1 | LEU | 262 | 3.087 | −25.453 | 19.624 | 1.00 | 14.90 |
| ATOM | 1946 | CD2 | LEU | 262 | 0.888 | −24.183 | 19.354 | 1.00 | 13.53 |
| ATOM | 1947 | N | TYR | 263 | 3.542 | −25.039 | 23.564 | 1.00 | 15.25 |
| ATOM | 1948 | CA | TYR | 263 | 3.919 | −24.065 | 24.584 | 1.00 | 15.16 |
| ATOM | 1949 | C | TYR | 263 | 4.302 | −22.728 | 23.956 | 1.00 | 15.72 |
| ATOM | 1950 | O | TYR | 263 | 5.120 | −22.677 | 23.003 | 1.00 | 14.62 |
| ATOM | 1951 | CB | TYR | 263 | 5.077 | −24.587 | 25.447 | 1.00 | 15.58 |
| ATOM | 1952 | CG | TYR | 263 | 5.483 | −23.585 | 26.531 | 1.00 | 15.88 |
| ATOM | 1953 | CD1 | TYR | 263 | 4.756 | −23.497 | 27.723 | 1.00 | 16.32 |
| ATOM | 1954 | CD2 | TYR | 263 | 6.569 | −22.711 | 26.348 | 1.00 | 16.40 |
| ATOM | 1955 | CE1 | TYR | 263 | 5.084 | −22.586 | 28.714 | 1.00 | 16.86 |
| ATOM | 1956 | CE2 | TYR | 263 | 6.921 | −21.768 | 27.353 | 1.00 | 16.45 |
| ATOM | 1957 | CZ | TYR | 263 | 6.164 | −21.716 | 28.523 | 1.00 | 17.81 |
| ATOM | 1958 | OH | TYR | 263 | 6.471 | −20.813 | 29.515 | 1.00 | 17.14 |
| ATOM | 1959 | N | TYR | 264 | 3.702 | −21.653 | 24.474 | 1.00 | 15.55 |
| ATOM | 1960 | CA | TYR | 264 | 4.011 | −20.289 | 24.036 | 1.00 | 16.18 |
| ATOM | 1961 | C | TYR | 264 | 5.281 | −19.781 | 24.724 | 1.00 | 17.43 |
| ATOM | 1962 | O | TYR | 264 | 5.224 | −19.170 | 25.807 | 1.00 | 16.74 |
| ATOM | 1963 | CB | TYR | 264 | 2.825 | −19.347 | 24.303 | 1.00 | 15.85 |
| ATOM | 1964 | CG | TYR | 264 | 1.822 | −19.345 | 23.171 | 1.00 | 16.22 |
| ATOM | 1965 | CD1 | TYR | 264 | 1.686 | −18.225 | 22.343 | 1.00 | 15.88 |
| ATOM | 1966 | CD2 | TYR | 264 | 1.038 | −20.480 | 22.898 | 1.00 | 16.63 |
| ATOM | 1967 | CE1 | TYR | 264 | 0.777 | −18.213 | 21.284 | 1.00 | 16.49 |
| ATOM | 1968 | CE2 | TYR | 264 | 0.121 | −20.491 | 21.830 | 1.00 | 16.72 |
| ATOM | 1969 | CZ | TYR | 264 | 0.002 | −19.351 | 21.027 | 1.00 | 16.56 |
| ATOM | 1970 | OH | TYR | 264 | −0.888 | −19.339 | 19.992 | 1.00 | 13.91 |
| ATOM | 1971 | N | GLY | 265 | 6.419 | −20.051 | 24.084 | 1.00 | 17.88 |
| ATOM | 1972 | CA | GLY | 265 | 7.737 | −19.690 | 24.603 | 1.00 | 18.81 |
| ATOM | 1973 | C | GLY | 265 | 8.784 | −20.725 | 24.193 | 1.00 | 19.16 |
| ATOM | 1974 | O | GLY | 265 | 8.562 | −21.555 | 23.296 | 1.00 | 18.85 |
| ATOM | 1975 | N | THR | 266 | 9.938 | −20.668 | 24.844 | 1.00 | 19.99 |
| ATOM | 1976 | CA | THR | 266 | 11.098 | −21.463 | 24.413 | 1.00 | 20.55 |
| ATOM | 1977 | C | THR | 266 | 11.074 | −22.859 | 25.039 | 1.00 | 20.56 |
| ATOM | 1978 | O | THR | 266 | 10.354 | −23.085 | 26.030 | 1.00 | 20.40 |
| ATOM | 1979 | CB | THR | 266 | 12.405 | −20.769 | 24.843 | 1.00 | 21.07 |
| ATOM | 1980 | CG2 | THR | 266 | 12.416 | −19.288 | 24.376 | 1.00 | 20.62 |
| ATOM | 1981 | OG1 | THR | 266 | 12.507 | −20.846 | 26.275 | 1.00 | 20.80 |
| ATOM | 1982 | N | PRO | 267 | 11.872 | −23.799 | 24.487 | 1.00 | 20.75 |
| ATOM | 1983 | CA | PRO | 267 | 11.957 | −25.142 | 25.079 | 1.00 | 20.82 |
| ATOM | 1984 | C | PRO | 267 | 12.366 | −25.163 | 26.562 | 1.00 | 21.54 |
| ATOM | 1985 | O | PRO | 267 | 11.874 | −26.013 | 27.331 | 1.00 | 21.28 |
| ATOM | 1986 | CB | PRO | 267 | 13.015 | −25.837 | 24.218 | 1.00 | 20.83 |
| ATOM | 1987 | CG | PRO | 267 | 12.905 | −25.160 | 22.885 | 1.00 | 20.35 |
| ATOM | 1988 | CD | PRO | 267 | 12.640 | −23.708 | 23.222 | 1.00 | 20.82 |
| ATOM | 1989 | N | GLU | 268 | 13.252 | −24.247 | 26.957 | 1.00 | 21.62 |
| ATOM | 1990 | CA | GLU | 268 | 13.730 | −24.178 | 28.353 | 1.00 | 22.56 |
| ATOM | 1991 | C | GLU | 268 | 12.616 | −23.665 | 29.266 | 1.00 | 21.72 |
| ATOM | 1992 | O | GLU | 268 | 12.432 | −24.170 | 30.372 | 1.00 | 21.12 |
| ATOM | 1993 | CB | GLU | 268 | 14.977 | −23.275 | 28.470 | 1.00 | 23.15 |
| ATOM | 1994 | CG | GLU | 268 | 16.238 | −23.776 | 27.697 | 1.00 | 26.41 |
| ATOM | 1995 | CD | GLU | 268 | 16.115 | −23.720 | 26.143 | 1.00 | 30.37 |
| ATOM | 1996 | OE1 | GLU | 268 | 15.364 | −22.877 | 25.572 | 1.00 | 30.01 |
| ATOM | 1997 | OE2 | GLU | 268 | 16.795 | −24.539 | 25.483 | 1.00 | 32.30 |
| ATOM | 1998 | N | GLN | 269 | 11.880 | −22.652 | 28.794 | 1.00 | 21.69 |
| ATOM | 1999 | CA | GLN | 269 | 10.702 | −22.144 | 29.515 | 1.00 | 21.55 |
| ATOM | 2000 | C | GLN | 269 | 9.613 | −23.217 | 29.623 | 1.00 | 21.27 |
| ATOM | 2001 | O | GLN | 269 | 8.964 | −23.360 | 30.669 | 1.00 | 21.92 |
| ATOM | 2002 | CB | GLN | 269 | 10.135 | −20.909 | 28.822 | 1.00 | 21.62 |
| ATOM | 2003 | CG | GLN | 269 | 10.910 | −19.641 | 29.093 | 1.00 | 23.28 |
| ATOM | 2004 | CD | GLN | 269 | 10.584 | −18.524 | 28.130 | 1.00 | 25.96 |
| ATOM | 2005 | NE2 | GLN | 269 | 10.820 | −17.301 | 28.570 | 1.00 | 27.15 |
| ATOM | 2006 | OE1 | GLN | 269 | 10.141 | −18.748 | 27.000 | 1.00 | 26.37 |
| ATOM | 2007 | N | TRP | 270 | 9.403 | −23.974 | 28.546 | 1.00 | 20.75 |
| ATOM | 2008 | CA | TRP | 270 | 8.436 | −25.075 | 28.608 | 1.00 | 20.09 |
| ATOM | 2009 | C | TRP | 270 | 8.795 | −26.049 | 29.745 | 1.00 | 19.89 |
| ATOM | 2010 | O | TRP | 270 | 7.964 | −26.335 | 30.634 | 1.00 | 20.49 |
| ATOM | 2011 | CB | TRP | 270 | 8.272 | −25.843 | 27.265 | 1.00 | 18.91 |
| ATOM | 2012 | CG | TRP | 270 | 7.501 | −27.113 | 27.548 | 1.00 | 17.87 |
| ATOM | 2013 | CD1 | TRP | 270 | 6.170 | −27.209 | 27.872 | 1.00 | 19.13 |
| ATOM | 2014 | CD2 | TRP | 270 | 8.040 | −28.427 | 27.692 | 1.00 | 18.30 |
| ATOM | 2015 | CE2 | TRP | 270 | 6.966 | −29.283 | 28.055 | 1.00 | 17.19 |
| ATOM | 2016 | CE3 | TRP | 270 | 9.324 | −28.979 | 27.519 | 1.00 | 17.07 |
| ATOM | 2017 | NE1 | TRP | 270 | 5.835 | −28.513 | 28.156 | 1.00 | 17.44 |
| ATOM | 2018 | CZ2 | TRP | 270 | 7.133 | −30.647 | 28.242 | 1.00 | 15.64 |
| ATOM | 2019 | CZ3 | TRP | 270 | 9.492 | −30.336 | 27.698 | 1.00 | 16.69 |
| ATOM | 2020 | CH2 | TRP | 270 | 8.400 | −31.165 | 28.057 | 1.00 | 17.60 |
| ATOM | 2021 | N | ARG | 271 | 10.029 | −26.556 | 29.711 | 1.00 | 19.71 |
| ATOM | 2022 | CA | ARG | 271 | 10.467 | −27.565 | 30.674 | 1.00 | 19.93 |
| ATOM | 2023 | C | ARG | 271 | 10.261 | −27.099 | 32.119 | 1.00 | 19.54 |
| ATOM | 2024 | O | ARG | 271 | 9.705 | −27.829 | 32.945 | 1.00 | 19.54 |
| ATOM | 2025 | CB | ARG | 271 | 11.914 | −28.016 | 30.410 | 1.00 | 19.88 |
| ATOM | 2026 | CG | ARG | 271 | 12.397 | −29.106 | 31.347 | 1.00 | 21.33 |
| ATOM | 2027 | CD | ARG | 271 | 11.524 | −30.343 | 31.196 | 1.00 | 23.50 |
| ATOM | 2028 | NE | ARG | 271 | 11.946 | −31.511 | 31.973 | 1.00 | 24.52 |
| ATOM | 2029 | CZ | ARG | 271 | 12.572 | −32.569 | 31.461 | 1.00 | 26.45 |
| ATOM | 2030 | NH1 | ARG | 271 | 12.905 | −32.600 | 30.169 | 1.00 | 25.38 |
| ATOM | 2031 | NH2 | ARG | 271 | 12.880 | −33.604 | 32.245 | 1.00 | 26.27 |
| ATOM | 2032 | N | ALA | 272 | 10.678 | −25.866 | 32.396 | 1.00 | 19.72 |
| ATOM | 2033 | CA | ALA | 272 | 10.488 | −25.254 | 33.694 | 1.00 | 18.93 |
| ATOM | 2034 | C | ALA | 272 | 8.999 | −25.238 | 34.054 | 1.00 | 18.97 |
| ATOM | 2035 | O | ALA | 272 | 8.622 | −25.641 | 35.145 | 1.00 | 17.90 |
| ATOM | 2036 | CB | ALA | 272 | 11.053 | −23.849 | 33.694 | 1.00 | 20.17 |
| ATOM | 2037 | N | ALA | 273 | 8.153 | −24.822 | 33.105 | 1.00 | 18.40 |
| ATOM | 2038 | CA | ALA | 273 | 6.708 | −24.702 | 33.357 | 1.00 | 17.40 |
| ATOM | 2039 | C | ALA | 273 | 6.022 | −26.057 | 33.582 | 1.00 | 17.02 |
| ATOM | 2040 | O | ALA | 273 | 5.064 | −26.145 | 34.359 | 1.00 | 16.50 |
| ATOM | 2041 | CB | ALA | 273 | 6.017 | −23.930 | 32.220 | 1.00 | 17.29 |
| ATOM | 2042 | N | PHE | 274 | 6.526 | −27.092 | 32.904 | 1.00 | 16.38 |
| ATOM | 2043 | CA | PHE | 274 | 5.925 | −28.430 | 32.907 | 1.00 | 16.17 |
| ATOM | 2044 | C | PHE | 274 | 6.490 | −29.327 | 33.989 | 1.00 | 16.50 |
| ATOM | 2045 | O | PHE | 274 | 5.884 | −30.348 | 34.310 | 1.00 | 15.77 |
| ATOM | 2046 | CB | PHE | 274 | 6.097 | −29.124 | 31.543 | 1.00 | 15.48 |
| ATOM | 2047 | CG | PHE | 274 | 4.905 | −29.923 | 31.123 | 1.00 | 15.80 |
| ATOM | 2048 | CD1 | PHE | 274 | 3.703 | −29.285 | 30.786 | 1.00 | 15.84 |
| ATOM | 2049 | CD2 | PHE | 274 | 4.953 | −31.312 | 31.094 | 1.00 | 15.74 |
| ATOM | 2050 | CE1 | PHE | 274 | 2.589 | −30.023 | 30.418 | 1.00 | 14.84 |
| ATOM | 2051 | CE2 | PHE | 274 | 3.845 | −32.073 | 30.716 | 1.00 | 15.60 |
| ATOM | 2052 | CZ | PHE | 274 | 2.657 | −31.430 | 30.374 | 1.00 | 17.04 |
| ATOM | 2053 | N | GLN | 275 | 7.634 | −28.943 | 34.560 | 1.00 | 16.41 |
| ATOM | 2054 | CA | GLN | 275 | 8.334 | −29.781 | 35.569 | 1.00 | 17.07 |
| ATOM | 2055 | C | GLN | 275 | 7.458 | −30.240 | 36.757 | 1.00 | 17.17 |
| ATOM | 2056 | O | GLN | 275 | 7.588 | −31.389 | 37.196 | 1.00 | 17.82 |
| ATOM | 2057 | CB | GLN | 275 | 9.616 | −29.077 | 36.095 | 1.00 | 17.02 |
| ATOM | 2058 | CG | GLN | 275 | 10.625 | −30.022 | 36.788 | 1.00 | 18.26 |
| ATOM | 2059 | CD | GLN | 275 | 11.071 | −31.157 | 35.871 | 1.00 | 19.38 |
| ATOM | 2060 | NE2 | GLN | 275 | 10.994 | −32.384 | 36.358 | 1.00 | 17.69 |
| ATOM | 2061 | OE1 | GLN | 275 | 11.468 | −30.922 | 34.725 | 1.00 | 24.03 |
| ATOM | 2062 | N | PRO | 276 | 6.573 | −29.357 | 37.271 | 1.00 | 17.00 |
| ATOM | 2063 | CA | PRO | 276 | 5.702 | −29.753 | 38.362 | 1.00 | 17.12 |
| ATOM | 2064 | C | PRO | 276 | 4.846 | −30.967 | 38.051 | 1.00 | 16.98 |
| ATOM | 2065 | O | PRO | 276 | 4.604 | −31.772 | 38.944 | 1.00 | 17.26 |
| ATOM | 2066 | CB | PRO | 276 | 4.838 | −28.494 | 38.592 | 1.00 | 16.97 |
| ATOM | 2067 | CG | PRO | 276 | 5.759 | −27.380 | 38.247 | 1.00 | 17.06 |
| ATOM | 2068 | CD | PRO | 276 | 6.456 | −27.903 | 37.005 | 1.00 | 17.24 |
| ATOM | 2069 | N | LEU | 277 | 4.414 | −31.107 | 36.798 | 1.00 | 16.79 |
| ATOM | 2070 | CA | LEU | 277 | 3.657 | −32.268 | 36.349 | 1.00 | 15.99 |
| ATOM | 2071 | C | LEU | 277 | 4.570 | −33.487 | 36.178 | 1.00 | 16.59 |
| ATOM | 2072 | O | LEU | 277 | 4.247 | −34.589 | 36.634 | 1.00 | 16.48 |
| ATOM | 2073 | CB | LEU | 277 | 2.931 | −31.968 | 35.026 | 1.00 | 15.83 |
| ATOM | 2074 | CG | LEU | 277 | 2.116 | −33.147 | 34.426 | 1.00 | 16.90 |
| ATOM | 2075 | CD1 | LEU | 277 | 1.299 | −33.855 | 35.512 | 1.00 | 14.34 |
| ATOM | 2076 | CD2 | LEU | 277 | 1.180 | −32.677 | 33.283 | 1.00 | 17.53 |
| ATOM | 2077 | N | LEU | 278 | 5.684 | −33.292 | 35.474 | 1.00 | 16.91 |
| ATOM | 2078 | CA | LEU | 278 | 6.724 | −34.319 | 35.324 | 1.00 | 18.10 |
| ATOM | 2079 | C | LEU | 278 | 7.106 | −34.932 | 36.686 | 1.00 | 18.11 |
| ATOM | 2080 | O | LEU | 278 | 7.245 | −36.153 | 36.802 | 1.00 | 18.35 |
| ATOM | 2081 | CB | LEU | 278 | 7.942 | −33.698 | 34.629 | 1.00 | 17.90 |
| ATOM | 2082 | CG | LEU | 278 | 8.103 | −33.804 | 33.105 | 1.00 | 21.44 |
| ATOM | 2083 | CD1 | LEU | 278 | 8.906 | −32.618 | 32.566 | 1.00 | 22.83 |
| ATOM | 2084 | CD2 | LEU | 278 | 6.826 | −33.995 | 32.295 | 1.00 | 20.70 |
| ATOM | 2085 | N | ASP | 279 | 7.223 | −34.084 | 37.714 | 1.00 | 18.71 |
| ATOM | 2086 | CA | ASP | 279 | 7.501 | −34.535 | 39.097 | 1.00 | 19.67 |
| ATOM | 2087 | C | ASP | 279 | 6.415 | −35.403 | 39.726 | 1.00 | 19.66 |
| ATOM | 2088 | O | ASP | 279 | 6.704 | −36.187 | 40.635 | 1.00 | 20.49 |
| ATOM | 2089 | CB | ASP | 279 | 7.802 | −33.353 | 40.032 | 1.00 | 18.94 |
| ATOM | 2090 | CG | ASP | 279 | 9.073 | −32.618 | 39.646 | 1.00 | 21.19 |
| ATOM | 2091 | OD1 | ASP | 279 | 9.933 | −33.246 | 38.991 | 1.00 | 18.99 |
| ATOM | 2092 | OD2 | ASP | 279 | 9.196 | −31.405 | 39.961 | 1.00 | 23.21 |
| ATOM | 2093 | N | THR | 280 | 5.183 | −35.288 | 39.252 | 1.00 | 18.68 |
| ATOM | 2094 | CA | THR | 280 | 4.082 | −36.067 | 39.851 | 1.00 | 18.53 |
| ATOM | 2095 | C | THR | 280 | 3.568 | −37.198 | 38.965 | 1.00 | 18.55 |
| ATOM | 2096 | O | THR | 280 | 2.685 | −37.955 | 39.363 | 1.00 | 19.30 |
| ATOM | 2097 | CB | THR | 280 | 2.923 | −35.150 | 40.238 | 1.00 | 18.38 |
| ATOM | 2098 | CG2 | THR | 280 | 3.344 | −34.272 | 41.388 | 1.00 | 18.27 |
| ATOM | 2099 | OG1 | THR | 280 | 2.597 | −34.312 | 39.116 | 1.00 | 17.63 |
| ATOM | 2100 | N | LEU | 281 | 4.118 | −37.326 | 37.761 | 1.00 | 18.42 |
| ATOM | 2101 | CA | LEU | 281 | 3.771 | −38.438 | 36.880 | 1.00 | 18.19 |
| ATOM | 2102 | C | LEU | 281 | 4.364 | −39.763 | 37.380 | 1.00 | 19.21 |
| ATOM | 2103 | O | LEU | 281 | 5.442 | −39.773 | 37.987 | 1.00 | 19.12 |
| ATOM | 2104 | CB | LEU | 281 | 4.251 | −38.161 | 35.441 | 1.00 | 18.12 |
| ATOM | 2105 | CG | LEU | 281 | 3.427 | −37.190 | 34.565 | 1.00 | 17.84 |
| ATOM | 2106 | CD1 | LEU | 281 | 4.105 | −37.002 | 33.234 | 1.00 | 17.57 |
| ATOM | 2107 | CD2 | LEU | 281 | 2.025 | −37.709 | 34.331 | 1.00 | 16.38 |
| ATOM | 2108 | N | PRO | 282 | 3.666 | −40.890 | 37.116 | 1.00 | 19.75 |
| ATOM | 2109 | CA | PRO | 282 | 4.203 | −42.217 | 37.428 | 1.00 | 20.06 |
| ATOM | 2110 | C | PRO | 282 | 5.544 | −42.361 | 36.715 | 1.00 | 21.17 |
| ATOM | 2111 | O | PRO | 282 | 5.701 | −41.873 | 35.577 | 1.00 | 21.54 |
| ATOM | 2112 | CB | PRO | 282 | 3.182 | −43.170 | 36.794 | 1.00 | 20.03 |
| ATOM | 2113 | CG | PRO | 282 | 1.881 | −42.381 | 36.769 | 1.00 | 20.12 |
| ATOM | 2114 | CD | PRO | 282 | 2.305 | −40.952 | 36.539 | 1.00 | 19.60 |
| ATOM | 2115 | N | ALA | 283 | 6.500 | −43.012 | 37.378 | 1.00 | 21.23 |
| ATOM | 2116 | CA | ALA | 283 | 7.849 | −43.157 | 36.851 | 1.00 | 21.95 |
| ATOM | 2117 | C | ALA | 283 | 7.824 | −43.973 | 35.575 | 1.00 | 21.64 |
| ATOM | 2118 | O | ALA | 283 | 6.951 | −44.828 | 35.393 | 1.00 | 21.75 |
| ATOM | 2119 | CB | ALA | 283 | 8.771 | −43.812 | 37.896 | 1.00 | 22.36 |
| ATOM | 2120 | N | GLY | 284 | 8.778 | −43.704 | 34.695 | 1.00 | 21.70 |
| ATOM | 2121 | CA | GLY | 284 | 8.869 | −44.446 | 33.442 | 1.00 | 21.75 |
| ATOM | 2122 | C | GLY | 284 | 8.506 | −43.616 | 32.222 | 1.00 | 21.77 |
| ATOM | 2123 | O | GLY | 284 | 8.772 | −44.032 | 31.087 | 1.00 | 22.04 |
| ATOM | 2124 | N | TYR | 285 | 7.925 | −42.435 | 32.427 | 1.00 | 20.98 |
| ATOM | 2125 | CA | TYR | 285 | 7.606 | −41.565 | 31.284 | 1.00 | 20.54 |
| ATOM | 2126 | C | TYR | 285 | 8.899 | −41.202 | 30.581 | 1.00 | 20.58 |
| ATOM | 2127 | O | TYR | 285 | 9.966 | −41.255 | 31.198 | 1.00 | 20.90 |
| ATOM | 2128 | CB | TYR | 285 | 6.858 | −40.310 | 31.734 | 1.00 | 20.00 |
| ATOM | 2129 | CG | TYR | 285 | 7.661 | −39.412 | 32.640 | 1.00 | 19.60 |
| ATOM | 2130 | CD1 | TYR | 285 | 8.519 | −38.439 | 32.109 | 1.00 | 18.08 |
| ATOM | 2131 | CD2 | TYR | 285 | 7.572 | −39.535 | 34.031 | 1.00 | 19.78 |
| ATOM | 2132 | CE1 | TYR | 285 | 9.274 | −37.595 | 32.958 | 1.00 | 20.35 |
| ATOM | 2133 | CE2 | TYR | 285 | 8.318 | −38.692 | 34.897 | 1.00 | 20.11 |
| ATOM | 2134 | CZ | TYR | 285 | 9.161 | −37.740 | 34.346 | 1.00 | 21.32 |
| ATOM | 2135 | OH | TYR | 285 | 9.896 | −36.937 | 35.173 | 1.00 | 23.19 |
| ATOM | 2136 | N | VAL | 286 | 8.821 | −40.868 | 29.294 | 1.00 | 20.21 |
| ATOM | 2137 | CA | VAL | 286 | 9.970 | −40.324 | 28.580 | 1.00 | 20.41 |
| ATOM | 2138 | C | VAL | 286 | 9.549 | −39.015 | 27.900 | 1.00 | 21.09 |
| ATOM | 2139 | O | VAL | 286 | 8.616 | −39.004 | 27.082 | 1.00 | 21.53 |
| ATOM | 2140 | CB | VAL | 286 | 10.553 | −41.314 | 27.497 | 1.00 | 20.48 |
| ATOM | 2141 | CG1 | VAL | 286 | 11.010 | −42.628 | 28.117 | 1.00 | 19.21 |
| ATOM | 2142 | CG2 | VAL | 286 | 11.733 | −40.657 | 26.727 | 1.00 | 19.91 |
| ATOM | 2143 | N | VAL | 287 | 10.231 | −37.924 | 28.241 | 1.00 | 20.97 |
| ATOM | 2144 | CA | VAL | 287 | 10.072 | −36.667 | 27.540 | 1.00 | 21.64 |
| ATOM | 2145 | C | VAL | 287 | 10.668 | −36.780 | 26.125 | 1.00 | 22.39 |
| ATOM | 2146 | O | VAL | 287 | 11.851 | −37.104 | 25.956 | 1.00 | 21.65 |
| ATOM | 2147 | CB | VAL | 287 | 10.725 | −35.496 | 28.302 | 1.00 | 21.95 |
| ATOM | 2148 | CG1 | VAL | 287 | 10.183 | −35.429 | 29.748 | 1.00 | 22.59 |
| ATOM | 2149 | CG2 | VAL | 287 | 10.475 | −34.169 | 27.555 | 1.00 | 20.44 |
| ATOM | 2150 | N | ASN | 288 | 9.817 | −36.559 | 25.118 | 1.00 | 22.94 |
| ATOM | 2151 | CA | ASN | 288 | 10.243 | −36.552 | 23.717 | 1.00 | 23.60 |
| ATOM | 2152 | C | ASN | 288 | 11.103 | −35.303 | 23.438 | 1.00 | 22.91 |
| ATOM | 2153 | O | ASN | 288 | 10.972 | −34.291 | 24.140 | 1.00 | 23.02 |
| ATOM | 2154 | CB | ASN | 288 | 9.012 | −36.563 | 22.779 | 1.00 | 23.88 |
| ATOM | 2155 | CG | ASN | 288 | 7.980 | −37.645 | 23.133 | 1.00 | 27.07 |
| ATOM | 2156 | ND2 | ASN | 288 | 6.705 | −37.354 | 22.826 | 1.00 | 28.05 |
| ATOM | 2157 | OD1 | ASN | 288 | 8.311 | −38.731 | 23.646 | 1.00 | 27.74 |
| ATOM | 2158 | N | PRO | 289 | 11.983 | −35.354 | 22.410 | 1.00 | 23.07 |
| ATOM | 2159 | CA | PRO | 289 | 12.630 | −34.092 | 22.001 | 1.00 | 22.77 |
| ATOM | 2160 | C | PRO | 289 | 11.575 | −33.016 | 21.671 | 1.00 | 22.32 |
| ATOM | 2161 | O | PRO | 289 | 10.512 | −33.321 | 21.127 | 1.00 | 22.33 |
| ATOM | 2162 | CB | PRO | 289 | 13.404 | −34.476 | 20.716 | 1.00 | 23.21 |
| ATOM | 2163 | CG | PRO | 289 | 13.564 | −35.964 | 20.769 | 1.00 | 23.15 |
| ATOM | 2164 | CD | PRO | 289 | 12.426 | −36.510 | 21.593 | 1.00 | 23.30 |
| ATOM | 2165 | N | THR | 290 | 11.852 | −31.772 | 22.025 | 1.00 | 22.19 |
| ATOM | 2166 | CA | THR | 290 | 10.951 | −30.671 | 21.676 | 1.00 | 22.32 |
| ATOM | 2167 | C | THR | 290 | 11.181 | −30.274 | 20.214 | 1.00 | 22.22 |
| ATOM | 2168 | O | THR | 290 | 12.208 | −30.630 | 19.626 | 1.00 | 22.33 |
| ATOM | 2169 | CB | THR | 290 | 11.245 | −29.434 | 22.537 | 1.00 | 22.21 |
| ATOM | 2170 | CG2 | THR | 290 | 10.882 | −29.664 | 24.013 | 1.00 | 20.75 |
| ATOM | 2171 | OG1 | THR | 290 | 12.639 | −29.128 | 22.418 | 1.00 | 23.72 |
| ATOM | 2172 | N | THR | 291 | 10.233 | −29.558 | 19.618 | 1.00 | 21.73 |
| ATOM | 2173 | CA | THR | 291 | 10.494 | −28.901 | 18.322 | 1.00 | 21.84 |
| ATOM | 2174 | C | THR | 291 | 10.180 | −27.418 | 18.414 | 1.00 | 21.35 |
| ATOM | 2175 | O | THR | 291 | 9.077 | −27.029 | 18.847 | 1.00 | 20.65 |
| ATOM | 2176 | CB | THR | 291 | 9.638 | −29.483 | 17.183 | 1.00 | 22.01 |
| ATOM | 2177 | CG2 | THR | 291 | 10.200 | −29.091 | 15.819 | 1.00 | 22.19 |
| ATOM | 2178 | OG1 | THR | 291 | 9.584 | −30.901 | 17.307 | 1.00 | 23.33 |
| ATOM | 2179 | N | SER | 292 | 11.143 | −26.605 | 17.988 | 1.00 | 20.87 |
| ATOM | 2180 | CA | SER | 292 | 10.983 | −25.173 | 17.920 | 1.00 | 20.75 |
| ATOM | 2181 | C | SER | 292 | 10.290 | −24.761 | 16.625 | 1.00 | 20.71 |
| ATOM | 2182 | O | SER | 292 | 10.675 | −25.201 | 15.529 | 1.00 | 20.51 |
| ATOM | 2183 | CB | SER | 292 | 12.343 | −24.486 | 18.076 | 1.00 | 21.18 |
| ATOM | 2184 | OG | SER | 292 | 12.838 | −24.754 | 19.390 | 1.00 | 23.56 |
| ATOM | 2185 | N | LEU | 293 | 9.268 | −23.913 | 16.758 | 1.00 | 19.60 |
| ATOM | 2186 | CA | LEU | 293 | 8.367 | −23.609 | 15.639 | 1.00 | 19.02 |
| ATOM | 2187 | C | LEU | 293 | 7.940 | −22.160 | 15.681 | 1.00 | 18.16 |
| ATOM | 2188 | O | LEU | 293 | 7.789 | −21.589 | 16.770 | 1.00 | 17.97 |
| ATOM | 2189 | CB | LEU | 293 | 7.084 | −24.472 | 15.715 | 1.00 | 18.73 |
| ATOM | 2190 | CG | LEU | 293 | 7.179 | −26.004 | 15.699 | 1.00 | 20.11 |
| ATOM | 2191 | CD1 | LEU | 293 | 5.907 | −26.639 | 16.253 | 1.00 | 19.90 |
| ATOM | 2192 | CD2 | LEU | 293 | 7.485 | −26.523 | 14.303 | 1.00 | 17.84 |
| ATOM | 2193 | N | ASN | 294 | 7.703 | −21.577 | 14.510 | 1.00 | 17.15 |
| ATOM | 2194 | CA | ASN | 294 | 6.896 | −20.366 | 14.449 | 1.00 | 16.98 |
| ATOM | 2195 | C | ASN | 294 | 5.416 | −20.724 | 14.237 | 1.00 | 16.63 |
| ATOM | 2196 | O | ASN | 294 | 5.063 | −21.916 | 14.158 | 1.00 | 15.87 |
| ATOM | 2197 | CB | ASN | 294 | 7.421 | −19.384 | 13.375 | 1.00 | 17.66 |
| ATOM | 2198 | CG | ASN | 294 | 7.267 | −19.920 | 11.965 | 1.00 | 19.77 |
| ATOM | 2199 | ND2 | ASN | 294 | 8.152 | −19.482 | 11.071 | 1.00 | 22.56 |
| ATOM | 2200 | OD1 | ASN | 294 | 6.377 | −20.721 | 11.676 | 1.00 | 20.07 |
| ATOM | 2201 | N | TRP | 295 | 4.557 | −19.699 | 14.152 | 1.00 | 15.72 |
| ATOM | 2202 | CA | TRP | 295 | 3.127 | −19.909 | 13.978 | 1.00 | 15.16 |
| ATOM | 2203 | C | TRP | 295 | 2.755 | −20.859 | 12.815 | 1.00 | 15.29 |
| ATOM | 2204 | O | TRP | 295 | 2.114 | −21.886 | 13.032 | 1.00 | 14.57 |
| ATOM | 2205 | CB | TRP | 295 | 2.390 | −18.574 | 13.782 | 1.00 | 14.17 |
| ATOM | 2206 | CG | TRP | 295 | 0.917 | −18.799 | 13.582 | 1.00 | 13.57 |
| ATOM | 2207 | CD1 | TRP | 295 | 0.194 | −18.610 | 12.430 | 1.00 | 12.66 |
| ATOM | 2208 | CD2 | TRP | 295 | −0.004 | −19.313 | 14.560 | 1.00 | 14.27 |
| ATOM | 2209 | CE2 | TRP | 295 | −1.280 | −19.384 | 13.942 | 1.00 | 14.31 |
| ATOM | 2210 | CE3 | TRP | 295 | 0.122 | −19.698 | 15.904 | 1.00 | 13.75 |
| ATOM | 2211 | NE1 | TRP | 295 | −1.135 | −18.953 | 12.641 | 1.00 | 14.43 |
| ATOM | 2212 | CZ2 | TRP | 295 | −2.433 | −19.809 | 14.639 | 1.00 | 14.97 |
| ATOM | 2213 | CZ3 | TRP | 295 | −1.023 | −20.118 | 16.599 | 1.00 | 16.67 |
| ATOM | 2214 | CH2 | TRP | 295 | −2.289 | −20.158 | 15.967 | 1.00 | 12.95 |
| ATOM | 2215 | N | ILE | 296 | 3.136 | −20.504 | 11.585 | 1.00 | 15.65 |
| ATOM | 2216 | CA | ILE | 296 | 2.687 | −21.281 | 10.418 | 1.00 | 16.08 |
| ATOM | 2217 | C | ILE | 296 | 3.261 | −22.708 | 10.432 | 1.00 | 16.23 |
| ATOM | 2218 | O | ILE | 296 | 2.612 | −23.678 | 10.010 | 1.00 | 16.02 |
| ATOM | 2219 | CB | ILE | 296 | 2.923 | −20.516 | 9.056 | 1.00 | 17.03 |
| ATOM | 2220 | CG1 | ILE | 296 | 2.096 | −21.130 | 7.920 | 1.00 | 16.87 |
| ATOM | 2221 | CG2 | ILE | 296 | 4.422 | −20.441 | 8.680 | 1.00 | 16.85 |
| ATOM | 2222 | CD1 | ILE | 296 | 0.575 | −21.076 | 8.137 | 1.00 | 17.69 |
| ATOM | 2223 | N | GLU | 297 | 4.481 | −22.835 | 10.942 | 1.00 | 15.74 |
| ATOM | 2224 | CA | GLU | 297 | 5.066 | −24.151 | 11.142 | 1.00 | 16.04 |
| ATOM | 2225 | C | GLU | 297 | 4.263 | −24.985 | 12.120 | 1.00 | 15.55 |
| ATOM | 2226 | O | GLU | 297 | 4.097 | −26.190 | 11.904 | 1.00 | 16.23 |
| ATOM | 2227 | CB | GLU | 297 | 6.515 | −24.012 | 11.609 | 1.00 | 16.59 |
| ATOM | 2228 | CG | GLU | 297 | 7.448 | −23.545 | 10.479 | 1.00 | 17.50 |
| ATOM | 2229 | CD | GLU | 297 | 8.840 | −23.138 | 10.966 | 1.00 | 21.48 |
| ATOM | 2230 | OE1 | GLU | 297 | 9.039 | −22.924 | 12.193 | 1.00 | 20.18 |
| ATOM | 2231 | OE2 | GLU | 297 | 9.740 | −22.999 | 10.098 | 1.00 | 22.22 |
| ATOM | 2232 | N | SER | 298 | 3.743 | −24.359 | 13.189 | 1.00 | 14.37 |
| ATOM | 2233 | CA | SER | 298 | 2.927 | −25.107 | 14.158 | 1.00 | 13.56 |
| ATOM | 2234 | C | SER | 298 | 1.619 | −25.579 | 13.517 | 1.00 | 13.08 |
| ATOM | 2235 | O | SER | 298 | 1.168 | −26.694 | 13.769 | 1.00 | 12.77 |
| ATOM | 2236 | CB | SER | 298 | 2.633 | −24.287 | 15.428 | 1.00 | 13.29 |
| ATOM | 2237 | OG | SER | 298 | 1.723 | −23.211 | 15.158 | 1.00 | 13.21 |
| ATOM | 2238 | N | VAL | 299 | 1.021 | −24.738 | 12.678 | 1.00 | 12.83 |
| ATOM | 2239 | CA | VAL | 299 | −0.177 | −25.143 | 11.934 | 1.00 | 13.52 |
| ATOM | 2240 | C | VAL | 299 | 0.078 | −26.415 | 11.109 | 1.00 | 14.02 |
| ATOM | 2241 | O | VAL | 299 | −0.720 | −27.366 | 11.161 | 1.00 | 13.57 |
| ATOM | 2242 | CB | VAL | 299 | −0.750 | −23.994 | 11.073 | 1.00 | 13.69 |
| ATOM | 2243 | CG1 | VAL | 299 | −0.998 | −22.774 | 11.952 | 1.00 | 13.00 |
| ATOM | 2244 | CG2 | VAL | 299 | −2.079 | −24.421 | 10.375 | 1.00 | 13.79 |
| ATOM | 2245 | N | LEU | 300 | 1.196 | −26.431 | 10.370 | 1.00 | 14.10 |
| ATOM | 2246 | CA | LEU | 300 | 1.569 | −27.616 | 9.605 | 1.00 | 14.83 |
| ATOM | 2247 | C | LEU | 300 | 1.842 | −28.798 | 10.541 | 1.00 | 15.36 |
| ATOM | 2248 | O | LEU | 300 | 1.412 | −29.939 | 10.266 | 1.00 | 15.83 |
| ATOM | 2249 | CB | LEU | 300 | 2.805 | −27.342 | 8.717 | 1.00 | 14.32 |
| ATOM | 2250 | CG | LEU | 300 | 3.241 | −28.511 | 7.819 | 1.00 | 15.80 |
| ATOM | 2251 | CD1 | LEU | 300 | 4.497 | −28.129 | 7.010 | 1.00 | 16.84 |
| ATOM | 2252 | CD2 | LEU | 300 | 2.097 | −28.969 | 6.886 | 1.00 | 15.74 |
| ATOM | 2253 | N | SER | 301 | 2.555 | −28.527 | 11.639 | 1.00 | 15.13 |
| ATOM | 2254 | CA | SER | 301 | 3.047 | −29.602 | 12.519 | 1.00 | 14.65 |
| ATOM | 2255 | C | SER | 301 | 1.952 | −30.501 | 13.082 | 1.00 | 14.91 |
| ATOM | 2256 | O | SER | 301 | 2.160 | −31.696 | 13.191 | 1.00 | 14.54 |
| ATOM | 2257 | CB | SER | 301 | 3.863 | −29.029 | 13.680 | 1.00 | 15.18 |
| ATOM | 2258 | OG | SER | 301 | 4.422 | −30.069 | 14.447 | 1.00 | 14.48 |
| ATOM | 2259 | N | TYR | 302 | 0.798 | −29.922 | 13.436 | 1.00 | 14.98 |
| ATOM | 2260 | CA | TYR | 302 | −0.290 | −30.713 | 14.040 | 0.50 | 14.82 |
| ATOM | 2261 | C | TYR | 302 | −1.422 | −31.036 | 13.050 | 1.00 | 14.62 |
| ATOM | 2262 | O | TYR | 302 | −2.475 | −31.539 | 13.454 | 1.00 | 14.83 |
| ATOM | 2263 | CB | TYR | 302 | −0.847 | −30.018 | 15.295 | 0.50 | 14.87 |
| ATOM | 2264 | CG | TYR | 302 | −0.142 | −30.381 | 16.596 | 0.50 | 15.67 |
| ATOM | 2265 | CD1 | TYR | 302 | −0.079 | −31.702 | 17.039 | 0.50 | 16.05 |
| ATOM | 2266 | CD2 | TYR | 302 | 0.454 | −29.400 | 17.383 | 0.50 | 15.97 |
| ATOM | 2267 | CE1 | TYR | 302 | 0.557 | −32.032 | 18.227 | 0.50 | 15.43 |
| ATOM | 2268 | CE2 | TYR | 302 | 1.095 | −29.720 | 18.567 | 0.50 | 15.51 |
| ATOM | 2269 | CZ | TYR | 302 | 1.145 | −31.033 | 18.988 | 0.50 | 16.25 |
| ATOM | 2270 | OH | TYR | 302 | 1.779 | −31.341 | 20.178 | 0.50 | 14.70 |
| ATOM | 2271 | N | SER | 303 | −1.181 | −30.790 | 11.750 | 1.00 | 14.88 |
| ATOM | 2272 | CA | SER | 303 | −2.203 | −30.943 | 10.691 | 0.50 | 15.37 |
| ATOM | 2273 | C | SER | 303 | −2.502 | −32.375 | 10.261 | 1.00 | 15.18 |
| ATOM | 2274 | O | SER | 303 | −3.610 | −32.664 | 9.755 | 1.00 | 14.29 |
| ATOM | 2275 | CB | SER | 303 | −1.781 | −30.181 | 9.425 | 0.50 | 15.49 |
| ATOM | 2276 | OG | SER | 303 | −1.846 | −28.790 | 9.633 | 0.50 | 16.96 |
| ATOM | 2277 | N | ASN | 304 | −1.499 | −33.247 | 10.404 | 1.00 | 16.01 |
| ATOM | 2278 | CA | ASN | 304 | −1.533 | −34.611 | 9.875 | 1.00 | 17.95 |
| ATOM | 2279 | C | ASN | 304 | −1.518 | −34.677 | 8.346 | 1.00 | 18.61 |
| ATOM | 2280 | O | ASN | 304 | −1.856 | −35.722 | 7.776 | 1.00 | 19.23 |
| ATOM | 2281 | CB | ASN | 304 | −2.719 | −35.413 | 10.442 | 1.00 | 18.26 |
| ATOM | 2282 | CG | ASN | 304 | −2.535 | −35.742 | 11.903 | 1.00 | 21.74 |
| ATOM | 2283 | ND2 | ASN | 304 | −3.597 | −35.579 | 12.703 | 1.00 | 24.06 |
| ATOM | 2284 | OD1 | ASN | 304 | −1.442 | −36.136 | 12.318 | 1.00 | 24.52 |
| ATOM | 2285 | N | PHE | 305 | −1.152 | −33.569 | 7.689 | 1.00 | 18.82 |
| ATOM | 2286 | CA | PHE | 305 | −1.037 | −33.520 | 6.217 | 1.00 | 19.17 |
| ATOM | 2287 | C | PHE | 305 | 0.261 | −32.852 | 5.820 | 1.00 | 19.08 |
| ATOM | 2288 | O | PHE | 305 | 0.924 | −32.221 | 6.656 | 1.00 | 18.78 |
| ATOM | 2289 | CB | PHE | 305 | −2.227 | −32.780 | 5.563 | 1.00 | 18.70 |
| ATOM | 2290 | CG | PHE | 305 | −3.508 | −33.524 | 5.668 | 1.00 | 21.19 |
| ATOM | 2291 | CD1 | PHE | 305 | −3.861 | −34.459 | 4.697 | 1.00 | 21.65 |
| ATOM | 2292 | CD2 | PHE | 305 | −4.349 | −33.343 | 6.774 | 1.00 | 22.68 |
| ATOM | 2293 | CE1 | PHE | 305 | −5.039 | −35.181 | 4.802 | 1.00 | 24.25 |
| ATOM | 2294 | CE2 | PHE | 305 | −5.548 | −34.069 | 6.898 | 1.00 | 24.78 |
| ATOM | 2295 | CZ | PHE | 305 | −5.894 | −34.995 | 5.914 | 1.00 | 25.80 |
| ATOM | 2296 | N | ASP | 306 | 0.625 | −32.995 | 4.543 | 1.00 | 19.59 |
| ATOM | 2297 | CA | ASP | 306 | 1.874 | −32.432 | 4.071 | 1.00 | 19.52 |
| ATOM | 2298 | C | ASP | 306 | 1.692 | −30.968 | 3.640 | 1.00 | 18.66 |
| ATOM | 2299 | O | ASP | 306 | 2.645 | −30.308 | 3.260 | 1.00 | 17.90 |
| ATOM | 2300 | CB | ASP | 306 | 2.514 | −33.309 | 2.977 | 1.00 | 20.54 |
| ATOM | 2301 | CG | ASP | 306 | 1.829 | −33.177 | 1.625 | 1.00 | 22.92 |
| ATOM | 2302 | OD1 | ASP | 306 | 0.729 | −32.590 | 1.528 | 1.00 | 21.49 |
| ATOM | 2303 | OD2 | ASP | 306 | 2.408 | −33.667 | 0.636 | 1.00 | 28.07 |
| ATOM | 2304 | N | HIS | 307 | 0.460 | −30.474 | 3.712 | 1.00 | 17.53 |
| ATOM | 2305 | CA | HIS | 307 | 0.205 | −29.042 | 3.546 | 1.00 | 16.98 |
| ATOM | 2306 | C | HIS | 307 | −1.050 | −28.669 | 4.329 | 1.00 | 16.17 |
| ATOM | 2307 | O | HIS | 307 | −1.837 | −29.552 | 4.703 | 1.00 | 16.29 |
| ATOM | 2308 | CB | HIS | 307 | 0.064 | −28.673 | 2.058 | 1.00 | 16.04 |
| ATOM | 2309 | CG | HIS | 307 | −1.232 | −29.109 | 1.445 | 1.00 | 16.60 |
| ATOM | 2310 | CD2 | HIS | 307 | −2.368 | −28.423 | 1.186 | 1.00 | 17.44 |
| ATOM | 2311 | ND1 | HIS | 307 | −1.468 | −30.399 | 1.020 | 1.00 | 18.69 |
| ATOM | 2312 | CE1 | HIS | 307 | −2.685 | −30.487 | 0.523 | 1.00 | 16.73 |
| ATOM | 2313 | NE2 | HIS | 307 | −3.252 | −29.300 | 0.609 | 1.00 | 17.79 |
| ATOM | 2314 | N | VAL | 308 | −1.241 | −27.373 | 4.573 | 1.00 | 16.20 |
| ATOM | 2315 | CA | VAL | 308 | −2.423 | −26.910 | 5.318 | 1.00 | 16.61 |
| ATOM | 2316 | C | VAL | 308 | −3.395 | −26.049 | 4.547 | 1.00 | 16.83 |
| ATOM | 2317 | O | VAL | 308 | −4.540 | −25.891 | 4.980 | 1.00 | 17.16 |
| ATOM | 2318 | CB | VAL | 308 | −2.059 | −26.177 | 6.633 | 1.00 | 17.02 |
| ATOM | 2319 | CG1 | VAL | 308 | −1.204 | −27.085 | 7.508 | 1.00 | 17.73 |
| ATOM | 2320 | CG2 | VAL | 308 | −1.369 | −24.867 | 6.377 | 1.00 | 15.36 |
| ATOM | 2321 | N | ASP | 309 | −2.968 | −25.476 | 3.422 | 1.00 | 16.40 |
| ATOM | 2322 | CA | ASP | 309 | −3.871 | −24.593 | 2.690 | 1.00 | 16.89 |
| ATOM | 2323 | C | ASP | 309 | −4.898 | −25.374 | 1.862 | 1.00 | 16.62 |
| ATOM | 2324 | O | ASP | 309 | −4.732 | −25.571 | 0.656 | 1.00 | 16.98 |
| ATOM | 2325 | CB | ASP | 309 | −3.087 | −23.605 | 1.835 | 1.00 | 17.38 |
| ATOM | 2326 | CG | ASP | 309 | −3.955 | −22.486 | 1.293 | 1.00 | 19.38 |
| ATOM | 2327 | OD1 | ASP | 309 | −5.153 | −22.372 | 1.651 | 1.00 | 20.20 |
| ATOM | 2328 | OD2 | ASP | 309 | −3.436 | −21.698 | 0.486 | 1.00 | 21.40 |
| ATOM | 2329 | N | PHE | 310 | −5.958 | −25.814 | 2.531 | 1.00 | 15.90 |
| ATOM | 2330 | CA | PHE | 310 | −7.011 | −26.599 | 1.906 | 1.00 | 16.51 |
| ATOM | 2331 | C | PHE | 310 | −8.127 | −25.733 | 1.367 | 1.00 | 16.41 |
| ATOM | 2332 | O | PHE | 310 | −8.732 | −24.926 | 2.116 | 1.00 | 16.50 |
| ATOM | 2333 | CB | PHE | 310 | −7.592 | −27.606 | 2.908 | 1.00 | 16.29 |
| ATOM | 2334 | CG | PHE | 310 | −6.844 | −28.909 | 2.964 | 1.00 | 17.16 |
| ATOM | 2335 | CD1 | PHE | 310 | −5.566 | −28.974 | 3.510 | 1.00 | 17.85 |
| ATOM | 2336 | CD2 | PHE | 310 | −7.422 | −30.078 | 2.474 | 1.00 | 16.94 |
| ATOM | 2337 | CE1 | PHE | 310 | −4.880 | −30.198 | 3.583 | 1.00 | 19.46 |
| ATOM | 2338 | CE2 | PHE | 310 | −6.737 | −31.301 | 2.538 | 1.00 | 21.11 |
| ATOM | 2339 | CZ | PHE | 310 | −5.467 | −31.353 | 3.098 | 1.00 | 18.95 |
| ATOM | 2340 | N | ILE | 311 | −8.391 | −25.891 | 0.070 | 1.00 | 16.25 |
| ATOM | 2341 | CA | ILE | 311 | −9.571 | −25.272 | −0.580 | 1.00 | 16.70 |
| ATOM | 2342 | C | ILE | 311 | −10.475 | −26.351 | −1.210 | 1.00 | 17.43 |
| ATOM | 2343 | O | ILE | 311 | −11.386 | −26.048 | −1.985 | 1.00 | 17.85 |
| ATOM | 2344 | CB | ILE | 311 | −9.167 | −24.219 | −1.636 | 1.00 | 17.00 |
| ATOM | 2345 | CG1 | ILE | 311 | −8.283 | −24.853 | −2.727 | 1.00 | 16.98 |
| ATOM | 2346 | CG2 | ILE | 311 | −8.441 | −23.032 | −0.964 | 1.00 | 14.61 |
| ATOM | 2347 | CD1 | ILE | 311 | −8.148 | −23.989 | −4.003 | 1.00 | 18.38 |
| ATOM | 2348 | N | THR | 312 | −10.197 | −27.601 | −0.851 | 1.00 | 17.37 |
| ATOM | 2349 | CA | THR | 312 | −10.946 | −28.790 | −1.231 | 1.00 | 18.13 |
| ATOM | 2350 | C | THR | 312 | −11.150 | −29.596 | 0.058 | 1.00 | 17.29 |
| ATOM | 2351 | O | THR | 312 | −10.475 | −29.313 | 1.052 | 1.00 | 16.68 |
| ATOM | 2352 | CB | THR | 312 | −10.126 | −29.680 | −2.216 | 1.00 | 18.47 |
| ATOM | 2353 | CG2 | THR | 312 | −10.084 | −29.072 | −3.598 | 1.00 | 19.79 |
| ATOM | 2354 | OG1 | THR | 312 | −8.789 | −29.796 | −1.725 | 1.00 | 21.70 |
| ATOM | 2355 | N | PRO | 313 | −12.069 | −30.597 | 0.054 | 1.00 | 16.70 |
| ATOM | 2356 | CA | PRO | 313 | −12.264 | −31.481 | 1.221 | 1.00 | 16.47 |
| ATOM | 2357 | C | PRO | 313 | −11.021 | −32.315 | 1.546 | 1.00 | 17.02 |
| ATOM | 2358 | O | PRO | 313 | −10.249 | −32.621 | 0.639 | 1.00 | 16.34 |
| ATOM | 2359 | CB | PRO | 313 | −13.387 | −32.436 | 0.774 | 1.00 | 16.35 |
| ATOM | 2360 | CG | PRO | 313 | −14.044 | −31.782 | −0.406 | 1.00 | 15.79 |
| ATOM | 2361 | CD | PRO | 313 | −12.999 | −30.915 | −1.051 | 1.00 | 17.08 |
| ATOM | 2362 | N | GLN | 314 | −10.848 | −32.726 | 2.807 | 1.00 | 16.77 |
| ATOM | 2363 | CA | GLN | 314 | −9.848 | −33.747 | 3.080 | 1.00 | 17.29 |
| ATOM | 2364 | C | GLN | 314 | −10.397 | −35.152 | 2.772 | 1.00 | 17.81 |
| ATOM | 2365 | O | GLN | 314 | −11.601 | −35.302 | 2.479 | 1.00 | 16.99 |
| ATOM | 2366 | CB | GLN | 314 | −9.202 | −33.631 | 4.472 | 1.00 | 18.05 |
| ATOM | 2367 | CG | GLN | 314 | −10.036 | −33.139 | 5.628 | 1.00 | 18.98 |
| ATOM | 2368 | CD | GLN | 314 | −9.233 | −33.096 | 6.931 | 1.00 | 19.79 |
| ATOM | 2369 | NE2 | GLN | 314 | −8.954 | −34.274 | 7.465 | 1.00 | 18.59 |
| ATOM | 2370 | OE1 | GLN | 314 | −8.863 | −32.013 | 7.451 | 1.00 | 17.24 |
| ATOM | 2371 | N | PRO | 315 | −9.515 | −36.181 | 2.799 | 1.00 | 17.48 |
| ATOM | 2372 | CA | PRO | 315 | −9.964 | −37.551 | 2.583 | 1.00 | 17.28 |
| ATOM | 2373 | C | PRO | 315 | −11.077 | −37.900 | 3.552 | 1.00 | 17.14 |
| ATOM | 2374 | O | PRO | 315 | −11.058 | −37.467 | 4.713 | 1.00 | 17.48 |
| ATOM | 2375 | CB | PRO | 315 | −8.697 | −38.385 | 2.864 | 1.00 | 17.65 |
| ATOM | 2376 | CG | PRO | 315 | −7.572 | −37.460 | 2.438 | 1.00 | 17.53 |
| ATOM | 2377 | CD | PRO | 315 | −8.044 | −36.099 | 2.910 | 1.00 | 18.11 |
| ATOM | 2378 | N | VAL | 316 | −12.036 | −38.687 | 3.077 | 1.00 | 16.17 |
| ATOM | 2379 | CA | VAL | 316 | −13.195 | −39.044 | 3.882 | 1.00 | 15.51 |
| ATOM | 2380 | C | VAL | 316 | −12.787 | −40.150 | 4.832 | 1.00 | 15.72 |
| ATOM | 2381 | O | VAL | 316 | −11.760 | −40.816 | 4.623 | 1.00 | 14.44 |
| ATOM | 2382 | CB | VAL | 316 | −14.383 | −39.522 | 3.003 | 1.00 | 16.04 |
| ATOM | 2383 | CG1 | VAL | 316 | −13.961 | −40.723 | 2.157 | 1.00 | 15.76 |
| ATOM | 2384 | CG2 | VAL | 316 | −14.902 | −38.387 | 2.097 | 1.00 | 14.33 |
| ATOM | 2385 | N | GLU | 317 | −13.594 | −40.331 | 5.878 | 1.00 | 14.70 |
| ATOM | 2386 | CA | GLU | 317 | −13.371 | −41.374 | 6.859 | 1.00 | 14.91 |
| ATOM | 2387 | C | GLU | 317 | −14.699 | −42.032 | 7.154 | 1.00 | 14.83 |
| ATOM | 2388 | O | GLU | 317 | −15.772 | −41.523 | 6.759 | 1.00 | 15.03 |
| ATOM | 2389 | CB | GLU | 317 | −12.769 | −40.794 | 8.152 | 1.00 | 14.45 |
| ATOM | 2390 | CG | GLU | 317 | −11.426 | −40.104 | 7.914 | 1.00 | 15.43 |
| ATOM | 2391 | CD | GLU | 317 | −10.744 | −39.601 | 9.200 | 1.00 | 17.62 |
| ATOM | 2392 | OE1 | GLU | 317 | −11.133 | −39.986 | 10.329 | 1.00 | 18.65 |
| ATOM | 2393 | OE2 | GLU | 317 | −9.820 | −38.780 | 9.063 | 1.00 | 20.66 |
| ATOM | 2394 | N | ASN | 318 | −14.634 | −43.145 | 7.880 | 1.00 | 14.58 |
| ATOM | 2395 | CA | ASN | 318 | −15.809 | −43.973 | 8.112 | 1.00 | 14.30 |
| ATOM | 2396 | C | ASN | 318 | −15.871 | −44.354 | 9.590 | 1.00 | 14.11 |
| ATOM | 2397 | O | ASN | 318 | −15.138 | −45.238 | 10.038 | 1.00 | 12.70 |
| ATOM | 2398 | CB | ASN | 318 | −15.708 | −45.209 | 7.202 | 1.00 | 14.80 |
| ATOM | 2399 | CG | ASN | 318 | −16.837 | −46.175 | 7.387 | 1.00 | 15.12 |
| ATOM | 2400 | ND2 | ASN | 318 | −16.547 | −47.459 | 7.170 | 1.00 | 18.09 |
| ATOM | 2401 | OD1 | ASN | 318 | −17.965 | −45.793 | 7.711 | 1.00 | 16.53 |
| ATOM | 2402 | N | PHE | 319 | −16.736 | −43.697 | 10.368 | 1.00 | 13.61 |
| ATOM | 2403 | CA | PHE | 319 | −16.503 | −43.734 | 11.812 | 1.00 | 13.44 |
| ATOM | 2404 | C | PHE | 319 | −17.713 | −43.387 | 12.659 | 1.00 | 13.97 |
| ATOM | 2405 | O | PHE | 319 | −18.767 | −42.953 | 12.142 | 1.00 | 14.03 |
| ATOM | 2406 | CB | PHE | 319 | −15.336 | −42.764 | 12.162 | 1.00 | 13.33 |
| ATOM | 2407 | CG | PHE | 319 | −15.700 | −41.279 | 11.994 | 1.00 | 13.12 |
| ATOM | 2408 | CD1 | PHE | 319 | −16.158 | −40.530 | 13.083 | 1.00 | 12.13 |
| ATOM | 2409 | CD2 | PHE | 319 | −15.596 | −40.654 | 10.752 | 1.00 | 10.79 |
| ATOM | 2410 | CE1 | PHE | 319 | −16.495 | −39.177 | 12.943 | 1.00 | 11.24 |
| ATOM | 2411 | CE2 | PHE | 319 | −15.939 | −39.287 | 10.601 | 1.00 | 11.29 |
| ATOM | 2412 | CZ | PHE | 319 | −16.385 | −38.560 | 11.708 | 1.00 | 10.56 |
| ATOM | 2413 | N | TYR | 320 | −17.535 | −43.580 | 13.966 | 1.00 | 13.04 |
| ATOM | 2414 | CA | TYR | 320 | −18.460 | −43.126 | 14.986 | 1.00 | 12.65 |
| ATOM | 2415 | C | TYR | 320 | −17.637 | −42.377 | 16.028 | 1.00 | 12.02 |
| ATOM | 2416 | O | TYR | 320 | −16.475 | −42.711 | 16.267 | 1.00 | 12.46 |
| ATOM | 2417 | CB | TYR | 320 | −19.190 | −44.296 | 15.649 | 1.00 | 12.49 |
| ATOM | 2418 | CG | TYR | 320 | −20.212 | −43.858 | 16.683 | 1.00 | 13.44 |
| ATOM | 2419 | CD1 | TYR | 320 | −21.221 | −42.932 | 16.361 | 1.00 | 12.88 |
| ATOM | 2420 | CD2 | TYR | 320 | −20.192 | −44.381 | 17.974 | 1.00 | 14.32 |
| ATOM | 2421 | CE1 | TYR | 320 | −22.169 | −42.536 | 17.295 | 1.00 | 11.11 |
| ATOM | 2422 | CE2 | TYR | 320 | −21.131 | −43.998 | 18.920 | 1.00 | 13.40 |
| ATOM | 2423 | CZ | TYR | 320 | −22.114 | −43.070 | 18.577 | 1.00 | 13.55 |
| ATOM | 2424 | OH | TYR | 320 | −23.042 | −42.696 | 19.527 | 1.00 | 10.33 |
| ATOM | 2425 | N | ALA | 321 | −18.229 | −41.355 | 16.631 | 1.00 | 11.18 |
| ATOM | 2426 | CA | ALA | 321 | −17.537 | −40.557 | 17.631 | 1.00 | 10.62 |
| ATOM | 2427 | C | ALA | 321 | −18.512 | −40.118 | 18.735 | 1.00 | 10.93 |
| ATOM | 2428 | O | ALA | 321 | −19.740 | −40.041 | 18.531 | 1.00 | 11.37 |
| ATOM | 2429 | CB | ALA | 321 | −16.881 | −39.338 | 16.972 | 1.00 | 9.18 |
| ATOM | 2430 | N | LYS | 322 | −17.952 | −39.805 | 19.895 | 1.00 | 10.98 |
| ATOM | 2431 | CA | LYS | 322 | −18.727 | −39.392 | 21.056 | 1.00 | 10.92 |
| ATOM | 2432 | C | LYS | 322 | −17.879 | −38.382 | 21.806 | 1.00 | 11.04 |
| ATOM | 2433 | O | LYS | 322 | −16.689 | −38.247 | 21.516 | 1.00 | 11.23 |
| ATOM | 2434 | CB | LYS | 322 | −19.040 | −40.579 | 21.959 | 1.00 | 10.35 |
| ATOM | 2435 | CG | LYS | 322 | −20.061 | −41.577 | 21.406 | 1.00 | 9.78 |
| ATOM | 2436 | CD | LYS | 322 | −20.560 | −42.570 | 22.499 | 1.00 | 11.31 |
| ATOM | 2437 | CE | LYS | 322 | −21.753 | −42.019 | 23.341 | 1.00 | 9.95 |
| ATOM | 2438 | NZ | LYS | 322 | −23.029 | −42.148 | 22.518 | 1.00 | 8.81 |
| ATOM | 2439 | N | SER | 323 | −18.476 | −37.704 | 22.785 | 1.00 | 11.47 |
| ATOM | 2440 | CA | SER | 323 | −17.767 | −36.694 | 23.554 | 1.00 | 11.67 |
| ATOM | 2441 | C | SER | 323 | −18.284 | −36.617 | 24.978 | 1.00 | 12.00 |
| ATOM | 2442 | O | SER | 323 | −19.363 | −37.119 | 25.288 | 1.00 | 10.18 |
| ATOM | 2443 | CB | SER | 323 | −17.869 | −35.299 | 22.887 | 1.00 | 12.33 |
| ATOM | 2444 | OG | SER | 323 | −19.143 | −34.670 | 23.113 | 1.00 | 13.50 |
| ATOM | 2445 | N | LEU | 324 | −17.484 | −35.958 | 25.817 | 1.00 | 12.00 |
| ATOM | 2446 | CA | LEU | 324 | −17.868 | −35.550 | 27.157 | 1.00 | 13.00 |
| ATOM | 2447 | C | LEU | 324 | −17.127 | −34.273 | 27.489 | 1.00 | 13.23 |
| ATOM | 2448 | O | LEU | 324 | −15.982 | −34.079 | 27.041 | 1.00 | 13.51 |
| ATOM | 2449 | CB | LEU | 324 | −17.505 | −36.609 | 28.194 | 1.00 | 12.45 |
| ATOM | 2450 | CG | LEU | 324 | −18.377 | −37.861 | 28.245 | 1.00 | 13.87 |
| ATOM | 2451 | CD1 | LEU | 324 | −17.745 | −38.925 | 29.192 | 1.00 | 13.49 |
| ATOM | 2452 | CD2 | LEU | 324 | −19.833 | −37.528 | 28.644 | 1.00 | 15.06 |
| ATOM | 2453 | N | THR | 325 | −17.786 | −33.408 | 28.260 | 1.00 | 12.84 |
| ATOM | 2454 | CA | THR | 325 | −17.124 | −32.302 | 28.912 | 1.00 | 13.22 |
| ATOM | 2455 | C | THR | 325 | −17.381 | −32.526 | 30.426 | 1.00 | 13.13 |
| ATOM | 2456 | O | THR | 325 | −18.507 | −32.777 | 30.830 | 1.00 | 13.82 |
| ATOM | 2457 | CB | THR | 325 | −17.654 | −30.927 | 28.420 | 1.00 | 13.16 |
| ATOM | 2458 | CG2 | THR | 325 | −17.447 | −30.763 | 26.861 | 1.00 | 11.62 |
| ATOM | 2459 | OG1 | THR | 325 | −19.065 | −30.813 | 28.719 | 1.00 | 13.82 |
| ATOM | 2460 | N | LEU | 326 | −16.339 | −32.457 | 31.244 | 1.00 | 13.14 |
| ATOM | 2461 | CA | LEU | 326 | −16.489 | −32.766 | 32.677 | 1.00 | 13.44 |
| ATOM | 2462 | C | LEU | 326 | −15.938 | −31.670 | 33.540 | 1.00 | 14.09 |
| ATOM | 2463 | O | LEU | 326 | −14.879 | −31.108 | 33.232 | 1.00 | 13.25 |
| ATOM | 2464 | CB | LEU | 326 | −15.746 | −34.053 | 33.052 | 1.00 | 12.95 |
| ATOM | 2465 | CG | LEU | 326 | −15.877 | −35.288 | 32.150 | 1.00 | 13.49 |
| ATOM | 2466 | CD1 | LEU | 326 | −14.817 | −36.335 | 32.585 | 1.00 | 12.48 |
| ATOM | 2467 | CD2 | LEU | 326 | −17.309 | −35.865 | 32.217 | 1.00 | 10.47 |
| ATOM | 2468 | N | LYS | 327 | −16.634 | −31.394 | 34.653 | 1.00 | 14.76 |
| ATOM | 2469 | CA | LYS | 327 | −16.051 | −30.544 | 35.685 | 1.00 | 15.47 |
| ATOM | 2470 | C | LYS | 327 | −14.786 | −31.192 | 36.224 | 1.00 | 14.84 |
| ATOM | 2471 | O | LYS | 327 | −13.796 | −30.512 | 36.459 | 1.00 | 15.18 |
| ATOM | 2472 | CB | LYS | 327 | −17.037 | −30.284 | 36.838 | 1.00 | 15.19 |
| ATOM | 2473 | CG | LYS | 327 | −18.232 | −29.494 | 36.396 | 1.00 | 18.40 |
| ATOM | 2474 | CD | LYS | 327 | −19.114 | −29.079 | 37.559 | 1.00 | 21.42 |
| ATOM | 2475 | CE | LYS | 327 | −20.305 | −28.298 | 37.034 | 1.00 | 23.78 |
| ATOM | 2476 | NZ | LYS | 327 | −21.449 | −28.361 | 38.006 | 1.00 | 24.91 |
| ATOM | 2477 | N | SER | 328 | −14.836 | −32.503 | 36.425 | 1.00 | 13.99 |
| ATOM | 2478 | CA | SER | 328 | −13.713 | −33.265 | 36.966 | 1.00 | 14.72 |
| ATOM | 2479 | C | SER | 328 | −13.859 | −34.700 | 36.590 | 1.00 | 15.01 |
| ATOM | 2480 | O | SER | 328 | −14.982 | −35.224 | 36.556 | 1.00 | 15.55 |
| ATOM | 2481 | CB | SER | 328 | −13.712 | −33.233 | 38.500 | 1.00 | 14.83 |
| ATOM | 2482 | OG | SER | 328 | −12.555 | −33.905 | 39.007 | 1.00 | 15.11 |
| ATOM | 2483 | N | ILE | 329 | −12.736 | −35.380 | 36.396 | 1.00 | 15.16 |
| ATOM | 2484 | CA | ILE | 329 | −12.812 | −36.827 | 36.173 | 1.00 | 16.08 |
| ATOM | 2485 | C | ILE | 329 | −12.558 | −37.636 | 37.469 | 1.00 | 16.69 |
| ATOM | 2486 | O | ILE | 329 | −12.697 | −38.862 | 37.494 | 1.00 | 16.94 |
| ATOM | 2487 | CB | ILE | 329 | −11.879 | −37.281 | 34.977 | 1.00 | 15.82 |
| ATOM | 2488 | CG1 | ILE | 329 | −12.436 | −38.562 | 34.318 | 1.00 | 16.78 |
| ATOM | 2489 | CG2 | ILE | 329 | −10.409 | −37.357 | 35.426 | 1.00 | 14.37 |
| ATOM | 2490 | CD1 | ILE | 329 | −11.645 | −39.081 | 33.097 | 1.00 | 20.11 |
| ATOM | 2491 | N | LYS | 330 | −12.194 | −36.942 | 38.542 | 1.00 | 17.71 |
| ATOM | 2492 | CA | LYS | 330 | −11.808 | −37.621 | 39.801 | 1.00 | 18.62 |
| ATOM | 2493 | C | LYS | 330 | −12.913 | −38.543 | 40.342 | 1.00 | 18.71 |
| ATOM | 2494 | O | LYS | 330 | −14.099 | −38.317 | 40.126 | 1.00 | 19.33 |
| ATOM | 2495 | CB | LYS | 330 | −11.347 | −36.605 | 40.866 | 1.00 | 17.31 |
| ATOM | 2496 | CG | LYS | 330 | −10.047 | −35.912 | 40.493 | 1.00 | 17.05 |
| ATOM | 2497 | CD | LYS | 330 | −9.664 | −34.855 | 41.521 | 1.00 | 17.24 |
| ATOM | 2498 | CE | LYS | 330 | −8.326 | −34.239 | 41.162 | 1.00 | 16.56 |
| ATOM | 2499 | NZ | LYS | 330 | −7.919 | −33.195 | 42.140 | 1.00 | 13.87 |
| ATOM | 2500 | N | GLY | 331 | −12.509 | −39.601 | 41.016 | 1.00 | 19.67 |
| ATOM | 2501 | CA | GLY | 331 | −13.461 | −40.421 | 41.771 | 1.00 | 20.21 |
| ATOM | 2502 | C | GLY | 331 | −13.767 | −41.708 | 41.037 | 1.00 | 20.74 |
| ATOM | 2503 | O | GLY | 331 | −12.888 | −42.333 | 40.419 | 1.00 | 19.48 |
| ATOM | 2504 | N | ASP | 332 | −15.027 | −42.113 | 41.090 | 1.00 | 21.45 |
| ATOM | 2505 | CA | ASP | 332 | −15.400 | −43.334 | 40.399 | 1.00 | 22.49 |
| ATOM | 2506 | C | ASP | 332 | −15.297 | −43.197 | 38.882 | 1.00 | 22.11 |
| ATOM | 2507 | O | ASP | 332 | −15.039 | −44.189 | 38.203 | 1.00 | 23.07 |
| ATOM | 2508 | CB | ASP | 332 | −16.778 | −43.814 | 40.837 | 1.00 | 23.08 |
| ATOM | 2509 | CG | ASP | 332 | −16.737 | −44.509 | 42.196 | 1.00 | 26.47 |
| ATOM | 2510 | OD1 | ASP | 332 | −15.637 | −44.936 | 42.625 | 1.00 | 29.12 |
| ATOM | 2511 | OD2 | ASP | 332 | −17.796 | −44.624 | 42.837 | 1.00 | 29.41 |
| ATOM | 2512 | N | ALA | 333 | −15.453 | −41.972 | 38.374 | 1.00 | 21.28 |
| ATOM | 2513 | CA | ALA | 333 | −15.429 | −41.704 | 36.930 | 1.00 | 20.38 |
| ATOM | 2514 | C | ALA | 333 | −14.113 | −42.166 | 36.310 | 1.00 | 19.35 |
| ATOM | 2515 | O | ALA | 333 | −14.113 | −42.917 | 35.333 | 1.00 | 20.03 |
| ATOM | 2516 | CB | ALA | 333 | −15.675 | −40.216 | 36.650 | 1.00 | 20.14 |
| ATOM | 2517 | N | VAL | 334 | −12.991 | −41.741 | 36.883 | 1.00 | 18.66 |
| ATOM | 2518 | CA | VAL | 334 | −11.700 | −42.156 | 36.337 | 1.00 | 18.32 |
| ATOM | 2519 | C | VAL | 334 | −11.443 | −43.652 | 36.584 | 1.00 | 18.38 |
| ATOM | 2520 | O | VAL | 334 | −10.866 | −44.332 | 35.741 | 1.00 | 18.42 |
| ATOM | 2521 | CB | VAL | 334 | −10.502 | −41.274 | 36.809 | 1.00 | 17.77 |
| ATOM | 2522 | CG1 | VAL | 334 | −9.249 | −41.637 | 36.028 | 1.00 | 17.01 |
| ATOM | 2523 | CG2 | VAL | 334 | −10.277 | −41.366 | 38.346 | 1.00 | 18.14 |
| ATOM | 2524 | N | LYS | 335 | −11.880 | −44.151 | 37.737 | 1.00 | 18.66 |
| ATOM | 2525 | CA | LYS | 335 | −11.788 | −45.574 | 38.032 | 1.00 | 19.04 |
| ATOM | 2526 | C | LYS | 335 | −12.510 | −46.383 | 36.962 | 1.00 | 18.07 |
| ATOM | 2527 | O | LYS | 335 | −11.955 | −47.332 | 36.434 | 1.00 | 18.24 |
| ATOM | 2528 | CB | LYS | 335 | −12.382 | −45.885 | 39.418 | 1.00 | 18.85 |
| ATOM | 2529 | CG | LYS | 335 | −12.009 | −47.280 | 39.912 | 1.00 | 22.54 |
| ATOM | 2530 | CD | LYS | 335 | −12.839 | −47.652 | 41.164 | 1.00 | 29.88 |
| ATOM | 2531 | CE | LYS | 335 | −12.903 | −46.464 | 42.162 | 1.00 | 33.16 |
| ATOM | 2532 | NZ | LYS | 335 | −13.640 | −46.808 | 43.421 | 1.00 | 36.00 |
| ATOM | 2533 | N | ASN | 336 | −13.745 | −45.992 | 36.648 | 1.00 | 17.74 |
| ATOM | 2534 | CA | ASN | 336 | −14.539 | −46.679 | 35.635 | 1.00 | 17.34 |
| ATOM | 2535 | C | ASN | 336 | −13.901 | −46.484 | 34.267 | 1.00 | 17.24 |
| ATOM | 2536 | O | ASN | 336 | −13.834 | −47.425 | 33.485 | 1.00 | 16.89 |
| ATOM | 2537 | CB | ASN | 336 | −15.981 | −46.158 | 35.604 | 1.00 | 17.56 |
| ATOM | 2538 | CG | ASN | 336 | −16.730 | −46.419 | 36.894 | 1.00 | 18.46 |
| ATOM | 2539 | ND2 | ASN | 336 | −17.891 | −45.773 | 37.068 | 1.00 | 17.92 |
| ATOM | 2540 | OD1 | ASN | 336 | −16.278 | −47.204 | 37.727 | 1.00 | 20.69 |
| ATOM | 2541 | N | PHE | 337 | −13.443 | −45.265 | 33.980 | 1.00 | 16.68 |
| ATOM | 2542 | CA | PHE | 337 | −12.827 | −44.965 | 32.680 | 1.00 | 16.30 |
| ATOM | 2543 | C | PHE | 337 | −11.653 | −45.909 | 32.415 | 1.00 | 16.48 |
| ATOM | 2544 | O | PHE | 337 | −11.549 | −46.516 | 31.349 | 1.00 | 15.96 |
| ATOM | 2545 | CB | PHE | 337 | −12.367 | −43.498 | 32.621 | 1.00 | 15.52 |
| ATOM | 2546 | CG | PHE | 337 | −11.623 | −43.133 | 31.346 | 1.00 | 16.36 |
| ATOM | 2547 | CD1 | PHE | 337 | −12.304 | −42.615 | 30.256 | 1.00 | 16.11 |
| ATOM | 2548 | CD2 | PHE | 337 | −10.238 | −43.306 | 31.243 | 1.00 | 16.38 |
| ATOM | 2549 | CE1 | PHE | 337 | −11.619 | −42.269 | 29.084 | 1.00 | 15.61 |
| ATOM | 2550 | CE2 | PHE | 337 | −9.550 | −42.968 | 30.075 | 1.00 | 15.64 |
| ATOM | 2551 | CZ | PHE | 337 | −10.248 | −42.441 | 28.996 | 1.00 | 15.57 |
| ATOM | 2552 | N | VAL | 338 | −10.770 | −46.017 | 33.410 | 1.00 | 17.29 |
| ATOM | 2553 | CA | VAL | 338 | −9.555 | −46.828 | 33.327 | 1.00 | 17.53 |
| ATOM | 2554 | C | VAL | 338 | −9.842 | −48.349 | 33.304 | 1.00 | 17.97 |
| ATOM | 2555 | O | VAL | 338 | −9.137 | −49.095 | 32.615 | 1.00 | 17.17 |
| ATOM | 2556 | CB | VAL | 338 | −8.566 | −46.420 | 34.446 | 1.00 | 18.12 |
| ATOM | 2557 | CG1 | VAL | 338 | −7.405 | −47.403 | 34.552 | 1.00 | 15.51 |
| ATOM | 2558 | CG2 | VAL | 338 | −8.028 | −44.997 | 34.160 | 1.00 | 18.89 |
| ATOM | 2559 | N | ASP | 339 | −10.873 | −48.791 | 34.044 | 1.00 | 17.84 |
| ATOM | 2560 | CA | ASP | 339 | −11.315 | −50.204 | 33.997 | 1.00 | 18.20 |
| ATOM | 2561 | C | ASP | 339 | −11.667 | −50.611 | 32.554 | 1.00 | 18.34 |
| ATOM | 2562 | O | ASP | 339 | −11.192 | −51.626 | 32.041 | 1.00 | 17.90 |
| ATOM | 2563 | CB | ASP | 339 | −12.499 | −50.441 | 34.949 | 1.00 | 17.87 |
| ATOM | 2564 | CG | ASP | 339 | −12.053 | −50.690 | 36.413 | 1.00 | 19.86 |
| ATOM | 2565 | OD1 | ASP | 339 | −10.851 | −50.922 | 36.674 | 1.00 | 19.62 |
| ATOM | 2566 | OD2 | ASP | 339 | −12.912 | −50.651 | 37.308 | 1.00 | 21.19 |
| ATOM | 2567 | N | TYR | 340 | −12.470 | −49.772 | 31.896 | 1.00 | 18.34 |
| ATOM | 2568 | CA | TYR | 340 | −12.890 | −49.982 | 30.515 | 1.00 | 17.71 |
| ATOM | 2569 | C | TYR | 340 | −11.712 | −49.868 | 29.554 | 1.00 | 17.74 |
| ATOM | 2570 | O | TYR | 340 | −11.559 | −50.668 | 28.624 | 1.00 | 17.74 |
| ATOM | 2571 | CB | TYR | 340 | −13.945 | −48.913 | 30.181 | 1.00 | 17.88 |
| ATOM | 2572 | CG | TYR | 340 | −14.843 | −49.258 | 29.011 | 1.00 | 16.08 |
| ATOM | 2573 | CD1 | TYR | 340 | −16.060 | −49.903 | 29.225 | 1.00 | 15.03 |
| ATOM | 2574 | CD2 | TYR | 340 | −14.474 | −48.956 | 27.699 | 1.00 | 16.81 |
| ATOM | 2575 | CE1 | TYR | 340 | −16.905 | −50.216 | 28.178 | 1.00 | 15.98 |
| ATOM | 2576 | CE2 | TYR | 340 | −15.317 | −49.277 | 26.624 | 1.00 | 14.66 |
| ATOM | 2577 | CZ | TYR | 340 | −16.531 | −49.899 | 26.881 | 1.00 | 17.75 |
| ATOM | 2578 | OH | TYR | 340 | −17.382 | −50.238 | 25.850 | 1.00 | 20.56 |
| ATOM | 2579 | N | TYR | 341 | −10.867 | −48.871 | 29.794 | 1.00 | 17.56 |
| ATOM | 2580 | CA | TYR | 341 | −9.662 | −48.635 | 28.994 | 1.00 | 17.64 |
| ATOM | 2581 | C | TYR | 341 | −8.904 | −49.958 | 28.835 | 1.00 | 18.05 |
| ATOM | 2582 | O | TYR | 341 | −8.582 | −50.382 | 27.707 | 1.00 | 17.79 |
| ATOM | 2583 | CB | TYR | 341 | −8.790 | −47.617 | 29.737 | 1.00 | 16.64 |
| ATOM | 2584 | CG | TYR | 341 | −7.617 | −47.009 | 29.008 | 1.00 | 16.23 |
| ATOM | 2585 | CD1 | TYR | 341 | −6.896 | −45.969 | 29.609 | 1.00 | 13.34 |
| ATOM | 2586 | CD2 | TYR | 341 | −7.212 | −47.443 | 27.723 | 1.00 | 14.37 |
| ATOM | 2587 | CE1 | TYR | 341 | −5.791 | −45.392 | 28.976 | 1.00 | 14.65 |
| ATOM | 2588 | CE2 | TYR | 341 | −6.099 | −46.845 | 27.070 | 1.00 | 14.83 |
| ATOM | 2589 | CZ | TYR | 341 | −5.393 | −45.825 | 27.721 | 1.00 | 14.30 |
| ATOM | 2590 | OH | TYR | 341 | −4.324 | −45.185 | 27.110 | 1.00 | 14.94 |
| ATOM | 2591 | N | PHE | 342 | −8.638 | −50.606 | 29.970 | 1.00 | 18.06 |
| ATOM | 2592 | CA | PHE | 342 | −7.768 | −51.803 | 29.995 | 1.00 | 18.45 |
| ATOM | 2593 | C | PHE | 342 | −8.496 | −53.104 | 29.698 | 1.00 | 17.79 |
| ATOM | 2594 | O | PHE | 342 | −7.958 | −53.943 | 28.976 | 1.00 | 16.77 |
| ATOM | 2595 | CB | PHE | 342 | −6.937 | −51.886 | 31.300 | 1.00 | 18.12 |
| ATOM | 2596 | CG | PHE | 342 | −5.770 | −50.942 | 31.311 | 1.00 | 18.93 |
| ATOM | 2597 | CD1 | PHE | 342 | −4.495 | −51.383 | 30.959 | 1.00 | 20.62 |
| ATOM | 2598 | CD2 | PHE | 342 | −5.957 | −49.588 | 31.614 | 1.00 | 20.32 |
| ATOM | 2599 | CE1 | PHE | 342 | −3.402 | −50.486 | 30.925 | 1.00 | 20.55 |
| ATOM | 2600 | CE2 | PHE | 342 | −4.882 | −48.688 | 31.578 | 1.00 | 19.90 |
| ATOM | 2601 | CZ | PHE | 342 | −3.597 | −49.143 | 31.249 | 1.00 | 18.78 |
| ATOM | 2602 | N | ASP | 343 | −9.704 | −53.262 | 30.244 | 1.00 | 17.74 |
| ATOM | 2603 | CA | ASP | 343 | −10.477 | −54.513 | 30.081 | 1.00 | 18.86 |
| ATOM | 2604 | C | ASP | 343 | −11.251 | −54.626 | 28.758 | 1.00 | 18.75 |
| ATOM | 2605 | O | ASP | 343 | −11.519 | −55.746 | 28.292 | 1.00 | 18.42 |
| ATOM | 2606 | CB | ASP | 343 | −11.447 | −54.739 | 31.248 | 1.00 | 18.34 |
| ATOM | 2607 | CG | ASP | 343 | −10.743 | −54.751 | 32.604 | 1.00 | 21.41 |
| ATOM | 2608 | OD1 | ASP | 343 | −9.516 | −55.003 | 32.647 | 1.00 | 22.03 |
| ATOM | 2609 | OD2 | ASP | 343 | −11.415 | −54.480 | 33.623 | 1.00 | 21.49 |
| ATOM | 2610 | N | VAL | 344 | −11.619 | −53.474 | 28.173 | 1.00 | 18.81 |
| ATOM | 2611 | CA | VAL | 344 | −12.355 | −53.437 | 26.883 | 1.00 | 17.56 |
| ATOM | 2612 | C | VAL | 344 | −11.544 | −52.774 | 25.757 | 1.00 | 17.54 |
| ATOM | 2613 | O | VAL | 344 | −11.083 | −53.450 | 24.826 | 1.00 | 17.43 |
| ATOM | 2614 | CB | VAL | 344 | −13.746 | −52.758 | 27.020 | 1.00 | 17.47 |
| ATOM | 2615 | CG1 | VAL | 344 | −14.505 | −52.839 | 25.680 | 1.00 | 16.40 |
| ATOM | 2616 | CG2 | VAL | 344 | −14.578 | −53.414 | 28.173 | 1.00 | 16.80 |
| ATOM | 2617 | N | SER | 345 | −11.352 | −51.459 | 25.846 | 1.00 | 16.22 |
| ATOM | 2618 | CA | SER | 345 | −10.757 | −50.708 | 24.741 | 1.00 | 16.60 |
| ATOM | 2619 | C | SER | 345 | −9.457 | −51.295 | 24.192 | 1.00 | 16.10 |
| ATOM | 2620 | O | SER | 345 | −9.297 | −51.412 | 22.978 | 1.00 | 15.51 |
| ATOM | 2621 | CB | SER | 345 | −10.563 | −49.242 | 25.129 | 1.00 | 15.73 |
| ATOM | 2622 | OG | SER | 345 | −11.712 | −48.791 | 25.806 | 1.00 | 17.03 |
| ATOM | 2623 | N | ASN | 346 | −8.538 | −51.656 | 25.084 | 1.00 | 16.61 |
| ATOM | 2624 | CA | ASN | 346 | −7.213 | −52.153 | 24.662 | 1.00 | 17.90 |
| ATOM | 2625 | C | ASN | 346 | −7.282 | −53.542 | 24.003 | 1.00 | 17.47 |
| ATOM | 2626 | O | ASN | 346 | −6.323 | −53.980 | 23.353 | 1.00 | 17.26 |
| ATOM | 2627 | CB | ASN | 346 | −6.182 | −52.122 | 25.819 | 1.00 | 17.63 |
| ATOM | 2628 | CG | ASN | 346 | −5.690 | −50.697 | 26.155 | 1.00 | 20.65 |
| ATOM | 2629 | OD1 | ASN | 346 | −6.118 | −49.699 | 25.512 | 1.00 | 20.52 |
| ATOM | 2630 | ND2 | ASN | 346 | −4.861 | −50.563 | 27.103 | 1.00 | 24.61 |
| ATOM | 2631 | N | LYS | 347 | −8.432 | −54.198 | 24.138 | 1.00 | 18.34 |
| ATOM | 2632 | CA | LYS | 347 | −8.700 | −55.488 | 23.472 | 1.00 | 19.60 |
| ATOM | 2633 | C | LYS | 347 | −9.493 | −55.319 | 22.165 | 1.00 | 19.78 |
| ATOM | 2634 | O | LYS | 347 | −9.765 | −56.307 | 21.451 | 1.00 | 18.92 |
| ATOM | 2635 | CB | LYS | 347 | −9.413 | −56.441 | 24.432 | 1.00 | 19.47 |
| ATOM | 2636 | CG | LYS | 347 | −8.551 | −56.782 | 25.637 | 1.00 | 22.73 |
| ATOM | 2637 | CD | LYS | 347 | −9.220 | −57.821 | 26.532 | 1.00 | 27.80 |
| ATOM | 2638 | CE | LYS | 347 | −8.775 | −57.679 | 27.993 | 1.00 | 30.84 |
| ATOM | 2639 | NZ | LYS | 347 | −9.881 | −58.068 | 28.960 | 1.00 | 31.21 |
| ATOM | 2640 | N | VAL | 348 | −9.863 | −54.073 | 21.855 | 1.00 | 19.81 |
| ATOM | 2641 | CA | VAL | 348 | −10.517 | −53.762 | 20.579 | 1.00 | 19.69 |
| ATOM | 2642 | C | VAL | 348 | −9.443 | −53.565 | 19.517 | 1.00 | 19.55 |
| ATOM | 2643 | O | VAL | 348 | −8.737 | −52.546 | 19.506 | 1.00 | 19.60 |
| ATOM | 2644 | CB | VAL | 348 | −11.481 | −52.535 | 20.660 | 1.00 | 19.33 |
| ATOM | 2645 | CG1 | VAL | 348 | −12.101 | −52.256 | 19.299 | 1.00 | 20.07 |
| ATOM | 2646 | CG2 | VAL | 348 | −12.613 | −52.799 | 21.624 | 1.00 | 18.14 |
| ATOM | 2647 | N | LYS | 349 | −9.332 | −54.534 | 18.610 | 1.00 | 20.42 |
| ATOM | 2648 | CA | LYS | 349 | −8.227 | −54.555 | 17.641 | 1.00 | 21.51 |
| ATOM | 2649 | C | LYS | 349 | −8.658 | −54.572 | 16.179 | 1.00 | 21.53 |
| ATOM | 2650 | O | LYS | 349 | −7.806 | −54.470 | 15.268 | 1.00 | 22.21 |
| ATOM | 2651 | CB | LYS | 349 | −7.310 | −55.766 | 17.909 | 1.00 | 22.48 |
| ATOM | 2652 | CG | LYS | 349 | −6.811 | −55.901 | 19.371 | 1.00 | 25.31 |
| ATOM | 2653 | CD | LYS | 349 | −5.800 | −54.788 | 19.781 | 1.00 | 29.56 |
| ATOM | 2654 | CE | LYS | 349 | −4.467 | −54.898 | 19.010 | 1.00 | 33.30 |
| ATOM | 2655 | NZ | LYS | 349 | −3.447 | −53.881 | 19.445 | 1.00 | 35.42 |
| ATOM | 2656 | N | ASP | 350 | −9.956 | −54.726 | 15.941 | 1.00 | 20.95 |
| ATOM | 2657 | CA | ASP | 350 | −10.467 | −54.838 | 14.585 | 1.00 | 20.99 |
| ATOM | 2658 | C | ASP | 350 | −10.606 | −53.498 | 13.834 | 1.00 | 20.63 |
| ATOM | 2659 | O | ASP | 350 | −10.867 | −53.481 | 12.614 | 1.00 | 19.98 |
| ATOM | 2660 | CB | ASP | 350 | −11.779 | −55.624 | 14.551 | 1.00 | 21.62 |
| ATOM | 2661 | CG | ASP | 350 | −12.892 | −54.969 | 15.340 | 1.00 | 25.12 |
| ATOM | 2662 | OD1 | ASP | 350 | −12.654 | −54.398 | 16.431 | 1.00 | 26.89 |
| ATOM | 2663 | OD2 | ASP | 350 | −14.048 | −55.053 | 14.869 | 1.00 | 31.22 |
| ATOM | 2664 | N | ARG | 351 | −10.419 | −52.378 | 14.536 | 1.00 | 18.43 |
| ATOM | 2665 | CA | ARG | 351 | −10.384 | −51.089 | 13.849 | 1.00 | 17.60 |
| ATOM | 2666 | C | ARG | 351 | −9.583 | −50.068 | 14.680 | 1.00 | 17.58 |
| ATOM | 2667 | O | ARG | 351 | −9.317 | −50.311 | 15.857 | 1.00 | 17.77 |
| ATOM | 2668 | CB | ARG | 351 | −11.818 | −50.598 | 13.564 | 1.00 | 16.24 |
| ATOM | 2669 | CG | ARG | 351 | −12.548 | −50.049 | 14.791 | 1.00 | 14.57 |
| ATOM | 2670 | CD | ARG | 351 | −13.017 | −51.136 | 15.754 | 1.00 | 12.79 |
| ATOM | 2671 | NE | ARG | 351 | −14.150 | −50.637 | 16.525 | 1.00 | 12.02 |
| ATOM | 2672 | CZ | ARG | 351 | −14.989 | −51.384 | 17.223 | 1.00 | 12.84 |
| ATOM | 2673 | NH1 | ARG | 351 | −14.848 | −52.718 | 17.245 | 1.00 | 14.46 |
| ATOM | 2674 | NH2 | ARG | 351 | −15.994 | −50.798 | 17.863 | 1.00 | 10.10 |
| ATOM | 2675 | N | PHE | 352 | −9.209 | −48.946 | 14.059 | 1.00 | 17.17 |
| ATOM | 2676 | CA | PHE | 352 | −8.533 | −47.855 | 14.752 | 1.00 | 17.03 |
| ATOM | 2677 | C | PHE | 352 | −9.502 | −47.125 | 15.710 | 1.00 | 16.90 |
| ATOM | 2678 | O | PHE | 352 | −10.671 | −46.867 | 15.371 | 1.00 | 17.53 |
| ATOM | 2679 | CB | PHE | 352 | −7.970 | −46.883 | 13.720 | 1.00 | 16.90 |
| ATOM | 2680 | CG | PHE | 352 | −7.295 | −45.681 | 14.311 | 1.00 | 17.08 |
| ATOM | 2681 | CD1 | PHE | 352 | −6.133 | −45.814 | 15.057 | 1.00 | 16.63 |
| ATOM | 2682 | CD2 | PHE | 352 | −7.842 | −44.404 | 14.129 | 1.00 | 18.31 |
| ATOM | 2683 | CE1 | PHE | 352 | −5.520 | −44.691 | 15.620 | 1.00 | 18.51 |
| ATOM | 2684 | CE2 | PHE | 352 | −7.241 | −43.273 | 14.677 | 1.00 | 17.34 |
| ATOM | 2685 | CZ | PHE | 352 | −6.069 | −43.418 | 15.419 | 1.00 | 18.25 |
| ATOM | 2686 | N | TRP | 353 | −9.029 | −46.812 | 16.909 | 1.00 | 15.84 |
| ATOM | 2687 | CA | TRP | 353 | −9.785 | −45.954 | 17.809 | 1.00 | 15.48 |
| ATOM | 2688 | C | TRP | 353 | −8.797 | −45.055 | 18.550 | 1.00 | 15.40 |
| ATOM | 2689 | O | TRP | 353 | −7.622 | −45.414 | 18.696 | 1.00 | 15.26 |
| ATOM | 2690 | CB | TRP | 353 | −10.682 | −46.751 | 18.784 | 1.00 | 14.67 |
| ATOM | 2691 | CG | TRP | 353 | −9.925 | −47.675 | 19.747 | 1.00 | 16.38 |
| ATOM | 2692 | CD1 | TRP | 353 | −9.779 | −49.046 | 19.640 | 1.00 | 16.68 |
| ATOM | 2693 | CD2 | TRP | 353 | −9.221 | −47.298 | 20.947 | 1.00 | 15.93 |
| ATOM | 2694 | CE2 | TRP | 353 | −8.673 | −48.484 | 21.504 | 1.00 | 16.91 |
| ATOM | 2695 | CE3 | TRP | 353 | −8.999 | −46.072 | 21.609 | 1.00 | 17.37 |
| ATOM | 2696 | NE1 | TRP | 353 | −9.036 | −49.526 | 20.689 | 1.00 | 15.78 |
| ATOM | 2697 | CZ2 | TRP | 353 | −7.916 | −48.484 | 22.707 | 1.00 | 14.94 |
| ATOM | 2698 | CZ3 | TRP | 353 | −8.242 | −46.063 | 22.783 | 1.00 | 14.81 |
| ATOM | 2699 | CH2 | TRP | 353 | −7.695 | −47.263 | 23.312 | 1.00 | 16.53 |
| ATOM | 2700 | N | PHE | 354 | −9.278 | −43.895 | 19.012 | 1.00 | 14.00 |
| ATOM | 2701 | CA | PHE | 354 | −8.534 | −43.076 | 19.958 | 1.00 | 13.32 |
| ATOM | 2702 | C | PHE | 354 | −9.459 | −42.388 | 20.977 | 1.00 | 12.98 |
| ATOM | 2703 | O | PHE | 354 | −10.676 | −42.335 | 20.789 | 1.00 | 13.13 |
| ATOM | 2704 | CB | PHE | 354 | −7.619 | −42.074 | 19.229 | 1.00 | 11.99 |
| ATOM | 2705 | CG | PHE | 354 | −8.352 | −40.961 | 18.518 | 1.00 | 14.22 |
| ATOM | 2706 | CD1 | PHE | 354 | −8.853 | −39.864 | 19.230 | 1.00 | 13.54 |
| ATOM | 2707 | CD2 | PHE | 354 | −8.510 | −41.000 | 17.117 | 1.00 | 15.54 |
| ATOM | 2708 | CE1 | PHE | 354 | −9.523 | −38.812 | 18.554 | 1.00 | 13.38 |
| ATOM | 2709 | CE2 | PHE | 354 | −9.148 | −39.959 | 16.426 | 1.00 | 15.22 |
| ATOM | 2710 | CZ | PHE | 354 | −9.666 | −38.870 | 17.142 | 1.00 | 16.28 |
| ATOM | 2711 | N | TYR | 355 | −8.875 | −41.906 | 22.070 | 1.00 | 12.47 |
| ATOM | 2712 | CA | TYR | 355 | −9.502 | −40.870 | 22.886 | 1.00 | 12.82 |
| ATOM | 2713 | C | TYR | 355 | −8.495 | −39.724 | 22.982 | 1.00 | 12.86 |
| ATOM | 2714 | O | TYR | 355 | −7.285 | −39.923 | 22.803 | 1.00 | 13.35 |
| ATOM | 2715 | CB | TYR | 355 | −9.880 | −41.360 | 24.298 | 1.00 | 12.05 |
| ATOM | 2716 | CG | TYR | 355 | −8.666 | −41.679 | 25.153 | 1.00 | 13.87 |
| ATOM | 2717 | CD1 | TYR | 355 | −7.968 | −40.668 | 25.811 | 1.00 | 12.84 |
| ATOM | 2718 | CD2 | TYR | 355 | −8.209 | −43.004 | 25.295 | 1.00 | 13.19 |
| ATOM | 2719 | CE1 | TYR | 355 | −6.860 | −40.955 | 26.580 | 1.00 | 14.06 |
| ATOM | 2720 | CE2 | TYR | 355 | −7.106 | −43.297 | 26.061 | 1.00 | 12.10 |
| ATOM | 2721 | CZ | TYR | 355 | −6.434 | −42.266 | 26.695 | 1.00 | 14.05 |
| ATOM | 2722 | OH | TYR | 355 | −5.319 | −42.523 | 27.454 | 1.00 | 15.44 |
| ATOM | 2723 | N | GLN | 356 | −9.010 | −38.528 | 23.252 | 1.00 | 13.22 |
| ATOM | 2724 | CA | GLN | 356 | −8.202 | −37.349 | 23.588 | 1.00 | 13.19 |
| ATOM | 2725 | C | GLN | 356 | −8.803 | −36.729 | 24.818 | 1.00 | 13.00 |
| ATOM | 2726 | O | GLN | 356 | −10.007 | −36.485 | 24.854 | 1.00 | 14.64 |
| ATOM | 2727 | CB | GLN | 356 | −8.196 | −36.322 | 22.447 | 1.00 | 12.53 |
| ATOM | 2728 | CG | GLN | 356 | −7.545 | −36.879 | 21.210 | 1.00 | 13.51 |
| ATOM | 2729 | CD | GLN | 356 | −7.694 | −36.022 | 19.989 | 1.00 | 14.74 |
| ATOM | 2730 | NE2 | GLN | 356 | −8.838 | −35.374 | 19.862 | 1.00 | 17.33 |
| ATOM | 2731 | OE1 | GLN | 356 | −6.798 | −35.968 | 19.141 | 1.00 | 16.64 |
| ATOM | 2732 | N | LEU | 357 | −7.970 | −36.505 | 25.832 | 1.00 | 12.75 |
| ATOM | 2733 | CA | LEU | 357 | −8.359 | −35.795 | 27.031 | 1.00 | 12.42 |
| ATOM | 2734 | C | LEU | 357 | −7.709 | −34.401 | 26.970 | 1.00 | 11.95 |
| ATOM | 2735 | O | LEU | 357 | −6.517 | −34.237 | 27.342 | 1.00 | 11.03 |
| ATOM | 2736 | CB | LEU | 357 | −7.949 | −36.582 | 28.273 | 1.00 | 12.76 |
| ATOM | 2737 | CG | LEU | 357 | −8.304 | −38.085 | 28.424 | 1.00 | 14.85 |
| ATOM | 2738 | CD1 | LEU | 357 | −7.947 | −38.646 | 29.812 | 1.00 | 15.76 |
| ATOM | 2739 | CD2 | LEU | 357 | −9.749 | −38.414 | 28.132 | 1.00 | 14.99 |
| ATOM | 2740 | N | ASP | 358 | −8.492 | −33.430 | 26.462 | 1.00 | 10.91 |
| ATOM | 2741 | CA | ASP | 358 | −7.998 | −32.066 | 26.268 | 0.50 | 11.35 |
| ATOM | 2742 | C | ASP | 358 | −8.161 | −31.221 | 27.521 | 1.00 | 10.91 |
| ATOM | 2743 | O | ASP | 358 | −9.205 | −31.228 | 28.155 | 1.00 | 9.76 |
| ATOM | 2744 | CB | ASP | 358 | −8.667 | −31.381 | 25.070 | 0.50 | 11.24 |
| ATOM | 2745 | CG | ASP | 358 | −8.131 | −31.891 | 23.746 | 0.50 | 13.18 |
| ATOM | 2746 | OD1 | ASP | 358 | −8.538 | −32.998 | 23.345 | 0.50 | 13.80 |
| ATOM | 2747 | OD2 | ASP | 358 | −7.282 | −31.209 | 23.122 | 0.50 | 14.13 |
| ATOM | 2748 | N | VAL | 359 | −7.094 | −30.502 | 27.861 | 1.00 | 11.16 |
| ATOM | 2749 | CA | VAL | 359 | −7.092 | −29.594 | 28.994 | 1.00 | 11.71 |
| ATOM | 2750 | C | VAL | 359 | −7.796 | −28.292 | 28.568 | 1.00 | 11.96 |
| ATOM | 2751 | O | VAL | 359 | −7.180 | −27.342 | 28.038 | 1.00 | 13.09 |
| ATOM | 2752 | CB | VAL | 359 | −5.652 | −29.389 | 29.518 | 1.00 | 12.42 |
| ATOM | 2753 | CG1 | VAL | 359 | −5.598 | −28.310 | 30.590 | 1.00 | 12.22 |
| ATOM | 2754 | CG2 | VAL | 359 | −5.083 | −30.739 | 30.061 | 1.00 | 13.89 |
| ATOM | 2755 | N | HIS | 360 | −9.098 | −28.271 | 28.816 | 1.00 | 11.43 |
| ATOM | 2756 | CA | HIS | 360 | −10.028 | −27.239 | 28.326 | 1.00 | 12.34 |
| ATOM | 2757 | C | HIS | 360 | −10.136 | −26.104 | 29.342 | 1.00 | 12.60 |
| ATOM | 2758 | O | HIS | 360 | −9.976 | −24.939 | 29.010 | 1.00 | 13.20 |
| ATOM | 2759 | CB | HIS | 360 | −11.388 | −27.945 | 28.070 | 1.00 | 12.18 |
| ATOM | 2760 | CG | HIS | 360 | −12.528 | −27.049 | 27.682 | 1.00 | 13.03 |
| ATOM | 2761 | CD2 | HIS | 360 | −13.002 | −26.689 | 26.466 | 1.00 | 12.43 |
| ATOM | 2762 | ND1 | HIS | 360 | −13.415 | −26.524 | 28.605 | 1.00 | 12.00 |
| ATOM | 2763 | CE1 | HIS | 360 | −14.348 | −25.831 | 27.976 | 1.00 | 10.46 |
| ATOM | 2764 | NE2 | HIS | 360 | −14.131 | −25.928 | 26.677 | 1.00 | 13.68 |
| ATOM | 2765 | N | GLY | 361 | −10.376 | −26.453 | 30.603 | 1.00 | 13.50 |
| ATOM | 2766 | CA | GLY | 361 | −10.531 | −25.461 | 31.634 | 1.00 | 13.51 |
| ATOM | 2767 | C | GLY | 361 | −9.319 | −25.433 | 32.545 | 1.00 | 14.55 |
| ATOM | 2768 | O | GLY | 361 | −8.200 | −25.796 | 32.132 | 1.00 | 14.04 |
| ATOM | 2769 | N | GLY | 362 | −9.531 | −25.002 | 33.786 | 1.00 | 14.47 |
| ATOM | 2770 | CA | GLY | 362 | −8.424 | −24.840 | 34.725 | 1.00 | 15.30 |
| ATOM | 2771 | C | GLY | 362 | −8.171 | −23.372 | 35.006 | 1.00 | 15.57 |
| ATOM | 2772 | O | GLY | 362 | −8.582 | −22.488 | 34.231 | 1.00 | 14.90 |
| ATOM | 2773 | N | LYS | 363 | −7.480 | −23.114 | 36.117 | 1.00 | 16.86 |
| ATOM | 2774 | CA | LYS | 363 | −7.234 | −21.753 | 36.612 | 1.00 | 17.26 |
| ATOM | 2775 | C | LYS | 363 | −6.719 | −20.776 | 35.547 | 1.00 | 16.48 |
| ATOM | 2776 | O | LYS | 363 | −7.255 | −19.676 | 35.412 | 1.00 | 16.24 |
| ATOM | 2777 | CB | LYS | 363 | −6.253 | −21.777 | 37.799 | 1.00 | 17.84 |
| ATOM | 2778 | CG | LYS | 363 | −6.385 | −20.562 | 38.706 | 1.00 | 22.62 |
| ATOM | 2779 | CD | LYS | 363 | −5.034 | −19.941 | 39.020 | 1.00 | 29.97 |
| ATOM | 2780 | CE | LYS | 363 | −4.544 | −20.329 | 40.390 | 1.00 | 34.20 |
| ATOM | 2781 | NZ | LYS | 363 | −3.301 | −19.558 | 40.713 | 1.00 | 37.00 |
| ATOM | 2782 | N | ASN | 364 | −5.678 | −21.158 | 34.808 | 1.00 | 15.76 |
| ATOM | 2783 | CA | ASN | 364 | −5.061 | −20.221 | 33.846 | 1.00 | 15.36 |
| ATOM | 2784 | C | ASN | 364 | −5.504 | −20.425 | 32.385 | 1.00 | 14.73 |
| ATOM | 2785 | O | ASN | 364 | −4.915 | −19.852 | 31.450 | 1.00 | 14.04 |
| ATOM | 2786 | CB | ASN | 364 | −3.532 | −20.249 | 33.983 | 1.00 | 15.66 |
| ATOM | 2787 | CG | ASN | 364 | −3.068 | −20.055 | 35.444 | 1.00 | 18.27 |
| ATOM | 2788 | ND2 | ASN | 364 | −3.463 | −18.949 | 36.024 | 1.00 | 15.36 |
| ATOM | 2789 | OD1 | ASN | 364 | −2.403 | −20.926 | 36.040 | 1.00 | 20.32 |
| ATOM | 2790 | N | SER | 365 | −6.551 | −21.230 | 32.178 | 1.00 | 14.32 |
| ATOM | 2791 | CA | SER | 365 | −7.056 | −21.450 | 30.819 | 1.00 | 13.40 |
| ATOM | 2792 | C | SER | 365 | −7.825 | −20.229 | 30.332 | 1.00 | 13.75 |
| ATOM | 2793 | O | SER | 365 | −8.803 | −19.807 | 30.953 | 1.00 | 12.93 |
| ATOM | 2794 | CB | SER | 365 | −7.937 | −22.694 | 30.717 | 1.00 | 13.17 |
| ATOM | 2795 | OG | SER | 365 | −8.584 | −22.721 | 29.445 | 1.00 | 12.63 |
| ATOM | 2796 | N | GLN | 366 | −7.387 | −19.660 | 29.212 | 1.00 | 13.86 |
| ATOM | 2797 | CA | GLN | 366 | −8.109 | −18.533 | 28.609 | 1.00 | 14.88 |
| ATOM | 2798 | C | GLN | 366 | −9.583 | −18.870 | 28.220 | 1.00 | 14.85 |
| ATOM | 2799 | O | GLN | 366 | −10.473 | −18.002 | 28.219 | 1.00 | 14.73 |
| ATOM | 2800 | CB | GLN | 366 | −7.302 | −18.044 | 27.400 | 1.00 | 15.06 |
| ATOM | 2801 | CG | GLN | 366 | −7.590 | −16.627 | 26.970 | 1.00 | 17.29 |
| ATOM | 2802 | CD | GLN | 366 | −7.479 | −15.549 | 28.058 | 1.00 | 17.87 |
| ATOM | 2803 | NE2 | GLN | 366 | −6.605 | −15.713 | 29.050 | 1.00 | 18.06 |
| ATOM | 2804 | OE1 | GLN | 366 | −8.179 | −14.563 | 27.965 | 1.00 | 21.64 |
| ATOM | 2805 | N | VAL | 367 | −9.844 | −20.136 | 27.912 | 1.00 | 14.22 |
| ATOM | 2806 | CA | VAL | 367 | −11.206 | −20.572 | 27.606 | 1.00 | 14.91 |
| ATOM | 2807 | C | VAL | 367 | −12.181 | −20.209 | 28.743 | 1.00 | 15.85 |
| ATOM | 2808 | O | VAL | 367 | −13.340 | −19.828 | 28.483 | 1.00 | 15.18 |
| ATOM | 2809 | CB | VAL | 367 | −11.282 | −22.116 | 27.355 | 1.00 | 14.70 |
| ATOM | 2810 | CG1 | VAL | 367 | −12.759 | −22.580 | 27.251 | 1.00 | 13.77 |
| ATOM | 2811 | CG2 | VAL | 367 | −10.473 | −22.490 | 26.112 | 1.00 | 12.87 |
| ATOM | 2812 | N | THR | 368 | −11.678 | −20.291 | 29.979 | 1.00 | 16.97 |
| ATOM | 2813 | CA | THR | 368 | −12.472 | −20.107 | 31.225 | 1.00 | 18.53 |
| ATOM | 2814 | C | THR | 368 | −12.628 | −18.651 | 31.643 | 1.00 | 19.63 |
| ATOM | 2815 | O | THR | 368 | −13.341 | −18.365 | 32.599 | 1.00 | 21.06 |
| ATOM | 2816 | CB | THR | 368 | −11.827 | −20.830 | 32.457 | 1.00 | 18.40 |
| ATOM | 2817 | CG2 | THR | 368 | −11.579 | −22.261 | 32.174 | 1.00 | 15.77 |
| ATOM | 2818 | OG1 | THR | 368 | −10.593 | −20.168 | 32.860 | 1.00 | 20.22 |
| ATOM | 2819 | N | LYS | 369 | −11.940 | −17.735 | 30.971 | 1.00 | 19.88 |
| ATOM | 2820 | CA | LYS | 369 | −12.105 | −16.312 | 31.265 | 1.00 | 20.83 |
| ATOM | 2821 | C | LYS | 369 | −13.411 | −15.736 | 30.706 | 1.00 | 21.27 |
| ATOM | 2822 | O | LYS | 369 | −13.775 | −14.615 | 31.043 | 1.00 | 22.93 |
| ATOM | 2823 | CB | LYS | 369 | −10.903 | −15.493 | 30.782 | 1.00 | 20.85 |
| ATOM | 2824 | CG | LYS | 369 | −9.533 | −16.022 | 31.263 | 1.00 | 22.39 |
| ATOM | 2825 | CD | LYS | 369 | −9.456 | −16.078 | 32.777 | 1.00 | 24.58 |
| ATOM | 2826 | CE | LYS | 369 | −8.077 | −16.483 | 33.249 | 1.00 | 25.45 |
| ATOM | 2827 | NZ | LYS | 369 | −8.143 | −16.901 | 34.655 | 1.00 | 29.52 |
| ATOM | 2828 | N | VAL | 370 | −14.111 | −16.484 | 29.842 | 1.00 | 20.74 |
| ATOM | 2829 | CA | VAL | 370 | −15.483 | −16.141 | 29.473 | 1.00 | 18.58 |
| ATOM | 2830 | C | VAL | 370 | −16.401 | −17.043 | 30.312 | 1.00 | 18.50 |
| ATOM | 2831 | O | VAL | 370 | −16.096 | −18.215 | 30.525 | 1.00 | 17.57 |
| ATOM | 2832 | CB | VAL | 370 | −15.731 | −16.341 | 27.969 | 1.00 | 19.15 |
| ATOM | 2833 | CG1 | VAL | 370 | −17.193 | −16.082 | 27.601 | 1.00 | 16.98 |
| ATOM | 2834 | CG2 | VAL | 370 | −14.780 | −15.438 | 27.118 | 1.00 | 17.89 |
| ATOM | 2835 | N | THR | 371 | −17.509 | −16.513 | 30.823 | 1.00 | 17.19 |
| ATOM | 2836 | CA | THR | 371 | −18.406 | −17.377 | 31.612 | 1.00 | 16.76 |
| ATOM | 2837 | C | THR | 371 | −19.503 | −18.009 | 30.740 | 1.00 | 15.98 |
| ATOM | 2838 | O | THR | 371 | −19.747 | −17.568 | 29.625 | 1.00 | 14.46 |
| ATOM | 2839 | CB | THR | 371 | −19.100 | −16.598 | 32.721 | 1.00 | 17.01 |
| ATOM | 2840 | CG2 | THR | 371 | −18.085 | −15.789 | 33.604 | 1.00 | 18.37 |
| ATOM | 2841 | OG1 | THR | 371 | −20.028 | −15.700 | 32.118 | 1.00 | 16.95 |
| ATOM | 2842 | N | ASN | 372 | −20.202 | −19.004 | 31.274 | 1.00 | 16.00 |
| ATOM | 2843 | CA | ASN | 372 | −21.346 | −19.578 | 30.562 | 1.00 | 17.04 |
| ATOM | 2844 | C | ASN | 372 | −22.460 | −18.609 | 30.185 | 1.00 | 17.16 |
| ATOM | 2845 | O | ASN | 372 | −23.173 | −18.856 | 29.215 | 1.00 | 17.85 |
| ATOM | 2846 | CB | ASN | 372 | −21.946 | −20.743 | 31.345 | 1.00 | 16.80 |
| ATOM | 2847 | CG | ASN | 372 | −21.181 | −22.031 | 31.134 | 1.00 | 18.34 |
| ATOM | 2848 | ND2 | ASN | 372 | −21.380 | −22.995 | 32.043 | 1.00 | 18.04 |
| ATOM | 2849 | OD1 | ASN | 372 | −20.431 | −22.173 | 30.154 | 1.00 | 17.79 |
| ATOM | 2850 | N | ALA | 373 | −22.628 | −17.536 | 30.959 | 1.00 | 16.66 |
| ATOM | 2851 | CA | ALA | 373 | −23.678 | −16.554 | 30.689 | 1.00 | 17.20 |
| ATOM | 2852 | C | ALA | 373 | −23.321 | −15.670 | 29.506 | 1.00 | 16.68 |
| ATOM | 2853 | O | ALA | 373 | −24.210 | −15.123 | 28.873 | 1.00 | 17.50 |
| ATOM | 2854 | CB | ALA | 373 | −23.956 | −15.665 | 31.958 | 1.00 | 17.47 |
| ATOM | 2855 | N | GLU | 374 | −22.027 | −15.543 | 29.202 | 1.00 | 15.97 |
| ATOM | 2856 | CA | GLU | 374 | −21.561 | −14.527 | 28.246 | 1.00 | 16.21 |
| ATOM | 2857 | C | GLU | 374 | −21.698 | −14.870 | 26.755 | 1.00 | 14.83 |
| ATOM | 2858 | O | GLU | 374 | −21.745 | −13.966 | 25.924 | 1.00 | 14.17 |
| ATOM | 2859 | CB | GLU | 374 | −20.130 | −14.052 | 28.579 | 1.00 | 16.16 |
| ATOM | 2860 | CG | GLU | 374 | −20.076 | −13.326 | 29.923 | 1.00 | 20.12 |
| ATOM | 2861 | CD | GLU | 374 | −18.697 | −12.801 | 30.244 | 1.00 | 24.04 |
| ATOM | 2862 | OE1 | GLU | 374 | −17.723 | −13.579 | 30.258 | 1.00 | 22.84 |
| ATOM | 2863 | OE2 | GLU | 374 | −18.592 | −11.586 | 30.479 | 1.00 | 29.97 |
| ATOM | 2864 | N | THR | 375 | −21.759 | −16.168 | 26.436 | 1.00 | 14.07 |
| ATOM | 2865 | CA | THR | 375 | −22.007 | −16.648 | 25.072 | 1.00 | 12.21 |
| ATOM | 2866 | C | THR | 375 | −22.834 | −17.931 | 25.171 | 1.00 | 12.32 |
| ATOM | 2867 | O | THR | 375 | −23.018 | −18.478 | 26.266 | 1.00 | 12.40 |
| ATOM | 2868 | CB | THR | 375 | −20.684 | −16.939 | 24.274 | 1.00 | 11.82 |
| ATOM | 2869 | CG2 | THR | 375 | −19.657 | −15.836 | 24.415 | 1.00 | 11.08 |
| ATOM | 2870 | OG1 | THR | 375 | −20.095 | −18.167 | 24.726 | 1.00 | 11.57 |
| ATOM | 2871 | N | ALA | 376 | −23.372 | −18.398 | 24.043 | 1.00 | 11.90 |
| ATOM | 2872 | CA | ALA | 376 | −24.139 | −19.648 | 24.020 | 1.00 | 11.74 |
| ATOM | 2873 | C | ALA | 376 | −23.307 | −20.865 | 24.476 | 1.00 | 11.41 |
| ATOM | 2874 | O | ALA | 376 | −23.867 | −21.849 | 25.004 | 1.00 | 11.17 |
| ATOM | 2875 | CB | ALA | 376 | −24.700 | −19.901 | 22.602 | 1.00 | 11.70 |
| ATOM | 2876 | N | TYR | 377 | −21.987 | −20.799 | 24.253 | 1.00 | 10.95 |
| ATOM | 2877 | CA | TYR | 377 | −21.052 | −21.884 | 24.630 | 1.00 | 11.04 |
| ATOM | 2878 | C | TYR | 377 | −21.219 | −22.243 | 26.120 | 1.00 | 10.90 |
| ATOM | 2879 | O | TYR | 377 | −21.009 | −21.379 | 26.984 | 1.00 | 11.00 |
| ATOM | 2880 | CB | TYR | 377 | −19.611 | −21.480 | 24.338 | 1.00 | 10.52 |
| ATOM | 2881 | CG | TYR | 377 | −18.642 | −22.643 | 24.423 | 1.00 | 11.61 |
| ATOM | 2882 | CD1 | TYR | 377 | −18.511 | −23.548 | 23.364 | 1.00 | 10.44 |
| ATOM | 2883 | CD2 | TYR | 377 | −17.878 | −22.857 | 25.581 | 1.00 | 12.22 |
| ATOM | 2884 | CE1 | TYR | 377 | −17.617 | −24.622 | 23.442 | 1.00 | 11.51 |
| ATOM | 2885 | CE2 | TYR | 377 | −17.001 | −23.916 | 25.675 | 1.00 | 11.24 |
| ATOM | 2886 | CZ | TYR | 377 | −16.870 | −24.800 | 24.602 | 1.00 | 11.89 |
| ATOM | 2887 | OH | TYR | 377 | −15.988 | −25.854 | 24.696 | 1.00 | 10.63 |
| ATOM | 2888 | N | PRO | 378 | −21.621 | −23.498 | 26.414 | 1.00 | 10.59 |
| ATOM | 2889 | CA | PRO | 378 | −22.058 | −23.871 | 27.770 | 1.00 | 11.00 |
| ATOM | 2890 | C | PRO | 378 | −21.028 | −24.636 | 28.594 | 1.00 | 11.65 |
| ATOM | 2891 | O | PRO | 378 | −21.357 | −25.118 | 29.697 | 1.00 | 12.15 |
| ATOM | 2892 | CB | PRO | 378 | −23.217 | −24.825 | 27.480 | 1.00 | 10.96 |
| ATOM | 2893 | CG | PRO | 378 | −22.728 | −25.578 | 26.239 | 1.00 | 10.06 |
| ATOM | 2894 | CD | PRO | 378 | −21.839 | −24.602 | 25.463 | 1.00 | 9.79 |
| ATOM | 2895 | N | HIS | 379 | −19.806 | −24.783 | 28.083 | 1.00 | 10.90 |
| ATOM | 2896 | CA | HIS | 379 | −18.853 | −25.651 | 28.776 | 1.00 | 10.77 |
| ATOM | 2897 | C | HIS | 379 | −17.766 | −24.880 | 29.536 | 1.00 | 11.25 |
| ATOM | 2898 | O | HIS | 379 | −16.684 | −25.419 | 29.785 | 1.00 | 10.54 |
| ATOM | 2899 | CB | HIS | 379 | −18.199 | −26.623 | 27.792 | 1.00 | 10.35 |
| ATOM | 2900 | CG | HIS | 379 | −19.158 | −27.312 | 26.881 | 1.00 | 9.93 |
| ATOM | 2901 | CD2 | HIS | 379 | −19.324 | −27.230 | 25.540 | 1.00 | 9.52 |
| ATOM | 2902 | ND1 | HIS | 379 | −20.071 | −28.244 | 27.328 | 1.00 | 10.65 |
| ATOM | 2903 | CE1 | HIS | 379 | −20.765 | −28.704 | 26.300 | 1.00 | 10.75 |
| ATOM | 2904 | NE2 | HIS | 379 | −20.325 | −28.112 | 25.200 | 1.00 | 12.28 |
| ATOM | 2905 | N | ARG | 380 | −18.024 | −23.617 | 29.879 | 1.00 | 11.81 |
| ATOM | 2906 | CA | ARG | 380 | −16.962 | −22.805 | 30.524 | 1.00 | 12.62 |
| ATOM | 2907 | C | ARG | 380 | −16.551 | −23.315 | 31.927 | 1.00 | 13.59 |
| ATOM | 2908 | O | ARG | 380 | −15.454 | −22.985 | 32.401 | 1.00 | 14.20 |
| ATOM | 2909 | CB | ARG | 380 | −17.358 | −21.320 | 30.595 | 1.00 | 11.97 |
| ATOM | 2910 | CG | ARG | 380 | −17.756 | −20.720 | 29.255 | 1.00 | 11.19 |
| ATOM | 2911 | CD | ARG | 380 | −16.563 | −20.569 | 28.259 | 1.00 | 10.22 |
| ATOM | 2912 | NE | ARG | 380 | −17.053 | −19.872 | 27.075 | 1.00 | 10.08 |
| ATOM | 2913 | CZ | ARG | 380 | −16.323 | −19.370 | 26.075 | 1.00 | 11.70 |
| ATOM | 2914 | NH1 | ARG | 380 | −14.983 | −19.426 | 26.071 | 1.00 | 9.55 |
| ATOM | 2915 | NH2 | ARG | 380 | −16.970 | −18.792 | 25.056 | 1.00 | 9.82 |
| ATOM | 2916 | N | ASP | 381 | −17.434 | −24.108 | 32.564 | 1.00 | 14.27 |
| ATOM | 2917 | CA | ASP | 381 | −17.225 | −24.667 | 33.899 | 1.00 | 14.62 |
| ATOM | 2918 | C | ASP | 381 | −16.708 | −26.102 | 33.815 | 1.00 | 14.71 |
| ATOM | 2919 | O | ASP | 381 | −16.773 | −26.854 | 34.783 | 1.00 | 14.74 |
| ATOM | 2920 | CB | ASP | 381 | −18.541 | −24.607 | 34.726 | 1.00 | 15.13 |
| ATOM | 2921 | CG | ASP | 381 | −19.703 | −25.363 | 34.064 | 1.00 | 16.79 |
| ATOM | 2922 | OD1 | ASP | 381 | −19.594 | −25.748 | 32.861 | 1.00 | 18.70 |
| ATOM | 2923 | OD2 | ASP | 381 | −20.741 | −25.588 | 34.740 | 1.00 | 18.06 |
| ATOM | 2924 | N | LYS | 382 | −16.225 | −26.499 | 32.639 | 1.00 | 14.45 |
| ATOM | 2925 | CA | LYS | 382 | −15.716 | −27.867 | 32.433 | 1.00 | 14.55 |
| ATOM | 2926 | C | LYS | 382 | −14.212 | −27.847 | 32.219 | 1.00 | 14.32 |
| ATOM | 2927 | O | LYS | 382 | −13.714 | −27.125 | 31.349 | 1.00 | 14.77 |
| ATOM | 2928 | CB | LYS | 382 | −16.391 | −28.550 | 31.229 | 1.00 | 14.23 |
| ATOM | 2929 | CG | LYS | 382 | −17.940 | −28.436 | 31.199 | 1.00 | 16.65 |
| ATOM | 2930 | CD | LYS | 382 | −18.576 | −28.992 | 32.510 | 1.00 | 16.25 |
| ATOM | 2931 | CE | LYS | 382 | −20.037 | −29.368 | 32.365 | 1.00 | 14.40 |
| ATOM | 2932 | NZ | LYS | 382 | −20.910 | −28.180 | 32.189 | 1.00 | 16.42 |
| ATOM | 2933 | N | LEU | 383 | −13.500 | −28.660 | 32.998 | 1.00 | 13.77 |
| ATOM | 2934 | CA | LEU | 383 | −12.060 | −28.782 | 32.896 | 1.00 | 13.16 |
| ATOM | 2935 | C | LEU | 383 | −11.615 | −29.633 | 31.705 | 1.00 | 13.03 |
| ATOM | 2936 | O | LEU | 383 | −10.641 | −29.290 | 31.005 | 1.00 | 13.46 |
| ATOM | 2937 | CB | LEU | 383 | −11.482 | −29.360 | 34.209 | 1.00 | 13.52 |
| ATOM | 2938 | CG | LEU | 383 | −9.945 | −29.317 | 34.289 | 1.00 | 13.26 |
| ATOM | 2939 | CD1 | LEU | 383 | −9.453 | −28.894 | 35.663 | 1.00 | 15.83 |
| ATOM | 2940 | CD2 | LEU | 383 | −9.340 | −30.651 | 33.910 | 1.00 | 13.22 |
| ATOM | 2941 | N | TRP | 384 | −12.303 | −30.754 | 31.498 | 1.00 | 12.52 |
| ATOM | 2942 | CA | TRP | 384 | −11.945 | −31.712 | 30.464 | 1.00 | 12.34 |
| ATOM | 2943 | C | TRP | 384 | −12.889 | −31.626 | 29.261 | 1.00 | 12.24 |
| ATOM | 2944 | O | TRP | 384 | −14.118 | −31.616 | 29.417 | 1.00 | 12.86 |
| ATOM | 2945 | CB | TRP | 384 | −12.086 | −33.138 | 31.020 | 1.00 | 12.18 |
| ATOM | 2946 | CG | TRP | 384 | −11.047 | −33.538 | 32.049 | 1.00 | 13.65 |
| ATOM | 2947 | CD1 | TRP | 384 | −11.259 | −33.778 | 33.388 | 1.00 | 13.54 |
| ATOM | 2948 | CD2 | TRP | 384 | −9.649 | −33.777 | 31.818 | 1.00 | 14.97 |
| ATOM | 2949 | CE2 | TRP | 384 | −9.072 | −34.148 | 33.071 | 1.00 | 14.69 |
| ATOM | 2950 | CE3 | TRP | 384 | −8.818 | −33.688 | 30.688 | 1.00 | 14.15 |
| ATOM | 2951 | NE1 | TRP | 384 | −10.079 | −34.141 | 34.002 | 1.00 | 13.78 |
| ATOM | 2952 | CZ2 | TRP | 384 | −7.702 | −34.438 | 33.219 | 1.00 | 15.45 |
| ATOM | 2953 | CZ3 | TRP | 384 | −7.445 | −33.995 | 30.836 | 1.00 | 16.00 |
| ATOM | 2954 | CH2 | TRP | 384 | −6.906 | −34.359 | 32.096 | 1.00 | 16.28 |
| ATOM | 2955 | N | LEU | 385 | −12.309 | −31.613 | 28.069 | 1.00 | 12.13 |
| ATOM | 2956 | CA | LEU | 385 | −13.033 | −31.850 | 26.824 | 1.00 | 12.00 |
| ATOM | 2957 | C | LEU | 385 | −12.500 | −33.191 | 26.268 | 1.00 | 11.75 |
| ATOM | 2958 | O | LEU | 385 | −11.295 | −33.340 | 26.033 | 1.00 | 11.54 |
| ATOM | 2959 | CB | LEU | 385 | −12.805 | −30.699 | 25.818 | 1.00 | 11.71 |
| ATOM | 2960 | CG | LEU | 385 | −13.160 | −31.008 | 24.345 | 1.00 | 13.27 |
| ATOM | 2961 | CD1 | LEU | 385 | −14.623 | −31.343 | 24.198 | 1.00 | 12.98 |
| ATOM | 2962 | CD2 | LEU | 385 | −12.798 | −29.890 | 23.414 | 1.00 | 12.58 |
| ATOM | 2963 | N | ILE | 386 | −13.397 | −34.152 | 26.074 | 1.00 | 10.97 |
| ATOM | 2964 | CA | ILE | 386 | −12.987 | −35.516 | 25.788 | 1.00 | 10.66 |
| ATOM | 2965 | C | ILE | 386 | −13.607 | −35.932 | 24.473 | 1.00 | 10.65 |
| ATOM | 2966 | O | ILE | 386 | −14.821 | −35.778 | 24.273 | 1.00 | 10.80 |
| ATOM | 2967 | CB | ILE | 386 | −13.409 | −36.498 | 26.919 | 1.00 | 10.16 |
| ATOM | 2968 | CG1 | ILE | 386 | −12.769 | −36.081 | 28.260 | 1.00 | 10.96 |
| ATOM | 2969 | CG2 | ILE | 386 | −13.024 | −37.954 | 26.577 | 1.00 | 7.42 |
| ATOM | 2970 | CD1 | ILE | 386 | −13.355 | −36.812 | 29.510 | 1.00 | 10.48 |
| ATOM | 2971 | N | GLN | 387 | −12.761 | −36.431 | 23.582 | 1.00 | 10.63 |
| ATOM | 2972 | CA | GLN | 387 | −13.219 | −37.035 | 22.338 | 1.00 | 10.19 |
| ATOM | 2973 | C | GLN | 387 | −12.957 | −38.539 | 22.336 | 1.00 | 11.02 |
| ATOM | 2974 | O | GLN | 387 | −11.880 | −38.988 | 22.746 | 1.00 | 11.52 |
| ATOM | 2975 | CB | GLN | 387 | −12.548 | −36.379 | 21.127 | 1.00 | 10.00 |
| ATOM | 2976 | CG | GLN | 387 | −13.024 | −37.011 | 19.787 | 1.00 | 9.67 |
| ATOM | 2977 | CD | GLN | 387 | −12.641 | −36.210 | 18.569 | 1.00 | 12.60 |
| ATOM | 2978 | NE2 | GLN | 387 | −11.504 | −35.480 | 18.647 | 1.00 | 12.01 |
| ATOM | 2979 | OE1 | GLN | 387 | −13.341 | −36.253 | 17.551 | 1.00 | 13.36 |
| ATOM | 2980 | N | PHE | 388 | −13.962 | −39.296 | 21.877 | 1.00 | 11.77 |
| ATOM | 2981 | CA | PHE | 388 | −13.908 | −40.736 | 21.663 | 1.00 | 12.71 |
| ATOM | 2982 | C | PHE | 388 | −14.145 | −40.952 | 20.167 | 1.00 | 12.88 |
| ATOM | 2983 | O | PHE | 388 | −15.089 | −40.392 | 19.618 | 1.00 | 13.73 |
| ATOM | 2984 | CB | PHE | 388 | −15.057 | −41.425 | 22.438 | 1.00 | 12.96 |
| ATOM | 2985 | CG | PHE | 388 | −14.961 | −41.323 | 23.949 | 1.00 | 13.47 |
| ATOM | 2986 | CD1 | PHE | 388 | −14.168 | −42.216 | 24.680 | 1.00 | 16.13 |
| ATOM | 2987 | CD2 | PHE | 388 | −15.707 | −40.376 | 24.636 | 1.00 | 13.69 |
| ATOM | 2988 | CE1 | PHE | 388 | −14.106 | −42.133 | 26.067 | 1.00 | 16.97 |
| ATOM | 2989 | CE2 | PHE | 388 | −15.665 | −40.281 | 26.019 | 1.00 | 15.83 |
| ATOM | 2990 | CZ | PHE | 388 | −14.864 | −41.162 | 26.745 | 1.00 | 16.00 |
| ATOM | 2991 | N | TYR | 389 | −13.316 | −41.750 | 19.500 | 1.00 | 13.15 |
| ATOM | 2992 | CA | TYR | 389 | −13.374 | −41.865 | 18.017 | 1.00 | 13.47 |
| ATOM | 2993 | C | TYR | 389 | −13.132 | −43.322 | 17.616 | 1.00 | 13.84 |
| ATOM | 2994 | O | TYR | 389 | −12.208 | −43.941 | 18.128 | 1.00 | 14.49 |
| ATOM | 2995 | CB | TYR | 389 | −12.293 | −40.989 | 17.390 | 1.00 | 12.26 |
| ATOM | 2996 | CG | TYR | 389 | −12.503 | −40.513 | 15.950 | 1.00 | 12.41 |
| ATOM | 2997 | CD1 | TYR | 389 | −13.146 | −39.306 | 15.690 | 1.00 | 12.68 |
| ATOM | 2998 | CD2 | TYR | 389 | −11.997 | −41.228 | 14.857 | 1.00 | 13.93 |
| ATOM | 2999 | CE1 | TYR | 389 | −13.300 | −38.820 | 14.405 | 1.00 | 13.02 |
| ATOM | 3000 | CE2 | TYR | 389 | −12.153 | −40.736 | 13.537 | 1.00 | 11.80 |
| ATOM | 3001 | CZ | TYR | 389 | −12.809 | −39.527 | 13.338 | 1.00 | 14.18 |
| ATOM | 3002 | OH | TYR | 389 | −13.008 | −39.011 | 12.074 | 1.00 | 16.95 |
| ATOM | 3003 | N | ASP | 390 | −13.939 | −43.833 | 16.693 | 1.00 | 13.83 |
| ATOM | 3004 | CA | ASP | 390 | −13.986 | −45.258 | 16.368 | 1.00 | 14.38 |
| ATOM | 3005 | C | ASP | 390 | −14.051 | −45.395 | 14.846 | 1.00 | 14.28 |
| ATOM | 3006 | O | ASP | 390 | −15.109 | −45.233 | 14.238 | 1.00 | 14.43 |
| ATOM | 3007 | CB | ASP | 390 | −15.220 | −45.865 | 17.032 | 1.00 | 14.03 |
| ATOM | 3008 | CG | ASP | 390 | −15.301 | −47.383 | 16.900 | 1.00 | 16.74 |
| ATOM | 3009 | OD1 | ASP | 390 | −14.367 | −47.994 | 16.343 | 1.00 | 16.33 |
| ATOM | 3010 | OD2 | ASP | 390 | −16.332 | −47.958 | 17.345 | 1.00 | 16.65 |
| ATOM | 3011 | N | ARG | 391 | −12.918 | −45.675 | 14.230 | 1.00 | 13.72 |
| ATOM | 3012 | CA | ARG | 391 | −12.824 | −45.598 | 12.768 | 1.00 | 14.58 |
| ATOM | 3013 | C | ARG | 391 | −12.492 | −46.924 | 12.027 | 1.00 | 14.83 |
| ATOM | 3014 | O | ARG | 391 | −11.437 | −47.505 | 12.228 | 1.00 | 14.59 |
| ATOM | 3015 | CB | ARG | 391 | −11.794 | −44.539 | 12.381 | 1.00 | 13.67 |
| ATOM | 3016 | CG | ARG | 391 | −11.570 | −44.441 | 10.871 | 1.00 | 15.15 |
| ATOM | 3017 | CD | ARG | 391 | −10.752 | −43.230 | 10.515 | 1.00 | 19.65 |
| ATOM | 3018 | NE | ARG | 391 | −9.307 | −43.415 | 10.671 | 1.00 | 22.86 |
| ATOM | 3019 | CZ | ARG | 391 | −8.469 | −42.447 | 11.089 | 1.00 | 27.05 |
| ATOM | 3020 | NH1 | ARG | 391 | −8.925 | −41.221 | 11.459 | 1.00 | 24.48 |
| ATOM | 3021 | NH2 | ARG | 391 | −7.163 | −42.703 | 11.166 | 1.00 | 24.78 |
| ATOM | 3022 | N | TYR | 392 | −13.399 | −47.346 | 11.141 | 1.00 | 15.72 |
| ATOM | 3023 | CA | TYR | 392 | −13.183 | −48.456 | 10.214 | 1.00 | 15.93 |
| ATOM | 3024 | C | TYR | 392 | −12.604 | −47.965 | 8.875 | 1.00 | 16.91 |
| ATOM | 3025 | O | TYR | 392 | −12.596 | −46.750 | 8.584 | 1.00 | 17.06 |
| ATOM | 3026 | CB | TYR | 392 | −14.498 | −49.192 | 9.983 | 1.00 | 15.99 |
| ATOM | 3027 | CG | TYR | 392 | −14.904 | −50.083 | 11.143 | 1.00 | 16.17 |
| ATOM | 3028 | CD1 | TYR | 392 | −14.687 | −51.466 | 11.088 | 1.00 | 17.69 |
| ATOM | 3029 | CD2 | TYR | 392 | −15.493 | −49.548 | 12.294 | 1.00 | 15.42 |
| ATOM | 3030 | CE1 | TYR | 392 | −15.045 | −52.294 | 12.136 | 1.00 | 17.39 |
| ATOM | 3031 | CE2 | TYR | 392 | −15.867 | −50.362 | 13.356 | 1.00 | 17.01 |
| ATOM | 3032 | CZ | TYR | 392 | −15.620 | −51.753 | 13.262 | 1.00 | 16.69 |
| ATOM | 3033 | OH | TYR | 392 | −15.981 | −52.584 | 14.284 | 1.00 | 18.03 |
| ATOM | 3034 | N | ASP | 393 | −12.097 | −48.894 | 8.067 | 1.00 | 16.70 |
| ATOM | 3035 | CA | ASP | 393 | −11.563 | −48.532 | 6.756 | 1.00 | 17.61 |
| ATOM | 3036 | C | ASP | 393 | −12.706 | −48.105 | 5.843 | 1.00 | 17.70 |
| ATOM | 3037 | O | ASP | 393 | −13.850 | −48.499 | 6.059 | 1.00 | 17.92 |
| ATOM | 3038 | CB | ASP | 393 | −10.818 | −49.717 | 6.134 | 1.00 | 17.06 |
| ATOM | 3039 | CG | ASP | 393 | −9.563 | −50.045 | 6.874 | 0.50 | 18.46 |
| ATOM | 3040 | OD1 | ASP | 393 | −8.950 | −49.106 | 7.431 | 0.50 | 16.58 |
| ATOM | 3041 | OD2 | ASP | 393 | −9.200 | −51.243 | 6.903 | 0.50 | 20.65 |
| ATOM | 3042 | N | ASN | 394 | −12.397 | −47.329 | 4.814 | 1.00 | 18.62 |
| ATOM | 3043 | CA | ASN | 394 | −13.457 | −46.836 | 3.935 | 1.00 | 20.51 |
| ATOM | 3044 | C | ASN | 394 | −14.139 | −47.916 | 3.091 | 1.00 | 21.78 |
| ATOM | 3045 | O | ASN | 394 | −15.298 | −47.744 | 2.695 | 1.00 | 21.39 |
| ATOM | 3046 | CB | ASN | 394 | −12.986 | −45.646 | 3.089 | 1.00 | 20.21 |
| ATOM | 3047 | CG | ASN | 394 | −12.742 | −44.407 | 3.936 | 1.00 | 19.46 |
| ATOM | 3048 | ND2 | ASN | 394 | −11.937 | −43.471 | 3.419 | 1.00 | 16.34 |
| ATOM | 3049 | OD1 | ASN | 394 | −13.261 | −44.303 | 5.052 | 1.00 | 14.16 |
| ATOM | 3050 | N | ASN | 395 | −13.430 | −49.022 | 2.846 | 1.00 | 23.42 |
| ATOM | 3051 | CA | ASN | 395 | −14.039 | −50.185 | 2.181 | 1.00 | 25.04 |
| ATOM | 3052 | C | ASN | 395 | −14.745 | −51.179 | 3.125 | 1.00 | 25.26 |
| ATOM | 3053 | O | ASN | 395 | −15.077 | −52.292 | 2.705 | 1.00 | 25.53 |
| ATOM | 3054 | CB | ASN | 395 | −13.029 | −50.902 | 1.263 | 1.00 | 25.60 |
| ATOM | 3055 | CG | ASN | 395 | −11.772 | −51.385 | 2.004 | 1.00 | 29.64 |
| ATOM | 3056 | ND2 | ASN | 395 | −10.638 | −51.435 | 1.282 | 1.00 | 33.03 |
| ATOM | 3057 | OD1 | ASN | 395 | −11.815 | −51.718 | 3.196 | 1.00 | 31.46 |
| ATOM | 3058 | N | GLN | 396 | −14.978 | −50.791 | 4.384 | 1.00 | 24.67 |
| ATOM | 3059 | CA | GLN | 396 | −15.740 | −51.629 | 5.317 | 1.00 | 24.15 |
| ATOM | 3060 | C | GLN | 396 | −17.023 | −50.950 | 5.746 | 1.00 | 23.90 |
| ATOM | 3061 | O | GLN | 396 | −17.107 | −49.720 | 5.813 | 1.00 | 24.13 |
| ATOM | 3062 | CB | GLN | 396 | −14.946 | −51.927 | 6.596 | 1.00 | 24.66 |
| ATOM | 3063 | CG | GLN | 396 | −13.621 | −52.642 | 6.417 | 1.00 | 25.82 |
| ATOM | 3064 | CD | GLN | 396 | −12.850 | −52.769 | 7.735 | 1.00 | 27.97 |
| ATOM | 3065 | NE2 | GLN | 396 | −12.689 | −54.008 | 8.203 | 1.00 | 24.99 |
| ATOM | 3066 | OE1 | GLN | 396 | −12.394 | −51.760 | 8.322 | 1.00 | 27.22 |
| ATOM | 3067 | N | THR | 397 | −18.017 | −51.756 | 6.073 | 1.00 | 23.08 |
| ATOM | 3068 | CA | THR | 397 | −19.240 | −51.259 | 6.678 | 1.00 | 22.53 |
| ATOM | 3069 | C | THR | 397 | −18.994 | −51.091 | 8.158 | 1.00 | 22.32 |
| ATOM | 3070 | O | THR | 397 | −18.428 | −51.980 | 8.790 | 1.00 | 22.31 |
| ATOM | 3071 | CB | THR | 397 | −20.402 | −52.251 | 6.448 | 1.00 | 22.09 |
| ATOM | 3072 | CG2 | THR | 397 | −21.721 | −51.758 | 7.053 | 1.00 | 22.76 |
| ATOM | 3073 | OG1 | THR | 397 | −20.587 | −52.414 | 5.043 | 1.00 | 24.36 |
| ATOM | 3074 | N | TYR | 398 | −19.411 | −49.953 | 8.718 | 1.00 | 21.31 |
| ATOM | 3075 | CA | TYR | 398 | −19.392 | −49.808 | 10.159 | 1.00 | 20.42 |
| ATOM | 3076 | C | TYR | 398 | −20.464 | −50.726 | 10.764 | 1.00 | 20.63 |
| ATOM | 3077 | O | TYR | 398 | −21.640 | −50.524 | 10.505 | 1.00 | 19.65 |
| ATOM | 3078 | CB | TYR | 398 | −19.652 | −48.349 | 10.578 | 1.00 | 20.14 |
| ATOM | 3079 | CG | TYR | 398 | −19.429 | −48.142 | 12.060 | 1.00 | 17.88 |
| ATOM | 3080 | CD1 | TYR | 398 | −18.207 | −47.649 | 12.536 | 1.00 | 14.88 |
| ATOM | 3081 | CD2 | TYR | 398 | −20.413 | −48.470 | 12.986 | 1.00 | 14.35 |
| ATOM | 3082 | CE1 | TYR | 398 | −17.983 | −47.475 | 13.889 | 1.00 | 11.11 |
| ATOM | 3083 | CE2 | TYR | 398 | −20.195 | −48.293 | 14.352 | 1.00 | 12.87 |
| ATOM | 3084 | CZ | TYR | 398 | −18.972 | −47.807 | 14.790 | 1.00 | 10.83 |
| ATOM | 3085 | OH | TYR | 398 | −18.740 | −47.655 | 16.151 | 1.00 | 10.65 |
| ATOM | 3086 | N | PRO | 399 | −20.063 | −51.707 | 11.612 | 1.00 | 21.33 |
| ATOM | 3087 | CA | PRO | 399 | −21.022 | −52.668 | 12.169 | 1.00 | 21.32 |
| ATOM | 3088 | C | PRO | 399 | −22.019 | −52.112 | 13.164 | 1.00 | 22.15 |
| ATOM | 3089 | O | PRO | 399 | −21.668 | −51.338 | 14.056 | 1.00 | 21.71 |
| ATOM | 3090 | CB | PRO | 399 | −20.113 | −53.722 | 12.841 | 1.00 | 21.50 |
| ATOM | 3091 | CG | PRO | 399 | −18.840 | −53.639 | 12.102 | 1.00 | 21.05 |
| ATOM | 3092 | CD | PRO | 399 | −18.679 | −52.144 | 11.879 | 1.00 | 21.25 |
| ATOM | 3093 | N | GLU | 400 | −23.265 | −52.545 | 13.012 | 1.00 | 23.14 |
| ATOM | 3094 | CA | GLU | 400 | −24.370 | −52.163 | 13.895 | 1.00 | 24.36 |
| ATOM | 3095 | C | GLU | 400 | −24.030 | −52.409 | 15.374 | 1.00 | 24.36 |
| ATOM | 3096 | O | GLU | 400 | −24.441 | −51.664 | 16.264 | 1.00 | 24.33 |
| ATOM | 3097 | CB | GLU | 400 | −25.642 | −52.934 | 13.463 | 1.00 | 24.69 |
| ATOM | 3098 | CG | GLU | 400 | −25.844 | −54.323 | 14.146 | 1.00 | 27.18 |
| ATOM | 3099 | CD | GLU | 400 | −26.052 | −55.505 | 13.208 | 0.50 | 27.05 |
| ATOM | 3100 | OE1 | GLU | 400 | −25.203 | −55.757 | 12.308 | 0.50 | 24.50 |
| ATOM | 3101 | OE2 | GLU | 400 | −27.057 | −56.219 | 13.433 | 0.50 | 28.48 |
| ATOM | 3102 | N | THR | 401 | −23.240 | −53.446 | 15.632 | 1.00 | 25.12 |
| ATOM | 3103 | CA | THR | 401 | −22.878 | −53.814 | 17.014 | 1.00 | 25.31 |
| ATOM | 3104 | C | THR | 401 | −21.731 | −52.975 | 17.605 | 1.00 | 24.52 |
| ATOM | 3105 | O | THR | 401 | −21.575 | −52.908 | 18.838 | 1.00 | 24.35 |
| ATOM | 3106 | CB | THR | 401 | −22.546 | −55.316 | 17.106 | 1.00 | 26.22 |
| ATOM | 3107 | CG2 | THR | 401 | −23.840 | −56.136 | 16.981 | 1.00 | 27.60 |
| ATOM | 3108 | OG1 | THR | 401 | −21.658 | −55.687 | 16.031 | 1.00 | 28.11 |
| ATOM | 3109 | N | SER | 402 | −20.967 | −52.303 | 16.742 | 1.00 | 23.15 |
| ATOM | 3110 | CA | SER | 402 | −19.782 | −51.540 | 17.185 | 1.00 | 21.89 |
| ATOM | 3111 | C | SER | 402 | −20.097 | −50.306 | 18.051 | 1.00 | 20.86 |
| ATOM | 3112 | O | SER | 402 | −19.260 | −49.896 | 18.875 | 1.00 | 20.13 |
| ATOM | 3113 | CB | SER | 402 | −18.894 | −51.180 | 15.999 | 1.00 | 21.32 |
| ATOM | 3114 | OG | SER | 402 | −18.401 | −52.369 | 15.398 | 1.00 | 22.87 |
| ATOM | 3115 | N | PHE | 403 | −21.292 | −49.733 | 17.892 | 1.00 | 20.95 |
| ATOM | 3116 | CA | PHE | 403 | −21.675 | −48.507 | 18.633 | 1.00 | 20.59 |
| ATOM | 3117 | C | PHE | 403 | −21.519 | −48.711 | 20.152 | 1.00 | 20.75 |
| ATOM | 3118 | O | PHE | 403 | −21.132 | −47.801 | 20.879 | 1.00 | 20.41 |
| ATOM | 3119 | CB | PHE | 403 | −23.115 | −48.060 | 18.331 | 1.00 | 20.83 |
| ATOM | 3120 | CG | PHE | 403 | −23.372 | −47.697 | 16.875 | 1.00 | 20.08 |
| ATOM | 3121 | CD1 | PHE | 403 | −24.168 | −48.511 | 16.071 | 1.00 | 20.36 |
| ATOM | 3122 | CD2 | PHE | 403 | −22.824 | −46.554 | 16.322 | 1.00 | 16.47 |
| ATOM | 3123 | CE1 | PHE | 403 | −24.422 | −48.183 | 14.717 | 1.00 | 22.00 |
| ATOM | 3124 | CE2 | PHE | 403 | −23.052 | −46.226 | 14.987 | 1.00 | 19.42 |
| ATOM | 3125 | CZ | PHE | 403 | −23.843 | −47.034 | 14.175 | 1.00 | 19.18 |
| ATOM | 3126 | N | LYS | 404 | −21.783 | −49.928 | 20.616 | 1.00 | 20.60 |
| ATOM | 3127 | CA | LYS | 404 | −21.722 | −50.222 | 22.055 | 1.00 | 20.55 |
| ATOM | 3128 | C | LYS | 404 | −20.312 | −49.990 | 22.674 | 1.00 | 19.17 |
| ATOM | 3129 | O | LYS | 404 | −20.200 | −49.734 | 23.872 | 1.00 | 18.28 |
| ATOM | 3130 | CB | LYS | 404 | −22.295 | −51.619 | 22.363 | 1.00 | 21.16 |
| ATOM | 3131 | CG | LYS | 404 | −21.419 | −52.786 | 21.965 | 1.00 | 24.36 |
| ATOM | 3132 | CD | LYS | 404 | −22.132 | −54.137 | 22.246 | 1.00 | 30.52 |
| ATOM | 3133 | CE | LYS | 404 | −21.119 | −55.305 | 22.411 | 1.00 | 32.43 |
| ATOM | 3134 | NZ | LYS | 404 | −20.332 | −55.601 | 21.164 | 1.00 | 32.05 |
| ATOM | 3135 | N | PHE | 405 | −19.270 | −50.036 | 21.840 | 1.00 | 17.56 |
| ATOM | 3136 | CA | PHE | 405 | −17.895 | −49.792 | 22.287 | 1.00 | 16.47 |
| ATOM | 3137 | C | PHE | 405 | −17.741 | −48.367 | 22.859 | 1.00 | 16.15 |
| ATOM | 3138 | O | PHE | 405 | −17.445 | −48.212 | 24.065 | 1.00 | 16.19 |
| ATOM | 3139 | CB | PHE | 405 | −16.878 | −50.098 | 21.169 | 1.00 | 16.10 |
| ATOM | 3140 | CG | PHE | 405 | −15.477 | −49.584 | 21.447 | 1.00 | 16.90 |
| ATOM | 3141 | CD1 | PHE | 405 | −14.874 | −49.771 | 22.696 | 1.00 | 16.48 |
| ATOM | 3142 | CD2 | PHE | 405 | −14.744 | −48.943 | 20.438 | 1.00 | 13.71 |
| ATOM | 3143 | CE1 | PHE | 405 | −13.577 | −49.248 | 22.962 | 1.00 | 16.84 |
| ATOM | 3144 | CE2 | PHE | 405 | −13.452 | −48.474 | 20.683 | 1.00 | 14.68 |
| ATOM | 3145 | CZ | PHE | 405 | −12.868 | −48.622 | 21.951 | 1.00 | 12.90 |
| ATOM | 3146 | N | LEU | 406 | −17.955 | −47.331 | 22.039 | 1.00 | 14.74 |
| ATOM | 3147 | CA | LEU | 406 | −17.876 | −45.967 | 22.590 | 1.00 | 14.07 |
| ATOM | 3148 | C | LEU | 406 | −19.019 | −45.674 | 23.576 | 1.00 | 13.96 |
| ATOM | 3149 | O | LEU | 406 | −18.806 | −44.983 | 24.562 | 1.00 | 13.16 |
| ATOM | 3150 | CB | LEU | 406 | −17.767 | −44.886 | 21.499 | 1.00 | 13.67 |
| ATOM | 3151 | CG | LEU | 406 | −16.562 | −44.940 | 20.536 | 1.00 | 12.79 |
| ATOM | 3152 | CD1 | LEU | 406 | −16.465 | −43.666 | 19.720 | 1.00 | 11.88 |
| ATOM | 3153 | CD2 | LEU | 406 | −15.245 | −45.193 | 21.264 | 1.00 | 14.07 |
| ATOM | 3154 | N | ASP | 407 | −20.215 | −46.208 | 23.327 | 1.00 | 14.10 |
| ATOM | 3155 | CA | ASP | 407 | −21.340 | −46.007 | 24.248 | 1.00 | 15.84 |
| ATOM | 3156 | C | ASP | 407 | −20.972 | −46.467 | 25.667 | 1.00 | 16.84 |
| ATOM | 3157 | O | ASP | 407 | −21.294 | −45.794 | 26.661 | 1.00 | 17.60 |
| ATOM | 3158 | CB | ASP | 407 | −22.571 | −46.807 | 23.792 | 1.00 | 16.02 |
| ATOM | 3159 | CG | ASP | 407 | −23.336 | −46.145 | 22.650 | 1.00 | 17.54 |
| ATOM | 3160 | OD1 | ASP | 407 | −22.969 | −45.020 | 22.225 | 1.00 | 14.78 |
| ATOM | 3161 | OD2 | ASP | 407 | −24.347 | −46.751 | 22.210 | 1.00 | 16.52 |
| ATOM | 3162 | N | GLY | 408 | −20.307 | −47.623 | 25.755 | 1.00 | 16.60 |
| ATOM | 3163 | CA | GLY | 408 | −19.915 | −48.180 | 27.044 | 1.00 | 16.65 |
| ATOM | 3164 | C | GLY | 408 | −18.837 | −47.352 | 27.732 | 1.00 | 16.42 |
| ATOM | 3165 | O | GLY | 408 | −18.881 | −47.171 | 28.940 | 1.00 | 17.61 |
| ATOM | 3166 | N | TRP | 409 | −17.876 | −46.828 | 26.973 | 1.00 | 15.67 |
| ATOM | 3167 | CA | TRP | 409 | −16.819 | −46.006 | 27.573 | 1.00 | 15.12 |
| ATOM | 3168 | C | TRP | 409 | −17.410 | −44.725 | 28.156 | 1.00 | 15.27 |
| ATOM | 3169 | O | TRP | 409 | −17.052 | −44.329 | 29.262 | 1.00 | 15.30 |
| ATOM | 3170 | CB | TRP | 409 | −15.713 | −45.673 | 26.556 | 1.00 | 14.80 |
| ATOM | 3171 | CG | TRP | 409 | −14.293 | −45.633 | 27.145 | 1.00 | 11.88 |
| ATOM | 3172 | CD1 | TRP | 409 | −13.921 | −45.533 | 28.484 | 1.00 | 11.39 |
| ATOM | 3173 | CD2 | TRP | 409 | −13.084 | −45.673 | 26.405 | 1.00 | 10.42 |
| ATOM | 3174 | CE2 | TRP | 409 | −12.013 | −45.607 | 27.335 | 1.00 | 11.07 |
| ATOM | 3175 | CE3 | TRP | 409 | −12.792 | −45.785 | 25.033 | 1.00 | 11.33 |
| ATOM | 3176 | NE1 | TRP | 409 | −12.550 | −45.512 | 28.595 | 1.00 | 9.31 |
| ATOM | 3177 | CZ2 | TRP | 409 | −10.684 | −45.644 | 26.935 | 1.00 | 10.89 |
| ATOM | 3178 | CZ3 | TRP | 409 | −11.468 | −45.796 | 24.636 | 1.00 | 10.78 |
| ATOM | 3179 | CH2 | TRP | 409 | −10.428 | −45.730 | 25.587 | 1.00 | 9.97 |
| ATOM | 3180 | N | VAL | 410 | −18.333 | −44.095 | 27.417 | 1.00 | 15.17 |
| ATOM | 3181 | CA | VAL | 410 | −18.962 | −42.861 | 27.867 | 1.00 | 15.25 |
| ATOM | 3182 | C | VAL | 410 | −19.838 | −43.138 | 29.103 | 1.00 | 15.93 |
| ATOM | 3183 | O | VAL | 410 | −19.820 | −42.383 | 30.077 | 1.00 | 15.09 |
| ATOM | 3184 | CB | VAL | 410 | −19.803 | −42.214 | 26.730 | 1.00 | 15.74 |
| ATOM | 3185 | CG1 | VAL | 410 | −20.776 | −41.193 | 27.286 | 1.00 | 15.56 |
| ATOM | 3186 | CG2 | VAL | 410 | −18.878 | −41.587 | 25.668 | 1.00 | 13.66 |
| ATOM | 3187 | N | ASN | 411 | −20.592 | −44.237 | 29.043 | 1.00 | 16.92 |
| ATOM | 3188 | CA | ASN | 411 | −21.426 | −44.675 | 30.160 | 1.00 | 18.32 |
| ATOM | 3189 | C | ASN | 411 | −20.574 | −44.969 | 31.406 | 1.00 | 17.97 |
| ATOM | 3190 | O | ASN | 411 | −21.003 | −44.704 | 32.533 | 1.00 | 17.50 |
| ATOM | 3191 | CB | ASN | 411 | −22.249 | −45.908 | 29.759 | 1.00 | 18.09 |
| ATOM | 3192 | CG | ASN | 411 | −22.844 | −46.634 | 30.971 | 1.00 | 24.11 |
| ATOM | 3193 | ND2 | ASN | 411 | −22.216 | −47.771 | 31.372 | 1.00 | 27.40 |
| ATOM | 3194 | OD1 | ASN | 411 | −23.841 | −46.179 | 31.551 | 1.00 | 25.92 |
| ATOM | 3195 | N | SER | 412 | −19.371 | −45.515 | 31.195 | 1.00 | 18.29 |
| ATOM | 3196 | CA | SER | 412 | −18.453 | −45.777 | 32.318 | 1.00 | 18.53 |
| ATOM | 3197 | C | SER | 412 | −18.216 | −44.499 | 33.124 | 1.00 | 18.46 |
| ATOM | 3198 | O | SER | 412 | −18.130 | −44.555 | 34.349 | 1.00 | 18.56 |
| ATOM | 3199 | CB | SER | 412 | −17.127 | −46.402 | 31.865 | 1.00 | 17.58 |
| ATOM | 3200 | OG | SER | 412 | −16.179 | −45.412 | 31.515 | 1.00 | 19.31 |
| ATOM | 3201 | N | VAL | 413 | −18.134 | −43.347 | 32.444 | 1.00 | 18.16 |
| ATOM | 3202 | CA | VAL | 413 | −17.917 | −42.077 | 33.138 | 1.00 | 17.19 |
| ATOM | 3203 | C | VAL | 413 | −19.220 | −41.492 | 33.690 | 1.00 | 18.17 |
| ATOM | 3204 | O | VAL | 413 | −19.272 | −41.092 | 34.869 | 1.00 | 17.99 |
| ATOM | 3205 | CB | VAL | 413 | −17.143 | −41.035 | 32.255 | 1.00 | 17.59 |
| ATOM | 3206 | CG1 | VAL | 413 | −15.760 | −41.544 | 31.890 | 1.00 | 16.71 |
| ATOM | 3207 | CG2 | VAL | 413 | −17.019 | −39.681 | 32.965 | 1.00 | 16.06 |
| ATOM | 3208 | N | THR | 414 | −20.277 | −41.466 | 32.866 | 1.00 | 18.65 |
| ATOM | 3209 | CA | THR | 414 | −21.510 | −40.750 | 33.229 | 1.00 | 19.19 |
| ATOM | 3210 | C | THR | 414 | −22.277 | −41.424 | 34.350 | 1.00 | 20.76 |
| ATOM | 3211 | O | THR | 414 | −22.894 | −40.731 | 35.180 | 1.00 | 20.89 |
| ATOM | 3212 | CB | THR | 414 | −22.454 | −40.450 | 32.039 | 1.00 | 18.43 |
| ATOM | 3213 | CG2 | THR | 414 | −21.747 | −39.611 | 30.967 | 1.00 | 18.20 |
| ATOM | 3214 | OG1 | THR | 414 | −22.920 | −41.665 | 31.453 | 1.00 | 19.64 |
| ATOM | 3215 | N | LYS | 415 | −22.225 | −42.760 | 34.395 | 1.00 | 21.17 |
| ATOM | 3216 | CA | LYS | 415 | −22.843 | −43.469 | 35.500 | 1.00 | 21.75 |
| ATOM | 3217 | C | LYS | 415 | −22.256 | −43.033 | 36.854 | 1.00 | 21.94 |
| ATOM | 3218 | O | LYS | 415 | −22.948 | −43.117 | 37.866 | 1.00 | 22.31 |
| ATOM | 3219 | CB | LYS | 415 | −22.795 | −44.988 | 35.296 | 1.00 | 22.02 |
| ATOM | 3220 | CG | LYS | 415 | −21.452 | −45.684 | 35.580 | 1.00 | 23.18 |
| ATOM | 3221 | CD | LYS | 415 | −21.618 | −47.195 | 35.427 | 1.00 | 26.05 |
| ATOM | 3222 | CE | LYS | 415 | −20.373 | −47.926 | 35.914 | 1.00 | 30.77 |
| ATOM | 3223 | NZ | LYS | 415 | −20.270 | −49.327 | 35.379 | 1.00 | 32.15 |
| ATOM | 3224 | N | ALA | 416 | −21.024 | −42.502 | 36.857 | 1.00 | 21.57 |
| ATOM | 3225 | CA | ALA | 416 | −20.359 | −42.077 | 38.101 | 1.00 | 21.57 |
| ATOM | 3226 | C | ALA | 416 | −20.602 | −40.620 | 38.509 | 1.00 | 21.72 |
| ATOM | 3227 | O | ALA | 416 | −20.083 | −40.159 | 39.539 | 1.00 | 22.29 |
| ATOM | 3228 | CB | ALA | 416 | −18.887 | −42.342 | 38.018 | 1.00 | 20.95 |
| ATOM | 3229 | N | LEU | 417 | −21.391 | −39.884 | 37.732 | 1.00 | 21.45 |
| ATOM | 3230 | CA | LEU | 417 | −21.484 | −38.424 | 37.951 | 1.00 | 21.09 |
| ATOM | 3231 | C | LEU | 417 | −22.908 | −37.905 | 38.047 | 1.00 | 21.57 |
| ATOM | 3232 | O | LEU | 417 | −23.809 | −38.451 | 37.407 | 1.00 | 22.16 |
| ATOM | 3233 | CB | LEU | 417 | −20.758 | −37.668 | 36.820 | 1.00 | 20.37 |
| ATOM | 3234 | CG | LEU | 417 | −19.242 | −37.822 | 36.629 | 1.00 | 19.60 |
| ATOM | 3235 | CD1 | LEU | 417 | −18.774 | −37.112 | 35.321 | 1.00 | 14.59 |
| ATOM | 3236 | CD2 | LEU | 417 | −18.475 | −37.305 | 37.849 | 1.00 | 19.60 |
| ATOM | 3237 | N | PRO | 418 | −23.126 | −36.831 | 38.822 | 1.00 | 22.25 |
| ATOM | 3238 | CA | PRO | 418 | −24.459 | −36.238 | 38.693 | 1.00 | 23.14 |
| ATOM | 3239 | C | PRO | 418 | −24.569 | −35.614 | 37.294 | 1.00 | 24.17 |
| ATOM | 3240 | O | PRO | 418 | −23.547 | −35.201 | 36.730 | 1.00 | 23.41 |
| ATOM | 3241 | CB | PRO | 418 | −24.478 | −35.161 | 39.771 | 1.00 | 23.26 |
| ATOM | 3242 | CG | PRO | 418 | −23.060 | −34.813 | 39.988 | 1.00 | 23.32 |
| ATOM | 3243 | CD | PRO | 418 | −22.226 | −36.029 | 39.668 | 1.00 | 21.88 |
| ATOM | 3244 | N | LYS | 419 | −25.782 | −35.576 | 36.741 | 1.00 | 24.99 |
| ATOM | 3245 | CA | LYS | 419 | −26.000 | −35.083 | 35.381 | 1.00 | 25.88 |
| ATOM | 3246 | C | LYS | 419 | −25.524 | −33.652 | 35.195 | 1.00 | 25.45 |
| ATOM | 3247 | O | LYS | 419 | −25.061 | −33.282 | 34.123 | 1.00 | 26.03 |
| ATOM | 3248 | CB | LYS | 419 | −27.463 | −35.248 | 34.961 | 1.00 | 26.40 |
| ATOM | 3249 | CG | LYS | 419 | −27.937 | −36.719 | 34.972 | 1.00 | 30.07 |
| ATOM | 3250 | CD | LYS | 419 | −29.314 | −36.879 | 34.313 | 1.00 | 37.47 |
| ATOM | 3251 | CE | LYS | 419 | −30.171 | −37.965 | 35.010 | 1.00 | 40.77 |
| ATOM | 3252 | NZ | LYS | 419 | −29.703 | −39.375 | 34.748 | 1.00 | 41.86 |
| ATOM | 3253 | N | SER | 420 | −25.579 | −32.860 | 36.257 | 1.00 | 24.82 |
| ATOM | 3254 | CA | SER | 420 | −25.150 | −31.474 | 36.195 | 1.00 | 23.93 |
| ATOM | 3255 | C | SER | 420 | −23.624 | −31.326 | 36.013 | 1.00 | 23.03 |
| ATOM | 3256 | O | SER | 420 | −23.140 | −30.243 | 35.739 | 1.00 | 23.24 |
| ATOM | 3257 | CB | SER | 420 | −25.595 | −30.751 | 37.470 | 1.00 | 24.05 |
| ATOM | 3258 | OG | SER | 420 | −24.748 | −31.128 | 38.550 | 1.00 | 25.14 |
| ATOM | 3259 | N | ASP | 421 | −22.870 | −32.409 | 36.196 | 1.00 | 22.14 |
| ATOM | 3260 | CA | ASP | 421 | −21.404 | −32.330 | 36.167 | 1.00 | 20.81 |
| ATOM | 3261 | C | ASP | 421 | −20.801 | −32.496 | 34.772 | 1.00 | 19.38 |
| ATOM | 3262 | O | ASP | 421 | −19.604 | −32.298 | 34.585 | 1.00 | 19.08 |
| ATOM | 3263 | CB | ASP | 421 | −20.784 | −33.367 | 37.103 | 1.00 | 20.65 |
| ATOM | 3264 | CG | ASP | 421 | −20.390 | −32.782 | 38.450 | 0.50 | 20.26 |
| ATOM | 3265 | OD1 | ASP | 421 | −20.912 | −31.709 | 38.819 | 0.50 | 19.62 |
| ATOM | 3266 | OD2 | ASP | 421 | −19.550 | −33.399 | 39.139 | 0.50 | 19.06 |
| ATOM | 3267 | N | TRP | 422 | −21.633 | −32.868 | 33.807 | 1.00 | 17.83 |
| ATOM | 3268 | CA | TRP | 422 | −21.123 | −33.259 | 32.510 | 1.00 | 16.22 |
| ATOM | 3269 | C | TRP | 422 | −22.025 | −32.800 | 31.375 | 1.00 | 15.97 |
| ATOM | 3270 | O | TRP | 422 | −23.241 | −32.669 | 31.547 | 1.00 | 15.18 |
| ATOM | 3271 | CB | TRP | 422 | −20.847 | −34.764 | 32.450 | 1.00 | 15.75 |
| ATOM | 3272 | CG | TRP | 422 | −22.072 | −35.686 | 32.511 | 1.00 | 16.51 |
| ATOM | 3273 | CD1 | TRP | 422 | −22.612 | −36.289 | 33.635 | 1.00 | 15.77 |
| ATOM | 3274 | CD2 | TRP | 422 | −22.863 | −36.140 | 31.396 | 1.00 | 14.21 |
| ATOM | 3275 | CE2 | TRP | 422 | −23.869 | −36.992 | 31.913 | 1.00 | 15.58 |
| ATOM | 3276 | CE3 | TRP | 422 | −22.834 | −35.885 | 30.014 | 1.00 | 13.56 |
| ATOM | 3277 | NE1 | TRP | 422 | −23.689 | −37.072 | 33.274 | 1.00 | 15.07 |
| ATOM | 3278 | CZ2 | TRP | 422 | −24.838 | −37.598 | 31.090 | 1.00 | 13.14 |
| ATOM | 3279 | CZ3 | TRP | 422 | −23.793 | −36.488 | 29.191 | 1.00 | 14.22 |
| ATOM | 3280 | CH2 | TRP | 422 | −24.790 | −37.334 | 29.739 | 1.00 | 14.53 |
| ATOM | 3281 | N | GLY | 423 | −21.405 | −32.550 | 30.219 | 1.00 | 14.83 |
| ATOM | 3282 | CA | GLY | 423 | −22.147 | −32.270 | 29.000 | 1.00 | 14.07 |
| ATOM | 3283 | C | GLY | 423 | −21.483 | −32.976 | 27.837 | 1.00 | 13.27 |
| ATOM | 3284 | O | GLY | 423 | −20.597 | −33.793 | 28.035 | 1.00 | 12.13 |
| ATOM | 3285 | N | MET | 424 | −21.920 | −32.642 | 26.628 | 1.00 | 12.71 |
| ATOM | 3286 | CA | MET | 424 | −21.334 | −33.166 | 25.404 | 1.00 | 12.35 |
| ATOM | 3287 | C | MET | 424 | −21.118 | −32.017 | 24.436 | 1.00 | 11.77 |
| ATOM | 3288 | O | MET | 424 | −21.801 | −31.028 | 24.510 | 1.00 | 12.24 |
| ATOM | 3289 | CB | MET | 424 | −22.256 | −34.243 | 24.798 | 1.00 | 11.84 |
| ATOM | 3290 | CG | MET | 424 | −22.306 | −35.513 | 25.662 | 1.00 | 12.28 |
| ATOM | 3291 | SD | MET | 424 | −23.232 | −36.862 | 24.945 | 1.00 | 12.38 |
| ATOM | 3292 | CE | MET | 424 | −22.559 | −38.219 | 25.908 | 1.00 | 12.90 |
| ATOM | 3293 | N | TYR | 425 | −20.169 | −32.164 | 23.518 | 1.00 | 12.16 |
| ATOM | 3294 | CA | TYR | 425 | −19.734 | −31.055 | 22.634 | 1.00 | 11.68 |
| ATOM | 3295 | C | TYR | 425 | −20.218 | −31.310 | 21.206 | 1.00 | 11.89 |
| ATOM | 3296 | O | TYR | 425 | −19.793 | −32.289 | 20.551 | 1.00 | 12.49 |
| ATOM | 3297 | CB | TYR | 425 | −18.211 | −30.973 | 22.717 | 1.00 | 11.07 |
| ATOM | 3298 | CG | TYR | 425 | −17.446 | −29.968 | 21.861 | 1.00 | 12.27 |
| ATOM | 3299 | CD1 | TYR | 425 | −17.775 | −28.612 | 21.844 | 1.00 | 11.54 |
| ATOM | 3300 | CD2 | TYR | 425 | −16.311 | −30.376 | 21.156 | 1.00 | 10.01 |
| ATOM | 3301 | CE1 | TYR | 425 | −17.003 | −27.695 | 21.095 | 1.00 | 11.66 |
| ATOM | 3302 | CE2 | TYR | 425 | −15.532 | −29.480 | 20.421 | 1.00 | 10.90 |
| ATOM | 3303 | CZ | TYR | 425 | −15.885 | −28.139 | 20.390 | 1.00 | 11.95 |
| ATOM | 3304 | OH | TYR | 425 | −15.115 | −27.257 | 19.629 | 1.00 | 11.31 |
| ATOM | 3305 | N | ILE | 426 | −21.118 | −30.444 | 20.722 | 1.00 | 11.23 |
| ATOM | 3306 | CA | ILE | 426 | −21.741 | −30.638 | 19.404 | 1.00 | 10.01 |
| ATOM | 3307 | C | ILE | 426 | −20.727 | −30.473 | 18.254 | 1.00 | 10.51 |
| ATOM | 3308 | O | ILE | 426 | −21.038 | −30.835 | 17.116 | 1.00 | 11.00 |
| ATOM | 3309 | CB | ILE | 426 | −22.979 | −29.701 | 19.212 | 1.00 | 10.44 |
| ATOM | 3310 | CG1 | ILE | 426 | −23.870 | −30.221 | 18.051 | 1.00 | 9.98 |
| ATOM | 3311 | CG2 | ILE | 426 | −22.507 | −28.252 | 19.050 | 1.00 | 8.28 |
| ATOM | 3312 | CD1 | ILE | 426 | −25.175 | −29.458 | 17.838 | 1.00 | 10.37 |
| ATOM | 3313 | N | ASN | 427 | −19.518 | −29.944 | 18.509 | 1.00 | 9.96 |
| ATOM | 3314 | CA | ASN | 427 | −18.482 | −30.055 | 17.441 | 1.00 | 9.81 |
| ATOM | 3315 | C | ASN | 427 | −17.953 | −31.495 | 17.265 | 1.00 | 9.82 |
| ATOM | 3316 | O | ASN | 427 | −17.349 | −31.822 | 16.233 | 1.00 | 9.58 |
| ATOM | 3317 | CB | ASN | 427 | −17.343 | −29.032 | 17.577 | 1.00 | 9.31 |
| ATOM | 3318 | CG | ASN | 427 | −17.638 | −27.719 | 16.828 | 1.00 | 9.25 |
| ATOM | 3319 | ND2 | ASN | 427 | −16.920 | −26.651 | 17.194 | 1.00 | 7.83 |
| ATOM | 3320 | OD1 | ASN | 427 | −18.516 | −27.663 | 15.948 | 1.00 | 9.38 |
| ATOM | 3321 | N | TYR | 428 | −18.229 | −32.356 | 18.248 | 1.00 | 9.53 |
| ATOM | 3322 | CA | TYR | 428 | −18.040 | −33.796 | 18.081 | 1.00 | 10.49 |
| ATOM | 3323 | C | TYR | 428 | −19.423 | −34.438 | 18.035 | 1.00 | 11.42 |
| ATOM | 3324 | O | TYR | 428 | −19.825 | −35.183 | 18.962 | 1.00 | 11.67 |
| ATOM | 3325 | CB | TYR | 428 | −17.199 | −34.362 | 19.226 | 1.00 | 10.33 |
| ATOM | 3326 | CG | TYR | 428 | −15.822 | −33.761 | 19.368 | 1.00 | 11.18 |
| ATOM | 3327 | CD1 | TYR | 428 | −15.135 | −33.271 | 18.246 | 1.00 | 12.54 |
| ATOM | 3328 | CD2 | TYR | 428 | −15.170 | −33.730 | 20.618 | 1.00 | 12.68 |
| ATOM | 3329 | CE1 | TYR | 428 | −13.859 | −32.736 | 18.350 | 1.00 | 15.18 |
| ATOM | 3330 | CE2 | TYR | 428 | −13.880 | −33.167 | 20.731 | 1.00 | 16.14 |
| ATOM | 3331 | CZ | TYR | 428 | −13.243 | −32.678 | 19.583 | 1.00 | 15.89 |
| ATOM | 3332 | OH | TYR | 428 | −11.974 | −32.149 | 19.639 | 1.00 | 21.25 |
| ATOM | 3333 | N | ALA | 429 | −20.186 | −34.073 | 16.995 | 1.00 | 12.05 |
| ATOM | 3334 | CA | ALA | 429 | −21.634 | −34.331 | 16.965 | 1.00 | 11.86 |
| ATOM | 3335 | C | ALA | 429 | −21.929 | −35.836 | 16.965 | 1.00 | 12.13 |
| ATOM | 3336 | O | ALA | 429 | −21.348 | −36.594 | 16.177 | 1.00 | 11.09 |
| ATOM | 3337 | CB | ALA | 429 | −22.272 | −33.683 | 15.752 | 1.00 | 12.57 |
| ATOM | 3338 | N | ASP | 430 | −22.852 | −36.252 | 17.834 | 1.00 | 11.79 |
| ATOM | 3339 | CA | ASP | 430 | −23.137 | −37.679 | 18.004 | 1.00 | 11.88 |
| ATOM | 3340 | C | ASP | 430 | −24.616 | −37.866 | 17.720 | 1.00 | 11.70 |
| ATOM | 3341 | O | ASP | 430 | −25.459 | −37.521 | 18.565 | 1.00 | 11.35 |
| ATOM | 3342 | CB | ASP | 430 | −22.757 | −38.100 | 19.440 | 1.00 | 12.34 |
| ATOM | 3343 | CG | ASP | 430 | −23.275 | −39.475 | 19.824 | 1.00 | 11.93 |
| ATOM | 3344 | OD1 | ASP | 430 | −23.885 | −40.180 | 18.977 | 1.00 | 12.21 |
| ATOM | 3345 | OD2 | ASP | 430 | −23.055 | −39.831 | 20.992 | 1.00 | 13.05 |
| ATOM | 3346 | N | PRO | 431 | −24.944 | −38.375 | 16.514 | 1.00 | 12.07 |
| ATOM | 3347 | CA | PRO | 431 | −26.359 | −38.344 | 16.094 | 1.00 | 12.51 |
| ATOM | 3348 | C | PRO | 431 | −27.271 | −39.329 | 16.800 | 1.00 | 13.91 |
| ATOM | 3349 | O | PRO | 431 | −28.501 | −39.270 | 16.581 | 1.00 | 14.31 |
| ATOM | 3350 | CB | PRO | 431 | −26.303 | −38.682 | 14.611 | 1.00 | 12.71 |
| ATOM | 3351 | CG | PRO | 431 | −25.007 | −39.490 | 14.440 | 1.00 | 11.99 |
| ATOM | 3352 | CD | PRO | 431 | −24.053 | −38.923 | 15.471 | 1.00 | 10.67 |
| ATOM | 3353 | N | ARG | 432 | −26.708 | −40.222 | 17.620 | 1.00 | 14.29 |
| ATOM | 3354 | CA | ARG | 432 | −27.525 | −41.301 | 18.235 | 1.00 | 15.68 |
| ATOM | 3355 | C | ARG | 432 | −28.145 | −40.868 | 19.575 | 1.00 | 16.10 |
| ATOM | 3356 | O | ARG | 432 | −28.039 | −41.561 | 20.595 | 1.00 | 17.06 |
| ATOM | 3357 | CB | ARG | 432 | −26.707 | −42.600 | 18.353 | 1.00 | 15.46 |
| ATOM | 3358 | CG | ARG | 432 | −26.378 | −43.196 | 16.976 | 1.00 | 16.28 |
| ATOM | 3359 | CD | ARG | 432 | −25.379 | −44.388 | 17.043 | 1.00 | 18.53 |
| ATOM | 3360 | NE | ARG | 432 | −25.908 | −45.547 | 17.767 | 1.00 | 17.86 |
| ATOM | 3361 | CZ | ARG | 432 | −25.628 | −45.854 | 19.038 | 1.00 | 22.65 |
| ATOM | 3362 | NH1 | ARG | 432 | −24.807 | −45.098 | 19.774 | 1.00 | 20.32 |
| ATOM | 3363 | NH2 | ARG | 432 | −26.169 | −46.946 | 19.592 | 1.00 | 22.03 |
| ATOM | 3364 | N | MET | 433 | −28.762 | −39.690 | 19.555 | 1.00 | 16.33 |
| ATOM | 3365 | CA | MET | 433 | −29.500 | −39.131 | 20.679 | 1.00 | 17.12 |
| ATOM | 3366 | C | MET | 433 | −30.712 | −38.385 | 20.103 | 1.00 | 18.04 |
| ATOM | 3367 | O | MET | 433 | −30.593 | −37.742 | 19.049 | 1.00 | 18.18 |
| ATOM | 3368 | CB | MET | 433 | −28.627 | −38.150 | 21.470 | 1.00 | 16.96 |
| ATOM | 3369 | CG | MET | 433 | −27.449 | −38.792 | 22.238 | 1.00 | 17.94 |
| ATOM | 3370 | SD | MET | 433 | −26.384 | −37.606 | 23.114 | 0.98 | 17.91 |
| ATOM | 3371 | CE | MET | 433 | −25.676 | −36.657 | 21.772 | 1.00 | 17.79 |
| ATOM | 3372 | N | ASP | 434 | −31.874 | −38.464 | 20.763 | 1.00 | 18.06 |
| ATOM | 3373 | CA | ASP | 434 | −33.027 | −37.759 | 20.249 | 1.00 | 18.77 |
| ATOM | 3374 | C | ASP | 434 | −32.843 | −36.258 | 20.454 | 1.00 | 18.44 |
| ATOM | 3375 | O | ASP | 434 | −31.907 | −35.843 | 21.143 | 1.00 | 18.42 |
| ATOM | 3376 | CB | ASP | 434 | −34.363 | −38.301 | 20.797 | 1.00 | 19.36 |
| ATOM | 3377 | CG | ASP | 434 | −34.590 | −38.013 | 22.288 | 1.00 | 22.72 |
| ATOM | 3378 | OD1 | ASP | 434 | −34.121 | −37.005 | 22.861 | 1.00 | 23.51 |
| ATOM | 3379 | OD2 | ASP | 434 | −35.326 | −38.814 | 22.898 | 1.00 | 29.83 |
| ATOM | 3380 | N | ARG | 435 | −33.711 | −35.460 | 19.829 | 1.00 | 17.48 |
| ATOM | 3381 | CA | ARG | 435 | −33.601 | −33.994 | 19.833 | 1.00 | 17.09 |
| ATOM | 3382 | C | ARG | 435 | −33.586 | −33.363 | 21.227 | 1.00 | 17.23 |
| ATOM | 3383 | O | ARG | 435 | −32.815 | −32.431 | 21.487 | 1.00 | 17.27 |
| ATOM | 3384 | CB | ARG | 435 | −34.745 | −33.381 | 18.997 | 1.00 | 16.50 |
| ATOM | 3385 | CG | ARG | 435 | −34.552 | −31.909 | 18.688 | 1.00 | 17.16 |
| ATOM | 3386 | CD | ARG | 435 | −35.558 | −31.402 | 17.669 | 1.00 | 16.21 |
| ATOM | 3387 | NE | ARG | 435 | −35.311 | −30.002 | 17.335 | 1.00 | 15.50 |
| ATOM | 3388 | CZ | ARG | 435 | −36.073 | −29.286 | 16.507 | 1.00 | 15.58 |
| ATOM | 3389 | NH1 | ARG | 435 | −37.147 | −29.842 | 15.942 | 1.00 | 12.20 |
| ATOM | 3390 | NH2 | ARG | 435 | −35.758 | −28.022 | 16.247 | 1.00 | 11.75 |
| ATOM | 3391 | N | ASP | 436 | −34.444 | −33.849 | 22.129 | 1.00 | 17.71 |
| ATOM | 3392 | CA | ASP | 436 | −34.487 | −33.289 | 23.482 | 1.00 | 18.18 |
| ATOM | 3393 | C | ASP | 436 | −33.253 | −33.640 | 24.299 | 1.00 | 17.85 |
| ATOM | 3394 | O | ASP | 436 | −32.693 | −32.784 | 24.985 | 1.00 | 18.79 |
| ATOM | 3395 | CB | ASP | 436 | −35.747 | −33.736 | 24.235 | 1.00 | 18.61 |
| ATOM | 3396 | CG | ASP | 436 | −37.014 | −33.258 | 23.576 | 1.00 | 20.48 |
| ATOM | 3397 | OD1 | ASP | 436 | −37.045 | −32.161 | 22.974 | 1.00 | 21.07 |
| ATOM | 3398 | OD2 | ASP | 436 | −37.990 | −34.013 | 23.639 | 1.00 | 26.60 |
| ATOM | 3399 | N | TYR | 437 | −32.843 | −34.899 | 24.246 | 1.00 | 17.45 |
| ATOM | 3400 | CA | TYR | 437 | −31.708 | −35.338 | 25.033 | 1.00 | 16.95 |
| ATOM | 3401 | C | TYR | 437 | −30.418 | −34.680 | 24.536 | 1.00 | 16.10 |
| ATOM | 3402 | O | TYR | 437 | −29.641 | −34.175 | 25.331 | 1.00 | 16.31 |
| ATOM | 3403 | CB | TYR | 437 | −31.592 | −36.866 | 25.001 | 1.00 | 17.16 |
| ATOM | 3404 | CG | TYR | 437 | −30.489 | −37.402 | 25.888 | 1.00 | 19.79 |
| ATOM | 3405 | CD1 | TYR | 437 | −30.698 | −37.606 | 27.254 | 1.00 | 20.87 |
| ATOM | 3406 | CD2 | TYR | 437 | −29.236 | −37.723 | 25.365 | 1.00 | 22.91 |
| ATOM | 3407 | CE1 | TYR | 437 | −29.693 | −38.111 | 28.070 | 1.00 | 21.57 |
| ATOM | 3408 | CE2 | TYR | 437 | −28.208 | −38.233 | 26.191 | 1.00 | 23.22 |
| ATOM | 3409 | CZ | TYR | 437 | −28.455 | −38.423 | 27.538 | 1.00 | 22.54 |
| ATOM | 3410 | OH | TYR | 437 | −27.455 | −38.913 | 28.356 | 1.00 | 23.00 |
| ATOM | 3411 | N | ALA | 438 | −30.191 | −34.718 | 23.223 | 1.00 | 15.45 |
| ATOM | 3412 | CA | ALA | 438 | −28.973 | −34.145 | 22.613 | 1.00 | 14.27 |
| ATOM | 3413 | C | ALA | 438 | −28.817 | −32.676 | 22.975 | 1.00 | 13.76 |
| ATOM | 3414 | O | ALA | 438 | −27.752 | −32.247 | 23.428 | 1.00 | 13.36 |
| ATOM | 3415 | CB | ALA | 438 | −29.032 | −34.294 | 21.124 | 1.00 | 13.76 |
| ATOM | 3416 | N | THR | 439 | −29.889 | −31.900 | 22.808 | 1.00 | 13.24 |
| ATOM | 3417 | CA | THR | 439 | −29.770 | −30.466 | 23.067 | 1.00 | 13.11 |
| ATOM | 3418 | C | THR | 439 | −29.593 | −30.214 | 24.550 | 1.00 | 13.36 |
| ATOM | 3419 | O | THR | 439 | −28.939 | −29.255 | 24.946 | 1.00 | 12.81 |
| ATOM | 3420 | CB | THR | 439 | −30.923 | −29.646 | 22.443 | 1.00 | 13.75 |
| ATOM | 3421 | CG2 | THR | 439 | −30.872 | −29.762 | 20.896 | 1.00 | 10.80 |
| ATOM | 3422 | OG1 | THR | 439 | −32.189 | −30.128 | 22.936 | 1.00 | 13.91 |
| ATOM | 3423 | N | LYS | 440 | −30.150 | −31.097 | 25.381 | 1.00 | 13.74 |
| ATOM | 3424 | CA | LYS | 440 | −29.929 | −30.990 | 26.819 | 1.00 | 14.76 |
| ATOM | 3425 | C | LYS | 440 | −28.442 | −31.155 | 27.162 | 1.00 | 14.19 |
| ATOM | 3426 | O | LYS | 440 | −27.918 | −30.396 | 27.949 | 1.00 | 14.04 |
| ATOM | 3427 | CB | LYS | 440 | −30.761 | −32.030 | 27.579 | 1.00 | 15.06 |
| ATOM | 3428 | CG | LYS | 440 | −30.972 | −31.717 | 29.072 | 1.00 | 20.07 |
| ATOM | 3429 | CD | LYS | 440 | −31.506 | −32.961 | 29.822 | 1.00 | 27.04 |
| ATOM | 3430 | CE | LYS | 440 | −30.386 | −33.992 | 30.024 | 1.00 | 30.55 |
| ATOM | 3431 | NZ | LYS | 440 | −30.831 | −35.396 | 29.775 | 1.00 | 32.26 |
| ATOM | 3432 | N | VAL | 441 | −27.761 | −32.152 | 26.587 | 1.00 | 14.15 |
| ATOM | 3433 | CA | VAL | 441 | −26.346 | −32.347 | 26.952 | 1.00 | 13.43 |
| ATOM | 3434 | C | VAL | 441 | −25.416 | −31.390 | 26.208 | 1.00 | 13.80 |
| ATOM | 3435 | O | VAL | 441 | −24.361 | −31.012 | 26.748 | 1.00 | 14.09 |
| ATOM | 3436 | CB | VAL | 441 | −25.868 | −33.827 | 26.818 | 1.00 | 14.00 |
| ATOM | 3437 | CG1 | VAL | 441 | −26.641 | −34.728 | 27.774 | 1.00 | 13.01 |
| ATOM | 3438 | CG2 | VAL | 441 | −26.008 | −34.338 | 25.375 | 1.00 | 11.05 |
| ATOM | 3439 | N | TYR | 442 | −25.789 | −30.992 | 24.981 | 1.00 | 13.53 |
| ATOM | 3440 | CA | TYR | 442 | −24.930 | −30.088 | 24.203 | 1.00 | 13.29 |
| ATOM | 3441 | C | TYR | 442 | −24.955 | −28.657 | 24.729 | 1.00 | 13.64 |
| ATOM | 3442 | O | TYR | 442 | −24.002 | −27.902 | 24.514 | 1.00 | 13.74 |
| ATOM | 3443 | CB | TYR | 442 | −25.344 | −30.029 | 22.729 | 1.00 | 13.14 |
| ATOM | 3444 | CG | TYR | 442 | −25.083 | −31.240 | 21.845 | 1.00 | 12.95 |
| ATOM | 3445 | CD1 | TYR | 442 | −23.980 | −32.090 | 22.014 | 1.00 | 10.18 |
| ATOM | 3446 | CD2 | TYR | 442 | −25.931 | −31.484 | 20.780 | 1.00 | 14.61 |
| ATOM | 3447 | CE1 | TYR | 442 | −23.777 | −33.196 | 21.137 | 1.00 | 11.08 |
| ATOM | 3448 | CE2 | TYR | 442 | −25.758 | −32.559 | 19.938 | 1.00 | 12.33 |
| ATOM | 3449 | CZ | TYR | 442 | −24.691 | −33.401 | 20.100 | 1.00 | 13.08 |
| ATOM | 3450 | OH | TYR | 442 | −24.597 | −34.405 | 19.179 | 1.00 | 12.23 |
| ATOM | 3451 | N | TYR | 443 | −26.046 | −28.263 | 25.398 | 1.00 | 13.83 |
| ATOM | 3452 | CA | TYR | 443 | −26.212 | −26.855 | 25.734 | 1.00 | 13.57 |
| ATOM | 3453 | C | TYR | 443 | −26.364 | −26.498 | 27.230 | 1.00 | 13.69 |
| ATOM | 3454 | O | TYR | 443 | −26.383 | −25.333 | 27.564 | 1.00 | 13.61 |
| ATOM | 3455 | CB | TYR | 443 | −27.317 | −26.223 | 24.880 | 1.00 | 13.61 |
| ATOM | 3456 | CG | TYR | 443 | −27.009 | −26.257 | 23.390 | 1.00 | 12.26 |
| ATOM | 3457 | CD1 | TYR | 443 | −25.886 | −25.596 | 22.872 | 1.00 | 11.85 |
| ATOM | 3458 | CD2 | TYR | 443 | −27.817 | −26.984 | 22.506 | 1.00 | 10.72 |
| ATOM | 3459 | CE1 | TYR | 443 | −25.586 | −25.647 | 21.490 | 1.00 | 11.77 |
| ATOM | 3460 | CE2 | TYR | 443 | −27.518 | −27.030 | 21.128 | 1.00 | 9.86 |
| ATOM | 3461 | CZ | TYR | 443 | −26.412 | −26.368 | 20.646 | 1.00 | 8.69 |
| ATOM | 3462 | OH | TYR | 443 | −26.136 | −26.386 | 19.308 | 1.00 | 8.65 |
| ATOM | 3463 | N | GLY | 444 | −26.454 | −27.498 | 28.102 | 1.00 | 14.56 |
| ATOM | 3464 | CA | GLY | 444 | −26.338 | −27.280 | 29.551 | 1.00 | 14.59 |
| ATOM | 3465 | C | GLY | 444 | −27.240 | −26.164 | 30.029 | 1.00 | 15.62 |
| ATOM | 3466 | O | GLY | 444 | −28.438 | −26.138 | 29.678 | 1.00 | 15.81 |
| ATOM | 3467 | N | GLU | 445 | −26.656 | −25.229 | 30.783 | 1.00 | 15.52 |
| ATOM | 3468 | CA | GLU | 445 | −27.411 | −24.160 | 31.460 | 1.00 | 16.76 |
| ATOM | 3469 | C | GLU | 445 | −27.939 | −23.099 | 30.474 | 1.00 | 16.90 |
| ATOM | 3470 | O | GLU | 445 | −28.760 | −22.266 | 30.831 | 1.00 | 16.82 |
| ATOM | 3471 | CB | GLU | 445 | −26.567 | −23.515 | 32.582 | 1.00 | 16.80 |
| ATOM | 3472 | CG | GLU | 445 | −25.396 | −22.627 | 32.103 | 1.00 | 18.87 |
| ATOM | 3473 | CD | GLU | 445 | −24.622 | −21.953 | 33.253 | 1.00 | 21.86 |
| ATOM | 3474 | OE1 | GLU | 445 | −23.675 | −22.562 | 33.797 | 1.00 | 23.03 |
| ATOM | 3475 | OE2 | GLU | 445 | −24.925 | −20.784 | 33.586 | 1.00 | 24.79 |
| ATOM | 3476 | N | ASN | 446 | −27.475 | −23.149 | 29.222 | 1.00 | 16.92 |
| ATOM | 3477 | CA | ASN | 446 | −27.879 | −22.152 | 28.226 | 1.00 | 16.32 |
| ATOM | 3478 | C | ASN | 446 | −29.044 | −22.579 | 27.325 | 1.00 | 16.47 |
| ATOM | 3479 | O | ASN | 446 | −29.512 | −21.785 | 26.516 | 1.00 | 16.53 |
| ATOM | 3480 | CB | ASN | 446 | −26.673 | −21.757 | 27.374 | 1.00 | 16.03 |
| ATOM | 3481 | CG | ASN | 446 | −25.708 | −20.866 | 28.126 | 1.00 | 17.45 |
| ATOM | 3482 | ND2 | ASN | 446 | −24.433 | −20.897 | 27.738 | 1.00 | 15.18 |
| ATOM | 3483 | OD1 | ASN | 446 | −26.107 | −20.142 | 29.042 | 1.00 | 18.14 |
| ATOM | 3484 | N | LEU | 447 | −29.494 | −23.827 | 27.448 | 1.00 | 15.82 |
| ATOM | 3485 | CA | LEU | 447 | −30.567 | −24.362 | 26.593 | 1.00 | 15.92 |
| ATOM | 3486 | C | LEU | 447 | −31.893 | −23.573 | 26.632 | 1.00 | 16.57 |
| ATOM | 3487 | O | LEU | 447 | −32.473 | −23.275 | 25.580 | 1.00 | 17.22 |
| ATOM | 3488 | CB | LEU | 447 | −30.830 | −25.849 | 26.901 | 1.00 | 14.99 |
| ATOM | 3489 | CG | LEU | 447 | −31.953 | −26.516 | 26.088 | 1.00 | 14.66 |
| ATOM | 3490 | CD1 | LEU | 447 | −31.649 | −26.496 | 24.596 | 1.00 | 12.53 |
| ATOM | 3491 | CD2 | LEU | 447 | −32.229 | −27.948 | 26.528 | 1.00 | 14.23 |
| ATOM | 3492 | N | ALA | 448 | −32.387 | −23.259 | 27.828 | 1.00 | 16.48 |
| ATOM | 3493 | CA | ALA | 448 | −33.665 | −22.526 | 27.950 | 1.00 | 16.42 |
| ATOM | 3494 | C | ALA | 448 | −33.625 | −21.193 | 27.196 | 1.00 | 16.19 |
| ATOM | 3495 | O | ALA | 448 | −34.563 | −20.861 | 26.476 | 1.00 | 16.35 |
| ATOM | 3496 | CB | ALA | 448 | −34.036 | −22.294 | 29.441 | 1.00 | 16.04 |
| ATOM | 3497 | N | ARG | 449 | −32.551 | −20.433 | 27.367 | 1.00 | 16.02 |
| ATOM | 3498 | CA | ARG | 449 | −32.381 | −19.176 | 26.639 | 1.00 | 16.84 |
| ATOM | 3499 | C | ARG | 449 | −32.295 | −19.393 | 25.108 | 1.00 | 16.41 |
| ATOM | 3500 | O | ARG | 449 | −32.855 | −18.607 | 24.320 | 1.00 | 17.15 |
| ATOM | 3501 | CB | ARG | 449 | −31.148 | −18.419 | 27.149 | 1.00 | 17.15 |
| ATOM | 3502 | CG | ARG | 449 | −30.918 | −17.090 | 26.471 | 1.00 | 18.71 |
| ATOM | 3503 | CD | ARG | 449 | −29.852 | −16.259 | 27.178 | 1.00 | 22.59 |
| ATOM | 3504 | NE | ARG | 449 | −29.401 | −15.158 | 26.306 | 1.00 | 24.39 |
| ATOM | 3505 | CZ | ARG | 449 | −28.581 | −14.176 | 26.675 | 1.00 | 24.83 |
| ATOM | 3506 | NH1 | ARG | 449 | −28.113 | −14.124 | 27.918 | 1.00 | 26.08 |
| ATOM | 3507 | NH2 | ARG | 449 | −28.256 | −13.225 | 25.802 | 1.00 | 25.64 |
| ATOM | 3508 | N | LEU | 450 | −31.630 | −20.471 | 24.707 | 1.00 | 15.42 |
| ATOM | 3509 | CA | LEU | 450 | −31.483 | −20.827 | 23.288 | 1.00 | 14.72 |
| ATOM | 3510 | C | LEU | 450 | −32.838 | −21.180 | 22.649 | 1.00 | 14.80 |
| ATOM | 3511 | O | LEU | 450 | −33.093 | −20.811 | 21.500 | 1.00 | 14.12 |
| ATOM | 3512 | CB | LEU | 450 | −30.471 | −21.990 | 23.135 | 1.00 | 14.17 |
| ATOM | 3513 | CG | LEU | 450 | −28.977 | −21.610 | 23.337 | 1.00 | 14.98 |
| ATOM | 3514 | CD1 | LEU | 450 | −28.081 | −22.835 | 23.347 | 1.00 | 15.45 |
| ATOM | 3515 | CD2 | LEU | 450 | −28.464 | −20.657 | 22.260 | 1.00 | 14.86 |
| ATOM | 3516 | N | GLN | 451 | −33.701 | −21.885 | 23.400 | 1.00 | 14.23 |
| ATOM | 3517 | CA | GLN | 451 | −35.043 | −22.263 | 22.917 | 1.00 | 15.86 |
| ATOM | 3518 | C | GLN | 451 | −35.922 | −21.025 | 22.672 | 1.00 | 16.09 |
| ATOM | 3519 | O | GLN | 451 | −36.648 | −20.954 | 21.668 | 1.00 | 16.63 |
| ATOM | 3520 | CB | GLN | 451 | −35.741 | −23.235 | 23.897 | 1.00 | 15.50 |
| ATOM | 3521 | CG | GLN | 451 | −35.051 | −24.610 | 24.014 | 1.00 | 16.77 |
| ATOM | 3522 | CD | GLN | 451 | −35.699 | −25.553 | 25.035 | 1.00 | 16.62 |
| ATOM | 3523 | NE2 | GLN | 451 | −36.265 | −24.987 | 26.076 | 1.00 | 15.75 |
| ATOM | 3524 | OE1 | GLN | 451 | −35.660 | −26.787 | 24.883 | 1.00 | 18.71 |
| ATOM | 3525 | N | LYS | 452 | −35.848 | −20.063 | 23.591 | 1.00 | 15.96 |
| ATOM | 3526 | CA | LYS | 452 | −36.526 | −18.789 | 23.415 | 0.50 | 16.09 |
| ATOM | 3527 | C | LYS | 452 | −35.996 | −18.066 | 22.165 | 1.00 | 15.94 |
| ATOM | 3528 | O | LYS | 452 | −36.787 | −17.589 | 21.328 | 1.00 | 16.17 |
| ATOM | 3529 | CB | LYS | 452 | −36.347 | −17.923 | 24.662 | 0.50 | 16.17 |
| ATOM | 3530 | CG | LYS | 452 | −36.896 | −18.563 | 25.943 | 0.50 | 17.78 |
| ATOM | 3531 | CD | LYS | 452 | −38.274 | −19.191 | 25.701 | 0.50 | 19.75 |
| ATOM | 3532 | CE | LYS | 452 | −39.410 | −18.177 | 25.813 | 0.50 | 21.41 |
| ATOM | 3533 | NZ | LYS | 452 | −40.088 | −18.234 | 27.156 | 0.50 | 22.49 |
| ATOM | 3534 | N | LEU | 453 | −34.669 | −17.991 | 22.041 | 1.00 | 14.64 |
| ATOM | 3535 | CA | LEU | 453 | −34.028 | −17.334 | 20.890 | 1.00 | 14.01 |
| ATOM | 3536 | C | LEU | 453 | −34.364 | −18.062 | 19.570 | 1.00 | 13.83 |
| ATOM | 3537 | O | LEU | 453 | −34.513 | −17.432 | 18.525 | 1.00 | 15.05 |
| ATOM | 3538 | CB | LEU | 453 | −32.518 | −17.264 | 21.130 | 1.00 | 13.65 |
| ATOM | 3539 | CG | LEU | 453 | −31.691 | −16.404 | 20.210 | 1.00 | 14.58 |
| ATOM | 3540 | CD1 | LEU | 453 | −32.010 | −14.917 | 20.514 | 1.00 | 15.38 |
| ATOM | 3541 | CD2 | LEU | 453 | −30.217 | −16.703 | 20.477 | 1.00 | 15.63 |
| ATOM | 3542 | N | LYS | 454 | −34.508 | −19.381 | 19.624 | 1.00 | 13.37 |
| ATOM | 3543 | CA | LYS | 454 | −34.953 | −20.172 | 18.478 | 1.00 | 14.09 |
| ATOM | 3544 | C | LYS | 454 | −36.385 | −19.789 | 18.047 | 1.00 | 14.93 |
| ATOM | 3545 | O | LYS | 454 | −36.660 | −19.628 | 16.854 | 1.00 | 14.70 |
| ATOM | 3546 | CB | LYS | 454 | −34.894 | −21.667 | 18.824 | 1.00 | 13.65 |
| ATOM | 3547 | CG | LYS | 454 | −35.149 | −22.608 | 17.658 | 1.00 | 14.04 |
| ATOM | 3548 | CD | LYS | 454 | −33.976 | −22.671 | 16.646 | 1.00 | 13.39 |
| ATOM | 3549 | CE | LYS | 454 | −34.342 | −23.616 | 15.540 | 1.00 | 14.68 |
| ATOM | 3550 | NZ | LYS | 454 | −33.258 | −23.807 | 14.526 | 1.00 | 18.60 |
| ATOM | 3551 | N | ALA | 455 | −37.290 | −19.644 | 19.022 | 1.00 | 15.13 |
| ATOM | 3552 | CA | ALA | 455 | −38.668 | −19.214 | 18.734 | 1.00 | 15.80 |
| ATOM | 3553 | C | ALA | 455 | −38.643 | −17.808 | 18.103 | 1.00 | 16.39 |
| ATOM | 3554 | O | ALA | 455 | −39.413 | −17.521 | 17.188 | 1.00 | 17.22 |
| ATOM | 3555 | CB | ALA | 455 | −39.554 | −19.251 | 20.015 | 1.00 | 14.34 |
| ATOM | 3556 | N | LYS | 456 | −37.744 | −16.945 | 18.567 | 1.00 | 16.53 |
| ATOM | 3557 | CA | LYS | 456 | −37.623 | −15.614 | 17.978 | 1.00 | 16.08 |
| ATOM | 3558 | C | LYS | 456 | −37.105 | −15.597 | 16.526 | 1.00 | 16.64 |
| ATOM | 3559 | O | LYS | 456 | −37.682 | −14.920 | 15.664 | 1.00 | 15.66 |
| ATOM | 3560 | CB | LYS | 456 | −36.740 | −14.720 | 18.831 | 1.00 | 15.78 |
| ATOM | 3561 | CG | LYS | 456 | −36.658 | −13.280 | 18.271 | 1.00 | 15.48 |
| ATOM | 3562 | CD | LYS | 456 | −36.011 | −12.293 | 19.207 | 1.00 | 14.05 |
| ATOM | 3563 | CE | LYS | 456 | −35.848 | −10.957 | 18.508 | 1.00 | 13.28 |
| ATOM | 3564 | NZ | LYS | 456 | −35.020 | −10.079 | 19.418 | 1.00 | 14.47 |
| ATOM | 3565 | N | PHE | 457 | −36.007 | −16.311 | 16.269 | 1.00 | 16.71 |
| ATOM | 3566 | CA | PHE | 457 | −35.313 | −16.236 | 14.966 | 1.00 | 16.59 |
| ATOM | 3567 | C | PHE | 457 | −35.632 | −17.300 | 13.937 | 1.00 | 16.76 |
| ATOM | 3568 | O | PHE | 457 | −35.583 | −17.022 | 12.738 | 1.00 | 17.03 |
| ATOM | 3569 | CB | PHE | 457 | −33.795 | −16.087 | 15.164 | 1.00 | 16.25 |
| ATOM | 3570 | CG | PHE | 457 | −33.417 | −14.719 | 15.617 | 1.00 | 15.69 |
| ATOM | 3571 | CD1 | PHE | 457 | −33.470 | −13.648 | 14.718 | 1.00 | 13.61 |
| ATOM | 3572 | CD2 | PHE | 457 | −33.102 | −14.471 | 16.955 | 1.00 | 13.07 |
| ATOM | 3573 | CE1 | PHE | 457 | −33.167 | −12.329 | 15.140 | 1.00 | 15.43 |
| ATOM | 3574 | CE2 | PHE | 457 | −32.792 | −13.163 | 17.396 | 1.00 | 13.17 |
| ATOM | 3575 | CZ | PHE | 457 | −32.827 | −12.087 | 16.502 | 1.00 | 12.42 |
| ATOM | 3576 | N | ASP | 458 | −35.942 | −18.514 | 14.397 | 1.00 | 17.11 |
| ATOM | 3577 | CA | ASP | 458 | −36.349 | −19.602 | 13.496 | 1.00 | 17.69 |
| ATOM | 3578 | C | ASP | 458 | −37.606 | −20.328 | 14.008 | 1.00 | 17.78 |
| ATOM | 3579 | O | ASP | 458 | −37.571 | −21.534 | 14.232 | 1.00 | 17.88 |
| ATOM | 3580 | CB | ASP | 458 | −35.188 | −20.583 | 13.250 | 1.00 | 17.33 |
| ATOM | 3581 | CG | ASP | 458 | −35.457 | −21.540 | 12.096 | 1.00 | 18.33 |
| ATOM | 3582 | OD1 | ASP | 458 | −36.344 | −21.248 | 11.257 | 1.00 | 19.81 |
| ATOM | 3583 | OD2 | ASP | 458 | −34.767 | −22.586 | 12.025 | 1.00 | 16.13 |
| ATOM | 3584 | N | PRO | 459 | −38.734 | −19.595 | 14.173 | 1.00 | 18.37 |
| ATOM | 3585 | CA | PRO | 459 | −39.946 | −20.229 | 14.723 | 1.00 | 18.61 |
| ATOM | 3586 | C | PRO | 459 | −40.540 | −21.357 | 13.852 | 1.00 | 18.59 |
| ATOM | 3587 | O | PRO | 459 | −41.154 | −22.296 | 14.397 | 1.00 | 18.78 |
| ATOM | 3588 | CB | PRO | 459 | −40.919 | −19.045 | 14.874 | 1.00 | 18.77 |
| ATOM | 3589 | CG | PRO | 459 | −40.463 | −18.067 | 13.821 | 1.00 | 17.95 |
| ATOM | 3590 | CD | PRO | 459 | −38.970 | −18.168 | 13.869 | 1.00 | 18.26 |
| ATOM | 3591 | N | THR | 460 | −40.327 | −21.312 | 12.536 | 1.00 | 17.80 |
| ATOM | 3592 | CA | THR | 460 | −40.826 | −22.393 | 11.686 | 1.00 | 17.53 |
| ATOM | 3593 | C | THR | 460 | −39.816 | −23.538 | 11.513 | 1.00 | 17.02 |
| ATOM | 3594 | O | THR | 460 | −40.045 | −24.448 | 10.718 | 1.00 | 16.45 |
| ATOM | 3595 | CB | THR | 460 | −41.270 | −21.902 | 10.296 | 1.00 | 17.66 |
| ATOM | 3596 | CG2 | THR | 460 | −42.266 | −20.726 | 10.416 | 1.00 | 17.04 |
| ATOM | 3597 | OG1 | THR | 460 | −40.115 | −21.497 | 9.541 | 1.00 | 19.31 |
| ATOM | 3598 | N | ASP | 461 | −38.705 | −23.496 | 12.252 | 1.00 | 17.03 |
| ATOM | 3599 | CA | ASP | 461 | −37.684 | −24.555 | 12.163 | 1.00 | 15.95 |
| ATOM | 3600 | C | ASP | 461 | −37.227 | −24.731 | 10.684 | 1.00 | 14.96 |
| ATOM | 3601 | O | ASP | 461 | −37.062 | −25.854 | 10.205 | 1.00 | 14.49 |
| ATOM | 3602 | CB | ASP | 461 | −38.296 | −25.850 | 12.692 | 1.00 | 16.81 |
| ATOM | 3603 | CG | ASP | 461 | −37.353 | −26.650 | 13.599 | 1.00 | 18.26 |
| ATOM | 3604 | OD1 | ASP | 461 | −36.192 | −26.258 | 13.822 | 1.00 | 19.89 |
| ATOM | 3605 | OD2 | ASP | 461 | −37.802 | −27.716 | 14.073 | 1.00 | 22.29 |
| ATOM | 3606 | N | ARG | 462 | −37.065 | −23.614 | 9.971 | 1.00 | 13.74 |
| ATOM | 3607 | CA | ARG | 462 | −36.639 | −23.607 | 8.571 | 0.50 | 13.90 |
| ATOM | 3608 | C | ARG | 462 | −35.271 | −24.274 | 8.425 | 1.00 | 13.96 |
| ATOM | 3609 | O | ARG | 462 | −34.962 | −24.931 | 7.414 | 1.00 | 14.37 |
| ATOM | 3610 | CB | ARG | 462 | −36.575 | −22.152 | 8.071 | 0.50 | 14.00 |
| ATOM | 3611 | CG | ARG | 462 | −36.004 | −21.960 | 6.667 | 0.50 | 12.63 |
| ATOM | 3612 | CD | ARG | 462 | −36.915 | −22.576 | 5.636 | 0.50 | 11.98 |
| ATOM | 3613 | NE | ARG | 462 | −38.256 | −22.015 | 5.711 | 0.50 | 11.97 |
| ATOM | 3614 | CZ | ARG | 462 | −39.370 | −22.711 | 5.521 | 0.50 | 11.52 |
| ATOM | 3615 | NH1 | ARG | 462 | −39.302 | −24.004 | 5.251 | 0.50 | 11.44 |
| ATOM | 3616 | NH2 | ARG | 462 | −40.550 | −22.115 | 5.608 | 0.50 | 10.34 |
| ATOM | 3617 | N | PHE | 463 | −34.446 | −24.097 | 9.443 | 1.00 | 13.13 |
| ATOM | 3618 | CA | PHE | 463 | −33.088 | −24.629 | 9.431 | 1.00 | 13.49 |
| ATOM | 3619 | C | PHE | 463 | −32.983 | −25.961 | 10.157 | 1.00 | 12.93 |
| ATOM | 3620 | O | PHE | 463 | −31.887 | −26.346 | 10.563 | 1.00 | 14.16 |
| ATOM | 3621 | CB | PHE | 463 | −32.110 | −23.607 | 10.028 | 1.00 | 12.40 |
| ATOM | 3622 | CG | PHE | 463 | −32.062 | −22.305 | 9.277 | 1.00 | 14.33 |
| ATOM | 3623 | CD1 | PHE | 463 | −31.630 | −22.263 | 7.952 | 1.00 | 14.19 |
| ATOM | 3624 | CD2 | PHE | 463 | −32.415 | −21.116 | 9.904 | 1.00 | 15.16 |
| ATOM | 3625 | CE1 | PHE | 463 | −31.579 | −21.056 | 7.261 | 1.00 | 14.35 |
| ATOM | 3626 | CE2 | PHE | 463 | −32.366 | −19.904 | 9.223 | 1.00 | 14.75 |
| ATOM | 3627 | CZ | PHE | 463 | −31.950 | −19.875 | 7.890 | 1.00 | 14.99 |
| ATOM | 3628 | N | TYR | 464 | −34.112 | −26.669 | 10.296 | 1.00 | 12.55 |
| ATOM | 3629 | CA | TYR | 464 | −34.177 | −27.934 | 11.038 | 1.00 | 11.93 |
| ATOM | 3630 | C | TYR | 464 | −33.055 | −28.932 | 10.762 | 1.00 | 12.44 |
| ATOM | 3631 | O | TYR | 464 | −32.630 | −29.144 | 9.603 | 1.00 | 11.73 |
| ATOM | 3632 | CB | TYR | 464 | −35.488 | −28.650 | 10.734 | 1.00 | 12.06 |
| ATOM | 3633 | CG | TYR | 464 | −35.585 | −30.040 | 11.330 | 1.00 | 12.33 |
| ATOM | 3634 | CD1 | TYR | 464 | −35.899 | −30.229 | 12.688 | 1.00 | 13.53 |
| ATOM | 3635 | CD2 | TYR | 464 | −35.354 | −31.166 | 10.550 | 1.00 | 12.91 |
| ATOM | 3636 | CE1 | TYR | 464 | −36.002 | −31.521 | 13.237 | 1.00 | 15.44 |
| ATOM | 3637 | CE2 | TYR | 464 | −35.437 | −32.458 | 11.097 | 1.00 | 15.31 |
| ATOM | 3638 | CZ | TYR | 464 | −35.768 | −32.633 | 12.420 | 1.00 | 15.02 |
| ATOM | 3639 | OH | TYR | 464 | −35.841 | −33.920 | 12.927 | 1.00 | 13.46 |
| ATOM | 3640 | N | TYR | 465 | −32.603 | −29.560 | 11.843 | 1.00 | 12.17 |
| ATOM | 3641 | CA | TYR | 465 | −31.877 | −30.815 | 11.764 | 1.00 | 12.42 |
| ATOM | 3642 | C | TYR | 465 | −32.156 | −31.564 | 13.066 | 1.00 | 12.54 |
| ATOM | 3643 | O | TYR | 465 | −32.607 | −30.951 | 14.045 | 1.00 | 13.50 |
| ATOM | 3644 | CB | TYR | 465 | −30.373 | −30.619 | 11.471 | 1.00 | 11.08 |
| ATOM | 3645 | CG | TYR | 465 | −29.536 | −29.898 | 12.529 | 1.00 | 12.63 |
| ATOM | 3646 | CD1 | TYR | 465 | −28.688 | −30.613 | 13.393 | 1.00 | 9.81 |
| ATOM | 3647 | CD2 | TYR | 465 | −29.565 | −28.502 | 12.653 | 1.00 | 10.65 |
| ATOM | 3648 | CE1 | TYR | 465 | −27.897 | −29.959 | 14.367 | 1.00 | 8.02 |
| ATOM | 3649 | CE2 | TYR | 465 | −28.774 | −27.843 | 13.626 | 1.00 | 9.18 |
| ATOM | 3650 | CZ | TYR | 465 | −27.929 | −28.571 | 14.462 | 1.00 | 9.53 |
| ATOM | 3651 | OH | TYR | 465 | −27.140 | −27.887 | 15.412 | 1.00 | 8.51 |
| ATOM | 3652 | N | PRO | 466 | −31.888 | −32.882 | 13.094 | 1.00 | 12.60 |
| ATOM | 3653 | CA | PRO | 466 | −32.405 | −33.630 | 14.239 | 1.00 | 12.52 |
| ATOM | 3654 | C | PRO | 466 | −31.835 | −33.257 | 15.612 | 1.00 | 12.93 |
| ATOM | 3655 | O | PRO | 466 | −32.390 | −33.698 | 16.631 | 1.00 | 13.39 |
| ATOM | 3656 | CB | PRO | 466 | −32.085 | −35.094 | 13.884 | 1.00 | 12.00 |
| ATOM | 3657 | CG | PRO | 466 | −32.072 | −35.108 | 12.375 | 1.00 | 13.19 |
| ATOM | 3658 | CD | PRO | 466 | −31.362 | −33.787 | 12.047 | 1.00 | 12.18 |
| ATOM | 3659 | N | GLN | 467 | −30.762 | −32.468 | 15.682 | 1.00 | 11.80 |
| ATOM | 3660 | CA | GLN | 467 | −30.333 | −32.019 | 17.008 | 1.00 | 11.28 |
| ATOM | 3661 | C | GLN | 467 | −30.291 | −30.502 | 17.130 | 1.00 | 11.47 |
| ATOM | 3662 | O | GLN | 467 | −29.546 | −29.954 | 17.963 | 1.00 | 11.21 |
| ATOM | 3663 | CB | GLN | 467 | −29.030 | −32.707 | 17.490 | 1.00 | 11.27 |
| ATOM | 3664 | CG | GLN | 467 | −29.146 | −34.235 | 17.532 | 1.00 | 11.39 |
| ATOM | 3665 | CD | GLN | 467 | −27.851 | −34.983 | 17.887 | 1.00 | 12.80 |
| ATOM | 3666 | NE2 | GLN | 467 | −28.013 | −36.198 | 18.436 | 1.00 | 13.01 |
| ATOM | 3667 | OE1 | GLN | 467 | −26.739 | −34.502 | 17.664 | 1.00 | 9.74 |
| ATOM | 3668 | N | ALA | 468 | −31.100 | −29.830 | 16.304 | 1.00 | 11.07 |
| ATOM | 3669 | CA | ALA | 468 | −31.373 | −28.393 | 16.468 | 1.00 | 11.86 |
| ATOM | 3670 | C | ALA | 468 | −32.091 | −28.184 | 17.800 | 1.00 | 12.47 |
| ATOM | 3671 | O | ALA | 468 | −32.860 | −29.048 | 18.235 | 1.00 | 12.71 |
| ATOM | 3672 | CB | ALA | 468 | −32.241 | −27.845 | 15.279 | 1.00 | 10.45 |
| ATOM | 3673 | N | VAL | 469 | −31.840 | −27.045 | 18.436 | 1.00 | 13.25 |
| ATOM | 3674 | CA | VAL | 469 | −32.615 | −26.603 | 19.592 | 1.00 | 14.80 |
| ATOM | 3675 | C | VAL | 469 | −34.111 | −26.440 | 19.190 | 1.00 | 15.43 |
| ATOM | 3676 | O | VAL | 469 | −34.399 | −25.921 | 18.115 | 1.00 | 15.44 |
| ATOM | 3677 | CB | VAL | 469 | −32.001 | −25.279 | 20.107 | 1.00 | 15.19 |
| ATOM | 3678 | CG1 | VAL | 469 | −32.894 | −24.571 | 21.096 | 1.00 | 16.11 |
| ATOM | 3679 | CG2 | VAL | 469 | −30.610 | −25.538 | 20.754 | 1.00 | 14.98 |
| ATOM | 3680 | N | ARG | 470 | −35.054 | −26.909 | 20.016 | 1.00 | 16.40 |
| ATOM | 3681 | CA | ARG | 470 | −36.489 | −26.670 | 19.738 | 1.00 | 17.27 |
| ATOM | 3682 | C | ARG | 470 | −36.879 | −25.213 | 19.954 | 1.00 | 17.85 |
| ATOM | 3683 | O | ARG | 470 | −36.452 | −24.612 | 20.934 | 1.00 | 17.64 |
| ATOM | 3684 | CB | ARG | 470 | −37.402 | −27.524 | 20.613 | 1.00 | 17.36 |
| ATOM | 3685 | CG | ARG | 470 | −37.350 | −28.987 | 20.344 | 1.00 | 18.24 |
| ATOM | 3686 | CD | ARG | 470 | −38.475 | −29.735 | 21.105 | 1.00 | 20.89 |
| ATOM | 3687 | NE | ARG | 470 | −38.316 | −31.177 | 20.922 | 1.00 | 19.26 |
| ATOM | 3688 | CZ | ARG | 470 | −38.724 | −31.831 | 19.841 | 1.00 | 21.47 |
| ATOM | 3689 | NH1 | ARG | 470 | −39.313 | −31.166 | 18.847 | 1.00 | 20.22 |
| ATOM | 3690 | NH2 | ARG | 470 | −38.548 | −33.147 | 19.752 | 1.00 | 19.68 |
| ATOM | 3691 | N | PRO | 471 | −37.677 | −24.639 | 19.030 | 1.00 | 18.88 |
| ATOM | 3692 | CA | PRO | 471 | −38.271 | −23.335 | 19.304 | 1.00 | 20.52 |
| ATOM | 3693 | C | PRO | 471 | −39.326 | −23.425 | 20.426 | 1.00 | 21.86 |
| ATOM | 3694 | O | PRO | 471 | −40.331 | −24.122 | 20.277 | 1.00 | 22.27 |
| ATOM | 3695 | CB | PRO | 471 | −38.950 | −22.958 | 17.979 | 1.00 | 19.71 |
| ATOM | 3696 | CG | PRO | 471 | −39.103 | −24.227 | 17.239 | 1.00 | 20.13 |
| ATOM | 3697 | CD | PRO | 471 | −37.982 | −25.119 | 17.673 | 1.00 | 18.95 |
| ATOM | 3698 | N | VAL | 472 | −39.089 | −22.733 | 21.535 | 1.00 | 23.68 |
| ATOM | 3699 | CA | VAL | 472 | −40.086 | −22.657 | 22.624 | 1.00 | 25.38 |
| ATOM | 3700 | C | VAL | 472 | −40.353 | −21.198 | 23.006 | 1.00 | 26.31 |
| ATOM | 3701 | O | VAL | 472 | −39.478 | −20.528 | 23.553 | 1.00 | 26.35 |
| ATOM | 3702 | CB | VAL | 472 | −39.655 | −23.474 | 23.875 | 1.00 | 25.42 |
| ATOM | 3703 | CG1 | VAL | 472 | −39.177 | −24.853 | 23.473 | 1.00 | 25.27 |
| ATOM | 3704 | CG2 | VAL | 472 | −40.815 | −23.584 | 24.872 | 1.00 | 25.30 |
| ATOM | 3705 | N | LYS | 473 | −41.562 | −20.724 | 22.687 | 0.50 | 26.98 |
| ATOM | 3706 | CA | LYS | 473 | −42.025 | −19.374 | 23.041 | 0.50 | 27.54 |
| ATOM | 3707 | C | LYS | 473 | −41.981 | −19.132 | 24.556 | 0.50 | 27.57 |
| ATOM | 3708 | O | LYS | 473 | −41.226 | −18.289 | 25.044 | 0.50 | 27.42 |
| ATOM | 3709 | CB | LYS | 473 | −43.457 | −19.149 | 22.527 | 0.50 | 27.60 |
| ATOM | 3710 | CG | LYS | 473 | −43.546 | −18.413 | 21.191 | 0.50 | 28.57 |
| ATOM | 3711 | CD | LYS | 473 | −44.966 | −17.906 | 20.919 | 0.50 | 29.65 |
| ATOM | 3712 | CE | LYS | 473 | −44.964 | −16.681 | 19.998 | 0.50 | 30.53 |
| ATOM | 3713 | NZ | LYS | 473 | −46.351 | −16.220 | 19.665 | 0.50 | 29.94 |
| TER | | | | | | | | | |
| ATOM | 3715 | P | FAD | 501 | −17.707 | −27.553 | 9.264 | 1.00 | 10.61 |
| ATOM | 3716 | O1P | FAD | 501 | −18.041 | −26.164 | 8.786 | 1.00 | 11.67 |
| ATOM | 3717 | O2P | FAD | 501 | −16.533 | −28.198 | 8.518 | 1.00 | 11.16 |
| ATOM | 3718 | O3P | FAD | 501 | −18.959 | −28.563 | 9.302 | 1.00 | 9.57 |
| ATOM | 3719 | C5* | FAD | 501 | −17.032 | −28.711 | 11.476 | 1.00 | 9.78 |
| ATOM | 3720 | O5* | FAD | 501 | −17.385 | −27.512 | 10.853 | 1.00 | 11.84 |
| ATOM | 3721 | C4* | FAD | 501 | −17.221 | −28.586 | 12.983 | 1.00 | 12.31 |
| ATOM | 3722 | O4* | FAD | 501 | −18.566 | −28.365 | 13.288 | 1.00 | 9.95 |
| ATOM | 3723 | C3* | FAD | 501 | −16.764 | −29.852 | 13.706 | 1.00 | 11.59 |
| ATOM | 3724 | O3* | FAD | 501 | −17.059 | −31.028 | 12.970 | 1.00 | 12.03 |
| ATOM | 3725 | C2* | FAD | 501 | −15.284 | −29.739 | 13.994 | 1.00 | 12.13 |
| ATOM | 3726 | O2* | FAD | 501 | −15.055 | −28.638 | 14.875 | 1.00 | 13.48 |
| ATOM | 3727 | C1* | FAD | 501 | −14.763 | −31.012 | 14.637 | 1.00 | 12.93 |
| ATOM | 3728 | N1 | FAD | 501 | −13.229 | −29.580 | 16.571 | 1.00 | 15.98 |
| ATOM | 3729 | C2 | FAD | 501 | −12.523 | −28.884 | 17.569 | 1.00 | 18.22 |
| ATOM | 3730 | O2 | FAD | 501 | −13.110 | −28.244 | 18.471 | 1.00 | 12.56 |
| ATOM | 3731 | N3 | FAD | 501 | −11.132 | −28.929 | 17.544 | 1.00 | 17.45 |
| ATOM | 3732 | C4 | FAD | 501 | −10.442 | −29.599 | 16.544 | 1.00 | 17.98 |
| ATOM | 3733 | C4A | FAD | 501 | −11.160 | −30.277 | 15.553 | 1.00 | 16.32 |
| ATOM | 3734 | O4 | FAD | 501 | −9.187 | −29.569 | 16.555 | 1.00 | 19.01 |
| ATOM | 3735 | C5A | FAD | 501 | −11.198 | −31.670 | 13.541 | 1.00 | 13.95 |
| ATOM | 3736 | N5 | FAD | 501 | −10.511 | −30.983 | 14.544 | 1.00 | 17.20 |
| ATOM | 3737 | C6 | FAD | 501 | −10.506 | −32.346 | 12.518 | 1.00 | 13.58 |
| ATOM | 3738 | C7 | FAD | 501 | −11.223 | −33.005 | 11.505 | 1.00 | 11.25 |
| ATOM | 3739 | C7M | FAD | 501 | −10.484 | −33.746 | 10.409 | 1.00 | 11.46 |
| ATOM | 3740 | C8 | FAD | 501 | −12.616 | −32.964 | 11.504 | 1.00 | 11.59 |
| ATOM | 3741 | C8M | FAD | 501 | −13.425 | −33.638 | 10.424 | 1.00 | 9.17 |
| ATOM | 3742 | C9 | FAD | 501 | −13.296 | −32.296 | 12.534 | 1.00 | 12.64 |
| ATOM | 3743 | C9A | FAD | 501 | −12.597 | −31.637 | 13.545 | 1.00 | 14.14 |
| ATOM | 3744 | C10 | FAD | 501 | −12.557 | −30.280 | 15.576 | 1.00 | 16.68 |
| ATOM | 3745 | N10 | FAD | 501 | −13.267 | −30.953 | 14.563 | 1.00 | 14.26 |
| ATOM | 3746 | C1* | FAD | 501 | −23.754 | −25.638 | 10.409 | 1.00 | 7.42 |
| ATOM | 3747 | C2 | FAD | 501 | −24.647 | −21.286 | 10.315 | 1.00 | 9.38 |
| ATOM | 3748 | C2* | FAD | 501 | −24.187 | −26.829 | 9.559 | 1.00 | 9.34 |
| ATOM | 3749 | C3* | FAD | 501 | −24.078 | −27.953 | 10.574 | 1.00 | 8.35 |
| ATOM | 3750 | C4 | FAD | 501 | −23.622 | −23.287 | 9.611 | 1.00 | 7.81 |
| ATOM | 3751 | C4* | FAD | 501 | −22.813 | −27.548 | 11.337 | 1.00 | 7.68 |
| ATOM | 3752 | C5 | FAD | 501 | −22.917 | −22.603 | 8.607 | 1.00 | 7.48 |
| ATOM | 3753 | C5* | FAD | 501 | −21.554 | −28.208 | 10.771 | 1.00 | 9.90 |
| ATOM | 3754 | C6 | FAD | 501 | −23.108 | −21.230 | 8.440 | 1.00 | 10.59 |
| ATOM | 3755 | C8 | FAD | 501 | −22.342 | −24.720 | 8.493 | 1.00 | 8.65 |
| ATOM | 3756 | N1 | FAD | 501 | −23.965 | −20.593 | 9.314 | 1.00 | 6.78 |
| ATOM | 3757 | N3 | FAD | 501 | −24.481 | −22.652 | 10.462 | 1.00 | 9.58 |
| ATOM | 3758 | N6 | FAD | 501 | −22.437 | −20.523 | 7.489 | 1.00 | 8.98 |
| ATOM | 3759 | N7 | FAD | 501 | −22.168 | −23.497 | 7.938 | 1.00 | 8.51 |
| ATOM | 3760 | N9 | FAD | 501 | −23.245 | −24.588 | 9.533 | 1.00 | 6.94 |
| ATOM | 3761 | O1 | FAD | 501 | −20.266 | −27.940 | 7.139 | 1.00 | 9.52 |
| ATOM | 3762 | O2 | FAD | 501 | −20.655 | −30.092 | 8.371 | 1.00 | 8.81 |
| ATOM | 3763 | O2* | FAD | 501 | −25.458 | −26.663 | 8.980 | 1.00 | 8.85 |
| ATOM | 3764 | O3* | FAD | 501 | −25.216 | −27.902 | 11.455 | 1.00 | 10.84 |
| ATOM | 3765 | O4* | FAD | 501 | −22.722 | −26.116 | 11.242 | 1.00 | 6.39 |
| ATOM | 3766 | O5* | FAD | 501 | −21.428 | −27.875 | 9.399 | 1.00 | 11.17 |
| ATOM | 3767 | P | FAD | 501 | −20.340 | −28.623 | 8.477 | 1.00 | 9.06 |
| TER | | | | | | | | | |
| ATOM | 3768 | C1 | ABL | 502 | −10.379 | −33.923 | 16.074 | 1.00 | 41.90 |
| ATOM | 3769 | C1A | ABL | 502 | −6.880 | −37.611 | 14.215 | 1.00 | 43.41 |
| ATOM | 3770 | O1 | ABL | 502 | −11.115 | −33.055 | 16.600 | 1.00 | 43.59 |
| ATOM | 3771 | C2 | ABL | 502 | −8.948 | −34.152 | 16.595 | 1.00 | 41.64 |
| ATOM | 3772 | C2A | ABL | 502 | −6.560 | −38.442 | 12.947 | 1.00 | 44.34 |
| ATOM | 3773 | O2 | ABL | 502 | −8.476 | −32.913 | 17.098 | 1.00 | 40.95 |
| ATOM | 3774 | O2A | ABL | 502 | −7.806 | −38.906 | 12.414 | 1.00 | 45.45 |
| ATOM | 3775 | C3 | ABL | 502 | −7.972 | −34.666 | 15.515 | 1.00 | 40.23 |
| ATOM | 3776 | C3A | ABL | 502 | −5.631 | −39.611 | 13.301 | 1.00 | 44.76 |
| ATOM | 3777 | O3 | ABL | 502 | −6.786 | −35.083 | 16.227 | 1.00 | 39.20 |
| ATOM | 3778 | O3A | ABL | 502 | −5.210 | −40.325 | 12.144 | 1.00 | 45.04 |
| ATOM | 3779 | C4 | ABL | 502 | −8.589 | −35.876 | 14.838 | 1.00 | 41.16 |
| ATOM | 3780 | C4A | ABL | 502 | −4.388 | −39.083 | 14.082 | 1.00 | 44.40 |
| ATOM | 3781 | O4 | ABL | 502 | −7.702 | −36.464 | 13.854 | 1.00 | 42.36 |
| ATOM | 3782 | O4A | ABL | 502 | −3.542 | −40.167 | 14.445 | 1.00 | 44.67 |
| ATOM | 3783 | C5 | ABL | 502 | −9.917 | −35.440 | 14.117 | 1.00 | 40.69 |
| ATOM | 3784 | C5A | ABL | 502 | −4.820 | −38.277 | 15.320 | 1.00 | 43.72 |
| ATOM | 3785 | N5 | ABL | 502 | −10.816 | −34.691 | 15.042 | 1.00 | 40.49 |
| ATOM | 3786 | O5A | ABL | 502 | −5.698 | −37.210 | 14.888 | 1.00 | 43.64 |
| ATOM | 3787 | C6 | ABL | 502 | −10.720 | −36.659 | 13.581 | 1.00 | 39.47 |
| ATOM | 3788 | C6A | ABL | 502 | −3.636 | −37.732 | 16.137 | 1.00 | 42.64 |
| ATOM | 3789 | O6 | ABL | 502 | −11.628 | −36.326 | 12.517 | 1.00 | 37.23 |
| ATOM | 3790 | O6A | ABL | 502 | −2.623 | −37.118 | 15.392 | 1.00 | 40.91 |
| END | | | | | | | | | |

1. An isolated variant carbohydrate oxidase comprising a substitution at one or more positions corresponding to positions 4, 15, 19, 21, 22, 27, 29, 30, 31, 43, 48, 52, 54, 57, 58, 59, 60, 62, 69, 70, 71, 72, 77, 80, 81, 85, 91, 93, 98, 105, 112, 114, 118, 122, 129, 130, 131, 132, 133, 134, 135, 138, 140, 146, 147, 148, 152, 153, 157, 169, 170, 174, 184, 188, 201, 213, 221, 222, 223, 224, 227, 228, 231, 235, 248, 249, 250, 251, 252, 253, 256, 258, 260, 268, 278, 287, 288, 300, 301, 302, 303, 304, 305, 307, 314, 315, 316, 317, 318, 319, 320, 321, 322, 323, 327, 330, 332, 342, 347, 349, 352, 353, 354, 355, 356, 357, 358, 363, 366, 368, 374, 382, 383, 385, 386, 387, 388, 389, 390, 391, 392, 393, 400, 403, 411, 415, 419, 420, 424, 425, 427, 428, 429, 433, 437, 440, 445, 456, 460, 472, and/or 473 of SEQ ID NO:2, wherein the variant carbohydrate oxidase comprises an amino acid sequence having at least 60% identity to SEQ ID NO:2 or the mature polypeptide encoded by SEQ ID NO:1, and wherein the variant has carbohydrate oxidase activity.

2. The variant of claim 1, wherein the variant is (a) a polypeptide comprising an amino acid sequence having at least at least 65% identity, preferably at least 70% identity, at least 75% identity, at least 80% identity, at least 85% identity, at least 90% identity, at least 95% identity, at least 96% identity, at least 97% identity, at least 98% identity, or at least 99% identity to SEQ ID NO:2 or the mature polypeptide encoded by SEQ ID NO:1; or (b) a polypeptide encoded by a polynucleotide that hybridizes under medium stringency conditions, preferably medium-high stringency conditions, or high stringency conditions with the mature polypeptide coding sequence of SEQ ID NO:1, or its complementary strand; or (c) a polypeptide encoded by a polynucleotide comprising a nucleotide sequence having at least 60% identity, preferably at least 65% identity, at least 70% identity, at least 75% identity, at least 80% identity, at least 85% identity, at least 90% identity, at least 95% identity, at least 96% identity, at least 97% identity, at least 98% identity, at least 99% identity to the mature polypeptide coding sequence of SEQ ID NO:1.

3. The variant of claim 1, wherein the variant is a variant of parent carbohydrate oxidase, and wherein the parent carbohydrate oxidase comprises or consists of a polypeptide having an amino acid sequence of SEQ ID NO:2.

4. The variant of claim 1, which comprises a substitution at a position corresponding to one or more of the following positions: 15, 22, 27, 54, 98, 188, 201, 222, and 460 using SEQ ID NO:2 for numbering.

5. The variant of claim 1, which comprises one or more of the following substitutions: D15N, Y22W, E27R, Q54D, N98D, K188R, K201R, N222D, and/or T460E.

6. The variant of claim 1, which has one or more improved properties compared to the parent carbohydrate oxidase of the variant enzyme, wherein the improved properties are selected from the group consisting of thermal activity, thermostability, pH activity, pH stability, substrate specificity, product specificity, and chemical stability.

7. The variant of claim 1, which has improved stability at high pH or at high temperature.

8. An isolated nucleotide sequence encoding the variant of claim 1.

9. A recombinant host cell comprising the nucleotide sequence of claim 8.

10. A method for producing a variant carbohydrate oxidase, comprising:
(a) cultivating the host cell of claim 9 under conditions suitable for the expression of the carbohydrate oxidase; and
(b) recovering the carbohydrate oxidase from the cultivation medium.

11. A process for preparing a dough, comprising adding to the dough a carbohydrate oxidase of claim 1.

12. The process of claim 11, further comprising baking the dough.

13. A dough composition comprising flour and a carbohydrate oxidase of claim 1.


SEQUENCE LISTING (22 sequences)

SEQ ID NO: 1 - DNA, 1553 bp, Microdochium nivale
  CDS (1)..(1011)
  Intron (1012)..(1076)
  CDS (1077)..(1550)
atgcgttctgcatttatcttggccctcggccttatcaccgccagcgctgacgctttagtcactcgcggtgccatcgaggcctgcctgtctgctgctggcgtcccgatcgatattcctggcactgccgactatgagcgcgatgtcgagcccttcaacatccgcctgccatacattcccaccgccattgctcagacgcagactactgctcacatccagtcggcagtccagtgcgccaagaagctcaacctcaaggtctctgccaagtctggtggtcacagctacgcctcgttcggctttggtggcgagaacggtcacctcatggtccagctcgaccgcatgattgatgtcatctcgtacaatgacaagactggcattgcccatgttgagcccggtgcccgcctcggacatctcgccaccgtcctcaacgacaagtacggccgtgccatctcccacggtacatgccctggtgtcggcatctccggccactttgcccacggcggcttcggcttcagctcgcacatgcacggtctggctgtcgactcggtcgtcggtgtcactgttgttcttgctgatggacgcatcgttgaggcttctgccactgagaatgctgacctcttctggggtatcaagggcgctggctccaacttcggcatcgttgctgtctggaagctcgccactttccctgctcccaaggttctcacccgctttggcgtcaccctcaactggaagaacaagacctctgccctcaagggcatcgaggctgttgaggactacgcccgctgggtcgccccccgcgaggtcaacttccgcattggagactacggcgctggtaacccgggtatcgagggtctctactacggcactcccgagcaatggcgtgcggctttccaacctctgctcgacactctgcctgctggatacgttgtcaacccgaccacctccttgaactggatcgagtcggtgctcagctactccaactttgaccatgtcgacttcattactcctcagcctgtaagtgttcaccgactttgcgctgggagaatgttttatgtcggctttactgactccctctacaggtcgagaacttctatgccaagagcttgacgctcaagagtatcaagggcgacgccgtcaagaactttgtcgactactactttgacgtgtccaacaaggttaaggaccgcttctggttctaccagctcgacgtgcacggcggcaagaactcgcaagtcaccaaggtcaccaacgccgagacagcctaccctcaccgcgacaagctctggctgatccagttctacgaccgctacgacaacaaccagacctacccggagacctcattcaagttcctcgacggctgggtcaactcggtcaccaaggctctccccaagtccgactggggcatgtacatcaactacgccgacccccgcatggaccgcgactacgccaccaaggtctactacggtgagaacctcgccaggctccagaagctcaaggccaagtttgatcccaccgaccgtttctactaccctcaggctgtccgccctgtcaaataa

SEQ ID NO: 2 - protein, 495 aa, Microdochium nivale
MRSAFILALGLITASADALVTRGAIEACLSAAGVPIDIPGTADYERDVEPFNIRLPYIPTAIAQTQTTAHIQSAVQCAKKLNLKVSAKSGGHSYASFGFGGENGHLMVQLDRMIDVISYNDKTGIAHVEPGARLGHLATVLNDKYGRAISHGTCPGVGISGHFAHGGFGFSSHMHGLAVDSVVGVTVVLADGRIVEASATENADLFWGIKGAGSNFGIVAVWKLATFPAPKVLTRFGVTLNWKNKTSALKGIEAVEDYARWVAPREVNFRIGDYGAGNPGIEGLYYGTPEQWRAAFQPLLDTLPAGYVVNPTTSLNWIESVLSYSNFDHVDFITPQPVENFYAKSLTLKSIKGDAVKNFVDYYFDVSNKVKDRFWFYQLDVHGGKNSQVTKVTNAETAYPHRDKLWLIQFYDRYDNNQTYPETSFKFLDGWVNSVTKALPKSDWGMYINYADPRMDRDYATKVYYGENLARLQKLKAKFDPTDRFYYPQAVRPVK

SEQ ID NO: 3 - DNA, 37 bp, Artificial
  Primer
ctgaggatccaccatgcgttctgcatttatcttggcc

SEQ ID NO: 4 - DNA, 37 bp, Artificial
  Primer
caggctgtccgccctgtcaaataacttaagacctggt

SEQ ID NO: 5 - DNA, 45 bp, Artificial
  Primer
ctgctgctggcgtcccgatcaacattcctggcactgccgactatg

SEQ ID NO: 6 - DNA, 30 bp, Artificial
  Primer
gatcgggacgccagcagcagacaggcaggc

SEQ ID NO: 7 - DNA, 44 bp, Artificial
  Primer
gatattcctggcactgccgactgggagcgcgatgtcgagccctt

SEQ ID NO: 8 - DNA, 31 bp, Artificial
  Primer
gtcggcagtgccaggaatatcgatcgggacg

SEQ ID NO: 9 - DNA, 44 bp, Artificial
  Primer
gccgactatgagcgcgatgtccgccccttcaacatccgcctgcc

SEQ ID NO: 10 - DNA, 32 bp, Artificial
  Primer
gacatcgcgctcatagtcggcagtgccaggaa

SEQ ID NO: 11 - DNA, 45 bp, Artificial
  Primer
gctcacatccagtcggcagtcgactgcgccaagaagctcaacctc

SEQ ID NO: 12 - DNA, 32 bp, Artificial
  Primer
gactgccgactggatgtgagcagtagtctgcg

SEQ ID NO: 13 - DNA, 48 bp, Artificial
  Primer
gcatgattgatgtcatctcgtacgacgacaagactggcattgcccatg

SEQ ID NO: 14 - DNA, 33 bp, Artificial
  Primer
gtacgagatgacatcaatcatgcggtcgagctg

SEQ ID NO: 15 - DNA, 44 bp, Artificial
  Primer
gctgacctcttctggggtatccgcggcgctggctccaacttcgg

SEQ ID NO: 16 - DNA, 33 bp, Artificial
  Primer
gataccccagaagaggtcagcattctcagtggc

SEQ ID NO: 17 - DNA, 44 bp, Artificial
  Primer
ttcggcatcgttgctgtctggcgcctcgccactttccctgctcc

SEQ ID NO: 18 - DNA, 32 bp, Artificial
  Primer
ccagacagcaacgatgccgaagttggagccag

SEQ ID NO: 19 - DNA, 45 bp, Artificial
  Primer
ggcgtcaccctcaactggaaggacaagacctctgccctcaagggc

SEQ ID NO: 20 - DNA, 32 bp, Artificial
  Primer
cttccagttgagggtgacgccaaagcgggtga

SEQ ID NO: 21 - DNA, 47 bp, Artificial
  Primer
gctcaaggccaagtttgatcccgaggaccgtttctactaccctcagg

SEQ ID NO: 22 - DNA, 33 bp, Artificial
  Primer
gggatcaaacttggccttgagcttctggagcct